(19) United States
(12) Patent Application Publication
(10) Pub. No.: US 2008/0194629 A1
(43) Pub. Date: Aug. 14, 2008

(54) 3-Mono-and 3,5-Disubstituted Piperidine Derivatives as Renin Inhibitors

(76) Inventors: Daniel Kaspar Baeschlin, Arlesheim (CH); Werner Breitenstein, Basel (CH); Claus Ehrhardt, Lorrach (DE); Juergen Klaus Maibaum, Weil-Haltingen (DE); Nils Ostermann, Binzen (DE); Juerg Zimmermann, Reinach BL (CH); Simon Rudisser, Basel (CH); Eric Vangrevelinghe, Huningue (FR); Osamu Irie, Ibaraki (JP); Ichiro Umemura, Ibaraki (JP); Masaki Suzuki, Ibaraki (JP); Muneto Mogi, Ibaraki (JP); Takanori Kanazawa, Ibaraki (JP); Fumiaki Yokokawa, Ibaraki (JP); Atsuko Nihonyanagi, Ibaraki (JP)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/913,492

(22) PCT Filed: May 2, 2006
(86) PCT No.: PCT/EP2006/004082

§ 371 Date: Nov. 2, 2007

(30) Foreign Application Priority Data
May 3, 2005 (GB) 0508992.5

(51) Int. Cl. A61K 31/453; C07D 401/12

(52) U.S. Cl. 514/321; 546/207

(57) ABSTRACT

The invention relates to 3,5-piperidine compounds, these compounds for use in the diagnostic and therapeutic treatment of a warm-blooded animal, especially for the treatment of a disease (=disorder) that depends on activity of renin; the use of a compound of that class for the preparation of a pharmaceutical formulation for the treatment of a disease that depends on activity of renin; the use of a compound of that class in the treatment of a disease that depends on activity of renin; pharmaceutical formulations comprising a 3,5-piperidine compound, and/or a method of treatment comprising administering a 3,5-piperidine compound, a method for the manufacture of a 3,5-piperidine compound, and novel intermediates and partial steps for their synthesis. Especially, the 3,5-piperidine compounds have the formula I, wherein the symbols have the meanings described in the specification.

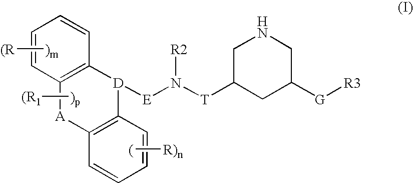

The invention relates to 3,5-piperidine compounds, these compounds for use in the diagnostic and therapeutic treatment of a warm-blooded animal, especially for the treatment of a disease (=disorder) that depends on activity of renin; the use of a compound of that class for the preparation of a pharmaceutical formulation for the treatment of a disease that depends on activity of renin; the use of a compound of that class in the treatment of a disease that depends on activity of renin; pharmaceutical formulations comprising a 3,5-piperidine compound, and/or a method of treatment comprising administering a 3,5-piperidine compound, a method for the manufacture of a 3,5-piperidine compound, and novel intermediates and partial steps for their synthesis.

Especially, the present invention relates to a compound of the formula I,

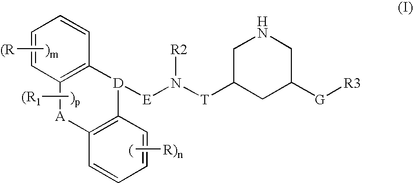

wherein
each R₁, independently of the others, (present if p>0) is a substituent selected from the group consisting of
a substituent of the formula —(C₀-C₇-alkylene)-(X)ᵣ—(C₁-C₇alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below;
C₂-C₇-alkenyl, C₂-C₇-alkynyl, phenyl, naphthyl, heterocyclyl, phenyl- or naphthyl- or heterocyclyl-C₁-C₇alkyl or —C₁-C₇-alkyloxy, di-(naphthyl- or phenyl)-amino-C₁-C₇alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties; phenyl- or naphthyl-C₁-C₇-alkylsulfonylamino-C₁-C₇-alkyl, carboxy-C₁-C₇-alkyl, halo, hydroxy, phenyl-C₁-C₇-alkoxy wherein phenyl is unsubstituted or substituted by C₁-C₇-alkoxy and/or halo, halo-C₁-C₇-alkoxy, phenyl- or naphthyloxy, phenyl- or naphthyl-C₁-C₇-alkyloxy, phenyl- or naphthyl-oxy-C₁-C₇alkyloxy, benzoyl- or naphthoyloxy, halo-C₁-C₇-alkylthio, phenyl- or naphthylthio, phenyl- or naphthyl-C₁-C₇-alkylthio, benzoyl- or naphthoylthio, nitro, amino, di-(naphthyl- or phenyl-C₁-C₇alkyl)-amino, benzoyl- or naphthoylamino, phenyl- or naphthylsulfonylamino wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfonylamino, carboxyl, (N,N-) di-(C₁-C₇alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N-) di-(C₁-C₇-alkyl)-amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇-alkyl)aminocarbonyl, cyano, sulfenyl, sulfinyl, C₁-C₇-alkylsulfinyl, phenyl- or naphthylsulfinyl wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfinyl, sulfonyl, C₁-C₇alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl;
R2 is hydrogen, unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
R3 is hydrogen, unsubstituted or substituted alkyl, substituted or unsubstituted aryl, unsubstituted or substituted heterocyclyl, unsubstituted or substituted cycloalkyl, unsubstituted or substituted aryl-alkyl, unsubstituted or substituted heterocyclyl-alkyl, unsubstituted or substituted cycloalkyl-alkyl, or, if G is oxy, thio or unsubstituted or substituted imino, has one of the meanings just mentioned or is acyl;
R is (if more than one R is present, independently of each other) selected from C₁-C₇alkyl, halo-C₁-C₇-alkyl, halo, hydroxy, C₁-C₇-alkoxy, phenoxy, phenyl-C₁-C₇-alkyloxy, C₁-C₇-alkanoyloxy, amino, N-mono- or N,N-di-(C₁-C₇-alkyl, alkanoyl, benzoyl, phenyl and/or phenyl-C₁-C₇-alkyl)-amino, carboxy, C₁-C₇-alkyloxycarbonyl, phenoxycarbonyl, phenyl-C₁-C₇-alkyl-oxycarbonyl, carbamoyl, N-mono- or N,N-di-(C₁-C₇-alkyl, phenyl and/or phenyl-C₁-C₇-alkyl)carbamoyl, sulfamoyl, N-mono- or N,N-di-(C₁-C₇-alkyl, phenyl and/or phenyl-C₁-C₇-alkyl)sulfamoyl, nitro and cyano; where, if p is zero, at least one R, preferably not more than one R, can be R₁ as defined above;
A is NH, CH₂, S(O)₀₋₂, O, CH═CH, CH₂CH₂, CH₂O, CH₂S(O)₀₋₂, CH₂NH, C(═O)NH or SO₂NH, where in each case H is unreplaced or one H can be replaced by a moiety Rx selected from C₁-C₇-alkyl, especially methyl, ethyl or propyl, hydroxy-C₁-C₇-alkyl, such as hydroxymethyl, C₁-C₄-alkoxy-C₁-C₄-alkyl, such as methoxymethyl, hydroxy, halo, such as fluoro, chloro or bromo, C₁-C₇-alkoxy, such as methoxy, ethoxy or propoxy, halo-C₁-C₇-alkyl, such as trifluoromethyl, amino, N-mono- or N,N-di-(C₁-C₄-alkyl)-amino, such as N-mono- or N,N-dimethylamino, C₁-C₄-alkoxycarbonyl, such as tert-butoxycarbonyl, C₃-C₇-cycloalkyl, such as cyclopropyl or cyclobutyl, or C₃-C₇-cycloalkyl-C₁-C₄-alkyl, such as cyclopropylmethyl;
D is N, CH, CH═C, CH₂CH, CHO, CHS(O)₀₋₂, CH₂N, NHCH, C(═O)N or SO₂N, where in each case a H if present is unreplaced or one or two (preferably one) can be replaced by a moiety R₁ as defined above if p is 1 or 2, preferably 1;
E is carbonyl (C(═O)) or unsubstituted or (halo, hydroxy, C₁-C₇-alkyloxy, phenoxy, phenyl-C₁-C₇-alkyloxy, C₁-C₇-alkanoyloxy or benzoyloxy)-substituted C₁-C₇-alkylene;
T is carbonyl or methylene;
G is an oxy (O), thio (S) or unsubstituted (NH) or substituted (NR4) imino, C(═O)NH (bound at the left side carbonyl to the piperidine ring in formula I, on the right side NH to R₃ in formula I) or C(═O)NR4 (bound at the left side carbonyl to the piperidine ring in formula I, on the right side NR4 to R₃ in formula I), wherein R₄ is an imino substituent (as defined for substituted imino);
or G-R3 together is hydrogen;
m is 0 (zero) to 4;
n is 0 (zero) to 4; and
p is 0 (zero), 1 or 2, preferably 0 or 1;
or a (preferably pharmaceutically acceptable) salt thereof.

The compounds of the present invention exhibit inhibitory activity on the natural enzyme renin. Thus, compounds of formula I may be employed for the treatment (this term also including prophylaxis) of one or more disorders or diseases selected from, inter alia, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, especially as far as these diseases can be modulated by renin inhibition.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo; where halo is mentioned in connection with another moiety, e.g. in halo-C₁-C₇-alkyl, halo-C₁-C₇-alkoxy, halo-C₁-C₇alkanoyl or halo-aryl, also if not explicitly mentioned this can mean that one or more (e.g. up to three) halogen atoms are present; as detailed example for halo-C₁-C₇-alkyl, trifluoromethyl, 2,2-difluoroethyl or 2,2,2-trifluoroethyl can be mentioned.

A substituent R₁ of the formula —(C₀-C₇-alkylene)-(X)ᵣ—(C₁-C₇-alkylene)-(Y)ₛ—(C₀-C₇-alkylene)H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇alkyl, phenyl, naphthyl, phenyl- or naphthyl-C₁-C₇-alkyl and halo-C₁-C₇-alkyl; is preferably C₁-C₇-alkyl, such as methyl, ethyl, n-propyl, isopropyl, n-butyl, isobutyl, sec-butyl or tert-butyl, hydroxy-C₁-C₇alkyl, C₁-C₇-alkoxy-C₁-C₇-alkyl, such as 3-methoxypropyl or 2-methoxyethyl, C₁-C₇alkoxy-C₁-C₇-alkoxy-C₁-C₇-alkyl, C₁-C₇alkanoyloxy-C₁-C₇-alkyl, C₁-C₇alkyloxycarbonyl-C₁-C₇alkyl, amino-C₁-C₇-alkyl, such as aminomethyl, (N-) mono- or (N,N-) di-(C₁-C₇-alkyl)amino-C₁-C₇-alkyl, C₁-C₇-alkoxy-C₁-C₇-alkylamino-C₁-C₇-alkyl, mono-(naphthyl- or phenyl)amino-C₁-C₇-alkyl, mono-(naphthyl- or phenyl-C₁-C₇alkyl)-amino-C₁-C₇-alkyl, C₁-C₇-alkanoylamino-C₁-C₇alkyl, C₁-C₇-alkyl-O—CO—NH—C₁-C₇-alkyl, C₁-C₇-alkylsulfonylamino-C₁-C₇-alkyl, C₁-C₇alkyl-NH—CO—NH—C₁-C₇-alkyl, C₁-C₇alkyl-NH—SO₂—NH—C₁-C₇-alkyl, C₁-C₇-alkoxy, hydroxy-C₁-C₇alkoxy, C₁-C₇-alkoxy-C₁-C₇-alkoxy, C₁-C₇-alkanoylamino-C₁-C₇-alkyloxy, carboxy-C₁-C₇-alkyloxy, C₁-C₇-alkyloxycarbonyl-C₁-C₇-alkoxy, mono- or di-(C₁-C₇-alkyl)-aminocarbonyl-C₁-C₇alkyloxy, C₁-C₇-alkanoyloxy, mono- or di-(C₁-C₇-alkyl)-amino, mono-di-(naphthyl- or phenyl-C₁-C₇alkyl)-amino, N-mono-C₁-C₇-alkoxy-C₁-C₇alkylamino, C₁-C₇-alkanoylamino, C₁-C₇-alkylsulfonylamino, C₁-C₇-alkyl-carbonyl, halo-C₁-C₇alkylcarbonyl, hydroxy-C₁-C₇-alkylcarbonyl, C₁-C₇-alkoxy-C₁-C₇-alkylcarbonyl, amino-C₁-C₇-alkylcarbonyl, (N-) mono- or (N,N-) di-(C₁-C₇alkyl)-amino-C₁-C₇-alkylcarbonyl, C₁-C₇-alkanoylamino-C₁-C₇-alkylcarbonyl, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N-) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)aminosulfonyl.

Further alternatives for R₁ are selected from the group consisting of C₂-C₇-alkenyl, C₂-C₇-alkynyl, phenyl, naphthyl, heterocyclyl, especially as defined below for heterocyclyl, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, 3-C₁-C₇-alkyl-oxetidinyl, pyridyl, pyrimidinyl, morpholino, piperidinyl, piperazinyl, pyrrolidinyl, tetrahydrofuran-onyl, tetrahydro-pyranyl, indolyl, indazolyl, 1H-indazolyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, benzo[1,2,5]oxadiazolyl or 2H,3H-1,4-benzodioxinyl, phenyl- or naphthyl- or heterocyclyl-C₁-C₇-alkyl or —C₁-C₇-alkyloxy wherein heterocyclyl is as defined below, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇alkyl or naphthyl-C₁-C₇-alkyl)-pyrazolidinonyl, triazolyl, tetrazolyl, oxetidinyl, pyridyl, pyrimidinyl, morpholino, piperidinyl, piperazinyl, tetrahydrofuran-onyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl- or benzo[1,2,5]oxadiazolyl; such as benzyl or naphthylmethyl or -ethyl, halo-C₁-C₇-alkyl, such as trifluoromethyl, phenyloxy- or naphthyloxy-C₁-C₇-alkyl, phenyl-C₁-C₇alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇alkyl, phenyl- or naphthylsulfonylamino-C₁-C₇alkyl wherein phenyl or naphthyl is unsubstituted or substituted by one or more, especially one to three, C₁-C₇-alkyl moieties, phenyl- or naphthyl-C₁-C₇alkylsulfonylamino-C₁-C₇-alkyl, carboxy-C₁-C₇-alkyl, halo, especially fluoro or chloro, hydroxy, phenyl-C₁-C₇-alkoxy wherein phenyl is unsubstituted or substituted by C₁-C₇-alkoxy and/or halo, halo-C₁-C₇alkoxy, such as trifluoromethoxy, phenyl- or naphthyloxy, phenyl- or naphthyl-C₁-C₇-alkyloxy, phenyl- or naphthyl-oxy-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkoxy-C₁-C₇-alkyl or C₁-C₇alkyl moieties, phenyl- or naphthyl-C₁-C₇-alkylsulfonylamino, carboxyl, (N,N-) di(C₁-C₇alkyl)-amino-C₁-C₇alkoxycarbonyl, halo-C₁-C₇alkoxycarbonyl, phenyl- or naphthyloxycarbonyl, phenyl- or naphthyl-C₁-C₇alkoxycarbonyl, (N,N-) di-(C₁-C₇-alkyl)-amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇ alkyl)-aminocarbonyl, cyano, C₁-C₇alkylene which is unsubstituted or substituted by up to four C₁-C₇-alkyl substituents and bound to two adjacent ring atoms of the aryl moiety, C₂-C₇-alkenylene or -alkynylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇alkoxy-C₁-C₇-alkyl or C₁-C₇-alkyl moieties, phenyl- or naphthyl-C₁-C₇-alkylsulfinyl, sulfonyl, C₁-C₇alkylsulfonyl, halo-C₁-C₇-alkylsulfonyl, hydroxy-C₁-C₇alkylsulfonyl, C₁-C₇-alkoxy-C₁-C₇-alkylsulfonyl, amino-C₁-C₇alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)-aminosulfonyl; most especially R₁ is C₁-C₇-alkyl, hydroxy-C₁-C₇-alkyl, C₁-C₇alkoxy-C₁-C₇alkyl, C₁-C₇-alkoxy-C₁-C₇-alkoxy-C₁-C₇alkyl, amino-C₁-C₇-alkyl, C₁-C₇-alkoxy-C₁-C₇-alkylamino-C₁-C₇-alkyl, carboxy-C₁-C₇alkyl, C₁-C₇-alkoxycarbonyl-C₁-C₇alkyl, halo, especially fluoro, chloro or bromo, hydroxy, C₁-C₇-alkoxy, hydroxy-C₁-C₇-alkoxy C₁-C₇-alkoxy-C₁-C₇-alkoxy, amino-C₁-C₇-alkoxy, N—C₁-C₇-alkanoylamino-C₁-C₇alkoxy, carboxyl-C₁-C₇alkyloxy, C₁-C₇-alkoxycarbonyl-C₁-C₇-alkyloxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇-alkoxy, 4-C₁-C₇-alkylpiperidin-1-yl, nitro or cyano.

Unsubstituted or substituted alkyl is preferably C₁-C₂₀-alkyl, more preferably C₁-C₇-alkyl, that is straight-chained or branched (one or, if desired and possible, more times), and which is unsubstituted or substituted by one or more, e.g. up to three moieties independently selected from unsubstituted or substituted aryl as described below, especially phenyl or naphthyl each of which is unsubstituted or substituted as described below for unsubstituted or substituted aryl, unsubstituted or substituted heterocyclyl as described below, especially pyrrolyl, furanyl, thienyl, pyrazolyl, triazolyl, tetrazolyl, oxetidinyl, 3-(C₁-C₇-alkyl)-oxetidinyl, pyridyl, pyrimidinyl, morpholino, thiomorpholino, piperidinyl, piperazinyl, pyrrolidinyl, tetrahydrofuran-onyl, tetrahydro-pyranyl, indolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl and benzo[1,2,5]oxadiazolyl each of which is unsubstituted or substituted as described below for unsubstituted or substituted heterocyclyl, unsubstituted or substituted cycloalkyl as described below, especially cyclopropyl, cyclobutyl, cyclopentyl or cyclohexyl each of which is unsubstituted or substituted as described below for unsubstituted or substituted cycloalkyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkylthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di(C₁-C₇alkyl and/or C₁-C₇alkoxy-C₁-C₇alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇alkyl-, naphthyl- and/or phenyl-C₁-C₇-alkyl)-aminocarbonyl, cyano, C₁-C₇-alkenylene or alkynylene, C₁-C₇-alkylenedioxy, sulfenyl (—S—OH), sulfinyl (—S(═O)—OH), C₁-C₇-alkylsulfinyl (C₁-C₇-alkyl-S(═O)—), phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl (—S(O)₂OH), C₁-C₇-alkylsulfonyl (C₁-C₇-alkyl-SO₂—), phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl, naphthyl, phenyl-C₁-C₇-alkyl or naphthyl-C₁-C₇-alkyl)-aminosulfonyl.

Unsubstituted or substituted alkenyl preferably has 2 to 20 carbon atoms and includes one or more double bonds, and is more preferably C₂-C₇-alkenyl that is unsubstituted or substituted as described above for unsubstituted or substituted alkyl. Examples are vinyl or allyl.

Unsubstituted or substituted alkynyl preferably has 2 to 20 carbon atoms and includes one or more triple bonds, and is more preferably C₂-C₇-alkynyl that is unsubstituted or substituted as described above for unsubstituted or substituted alkyl. An example is prop-2-ynyl.

Unsubstituted or substituted aryl preferably is a mono- or polycyclic, especially monocyclic, bicyclic or tricyclic aryl moiety with 6 to 22 carbon atoms, especially phenyl (very preferred), naphthyl (very preferred), indenyl, fluorenyl, acenapthylenyl, phenylenyl or phenanthryl, and is unsubstituted or substituted by one or more, especially one to three, moieties, preferably independently selected from the group consisting of

a substituent of the formula —(C₀-C₇-alkylene)-(K)ₚ—(C₁-C₇-alkylene)-(L)_{q}-(C₀-C₇-alkylene)-H where C₀-alkylene means that a bond is present instead of bound alkylene, p and q, each independently of the other, are 0 or 1 and each of K and L, if present and independently of the others, is —O—, —NM-, —S—, —C(═O), —C(═S), —O—CO—, —CO—O—, —NM-CO—; —CO—NM-; —NM-SO₂—, —SO₂—NM; —NM-CO—NM-, —NM-CO—O—, —O—CO—NM-, —NM-SO₂—NM- wherein M is hydrogen or unsubstituted or substituted alkyl as defined below; especially selected from C₁-C₇-alkyl, phenyl, naphthyl, phenyl- or naphthyl-C₁-C₇-alkyl and halo-C₁-C₇-alkyl; e.g. C₁-C₇alkyl, such as methyl, ethyl, n-propyl, isopropyl, n-butyl, isobutyl, sec-butyl or tert-butyl, hydroxy-C₁-C₇alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇alkanoyloxy-C₁-C₇alkyl, C₁-C₇-alkyloxycarbonyl-C₁-C₇alkyl, amino-C₁-C₇-alkyl, such as aminomethyl, (N-) mono- or (N,N-) di-(C₁-C₇alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇alkyl, C₁-C₇-alkanoylamino-C₁-C₇-alkyl, C₁-C₇-alkyl-O—CO—NH—C₁-C₇alkyl, C₁-C₇alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇alkoxy, hydroxy-C₁-C₇-alkoxy, C₁-C₇alkoxy-C₁-C₇alkoxy, C₁-C₇-alkanoylamino-C₁-C₇-alkyloxy, carboxy-C₁-C₇alkyloxy, C₁-C₇-alkyloxycarbonyl-C₁-C₇alkoxy, mono- or di-(C₁-C₇alkyl)-aminocarbonyl-C₁-C₇alkyloxy, C₁-C₇alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇alkylsulfonylamino, C₁-C₇alkyl-carbonyl, halo-C₁-C₇-alkylcarbonyl, hydroxy-C₁-C₇alkylcarbonyl, C₁-C₇alkoxy-C₁-C₇-alkylcarbonyl, amino-C₁-C₇-alkylcarbonyl, (N-) mono- or (N,N-) di-(C₁-C₇-alkyl)-amino-C₁-C₇alkylcarbonyl, C₁-C₇-alkanoylamino-C₁-C₇-alkylcarbonyl, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N-) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)-aminosulfonyl;
from C₂-C₇-alkenyl, C₂-C₇-alkynyl, phenyl, naphthyl, heterocyclyl, especially as defined below for heterocyclyl, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)pyrazolidinonyl, triazolyl, tetrazolyl, oxetidinyl, 3-C₁-C₇-alkyl-oxetidinyl, pyridyl, pyrimidinyl, morpholino, piperidinyl, piperazinyl, pyrrolidinyl, tetrahydrofuran-onyl, tetrahydro-pyranyl, indolyl, indazolyl, 1H-indazolyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, benzo[1,2,5]oxadiazolyl or 2H,3H-1,4-benzodioxinyl, phenyl- or naphthyl- or heterocyclyl-C₁-C₇-alkyl or —C₁-C₇-alkyloxy wherein heterocyclyl is as defined below, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, pyridyl, pyrimidinyl, morpholino, piperidinyl, piperazinyl, tetrahydrofuran-onyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl- or benzo[1,2,5]oxadiazolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxy-C₁-C₇-alkyl, halo, especially fluoro or chloro, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, phenyl- or naphthyl-oxy-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonylamino, carboxyl, (N,N-) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N-) di-(C₁-C₇-alkyl)-amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇-alkyl)-aminocarbonyl, cyano, C₁-C₇alkylene which is unsubstituted or substituted by up to four C₁-C₇-alkyl substituents and bound to two adjacent ring atoms of the aryl moiety, C₂-C₇-alkenylene or -alkynylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)aminosulfonyl. Especially preferably aryl is phenyl or naphthyl, each of which is unsubstituted or substituted by one or more, e.g. up to three, substituents independently selected from the group consisting of C₁-C₇alkyl, hydroxy-C₁-C₇-alkyl, C₁-C₇-alkoxy-C₁-C₇alkyl, C₁-C₇-alkoxy-C₁-C₇alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxycarbonyl-C₁-C₇-alkyl, halo, especially fluoro, chloro or bromo, hydroxy, C₁-C₇alkoxy, hydroxy-C₁-C₇alkoxy C₁-C₇-alkoxy-C₁-C₇alkoxy, amino-C₁-C₇alkoxy, N—C₁-C₇-alkanoylamino-C₁-C₇-alkoxy, carboxyl-C₁-C₇-alkyloxy, C₁-C₇alkoxycarbonyl-C₁-C₇-alkyloxy, carbamoyl-C₁-C₇alkoxy, N-mono- or N,N-di-(C₁-C₇alkyl)-carbamoyl-C₁-C₇-alkoxy, morpholino-C₁-C₇-alkoxy, pyridyl-C₁-C₇alkoxy, amino, C₁-C₇alkanoylamino, C₁-C₇alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇alkoxy, 4-C₁-C₇-alkylpiperidin-1-yl, nitro and cyano.

Unsubstituted or substituted heterocyclyl is preferably a mono- or polycyclic, preferably a mono- or bi- or tricyclic-, unsaturated, partially saturated or saturated ring system with preferably 3 to 22 (more preferably 3 to 14) ring atoms and with one or more, preferably one to four, heteroatoms independently selected from nitrogen (═N—, —NH— or substituted —NH—), oxygen, sulfur (—S—, —S(═O)— or —S—(═O)₂—), and is unsubstituted or substituted by one or more, e.g. up to three, substitutents preferably independently selected from the substitutents mentioned above for aryl and from oxo. Preferably, heterocyclyl (which is unsubstituted or substituted as just mentioned) is selected from the following moieties (the asterisk marks the end of the bond binding to the rest of the molecule of formula I):

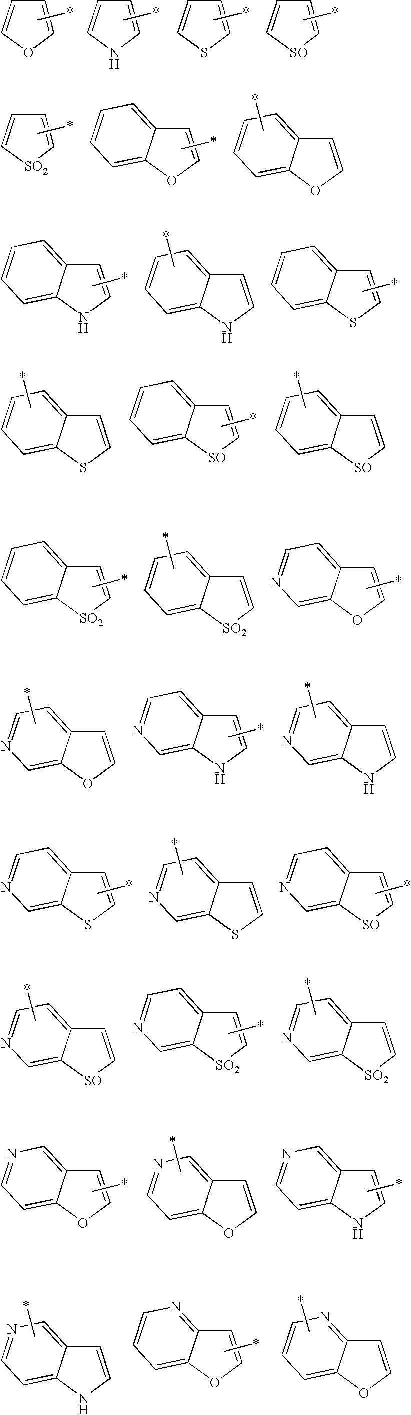

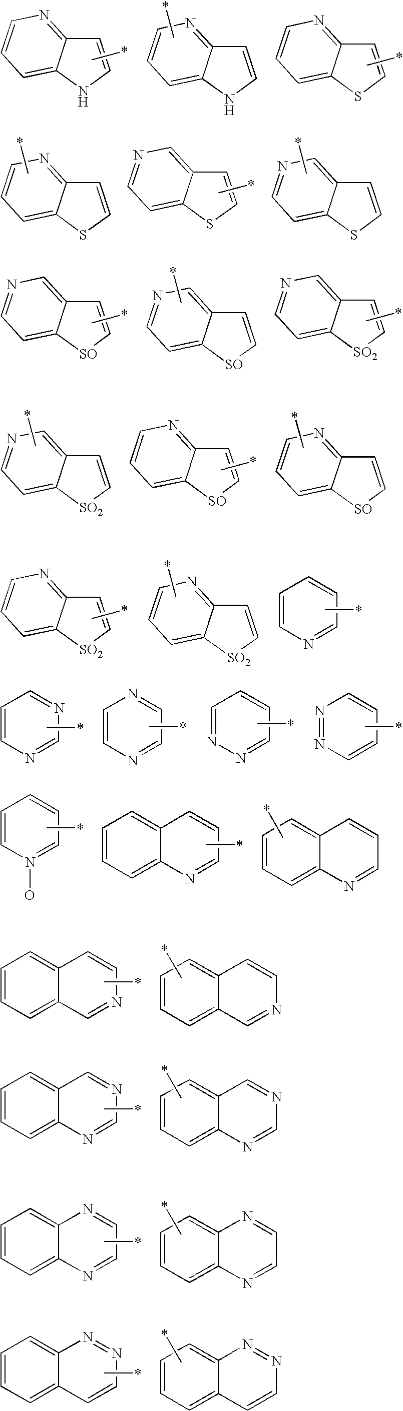

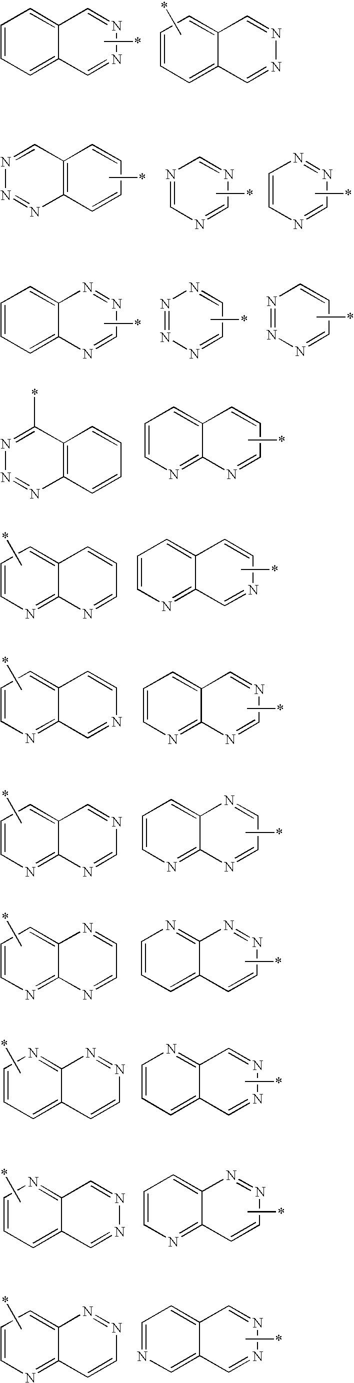

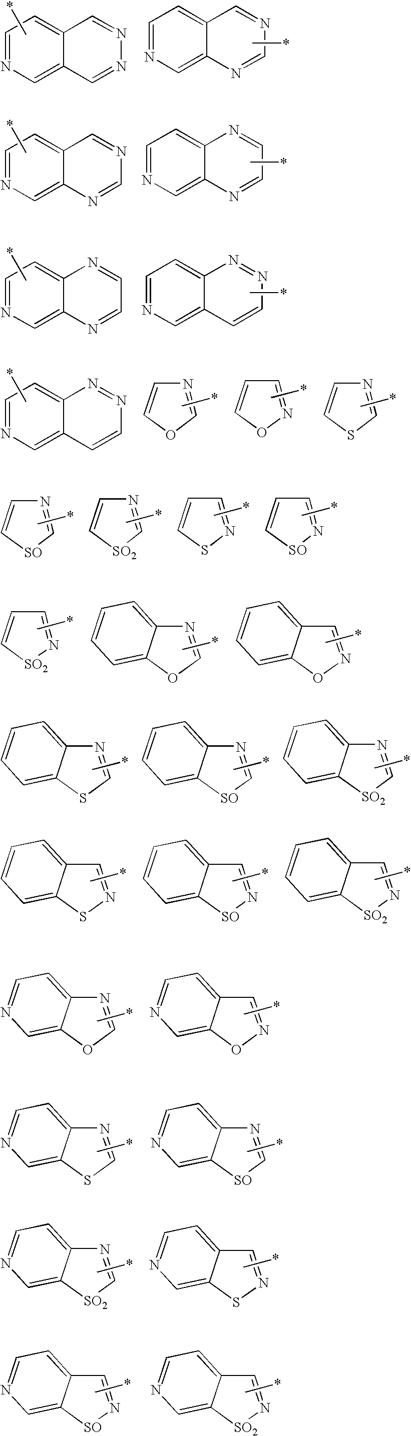

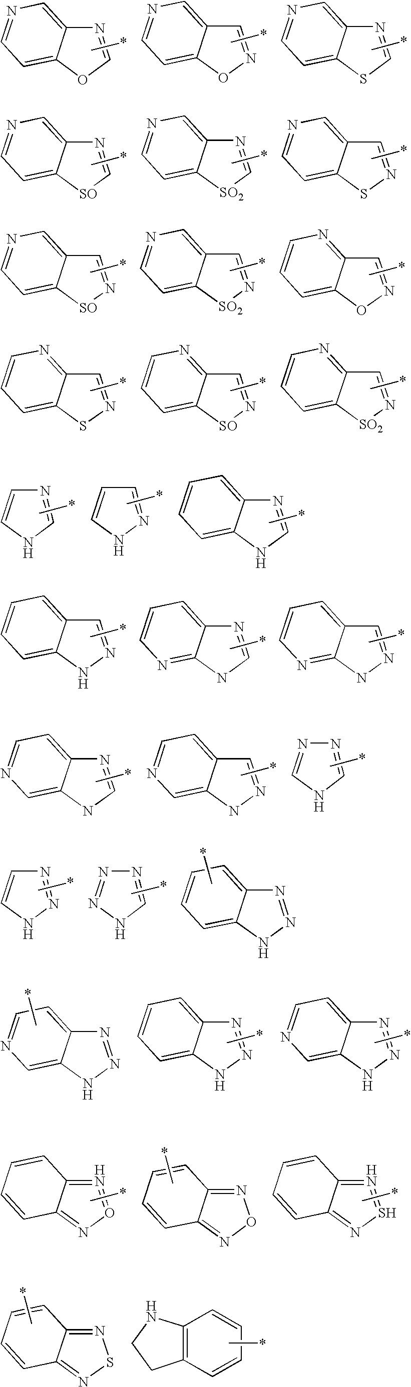

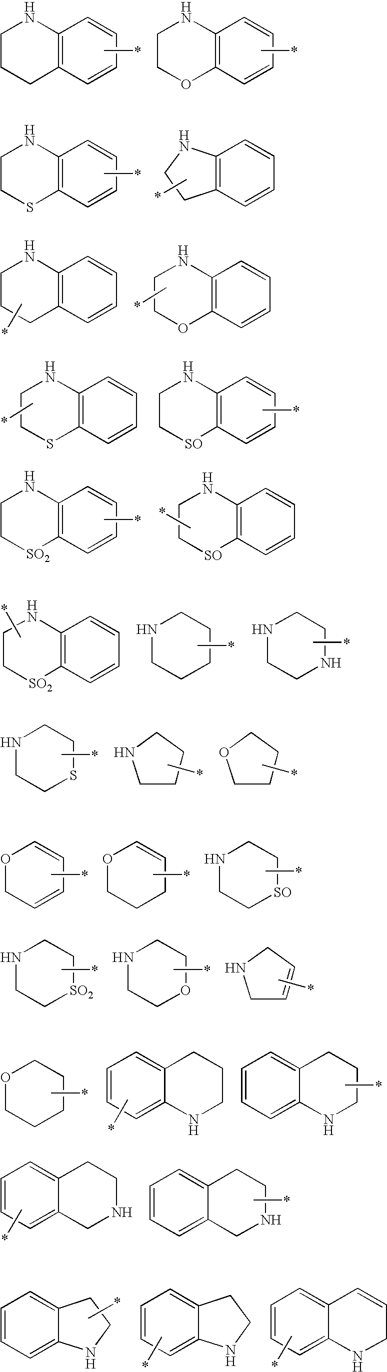

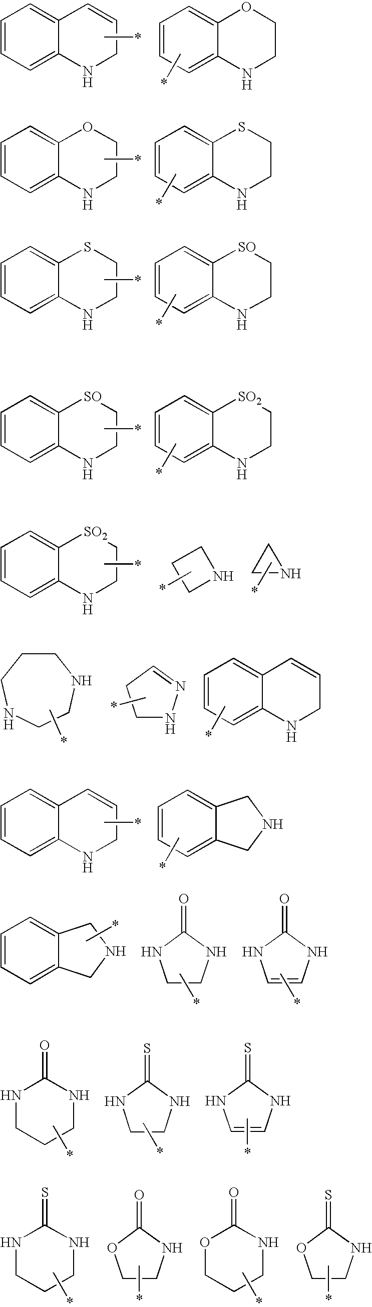

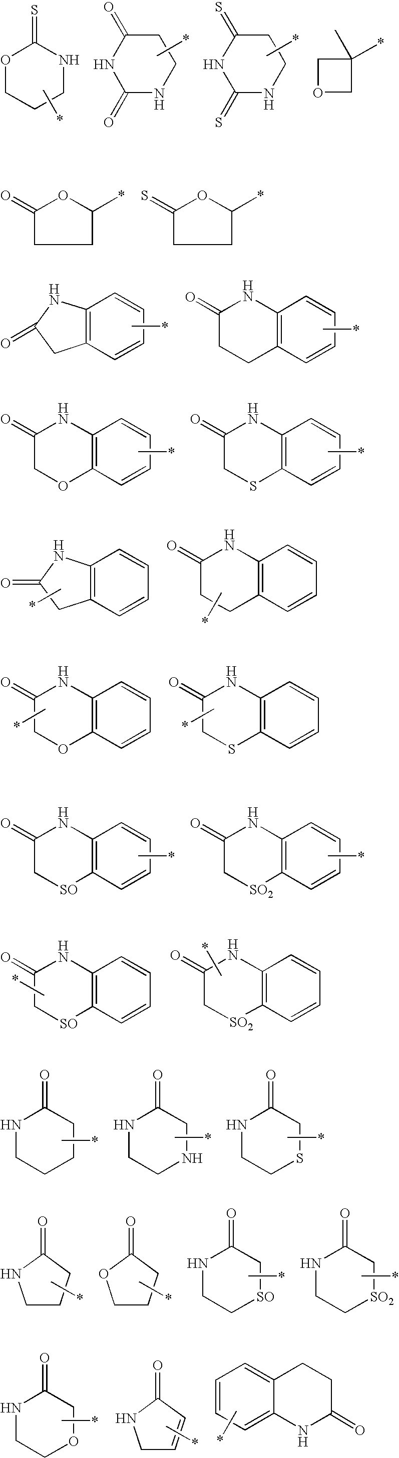

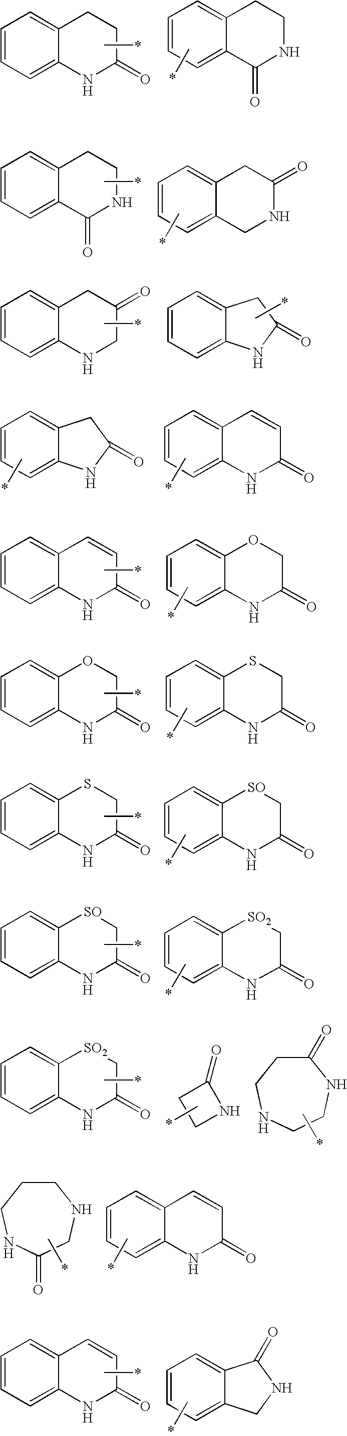

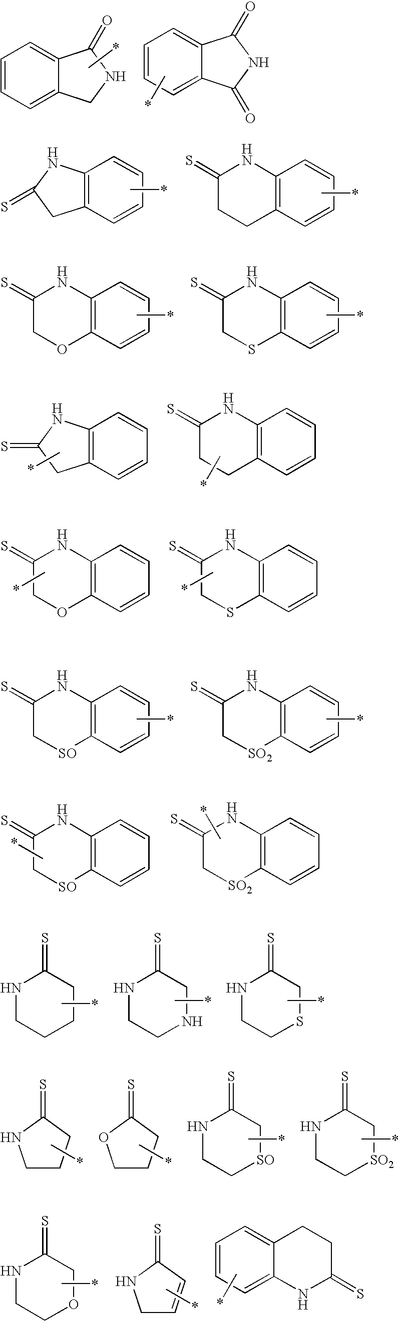

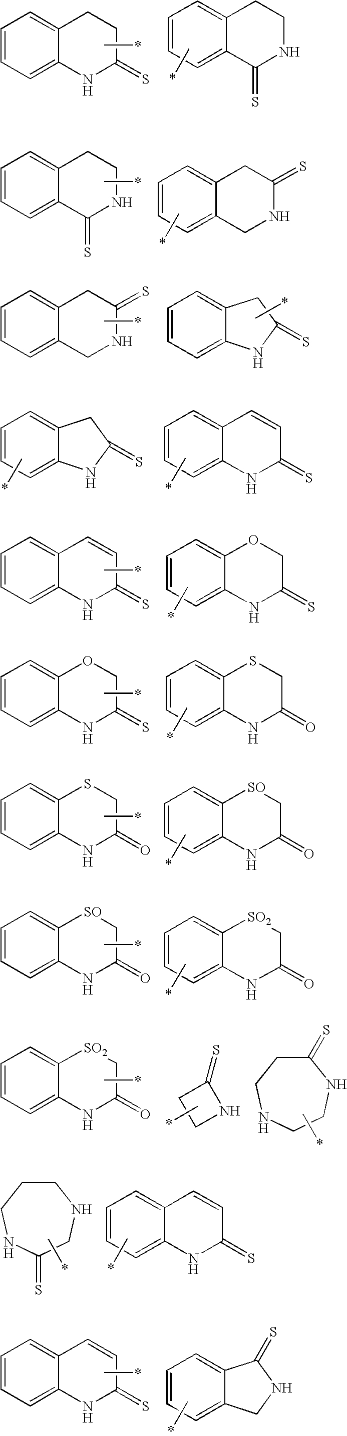

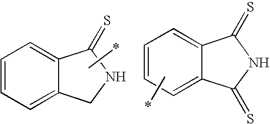

where in each case where an H is present the bond with the asterisk connecting the respective heterocyclyl moiety to the rest of the molecule the H may be replaced with said bond and/or the H may be replaced by a substituent, preferably as defined above. Especially preferred as heterocyclyl is pyrrolyl, furanyl, thienyl, pyrimidinyl, pyrazolyl, pyrazolidinonyl (=oxo-pyrazolidinyl), triazolyl, tetrazolyl, 1,3-oxazolyl, oxetidinyl, pyridyl, pyrimidinyl, morpholino, piperidinyl, piperazinyl, pyrrolidinyl, tetrahydrofuran-onyl (=oxo-tetrahydrofuranyl), tetrahydro-pyranyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl, benzo[1,2,5]oxadiazolyl, thiophenyl, pyridyl, indolyl, 1H-indazolyl, quinolyl, isoquinolyl or 1-benzothiophenyl; each of which is unsubstituted or substituted by one or more, e.g. up to three, substituents as mentioned above for substituted aryl, preferably independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇alkoxy-C₁-C₇-alkyl, amino-C₁-C₇-alkyl, C₁-C₇-alkoxy-C₁-C₇-alkylamino-C₁-C₇alkyl, carboxy-C₁-C₇alkyl, C₁-C₇-alkoxy-C₁-C₇-alkyl, halo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇alkoxy, N—C₁-C₇-alkylcarbamoyl-C₁-C₇-alkoxy, C₁-C₇-alkanoyl, C₁-C₇ alkoxy-C₁-C₇-alkanoyl, carboxy, carbamoyl and N—C₁-C₇-alkoxy-C₁-C₇-alkylcarbamoyl. In the case of heterocycles including an NH ring member, the substitutents, as far as bound via a carbon or oxygen atom, can preferably be bound at the nitrogen instead of the H.

Unsubstituted or substituted cycloalkyl is preferably mono- or polycyclic, more preferably monocyclic, C₃-C₁₀-cycloalkyl which may include one or more double (e.g. in cycloalkenyl) and/or triple bonds (e.g. in cycloalkynyl), and is unsubstituted or substituted by one or more, e.g. one to three substitutents preferably independently selected from those mentioned above as substituents for aryl. Preferred is cyclopropyl, cyclobutyl, cyclopentyl, cyclohexyl or cycloheptyl.

Acyl is preferably unsubstituted or substituted aryl-carbonyl or -sulfonyl, unsubstituted or substituted heterocyclylcarbonyl or -sulfonyl, unsubstituted or substituted cycloalkylcarbonyl or -sulfonyl, formyl or unsubstituted or substituted alkylcarbonyl or -sulfonyl, or (especially if G is oxy or preferably if it is NR4, especially imino (NH)) in the case of acyl R3 unsubstituted or substituted alkyloxycarbonyl or -oxysulfonyl, unsubstituted substituted aryl-oxycarbonyl or -oxysulfonyl, unsubstituted or substituted heterocyclyloxycarbonyl or -oxysulfonyl, unsubstituted or substituted cycloalkyloxycarbonyl or -oxysulfonyl, carbamoyl (less preferred), N-mono- or N,N-di-(unsubstituted or substituted aryl, unsubstituted or substituted heterocyclyl, unsubstituted or substituted cycloalkyl or unsubstituted or substituted alkyl)-aminocarbonyl, sulfamoyl (less preferred) or N-mono- or N,N-di-(unsubstituted or substituted aryl, unsubstituted or substituted heterocyclyl, unsubstituted or substituted cycloalkyl or unsubstituted or substituted alkyl)-amino-sulfonyl; with the proviso that in cases of -oxycarbonyl bound moieties G is NR4, preferably NH; wherein unsubstituted or substituted aryl, unsubstituted or substituted heterocyclyl, unsubstituted or substituted cycloalkyl and unsubstituted or substituted alkyl are preferably as described above. Preferred is C₁-C₇-alkanoyl, unsubstituted or mono-, di- or tri-(halo)-substituted benzoyl or naphthoyl, unsubstituted or phenyl-substituted pyrrolldinylcarbonyl, especially phenyl-pyrrolidinocarbonyl, C₁-C₇-alkylsulfonyl or (unsubstituted, halo- or C₁-C₇-alkyl-substituted) phenylsulfonyl, C₁-C₇-alkoxycarbonyl or phenyl-C₁-C₇-alkyloxycarbonyl.

“-Oxycarbonyl-” means —O—C(═O)—, “aminocarbonyl” means in the case of mono-substitution —NH—C(═O)—, in the case of double substitution also the second hydrogen is replaced by the corresponding moiety. For example, C₁-C₇-alkoxycarbonyl is C₁-C₇alkyl-O—C(═O)—, N,N-di(C₁-C₇-alkyl)aminocarbonyl is (C₁-C₇alkyl)₂N—C(═O)—.

Alkylene is especially C₁-C₇alkylene and can be branched or linear; preferred is methylene (CH₂), ethylene (CH₂CH₂), trimethylene (CH₂CH₂CH₂) or propylene (CH₃—CHCH₂).

In unsubstituted or substituted aryl-alkyl, unsubstituted or substituted heterocyclyl-alkyl or unsubstituted or substituted cycloalkyl-alkyl, the alkyl part is preferably C₁-C₇-alkyl, e.g. in aryl-C₁-C₇alkyl, heterocyclyl-C₁-C₇alkyl or cycloalkyl-C₁-C₇-alkyl.

In substituted imino NR4, an imino substituent R4 is preferably selected from acyl, especially C₁-C₇-alkanoyl, phenylcarbonyl (=benzoyl), C₁-C₇alkylsulfonyl or phenylsulfonyl wherein phenyl is unsubstituted or substituted by one to 3 C₁-C₇-alkyl groups, and especially from one or two moieties selected from alkyl, alkenyl, alkynyl, aryl, heterocyclyl and cycloalkyl each of which is unsubstituted or substituted and is preferably as described above for the corresponding unsubstituted or substituted moieties. Preferred as NR4 is C₁-C₇alkanoylimino, mono- or di-(phenyl, naphthyl, C₁-C₇-alkoxy-phenyl, C₁-C₇alkoxynaphthyl, naphthyl-C₁-C₇-alkyl or phenyl-C₁-C₇-alkyl)-carbonylimino (e.g. 4-methoxybenzoylimino), or especially mono- or di-(C₁-C₇-alkyl and/or C₁-C₇-alkoxy-C₁-C₇alkyl)-imino or mono- or di-(phenyl, naphthyl, C₁-C₇-alkoxy-phenyl, C₁-C₇-alkoxynaphthyl, phenyl-C₁-C₇-alkyl, naphthyl-C₁-C₇-alkyl, C₃-C₈-cycloalkyl, C₃-C₈-cycloalkyl-C₁-C₇alkyl, C₁-C₇-alkoxy-naphthyl-C₁-C₇-alkyl or C₁-C₇-alkoxy-phenyl-C₁-C₇alkyl)-imino.

Where groups such as C(═O)NH or CH₂CH₂ or atoms such as N are given, they are bound by the bonds indicated in formula I (or its precursors), therefore the bonds are not repeated.

The expression “where in each case H is unreplaced or can be replaced by a moiety R₁ as defined above if p is 1” means that an H in the respective moieties A or D is replaced by R₁.

In the moieties A and D, the bonds to the rest of the molecule are such that nitrogen (except if present as a salt where the nitrogen may also bind an additional hydrogen) is trivalent, oxygen is divalent and carbon is tetravalent. Sulfur as S is divalent, as S(═O) tetravalent and as S(O)₂ hexavalent. Thus, as A, for example, including the bonds shown in formula I, CH₂ stands for —CH₂—, S(O)₀₋₂ for —S(O)₀₋₂—, CH═CH for —CH═CH₂—, CH₂O for —CH₂O—, CH₂S(O)₀₋₂ for —CH₂S(O)₀₋₂—, CH₂NH for —CH₂—NH—, C(═O)NH for —C(═O)NH—, SO₂NH for —SO₂NH. As D, including the bonds shown in formula I, N stands for nitrogen with three bonds (two to form the ring, the third to bind to E), CH stands for CH with three further bonds (two to form the ring, the third to bind to E), in CH═C and CH₂—CH the left carbon has one further bond to form the ring and the right carbon has two further bonds ((one to complete the ring, the other to bind to E), in CHO the carbon has two further bonds (one to complete the ring, the other to E) and O one further bond to form the ring, in CHS(O)₀₋₂, the carbon has two further bonds (one to complete the ring, the other to E) and S one further bond to form the ring, in CH₂N the carbon has one further bond to complete the ring and N has two further bonds (one to form the ring, one to bind to E), in NHCH the nitrogen has one further bond to complete the ring and the carbon has two further bonds (one to complete the ring, one to bind to E), in C(═O)N the carbon has one further bond to complete the ring and the nitrogen has two further bonds (one to complete the ring, one to bind to E) and in SO₂N, the sulfur has one further bond to complete the ring and the nitrogen has two further bonds (one to complete the ring, the other to bind to E).

Generally, where substituents are present, they replace a hydrogen, e.g. in the case of R and/or R₁.

In one embodiment, the present invention is related to a compound of the formula I according to any of the preceding claims wherein each R₁, independently of the others, (present if p>0) is a substituent selected from the group consisting of

a substituent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —CO—NV— wherein V is hydrogen or unsubstituted or substituted alkyl as defined below; or
phenyl- or naphthyl- or heterocyclyl-C₁-C₇-alkyl;
R2 is hydrogen or unsubstituted or substituted alkyl;
R3 is unsubstituted or substituted alkyl, unsubstituted or substituted heterocyclyl, unsubstituted or substituted aryl-alkyl, unsubstituted or substituted heterocyclyl-alkyl, unsubstituted or substituted cycloalkyl alkyl or, if G is unsubstituted or substituted imino, has one of the meanings just mentioned or is acyl;
A is CH₂, O, CH═CH, or CH₂CH₂, where in each case H is unreplaced or one or two can be replaced by a moiety R₁ as defined above if p is 1;
D is N, CH, or NHCH, where in each case a H if present is unreplaced or one can be replaced by a moiety R₁ as defined above if p is 1;
E is unsubstituted or (halo, hydroxy, C₁-C₇-alkyloxy, phenoxy, phenyl-C₁-C₇-alkyloxy, C₁-C₇-alkanoyloxy or benzoyloxy)-substituted C₁-C₇alkylene;
T is carbonyl or methylene;
G is oxy, unsubstituted or substituted (NR4) imino, C(═O)NH or C(═O)NR4, wherein R4 is an imino substituent;
or G-R3 together is hydrogen;
m is 0; n is 0; and p is 0 (zero) or 1;
or a salt thereof.

The following preferred embodiments of the moieties and symbols in formula I can be employed independently of each other to replace more general definitions and thus to define specially preferred embodiments of the invention, where the remaining definitions of other moieties, respectively, can be kept broad as defined in embodiments of the inventions defined above of below.

### Preferred Definitions for R1

R₁ is preferably absent (p is zero) or is C₁-C₇alkyl, C₁-C₇-alkoxy-C₁-C₇-alkyl or phenyl-C₁-C₇-alkyl. R₁ can only be present if at least one of A or D (without R₁ being bound) can carry a (=at least one) hydrogen. R₁ then replaces a hydrogen.

In one embodiment, R1 is absent.

In a second embodiment, R1 is a substituent of the formula —(C₀-C₇-alkylene)-(X)ᵣ—(C₁-C₇-alkylene)-(Y)ₛ—(C₀-C₇-alkylene)H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV—, preferably —O—, —NV— and —CO—NV—, wherein V is hydrogen or unsubstituted or substituted alkyl as defined below, preferably C₁-C₇alkyl, such as methyl. Examples include

(a) —(C₁-C₇-alkyl such as methyl, ethyl, n-propyl, n-butyl, n-pentyl, n-hexyl or n-heptyl, preferably methyl, ethyl or n-hexyl, most preferably methyl or n-hexyl;
(b) —(C₁-C₇-alkylene)-O—C₁-C₇alkyl, such as —(C₁-C₇alkylene)O—C₁-C₄-alkyl, preferably —(C₁-C₅-alkylene)-O—C₁-C₃-alkyl, such as —CH₂CH₂CH₂OCH₃, —CH₂CH₂CH₂OCH₂CH₃, —CH₂CH₂CH₂CH₂OCH₃, —CH₂CH₂CH₂CH₂OCH₂CH₃, more preferably —CH₂CH₂CH₂OCH₃, —CH₂CH₂CH₂OCH₂CH₃, —CH₂CH₂CH₂CH₂OCH₃, most preferably —CH₂CH₂CH₂OCH₃;
(c) —(C₁-C₇alkylene)-OH, preferably —(C₁-C₅-alkylene)-OH, such as —CH₂CH₂CH₂OH, —CH₂CH₂CH₂CH₂OH, most preferably —CH₂CH₂CH₂OH;
(d) —(C₁-C₇-alkylene)-O—(C₁-C₇-alkylene)-O—C₁-C₇-alkyl, such as —(C₁-C₄-alkylene)-O—(C₁-C₄-alkylene)-O—C₁-C₄-alkyl, preferably —(C₁-C₂-alkylene)-O—(C₁-C₃-alkylene)-O—C₁-C₂-alkyl, such as —CH₂OCH₂CH₂OCH₃;
(e) —(C₁-C₇-alkylene)-C(O)N(C₁-C₄-alkyl)-C₁-C₇-alkyl, such as —(C₁-C₄-alkylene)-C(O)N(C₁-C₂-alkyl)-C₁-C₄-alkyl, preferably —CH₂CH₂CH₂C(O)N(methyl)-CH₃.

Most preferred are examples of (a) and (b).

In a third embodiment, R1 is phenyl-C₁-C₇alkyl, such as phenyl-C₁-C₄-alkyl, preferably phenyl-CH₂CH₂— or phenyl-CH₂CH₂CH₂—, whereby phenyl is unsubstituted or substituted with C₁-C₇alkyl, —O—C₁-C₇-alkyl, halo-C₁-C₇-alkyl, —O-halo-C₁-C₇-alkyl, halo, hydroxy, nitro, amino, amino-C₁-C₇alkyl, carboxyl, cyano, or hydroxy-C₁-C₇-alkyl, preferably unsubstituted.

In a fourth embodiment, R1 is heterocyclyl-C₁-C₇-alkyl, such as heterocyclyl-C₁-C₆-alkyl, preferably heterocyclyl-CH₂CH₂CH₂CH₂—, whereby heterocyclyl is preferably mono- or bicyclic, more preferably monocyclic, such as 5- or 6-membered ring, which is preferably aromatic or saturated, more preferably saturated, containing 1, 2 or 3, such as 2, heteroatoms selected from N and O. Preferred examples of heterocyclyl include morpholinyl, piperidinyl and piperazinyl, most preferably morpholinyl. The heterocyclic ring is unsubstituted or substituted with C₁-C₇-alkyl, —O—C₁-C₇-alkyl, halo-C₁-C₇-alkyl, —O-halo-C₁-C₇-alkyl, halo, hydroxy, nitro, amino, amino-C₁-C₇-alkyl, carboxyl, cyano, or hydroxy-C₁-C₇alkyl, preferably unsubstituted.

Most preferably R1 is as defined under (b).

R₁ is preferably bound to D in a compound of the formula I, as depicted in the following formula I* which shows a preferred class of compounds of the formula I,

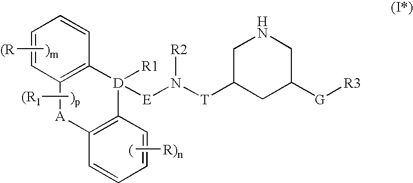

wherein R₁, R2, R3, R, A, D, E, T, G, m and n have the meanings given above or preferably below for a compound of the formula I; or a (preferably pharmaceutically acceptable) salt thereof.

Alternatively, R₁ can be absent (p=0) and one moiety R can have the meaning of R₁ as given hereinabove or hereinbelow, so that also a compound of the formula I**

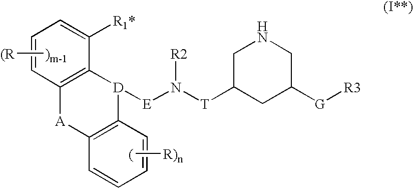

wherein R2, R3, R, A, D, E, T, G, m and n have the meanings given above or preferably below for a compound of the formula I wherein instead of one R a moiety R₁* is present that has the meanings of R₁ given above or preferably below for a compound of the formula I, or a pharmaceutically acceptable salt thereof.

### Preferred Definitions for R2

R2 is preferably hydrogen, C₁-C₇-alkyl or unsubstituted or substituted aryl-C₁-C₇-alkyl, e.g. hydrogen, C₁-C₄-alkyl or phenyl-C₁-C₄-alkyl wherein phenyl is unsubstituted or substituted by halo, especially chloro, more preferably R2 is hydrogen or, C₁-C₄-alkyl such as methyl. Hydrogen R2 is especially preferred.

### Preferred Definitions for R3 and G

R3 is as defined in the claims, preferably R³ is in a first embodiment acyl as defined herein, more preferably an acyl group as set forth below in embodiments (a) to (g):

(a) In one embodiment, R3 is unsubstituted or substituted aryl sulfonyl. Preferred examples for the aryl moiety of the acyl substituent are phenyl and naphthyl, more preferably phenyl. When the aryl moiety is substituted, it is preferably mono-, di- or tri-substituted, more preferably mono- or di-substituted. Suitable substituents for the aryl moiety are as defined herein, preferably C₁-C₇-alkyl, —O—C₁-C₇alkyl, halo-C₁-C₇-alkyl, —O-halo-C₁-C₇-alkyl, O-phenyl, halo, hydroxy, nitro, amino, amino-C₁-C₇-alkyl, carboxyl, cyano, hydroxy-C₁-C₇-alkyl, C₁-C₇-alkoxy-C₁-C₇-alkyl, C₁-C₇-alkoxy-C₁-C₇-alkoxy-C₁-C₇-alkyl, C₁-C₇-alkoxy-C₁-C₇-alkoxy, hydroxy-C₁-C₇-alkoxy, C₁-C₇-alkanoyloxy-C₁-C₇-alkyl, N—C₁-C₇-dialkylamino-C₁-C₇-alkoxy, C₁-C₇-alkanoylamino-C₁-C₇-alkyl, C₁-C₇-alkanoylamino, N—C₁-C₇alkoxy-C₁-C₇-alkylamino, N—C₁-C₇-alkanoyl-N—C₁-C₇-alkoxy-C₁-C₇-alkyl-amino, C₁-C₇alkylsulfonyl, carboxy-C₁-C₇-alkyl, C₁-C₇-alkoxycarbonyl-C₁-C₇-alkyl, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇alkyl, N—C₁-C₇-alkylcarbamoyl-C₁-C₇-alkyl, N—C₁-C₇-haloalkylcarbamoyl-C₁-C₇alkyl, carbamoyl-C₁-C₇alkoxy, N—C₁-C₇-alkylcarbamoyl-C₁-C₇-alkoxy, C₁-C₇-alkanoyl, C₁-C₇alkyloxy-C₁-C₇-alkanoyl, C₁-C₇-alkoxy-C₁-C₇-alkanoyl, carbamoyl and N—C₁-C₇alkoxy-C₁-C₇-alkylcarbamoyl, C₁-C₇-alkanoyl, C₁-C₇-alkylheterocyclyl and heterocyclyl, whereby heterocyclyl is preferably a monocyclic moiety with preferably a 5- or 6-membered ring, which may be saturated, partially unsaturated or aromatic, preferably saturated or aromatic, and containing preferably 1 or 2 heteroatoms selected from N and O;
more preferably C₁-C₇-alkyl, —O—C₁-C₇-alkyl, halo-C₁-C₇-alkyl, —O-halo-C₁-C₇-alkyl, O-phenyl, halo, hydroxy, cyano, C₁-C₇-alkoxy-C₁-C₇-alkoxy, hydroxy-C₁-C₇-alkoxy, N—C₁-C₇-dialkylamino-C₁-C₇alkoxy, C₁-C₇-alkanoyl, C₁-C₇-alkanoylamino, C₁-C₇-alkylsulfonyl, C₁-C₇-alkylheterocyclyl and heterocyclyl, whereby heterocyclyl is as defined above,
in particular, methyl, O-methyl, Cl, F, CN, OCF₃, OCHF₂, CF₃, NH(CO)CH₃, OPh, OH, C(O)CH₃, OCH₂CH₂CH₂N(CH₃)₂, OCH₂CH₂N(CH₃)₂, OCH₂CH₂CH₂OCH₃, OCH₂CH₂CH₂OH, CH₂-morpholino, methylsulfonyl, and pyrazolyl.
(b) In one embodiment, R3 is unsubstituted or substituted heterocyclyl sulfonyl. The heterocyclyl moiety is preferably mono- or bicyclic, more preferably bicyclic. Preferred are aromatic ring systems, or partially saturated ring systems, in particular whereby one of the rings is aromatic and the other is saturated or partially saturated, most preferred are partially saturated. The heterocyclyl moiety has preferably 1, 2 or 3, more preferably 1 or 2, most preferably 2, heteroatoms selected from O, N or S, more preferably O or N. The ring system may contain an oxo moiety. Particularly preferred examples include bicyclic 9- to 11-, preferably 10-, membered rings preferably containing 1 or 2 of a nitrogen or an oxygen atom, in particular, 2,3-dihydro-benzo[1,4]dioxinyl, 3,4-dihydro-2H-benzo[1,4]oxazinyl, 3,4-dihydro-1H-quinolin-2-onyl, 2,3-dihydrobenzofuranyl, 1,3-dihydro-indol-2-onyl, benzo[1,2,5]thiadiazolyl, 2,3-dihydro-1H-indolyl, benzothiophenyl, and 3,4-dihydro-2H-benzo[b][1,4]dioxepinyl, or monocyclic 5- or 6-membered rings, preferably containing an S or an N atom, in particular pyridyl and thiophenyl, where each heterocyclyl is unsubstituted or substituted by one or more, e.g. up to three, substituents independently selected from the group consisting of C₁-C₇-alkyl, hydroxy-C₁-C₇-alkyl, halo-C₁-C₇-alkyl, halo, hydroxy, C₁-C₇-alkoxy, C₁-C₇-alkoxy-C₁-C₇-alkoxy, carboxy-C₁-C₇-alkoxy, amino-C₁-C₇-alkoxy, N—C₁-C₇-alkanoylamino-C₁-C₇-alkoxy, carbamoyl-C₁-C₇-alkyl, carbamoyl-C₁-C₇alkoxy, C₁-C₇-alkanoyl, C₁-C₇-alkyloxy-C₁-C₇-alkanoyl, C₁-C₇-alkoxy-C₁-C₇-alkanoyl, carboxyl, carbamoyl and N—C₁-C₇-alkoxy-C₁-C₇-alkylcarbamoyl or heterocyclyl whereby heterocyclyl is preferably a monocyclic moiety with preferably a 5- or 6-membered ring, which may be saturated, partially unsaturated or aromatic, preferably aromatic, and containing preferably 1 or 2 heteroatoms selected from N and O; more preferably C₁-C₇-alkyl, halo-C₁-C₇-alkyl, C₁-C₇alkanoyl, or heterocyclyl as defined above, in particular methyl, CF₃, C(O)CH₃ and oxazolyl. Most preferably heterocyclyl is unsubstituted.
(c) In one embodiment, R3 is unsubstituted or substituted alkyl sulfonyl. Preferred examples for the alkyl moiety are branched or straight chain C₁-C₇-alkyl which may be substituted or unsubstituted. Preferred examples include methyl, ethyl, isopropyl, n-propyl, n-butyl, sec-butyl or tert-butyl, more preferably methyl, ethyl or isopropyl, most preferably methyl or ethyl. The alkyl moiety can be substituted. When the alkyl moiety is substituted, it is preferably mono-, di- or tri-substituted, more preferably mono- or tri-substituted. Suitable substituents for the alkyl moiety are as defined herein, preferably O—C₁-C₄-alkyl, halo, hydroxy, unsubstituted or substituted, preferably unsubstituted, phenyl, nitro, amino, amino-C₁-C₇-alkyl, N-mono- or N,N-carboxyl, and cyano, more preferably halo such as F or phenyl.
(d) In one embodiment, R3 is unsubstituted or substituted cycloalkyl sulfonyl. Preferred examples for the cycloalkyl moiety are C₃-C₈-alkyl which may be substituted or unsubstituted. Preferred examples include cyclopropyl, cyclopentyl and cyclohexyl, more preferably cyclopropyl. The cycloalkyl moiety is preferably unsubstituted.
(e) In one embodiment, R3 is unsubstituted or substituted alkyl carbonyl. Preferred examples for the alkyl moiety are branched or straight chain C₁-C₇alkyl which may be substituted or unsubstituted. Preferred examples include methyl, ethyl, isopropyl, n-propyl, n-butyl, sec-butyl or tert-butyl, more preferably methyl, ethyl or sec-butyl, most preferably methyl or sec-butyl. The alkyl moiety, in particular methyl, can be substituted. When the alkyl moiety is substituted, it is preferably mono-, di- or tri-substituted, more preferably mono-substituted. Suitable substituents for the alkyl moiety are as defined herein, preferably O—C₁-C₄-alkyl, halo, hydroxy, unsubstituted or substituted (e.g. with —C₁-C₄-alkyl, halo, hydroxy)phenyl, substituted or unsubstituted, preferably unsubstituted, heterocyclyl, nitro, amino, amino-C₁-C₇-alkyl, N-mono- or N,N-carboxyl, and cyano, whereby the heterocyclyl moiety is in this connection preferably mono-cyclic aromatic or saturated. Preferred are aromatic ring systems. The heterocyclyl moiety has preferably 1, 2 or 3, more preferably 1 or 2, most preferably 1, heteroatoms selected from O, N or S, more preferably S or N. Particularly preferred examples include 6-membered rings preferably containing a nitrogen atom, in particular pyridyl. More preferred substituents on alkyl are substituted or unsubstituted phenyl or pyridyl.
(f) In one embodiment, R3 is unsubstituted or substituted alkyloxycarbonyl. Preferred examples for the alkyl moiety are branched or straight chain C₁-C₇-alkyl which may be substituted or unsubstituted. Preferred examples include methyl, ethyl, isopropyl, n-propyl, n-butyl, sec-butyl or tert-butyl, more preferably methyl, ethyl or tert-butyl, most preferably methyl or tert-butyl. The alkyl moiety, in particular methyl, can be substituted. When the alkyl moiety is substituted, it is preferably mono-, di- or tri-substituted, more preferably mono-substituted. Suitable substituents for the alkyl moiety are as defined herein, preferably O—C₁-C₄-alkyl, halo, hydroxy, unsubstituted or substituted (e.g. with —C₁-C₄-alkyl, halo, hydroxy)phenyl, substituted or unsubstituted, more preferably unsubstituted, heterocyclyl, nitro, amino, amino-C₁-C₇-alkyl, N-mono- or N,N-carboxyl, and cyano, whereby the heterocyclyl moiety is in this connection preferably mono-cyclic aromatic or saturated. Preferred are saturated ring systems. The heterocyclyl moiety has preferably 1, 2 or 3, more preferably 1 or 2, most preferably 1, heteroatoms selected from O, N or S, more preferably O or N. Particularly preferred examples include 5 or 6-membered rings preferably containing an oxygen atom, in particular tetrahydrofuranyl or tetrahydropyranyl. More preferred substituents on alkyl are tetrahydrofuranyl or tetrahydropyranyl.
(g) In one embodiment, R3 is unsubstituted or substituted heterocyclyloxycarbonyl. Preferred examples for the heterocyclyl moiety are mono-cyclic aromatic or saturated rings. Preferred are saturated ring systems. The heterocyclyl moiety has preferably 1, 2 or 3, more preferably 1 or 2, most preferably 1, heteroatoms selected from O, N or S, more preferably O or N. Particularly preferred examples include 5 or 6-membered rings preferably containing an oxygen atom, in particular tetrahydrofuranyl or tetrahydropyranyl. More preferred substituents on alkyl are tetrahydrofuranyl or tetrahydropyranyl.

When R3 is acyl, R1 is preferably present and as defined herein. When R3 is acyl, T is preferably C(O). When R3 is acyl, G is preferably imino with R4 being H or C₁-C₇-alkyl.

In a second embodiment, R3 is unsubstituted or substituted alkyl. Preferred examples for alkyl are branched or straight chain C₁-C₇-alkyl which may be substituted or unsubstituted. Preferred examples include methyl, ethyl, isopropyl, n-propyl, n-butyl, sec-butyl, tert-butyl, or neopentyl, more preferably methyl, ethyl or isopropyl, sec-butyl, neopentyl most preferably methyl, ethyl, sec-butyl, neopentyl. The alkyl moiety can be substituted. When the alkyl moiety is substituted, it is preferably mono-, di- or tri-substituted, more preferably mono- or tri-substituted. Suitable substituents for the alkyl moiety are as defined herein, preferably O—C₁-C₄-alkyl, halo, hydroxy, unsubstituted or substituted, preferably unsubstituted, phenyl, nitro, amino, amino-C₁-C₇alkyl, C₁-C₇-mono- or dialkyl amino-C₁-C₇alkyl, N-mono- or N,N-carboxyl, and cyano. More preferably OH or amino-C₁-C₇-alkyl, C₁-C₇ mono- or dialkyl amino-C₁-C₇-alkyl, such as CH₂N(CH₃)₂. Most preferably, straight chain alkyl is unsubstituted and branched alkyl in substituted or unsubstituted.

When R3 is alkyl, R1 is preferably present and as defined herein. When R3 is alkyl, T is preferably C(O). When R3 is alkyl, E is preferably CH₂. When R3 is alkyl, G is preferably imino with R4 being H or C₁-C₇-alkyl, more preferably ethyl. Alternatively, when R3 is alkyl, G is preferably (CO)NR4 with R4 being H or C₁-C₇-alkyl. When R3 is alkyl the tricyclic moiety is preferably

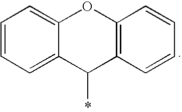

In a third embodiment, R3 is unsubstituted or substituted cycloalkyl alkyl. Examples include cycloalkyl C₁-C₄-alkyl, such as cycloalkyl C₁-C₂-alkyl, preferably cycloalkyl-CH₂—. Preferred examples for the cycloalkyl moiety are monocyclic rings, preferably C₃-C₇cycloalkyl, more preferably C₃, C₄, C₅ and C₆-cycloalkyl, most preferably cyclohexyl. The cycloalkyl moiety may be substituted or unsubstituted. When the cycloalkyl moiety is substituted, it is preferably mono-substituted. Suitable substituents for the cycloalkyl moiety are as defined herein, preferably C₁-C₇alkyl, O—C₁-C₄-alkyl, halo, hydroxy, unsubstituted or substituted phenyl, naphthyl, unsubstituted or substituted, preferably unsubstituted, phenyl- or naphthyloxy, unsubstituted or substituted, preferably unsubstituted, phenyl- or naphthyl-C₁-C₇-alkyloxy, nitro, amino, amino-C₁-C₇-alkyl, carboxyl, and cyano, most preferably phenyl or naphthyl. Most preferably, the cycloalkyl moiety is unsubstituted.

When R3 is cycloalkyl, R1 is preferably present and as defined herein. When R3 is cycloalkyl, T is preferably C(O). When R3 is cycloalkyl, E is preferably CH₂. When R3 is cycloalkyl, G is preferably (CO)NR4 with R4 being H or C₁-C₇-alkyl such as ethyl. When R3 is cycloalkyl the tricyclic moiety is preferably

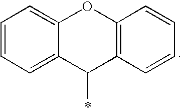

In a fourth embodiment, R3 is unsubstituted or substituted aryl alkyl. Examples include aryl C₁-C₄-alkyl, such as aryl C₁-C₃-alkyl, preferably aryl-CH₂— and aryl-CH₂—CH₂—. Preferred examples of the aryl moiety include phenyl or naphthyl, more preferably phenyl. When the aryl moiety is substituted, it is preferably mono- or di-substituted. Suitable substituents are as defined herein, preferably C₁-C₇-alkyl, —O—C₁-C₇alkyl, halo-C₁-C₇alkyl, halo, cyano, hydroxy-C₁-C₇-alkyl, C₁-C₇alkoxy-C₁-C₇-alkoxy, C₁-C₇-alkanoylamino-C₁-C₇-alkyl, C₁-C₇ alkanoylamino, N—C₁-C₇alkoxy-C₁-C₇-alkyl-amino, N—C₁-C₇-alkanoyl-N—C₁-C₇-alkoxy-C₁-C₇-alkyl-amino, in particular, methyl, O-methyl, Cl, Br, CN, methoxypropyloxy, N(methoxypropyl)-amino, N(acetyl)-amino, and N(methoxypropyl)(acetyl)-amino. Most preferably aryl is unsubstituted or di-substituted with OMe.

When R3 is aryl alkyl, R1 is preferably present and as defined herein. When R3 is aryl alkyl, T is preferably C(O). When R3 is aryl alkyl, E is preferably CH₂. When R3 is aryl alkyl, G is preferably (CO)NR4 with R4 being H or C₁-C₇-alkyl such as methyl or ethyl. When R3 is aryl alkyl, the tricyclic moiety is preferably

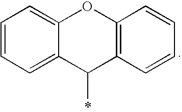

In a fifth embodiment, R3 is unsubstituted or substituted heterocyclyl alkyl. Examples include heterocyclyl C₁-C₄-alkyl, such as heterocyclyl C₁-C₃-alkyl, preferably heterocyclyl-CH₂— and heterocyclyl-CH₂—CH₂—. The heterocyclyl moiety preferably mono- or bicyclic. Preferred are saturated, aromatic, or partially saturated ring systems, in particular whereby one of the rings is aromatic and the other is saturated or partially saturated. The heterocyclyl moiety has preferably 1, 2 or 3, more preferably 1 or 2, heteroatoms selected from O, N or S, more preferably O or N. Particularly preferred examples include bicyclic preferably partially saturated 9- to 11-, preferably 10-, membered rings preferably containing an oxygen atom, in particular, 2,3-dihydro-benzo[1,4]dioxinyl, or monocyclic preferably aromatic or saturated 5- or 6-membered rings containing 1 or 2 heteroatoms selected from N and O, in particular, pyridyl, tetrahydrofuranyl, tetrahydropyranyl or [1,3]dioxalanyl, where each heterocyclyl is unsubstituted or substituted by one or more, e.g. up to three, substituents. Suitable substituents are as defined herein, preferably C₁-C₇-alkyl, —O—C₁-C₇-alkyl, halo-C₁-C₇-alkyl, halo, cyano, hydroxy-C₁-C₇-alkyl, C₁-C₇-alkoxy-C₁-C₇alkoxy, C₁-C₇alkanoylamino-C₁-C₇-alkyl, C₁-C₇-alkanoylamino, N—C₁-C₇-alkoxy-C₁-C₇-alkyl-amino, N—C₁-C₇-alkanoyl-N—C₁-C₇-alkoxy-C₁-C₇-alkyl-amino, in particular, methyl, O-methyl, Cl, Br, CN, methoxypropyloxy, N(methoxypropyl)-amino, N(acetyl)-amino, and N(methoxypropyl)(acetyl)-amino. Most preferably heterocyclyl is unsubstituted or substituted with OMe.

When R3 is heterocyclyl alkyl, R1 is preferably present and as defined herein. When R3 is heterocyclyl alkyl, T is preferably C(O). When R3 is heterocyclyl alkyl, E is preferably CH₂. When R3 is heterocyclyl alkyl, G is preferably (CO)NR4 with R4 being H or C₁-C₇-alkyl such as methyl, ethyl or propyl. When R3 is heterocyclyl alkyl the tricyclic moiety is preferably

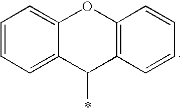

In a sixth embodiment, R3 is unsubstituted or substituted heterocyclyl. The heterocyclyl moiety preferably mono- or bicyclic, more preferably monocyclic. Preferred are saturated, aromatic, or partially saturated ring systems, in particular whereby one of the rings is aromatic and the other is saturated or partially saturated, most preferred is saturated. The heterocyclyl moiety has preferably 1, 2 or 3, more preferably 1 or 2, most preferably 1, heteroatoms selected from O, N or S, more preferably O or N. Particularly preferred examples include monocyclic preferably saturated 5- or 6-membered rings containing 1 or 2 heteroatoms selected from N and O, in particular, pyrrolidinyl and piperidinyl, where each heterocyclyl is unsubstituted or substituted by one or more, e.g. up to three, substituents. Suitable substituents are as defined herein, preferably C₁-C₇-alkyl, —O—C₁-C₇-alkyl, halo-C₁-C₇-alkyl, halo, cyano, hydroxy, hydroxy-C₁-C₇alkyl, C₁-C₇alkoxy-C₁-C₇alkoxy, C₁-C₇-alkoxy-C₁-C₇-alkyl, carboxy, C₁-C₇-alkoxycarbonyl, C₁-C₇-alkanoylamino-C₁-C₇-alkyl, C₁-C₇-alkanoylamino, N—C₁-C₇alkoxy-C₁-C₇-alkyl-amino, N—C₁-C₇alkanoyl-N—C₁-C₇alkoxy-C₁-C₇-alkyl-amino, in particular, hydroxyl, C₁-C₇-alkoxy-C₁-C₇-alkyl, C₁-C₇-alkanoylamino, C₁-C₇-alkoxycarbonyl. Most preferably heterocyclyl is unsubstituted or substituted with OH, —(CO)NH₂, —C(O)OMe or CH₂OMe.

When R3 is heterocyclyl, R1 is preferably present and as defined herein. When R3 is heterocyclyl, T is preferably C(O). When R3 is heterocyclyl, E is preferably CH₂. When R3 is heterocyclyl, G is preferably oxy. When R3 is heterocyclyl the tricyclic moiety is preferably

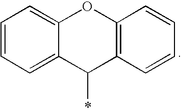

In one embodiment, G is preferably imino (NH), C(═O)NH or C(═O)NR4 wherein R4 is preferably C₁-C₄-alkyl or C₃-C₈-cycloalkyl-C₁-C₄-alkyl.

In a first embodiment G is oxy. When G is oxy, R1 is preferably present and as defined herein. When G is oxy, T is preferably C(O). When G is oxy, E is preferably CH₂. When G is oxy, R3 is preferably heterocyclyl. When G is oxy the tricyclic moiety is preferably

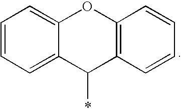

In a second embodiment, G is unsubstituted imino (NH).

In a third embodiment G is substituted imino (NR4) wherein R4 is preferably C₁-C₄-alkyl or C₃-C₈-cycloalkyl-C₁-C₄-alkyl. When G is substituted imino (NR4), R1 is preferably present and as defined herein. When G is substituted imino (NR4), T is preferably C(O). When G is substituted imino (NR4), E is preferably CH₂. When G is substituted imino (NR4), R3 is preferably acyl specifically as defined in (a) to (g), more preferably one of (a), (c), (e), (f) and (g), or is unsubstituted or substituted C₁-C₄-alkyl. When G is substituted imino (NR4) the tricyclic moiety is preferably

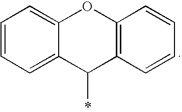

In a fourth embodiment, G is C(═O)NH or C(═O)NR4 wherein R4 is preferably C₁-C₄-alkyl or C₃-C₈-cycloalkyl-C₁-C₄-alkyl. When G is C(═O)NR4, R1 is preferably present and as defined herein. When G is C(═O)NH or C(═O)NR4, T is preferably C(O). When G is C(═O)NH or C(═O)NR4, E is preferably CH₂. When G is C(═O)NH or C(═O)NR4, R3 is preferably aryl alkyl, heterocyclyl alkyl, C₁-C₄-alkyl. Additionally, when G is C(═O)NH, R3 is also preferably cycloalkyl C₁-C₄-alkyl. When G is C(═O)NH or C(═O)NR4, the tricyclic moiety is preferably

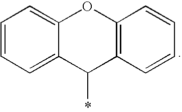

In one embodiment, R3 is preferably unsubstituted or substituted aryl, especially phenyl; or if G is NH or NR4, preferably NH, unsubstituted or substituted arylsulfonyl, e.g. C₁-C₇-alkyl-, halo- or (halo-C₁-C₇-alkyl)-phenylsulfonyl; or unsubstituted or substituted alkyloxycarbonyl, e.g. C₁-C₇-alkoxycarbonyl; or if G is C(═O)NH or C(═O)NR4, preferably C(═O)NH, C₁-C₇-alkyl or C₃-C₈-cycloalkyl-C₁-C₇-alkyl.

G is preferably imino (NH), C(═O)NH or C(═O)NR4 wherein R4 is preferably C₁-C₄-alkyl or C₃-C₈-cycloalkyl-C₁-C₄-alkyl.

In another possible preferred embodiment, G-R3 is hydrogen. However, preferred are compounds of the formula I wherein G-R3 has one of the meanings given in the present disclosure other than hydrogen.

### Preferred Definitions for R

R is selected from the group of moieties mentioned for R above or, if p=0, one or more, preferably 1 R can have one of the meanings given for R₁ above.

### Preferred Definitions for A and D

In the definition of A, CH₂O, CH₂S(O)₀₋₂, CH₂NH, C(═O)NH or SO₂NH includes both orientations of the respective moiety, that is, also the inverted orientation (as OCH₂, S(O)₀₋₂CH₂, NHCH₂, NHC(═O) or NHSO₂, respectively), but only one of these orientations if found in each molecule or single compound of formula I.

In the definition of D, CH═C*, CH₂CH*, *CHS(O)₀₋₂, CH₂N*, *CHNH, C(═O)N* or SO₂N* includes both possible orientations of the respective moiety, that is, also the inverted orientation (as *C═CH, *CHCH₂, S(O)₀₋₂C*H, *NCH₂, NHCH*, *NC(═O) or *NSO₂, respectively), but only one of these orientations if found in each molecule or single compound of formula I. The asterisk indicated the binding position of the bond to E.

Where a substituent Rx is present at A, this selected from C₁-C₇-alkyl#, hydroxy-C₁-C₇ alkyl#, C₁-C₄-alkoxy-C₁-C₄-alkyl#, hydroxy(#), halo, C₁-C₇-alkoxy(#), halo-C₁-C₇-alkyl#, amino(#), N-mono- or N,N-di-(C₁-C₄-alkyl)-amino(#), C₁-C₄-alkoxycarbonyl#, C₃-C₇-cycloalkyl# or C₃-C₇cycloalkyl-C₁-C₄-alkyl#, where especially only the moieties marked with # can be bound to a nitrogen, those marked with (#) with lower preference.

A is preferably O, CH₂ (methylene) or CH₂CH₂ (ethylene), most preferably O.

D is preferably CH, CR₁, N, CH═C or NHCH, most preferably CH or CR₁.

Very preferably, at least one heteroatom selected from O, S or N is present in the central ring with A and D in formula I, or this central ring has at least seven ring members.

Preferred examples of the rings formed by A an D are:

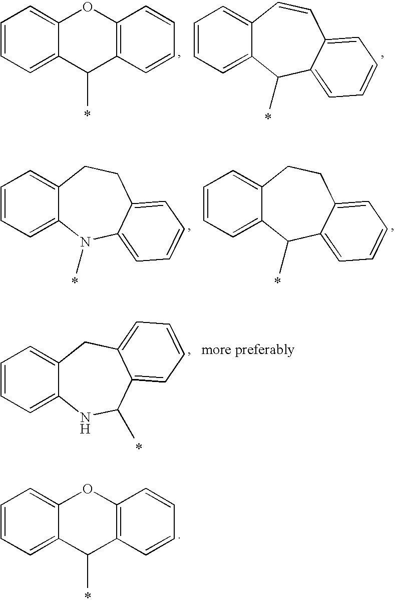

### Preferred Definitions of E and T

E is preferably methylene, ethylene, hydroxytrimethylene (especially 2-hydroxy-trimethylene) or carbonyl; preferably, if E is methylene, ethylene or hydroxytrimethylene, then T is methyllene or carbonyl; or if T is methylene, then E is carbonyl, methylene, ethylene or hydroxy-trimethylene. Most preferably, E is methylene.

T is preferably methyllene or carbonyl, most preferably carbonyl.

### Preferred Definitions of m, n and p

Each of m, n and p is preferably 0 or 1, more preferably either m is 0 (zero) and n is 1 or n is 1 and m is 0 (zero). In a preferred embodiment both n and m are 0. Preferably p is 0 or 1 more preferably 1.

In all definitions above the person having skill in the art will, without undue experimentation or considerations, be able to recognize which are relevant (e.g. those that are sufficiently stable for the manufacture of pharmaceuticals, e.g. having a half-life of more than 30 seconds or form stable tautomeric equilibria) and thus are preferably encompassed by the present claims and that only chemically feasible bonds and substitutions (e.g. in the case of double or triple bonds, hydrogen carrying amino or hydroxy groups and the like) are encompassed, as well as tautomeric forms where present. For example, preferably, for reasons of stability or chemical feasibility, in -G-R3 G and the atom binding as part of R3 are not simultaneously oxy plus oxy, thio plus oxy, oxy plus thio or thio plus thio. Substitutents binding via an O or S that is part of them are preferably not bound to nitrogen e.g. in rings.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous solutions, or can be isolated especially in solid, especially crystalline, form.

Such salts are formed, for example, as acid addition salts, preferably with organic or inorganic acids, from compounds of formula I with a basic nitrogen atom (e.g. imino or amino), especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds”, “starting materials” and “intermediates” hereinbefore and hereinafter, especially to the compound(s) of the formula I, is to be understood as referring also to one or more salts thereof or a mixture of a corresponding free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, starting materials, intermediates, salts, pharmaceutical preparations, diseases, disorders and the like, this is intended to mean one (preferred) or more single compound(s), salt(s), pharmaceutical preparation(s), disease(s), disorder(s) or the like, where the singular or the indefinite article (“a”, “an”) is used, this is intended to include the plural or preferably the singular.

The compounds of the present invention possess one or more or if G-R3 is other than hydrogen two or more asymmetric centers, depending on the choice of the substituents. The preferred absolute configurations are as indicated herein specifically. However, any possible isolated or pure diastereoisomers, enantiomers and geometric enantiomers, and mixtures thereof, e.g., racemates, are encompassed by the present invention.

As described herein above, the present invention provides 3,5-substituted piperidine derivatives of formula I, these compounds for use in the (prophylactic and/or therapeutic) treatment of a disease (=condition, disorder) in a warm-blooded animal, especially a human, preferably of a disease dependent on (especially inappropriate) renin activity, a pharmaceutical composition comprising a compound of the formula I, methods for preparing said compound or pharmaceutical preparation, and methods of treating conditions dependent on (especially inappropriate) renin activity by administration of a therapeutically effective amount of a compound of the formula I, or a pharmaceutical composition thereof.

“Inappropriate” renin activity preferably relates to a state of a warm-blooded animal, especially a human, where renin shows a renin activity that is too high in the given situation (e.g. due to one or more of misregulation, overexpression e.g. due to gene amplification or chromosome rearrangement or infection by microorganisms such as virus that express an aberrant gene, abnormal activity e.g. leading to an erroneous substrate specificity or a hyperactive renin e.g. produced in normal amounts, too low activity of renin activity product removing pathways, high substrate concentration and/or the like) and/or leads to or supports a renin dependent disease or disorder as mentioned above and below, e.g. by too high renin activity. Such inappropriate renin activity may, for example, comprise a higher than normal activity, or further an activity in the normal or even below the normal range which, however, due to preceding, parallel and or subsequent processes, e.g. signaling, regulatory effect on other processes, higher substrate or product concentration and the like, leads to direct or indirect support or maintenance of a disease or disorder, and/or an activity that supports the outbreak and/or presence of a disease or disorder in any other way. The inappropriate activity of renin may or may not be dependent on parallel other mechanisms supporting the disorder or disease, and/or the prophylactic or therapeutic effect may or may include other mechanisms in addition to inhibition of renin. Therefore “dependent” has to be read as “dependent inter alia”, (especially in cases where a disease or disorder is really exclusively dependent only on renin) preferably as “dependent mainly”, more preferably as “dependent essentially only”. A disease dependent on (especially inappropriate) activity of renin may also just be defined as one that responds to modulation of renin activity, especially responding in a beneficial way (e.g. lowering of the blood pressure and/or amelioration of the symptoms associated with any one or more of the other diseases mentioned herein) in case of renin inhibition.

Where a disease or disorder dependent on inappropriate activity of a renin is mentioned (such as in the definition of “use” in the following paragraph) and also especially where a compound of the formula I is mentioned for use in the diagnostic or therapeutic treatment which is preferably the treatment of a disease or disorder dependent on inappropriate renin activity, this refers preferably to any one or more diseases or disorders that depend on inappropriate activity of natural (especially human) renin and/or one or more altered, allelic or mutated forms thereof.

Where subsequently or above the term “use” is mentioned (as verb or noun) (relating to the use of a compound of the formula I or of a pharmaceutically acceptable salt thereof, or a method of use thereof), this (if not indicated differently or to be read differently in the context) includes any one or more of the following embodiments of the invention, respectively (if not stated otherwise): the use in the treatment of a disease or disorder that depends on (especially inappropriate) activity of renin, the use for the manufacture of pharmaceutical compositions for use in the treatment of a disease or disorder that depends on (especially inappropriate) activity of renin; a method of use of one or more compounds of the formula I in the treatment of a disease or disorder that depends on (especially inappropriate) activity of renin; a pharmaceutical preparation comprising one or more compounds of the formula I for the treatment of a disease or disorder that depends on (especially inappropriate) activity of renin; and one or more compounds of the formula I for use in the treatment of a disease or disorder in a warm-blooded animal, especially a human, preferably a disease that depends on (especially inappropriate) activity of renin; as appropriate and expedient, if not stated otherwise.

The terms “treat”, “treatment” or “therapy” refer to the prophylactic (e.g. delaying or pre-venting the onset of a disease or disorder) or preferably therapeutic (including but not limited to preventive, delay of onset and/or progression, palliative, curing, symptom-alleviating, symptom-reducing, patient condition ameliorating, renin-modulating and/or renin-inhibiting) treatment of said disease(s) or disorder(s), especially of the one or more disease or disorder mentioned above or below.

### PREFERRED EMBODIMENTS ACCORDING TO THE INVENTION

The groups of preferred embodiments of the invention mentioned below are not to be regarded as exclusive, rather, e.g., in order to replace general expressions or symbols with more specific definitions, parts of those groups of compounds can be interchanged or exchanged using the definitions given above, or omitted, as appropriate, and each of the more specific definitions, independent of any others, may be introduced independently of or together with one or more other more specific definitions for other more general expressions or symbols.

Particularly preferred is a compound of the formula I with a configuration given in the following formula IA:

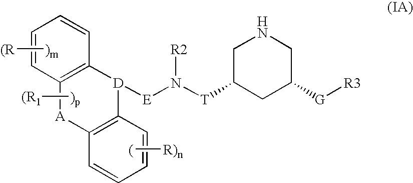

wherein R₁, R2, R3, R, A, D, E, T, G, m, n and p are as defined for a compound of the formula I, preferably as given under the preferred embodiments of a compound of the formula I, or a pharmaceutically acceptable salt thereof.

Preferred is a compound of the formula I wherein

R₁ if present (present if p=1) is preferably a substituent of the formula —(C₀-C₇-alkylene)(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen, C₁-C₇alkyl or phenyl- or naphthyl-C₁-C₇-alkyl;
or is C₂-C₇-alkenyl, C₂-C₇-alkynyl, phenyl, naphthyl, heterocyclyl, phenyl- or naphthyl- or heterocyclyl-C₁-C₇-alkyl or —C₁-C₇alkyloxy, di-(naphthyl- or phenyl)-amino-C₁-C₇-alkyl, di(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇alkyl wherein phenyl or naphthyl is unsubstituted or substituted by one or more, especially one to three, C₁-C₇alkyl moieties; phenyl- or naphthyl-C₁-C₇-alkylsulfonylamino-C₁-C₇-alkyl, carboxy-C₁-C₇-alkyl, halo, hydroxy, phenyl-C₁-C₇-alkoxy wherein phenyl is unsubstituted or substituted by C₁-C₇-alkoxy and/or halo, halo-C₁-C₇-alkoxy, phenyl- or naphthyloxy, phenyl- or naphthyl-C₁-C₇-alkyloxy, phenyl- or naphthyloxy-C₁-C₇-alkyloxy, benzoyl- or naphthoyloxy, halo-C₁-C₇-alkylthio, phenyl- or naphthylthio, phenyl- or naphthyl-C₁-C₇-alkylthio, benzoyl- or naphthoylthio, nitro, amino, di-(naphthyl- or phenyl-C₁-C₇alkyl)-amino, benzoyl- or naphthoylamino, phenyl- or naphthylsulfonylamino wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfonylamino, carboxyl, (N,N-) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N-) di-(C₁-C₇-alkyl)-amino-C₁-C₇alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇-alkyl)-aminocarbonyl, cyano, sulfenyl, sulfinyl, C₁-C₇-alkylsulfinyl, phenyl- or naphthylsulfinyl wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)aminosulfonyl;
R2 is hydrogen, C₁-C₇-alkyl or phenyl-C₁-C₇-alkyl wherein phenyl is unsubstituted or substituted by halo;
R3 is unsubstituted or substituted aryl, especially phenyl, unsubstituted or substituted C₃-C₈-cycloalkyl-C₁-C₇-alkyl, alkyl, especially C₁-C₇-alkyl, or, if G is NH, is unsubstituted or substituted arylsulfonyl, e.g. (C₁-C₇-alkyl)- or (halo-C₁-C₇-alkyl)-phenylsulfonyl, or alkoxycarbonyl, especially C₁-C₇-alkyloxycarbonyl;
R is C₁-C₄-alkyl, halo-C₁-C₄-alkyl, hydroxy, C₁-C₄-alkoxy, amino, N-mono- or N,N-di-(C₁-C₄-alkyl and/or alkanoyl)-amino, carbamoyl, sulfamoyl, cyano or especially halo; or, if p is zero, one R if present can be R₁ as defined above;
A is O, CH₂ or CH₂CH₂, where in each case an H is unreplaced or can be replaced by a moiety where in each case H is unreplaced (preferred) or one H can be replaced by a moiety Rx selected from C₁-C₄-alkyl, hydroxy-C₁-C₄-alkyl, C₁-C₄-alkoxy-C₁-C₄-alkyl, hydroxy, halo, C₁-C₄-alkoxy, halo-C₁-C₄-alkyl, amino, N-mono- or N,N-di-(C₁-C₄-alkyl)-amino, C₁-C₄-alkoxycarbonyl, C₃-C₇-cycloalkyl or C₃-C₇-cycloalkyl-C₁-C₄-alkyl;
D is N, CH, CH═C or NHCH, where in each case an H is unreplaced or can be replaced by a moiety R₁ as defined above if p is 1;
E is carbonyl or unsubstituted or (hydroxy or C₁-C₇-alkoxy)-substituted C₁-C₇alkylene;
T is carbonyl or methylene;
G is imino (NH) or C(═O)NH or C(═O)NR4 wherein R4 is C₁-C₇-alkyl or phenyl-C₁-C₇-alkyl;
or G-R3 together is hydrogen;
m is 0 or 1;
n is 0 or 1;
and p is 0 or 1;
or a pharmaceutically acceptable salt thereof.

More preferred is a compound of the formula I, wherein

R₁ if present (present if p=1) is C₁-C₇alkyl, C₁-C₇-alkoxy-C₁-C₇-alkyl or phenyl-C₁-C₇-alkyl; where if present R₁ is preferably bound as shown in formula I* above;
R2 is hydrogen or C₁-C₇-alkyl;
R3 is C₃-C₈-cycloalkyl-C₁-C₇-alkyl, especially cyclohexylmethyl, C₁-C₇-alkyl, especially methyl, or, if G is NH, is (C₁-C₇-alkyl)- or (halo-C₁-C₇alkyl)-phenylsulfonyl or C₁-C₇-alkoxycarbonyl;
R is halo, especially chloro;

### A is O, CH₂ or CH₂CH₂;

D is N, CH, CH═C or NHCH, where in each case an H is unreplaced or can be replaced by a moiety R₁ as defined above if p is 1;
E is carbonyl or unsubstituted or (hydroxy or C₁-C₇-alkoxy)-substituted C₁-C₇alkylene;
T is carbonyl or methylene;
G is imino (NH) or C(═O)NH;
m is 0 or 1;
n is 0 or 1;
and p is 0 or 1;
or a pharmaceutically acceptable salt thereof.

A different preferred group of compounds of the formula I refers to an analogue of the compounds described in the immediately preceding paragraph wherein R₁, R2, R, A, D, E, T, n, m and p are as defined there but G-R3 is hydrogen, or a pharmaceutically acceptable salt thereof.

Particular embodiments of the invention, especially of compounds of the formula I and/or salts thereof, are provided in the Examples—the invention thus, in a very preferred embodiment, relates to a compound of the formula I, or a salt thereof, selected from the compounds given in the Examples, as well as the use thereof.

### Process of Manufacture

A compound of formula I, or a salt thereof, is prepared analogously to methods that, for other compounds, are in principle known in the art, so that for the novel compounds of the formula I the process is novel at least as analogy process, especially as described or in analogy to methods described herein in the illustrative Examples, or modifications thereof, preferably in general by

(A) reacting a carbonic acid of the formula II,

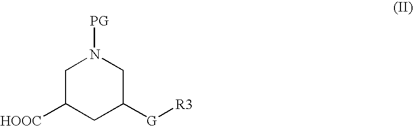

or a reactive derivative thereof, wherein R3 and G are as defined for a compound of the formula I and PG is a protecting group, especially tert-butoxycarbonyl or 9H-fluoren-9-ylmethoxycarbonyl, with an amine of the formula III,

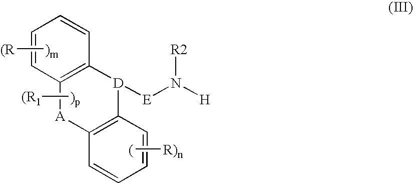

wherein R₁, R2, R, A, D, E, n, m and p are as defined for a compound of the formula I; or
(B) for the synthesis of a compound of the formula I wherein T is methylene and R₁, R2, R3, R, A, D, E, G, m, n and p have the meanings given above or below for a compound of the formula I, reacting an aldehyde of the formula IV,

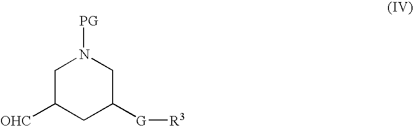

wherein R3 and G are as defined for a compound of the formula I and PG is a protecting group, especially tert-butoxycarbonyl or 9H-fluoren-9-ylmethoxycarbonyl, with an amino compound of the formula III as defined above under conditions for reductive amination;
or
   (C) for the synthesis of a compound of the formula I wherein G is imino, oxo or thio, reacting a compound of the formula V,

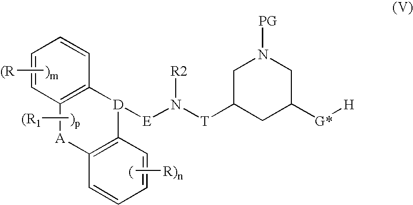

wherein R₁, R2, R, A, D, E, T, n, m and p are as defined for a compound of the formula I, G* is imino, oxy or thio and PG is a protecting group, especially tert-butoxycarbonyl or 9H-fluoren-9-ylmethoxycarbonyl, with a compound of the formula VI,

R3-LG (VI)

wherein R3 is as defined for a compound of the formula I and LG is a leaving group, or
(D) reacting a compound of the formula VII,

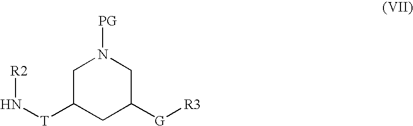

wherein R2, R3, G and T are as defined for a compound of the formula I and PG is a protecting group, with a compound of the formula VIII,

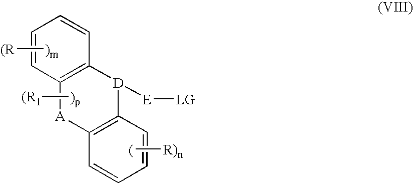

wherein R₁, R, A, D, E, m, n and p are as defined for a compound of the formula I and LG is a leaving group; or
(E) for the synthesis of a compound of the formula I wherein G is C(═O)NR4 or C(═O)NH and T is carboxy, reacting a compound of the formula IX,

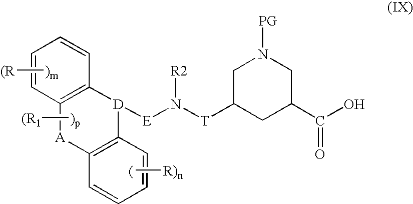

wherein R₁, R2, R, A, D, E, T, m, n and p are as defined for a compound of the formula I, with an amine of the formula X,

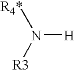

wherein R₄* is hydrogen or R4 as defined for a compound of the formula I hereinabove or hereinbelow and R3 is as defined for a compound of the formula I hereinabove or hereinbelow;
and, if desired, subsequent to any one or more of the process variants mentioned above converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;
where in any of the starting materials (especially of the formulae II to IV), in addition to specific protecting groups mentioned, further protecting groups may be present, and any protecting groups are removed at an appropriate stage in order to obtain a corresponding compound of the formula I, or a salt thereof.

### Preferred Reaction Conditions

The preferred reaction conditions for the reactions mentioned above, as well as for the transformations and conversions, are as follows (or analogous to methods used in the Examples or as described there)

The reaction under (A) between an acid of the formula II, or a reactive derivative thereof, and an amino compound of the formula III preferably takes place under customary condensation conditions, where among the possible reactive derivatives of an acid of the formula II reactive esters (such as the hydroxybenzotriazole (HOBT), pentafluorophenyl, 4-nitrophenyl or N-hydroxysuccinimide ester), acid halogenides (such as the acid chloride or bromide) or reactive anhydrides (such as mixed anhydrides with lower alkanoic acids or symmetric anhydrides) are preferred. Reactive carbonic acid derivatives can also and preferably be formed in situ. The reaction is carried out by dissolving the compounds of formulae II and III in a suitable solvent, for example a halogenated hydrocarbon, such as methylene chloride, N,N-dimethylformamide, N,N-dimethylacetamide, N-methyl-2-pyrrolidone or acetonitrile, or a mixture of two or more such solvents, and by the addition of a suitable base, for example triethylamine, diisopropylethylamine (DIEA) or N-methylmorpholine and, if the reactive derivative of the acid of the formula II is formed in situ, a suitable coupling agent that forms a preferred reactive derivative of the carbonic acid of formula III in situ, for example dicyclohexylcarbodiimide/1-hydroxybenzotriazole (DCC/HOBT); bis(2-oxo-3-oxazolidinyl)phosphinic chloride (BOPCl); O-(1,2-dihydro-2-oxo-1-pyridyl)-N,N,N′,N′-tetramethyluronium tetrafluoroborate (TPTU); O-benzotriazol-1-yl)-N,N,N′,N′-tetramethyluronium tetrafluoroborate (TBTU); (benzotriazol-1-yloxy)-tripyrrolidinophosphonium-hexafluorophosphate (PyBOP), O-(1H-6-chlorobenzotriazole-1-yl)-1,1,3,3-tetramethyluronium hexafluorophosphate, 1-(3-dimethylaminopropyl)-3-ethylcarbodiimide hydrochloride/hydroxybenzotriazole or/1-hydroxy-7-azabenzotriazole (EDC/HOBT or EDC/HOAt) or HOAt alone, or with (1-chloro-2-methyl-propenyl)-dimethylamine. For review of some other possible coupling agents, see e.g. Klauser; Bodansky, Synthesis 1972, 453-463. The reaction mixture is preferably stirred at a temperature of between approximately −20 and 50° C., especially between 0° C. and 30° C., e.g. at room temperature. The reaction is preferably carried out under an inert gas, e.g. nitrogen or argon.

In order to obtain a compound of the formula I if no further conversion is desired, the subsequent removal of a protecting group, e.g. PG, such as tert-butoxycarbonyl, benzyl, 9H-fluoren-9-ylmethoxycarbonyl or 2-(trimethylsilyl)-ethoxycarbonyl, takes place under standard conditions, see also the literature mentioned below under General Process Conditions. For example, tert-butoxycarbonyl is removed in the presence of an acid, e.g. a hydrohalic acid, such as HCl, in an appropriate solvent, e.g. an ether, such as dioxane, or an alcohol, e.g. isopropanol, at customary temperatures, e.g. at room temperature, the removal of benzyl can be achieved e.g. by reaction with ethylchloroformate in an appropriate solvent, e.g. toluene, at elevated temperatures, e.g. from 80 to 110° C., and subsequent removal of the resulting ethoxycarbonyl group by hydrolysis in the presence of a base, e.g. an alkali metal hydroxide, such as potassium hydroxide, in an appropriate solvent, e.g. in an alcohol, such as ethanol, at elevated temperatures, e.g. from 80 to 120° C., or by removal by means of trimethylsilyl trifluoroacetate in a tertiary nitrogen base, such as 2,6-lutidine, in the presence of an appropriate solvent, such as a halogenated hydrocarbon, e.g. methylene chloride, the removal of 2-(trimethylsilyl)-ethoxycarbonyl can be achieved, for example, by reaction with a tetra-lower alkylammonium fluoride, such as tetraethylammoniumfluoride, in an appropriate solvent or solvent mixture, e.g. a halogenated hydrocarbon, such as methylene chloride, and/or a nitrile, such as acetonitrile, preferably at elevated temperatures, e.g. under reflux conditions, and the removal of a 9H-fluoren-9-ylmethoxycarbonyl protecting group can be achieved in the presence of a secondary amine, especially piperidine, in an appropriate solvent, e.g. a halogenated hydrocarbons, such as methylene chloride, at preferred temperatures between 0 and 50° C., e.g. at about room temperature.

The reaction under (B) between an aldehyde of the formula IV with an amino compound of the formula III preferably takes place under customary conditions for reductive amination, e.g. in the presence of an appropriate reducing (e.g. hydrogenation) agent, such as hydrogen in the presence of a catalyst or a complex hydride, e.g. sodium triacetoxyborohydride or sodium cyanoborhydride, in an appropriate solvent, such as a halogenated hydrocarbon, e.g. methylene chloride or 1,2,-dichloroethane, and optionally a carbonic acid, e.g. acetic acid, at preferred temperatures between −10° C. and 50° C., e.g. from 0° C. to room temperature; where without further conversion the subsequent removal of protecting groups is required, this takes place e.g. as described above under (A) or as below under “General Process Conditions”.

The reaction under (C) above either, if R3 is acyl, especially with a carbonyl or sulfonyl group, takes place as described under (A) above under condensation conditions for the reaction of carbonic acids, especially where the leaving group LG is introduced in situ, or if R3 is acyl or has any other meaning given for R3 in a compound of the formula I and the leaving group LG is preferably selected from halo, e.g. chloro, from unsubstituted or substituted aryl-sulfonyloxy, such as toluolsulfonyloxy, from unsubstituted or substituted alkylsulfonyloxy, such as methylsulfonyloxy or trifluoromethylsulfonyloxy, and (if R3 is acyl) C₁-C₇-alkanoyloxy, e.g. acetyloxy, in the presence of a base, such as an alkali metal salt of a weaker acid, e.g. an alkali metal carbonate and/or an alkali metal hydrogencarbonate, such as sodium or potassium carbonate and/or sodium or potassium hydrogencarbonate (NaHCO₃ or KHCO₃) in an appropriate solvent, e.g. dioxane and/or H₂O, at preferred temperatures between −20 and 50° C., e.g. at −5 to 30° C.

The reaction under (D), if E is carbonyl, preferably takes place under conditions as described under (A) above, especially where the leaving group LG is introduced in situ, replacing an OH group present instead of the LG in a starting material of the formula VIII; or under conditions analogous to those described under (C) above. If E is methylene, then LG is preferably selected from halo, e.g. chloro, from unsubstituted or substituted aryl-sulfonyloxy, such as toluolsulfonyloxy, and from unsubstituted or substituted alkylsulfonyloxy, such as methylsulfonyloxy or trifluoromethylsulfonyloxy, and the reaction can, for example, take place in the presence of a base, such as an alkali metal salt of a weaker acid, e.g. an alkali metal carbonate and/or an alkali metal hydrogencarbonate, such as sodium or potassium carbonate and/or sodium or potassium hydrogencarbonate (NaHCO₃ or KHCO₃) in an appropriate solvent, e.g. dioxane and/or H₂O, at preferred temperatures between −20 and 50° C., e.g. at −5 to 30° C.

The reaction under (E) preferably takes place under conditions corresponding to those mentioned under process variant (A) above.

### Optional Reactions and Conversions

Compounds of the formula I, or protected forms thereof directly obtained according to any one of the preceding procedures or after introducing protecting groups anew, which are included subsequently as starting materials for conversions as well even if not mentioned specifically, can be converted into different compounds of the formula I according to known procedures, where required after removal of protecting groups.

Where R2 is hydrogen in a compound of the formula I, this can be converted into the corresponding compound wherein R2 has a meaning other than hydrogen given for compounds of the formula I by reaction with a compound of the formula XI,

R2*-Q (XI)

wherein R2* is defined as R2 in a compound of the formula I other than hydrogen and Q is a leaving group (e.g. as defined for LG under reactions (C) and (D) above), or wherein Q is —CHO (so that the compound of the formula XI is an aldehyde) and then R2* is the complementary moiety for a moiety R2 that includes a methylene group (resulting in a group R2 of the formula R2*-CH₂—) e.g. under reaction conditions as follows: The reductive amination preferably takes place under customary conditions for reductive amination, e.g. in the presence of an appropriate hydrogenation agent, such as hydrogen in the presence of a catalyst or a complex hydride, e.g. sodium triacetoxyborohydride or sodium cyanoborhydride, in an appropriate solvent, such as a halogenated hydrocarbon, e.g. methylene chloride or 1,2,dichloroethane, and optionally a carbonic acid, e.g. acetic acid, at preferred temperatures between −10° C. and 50° C., e.g. from 0° C. to room temperature.

Hydroxy substituents, e.g. as substitutents of aryl in alkyl substituted by aryl R2, R3 or in other aryl substituents, can be transformed into unsubstituted or substituted alkoxy, e.g. by alkylation reaction with the corresponding unsubstituted or substituted alkylhalogenide, e.g. iodide, in the presence of a base, e.g. potassium carbonate, in an appropriate solvent, e.g. N,N-dimethylformamide, e.g. at preferred temperatures between 0 and 50° C.

Carboxy substitutents can be converted into esterified carboxy by reaction with corresponding alcohols, e.g. C₁-C₇-alkanols, or into amidated carboxy by reaction with corresponding amines, e.g. under condensation conditions analogous to those described above under reaction (A).

Esterified carboxy substituents can be converted into free carboxy by hydrolysis, e.g. in the presence of a base, such as potassium hydroxide, in an appropriate solvent, e.g. tetrahydrofurane, preferably at elevated temperatures, e.g. from 50° C. to the reflux temperature of the reaction mixture.

A moiety -G-R3 wherein G is O and R3 is hydrogen can be converted into amino by first converting the —OH into a leaving group, e.g. by halogenation or preferably by reaction with an organic sulfonylhalogenide, such as methylsulfonylchloride, in the presence of a tertiary nitrogen base, such as triethylamine, and in the presence of an appropriate solvent, e.g. dichloromethane, preferably at lower temperatures, e.g. in the range from −30 to 20° C., followed by reaction with an alkali metal azide, e.g. sodium azide, in an appropriate solvent, such as dichloromethane, in the presence of a tertiary nitrogen base, e.g. triethylamine, and preferably at lower temperatures, e.g. in the range from −30 to 20° C. to give the corresponding azido group, which is then converted into the amino group e.g. by reaction with triphenylphosphine in an appropriate solvent, e.g. tetrahydrofurane in the presence of water, at preferably lower temperatures, e.g in the range from −30 to 20° C.

A group -G-R3 wherein G is NH and R5 is H (thus being amino) can be converted into the corresponding group wherein G is NH and R3 is unsubstituted or substituted alkyl or acyl by alkylation or acylation. For example, acylation may take place using the corresponding acid halogenide (e.g. the chloride) in the presence of a tertiary nitrogen base, such as triethylamine, in an appropriate solvent, such as dichloromethane, preferably at lower temperatures, e.g. in the range from −30 to 20° C.

Carbonyl groups such as carbonyl E, T or G can be converted to the corresponding methyllene, especially by treatment with a complex hydride, e.g. borane dimethylsulfide complex, in an appropriate solvent, such as an ether, e.g. tetrahydrofurane, at preferred temperatures between room temperature and the reflux temperature of the reaction mixture or at 140-150° C.

In some cases, the conversions preferably take place with compounds of the formula I in protected form; the subsequent removal of protecting group can be achieved as described above for reaction (A) and below under “General Process Conditions”, yielding a corresponding compound of the formula I.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers or enantiomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

Intermediates and final products can be worked up and/or purified according to standard methods, e.g. using chromatographic methods, distribution methods, (re-) crystallization, and the like.

### Starting Materials

In the subsequent description of starting materials and intermediates and their synthesis, R₁, R2, R3, R4, R₄*, R, A, D, E, G, G*, T, n, m, p, PG, LG and/or Q, have the meanings given above or especially in the Examples for the respective starting materials or intermediates, if not indicated otherwise directly or by the context. Protecting groups, if not specifically mentioned, can be introduced and removed at appropriate steps in order to prevent functional groups, the reaction of which is not desired in the corresponding reaction step or steps, employing protecting groups, methods for their introduction and their removal are as described above or below, e.g. in the references mentioned under “General Process Conditions”. The person skilled in the art will readily be able to decide whether and which protecting groups are useful or required.

A compound of the formula II wherein G is imino, oxy or thio and R3 is acyl can, for example, be prepared by reacting a compound of the formula XII,

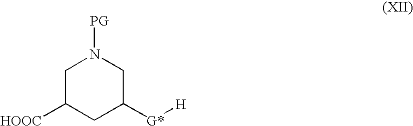

wherein G* is imino, oxy or thio, with an acyl compound of the formula VI as defined above under reaction (C) and under reaction conditions as mentioned under (C) above. The compound of the formula XII can be prepared from a corresponding compound of the formula XIII,

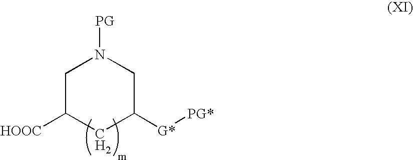

wherein PG* is a protecting group (which may also be a moiety R3 itself which is then not a protecting group as its removal is not desired), e.g. tert-butoxycarbonyl, by removal of the protecting group PG* under standard conditions, e.g. as described above under process (A) or below under “General Process Conditions”.

If PG* is not to be removed but is a moiety R3 itself, the compound of the formula XIII is itself a compound of the formula II.

A compound of the formula XIII can, for example, be prepared by reducing a pyridine compound of the formula XIV,

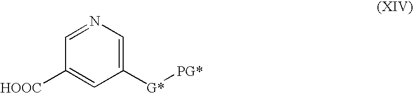

wherein G* and PG* are as defined for a compound of the formula XIII, in the presence of an appropriate reductant, especially hydrogen in the presence of a catalyst, such as Rh and/or Pt oxides, e.g. Nihismura's catalyst ([Rh(III)oxide/Pt(IV) oxide hydrate) in an appropriate solvent, e.g. water in the presence of ammonium hydroxide, at preferred temperatures e.g. in the range from 0 to 50° C., e.g. at about room temperature, giving a compound of the formula XV,

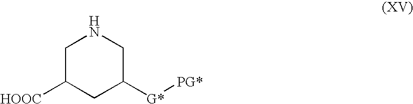

or a salt thereof, which is then protected by introduction of a protecting group PG, e.g. Fmoc, under customary conditions, e.g. as described below under “General Process Conditions”, e.g. by reaction of N-Fmoc-succinimide in the presence of a base, e.g. sodium hydrogencarbonate, in an appropriate solvent, e.g. water and/or tetrahydrofurane, thus yielding the corresponding compound of the formula XIII.

Compounds of the formula II wherein G-R3 is as defined for compounds of the formula I can be prepared analogous to compounds of the formula XIII starting from analogues of a compound of the formula XIV wherein instead of G*-PG* a moiety G-R3 is present as defined for a compound of the formula I.

If desired or synthetically useful, PG in any one compound of the formula II (or in any other intermediate useful in the synthesis of a compound of the formula I according to the invention where it is present) can be replaced by a different protecting group; for example, Fmoc as PG can first be removed, e.g. as described above under process (A), and then be replaced with Boc, e.g. using di-tert-butylcarbonate in the presence of a base, such as potassium hydrogencarbonate, in an appropriate solvent, e.g. dioxane, e.g. at temperatures in the range from 0 to 50° C., such as at about room temperature; or benzyl may be removed by hydrogenation in the presence of an appropriate noble metal catalyst, e.g. Pd(OH)₂ on charcoal, in an appropriate solvent, e.g. an alcohol, such as ethanol, and then replaced with Boc by reaction of the product as just described.

An aldehyde compound of the formula IV can, for example be prepared from a compound of the formula II by reduction of the carboxy group to the formyl group, e.g. by first reducing it to a hydroxymethyl group with an appropriate complex hydride, such as borane dimethylsulfide complex, in an appropriate solvent, e.g. THF, at lower temperatures, e.g. from −50 to 10° C., and subsequent oxidation of the hydroxymethyl group with an appropriate oxidant, e.g. Dess-Martin periodinane, in an appropriate solvent, e.g. dichloromethane, at preferred temperatures in the range from 0 to 50° C., e.g. at about room temperature.

A compound of the formula VII can, for example, be prepared starting from a compound of the formula IV by reacting it with an amine of the formula XVI,

R2-NH₂ (XVI)

Under conditions of reductive amination, e.g. analogous to those mentioned under reaction (B) above.

Starting materials of the formula III can, for example, be prepared starting from a compound of the formula XVII,

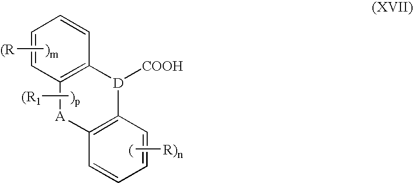

or a reactive derivative thereof, wherein R₁, R, A, D, m, n and p are as defined for a compound of the formula I, by first reacting them under condensation conditions analogous to those given under (A) above, where the reactive derivatives of the carbonic acid of the formula XXVII may be of the types as given under reaction (A) above and can also be provided in situ as described under reaction (A) above, with a compound of the formula XVI described above; this results in a compound of the formula III wherein E is carbonyl. In order to achieve a corresponding compound of the formula III wherein E is methylene, the carbonyl can be reduced by reaction with a reductant, e.g. a complex hydride, for example lithium aluminumhydride, preferably in the presence of a Lewis base, such as aluminium chloride, in an appropriate solvent, such as methyllene chloride, at preferred temperatures in the range from −10 to 60° C., or with borane dimethylsulfide complex in an appropriate solvent, e.g. THF, at elevated temperatures, e.g. from 100 to 160° C.;

Where instead of a group R2 in formula XVI a corresponding compound is used wherein this group is replaced with a protecting group, this protecting group can then be removed, e.g. a benzyl group by catalytic hydrogenation, e.g. with hydrogen in the presence of a noble metal catalyst, such as palladium on charcoal in an appropriate solvent, such as an alcohol, e.g. methanol or ethanol, in the absence or presence of a corresponding ammonium alcoholate, at preferred temperatures in the range from 0 to 80° C., e.g. from room temperature to 60° C., thus providing a corresponding compound of the formula III wherein R2 is hydrogen.

A compound of the formula XVII, wherein D is preferably CH, E is carbonyl and wherein R₁ is bound at the carbon atom binding to E and which thus has the formula XVIIA,

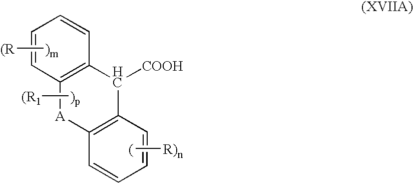

can be reacted to the corresponding ester of the formula XVIII,

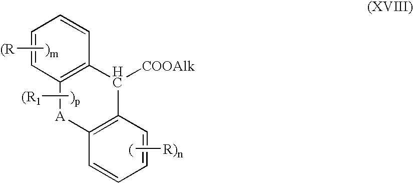

wherein Alk is unsubstituted or substituted alkyl, preferably C₁-C₇alkyl, C₁-C₇-alkoxy-C₁-C₇-alkyl or phenyl-C₁-C₇alkyl, for example by reaction in the presence or absence of cesium carbonate, in an appropriate solvent, such as N,N-di-(methyl)-formamide, at preferred temperatures in the range from 0 to 50° C., e.g. at about room temperature, with the corresponding unsubstituted or substituted alkyl halogenide, e.g. Alk-I or Alk-Br, such as C₁-C₇-alkyliodide, C₁-C₇-alkoxy-C₁-C₇-alkyl-iodide or phenyl-C₁-C₇alkyl-iodide

E.g. starting from a compound of the formula XVIII, it is then possible to obtain the corresponding compound of the formula XIX,

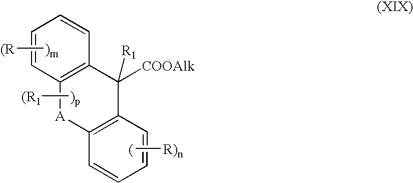

by reacting with a compound of the formula XX,

R₁-Hal (XX)

wherein Hal is halogen, such as bromo or iodo, in the presence of a strong base, e.g. lithium diisopropylamine, in an appropriate solvent, such as tetrahydrofurane and/or 4-methylphosphotriamide, at low temperatures, e.g. from −90 to −30° C., e.g. at about 78° C.; from a compound of the formula XIX thus obtained, the Alk can then be cleaved off, e.g. in the presence of an alkali metal hydroxide, such as sodium hydroxide, in an appropriate solvent, e.g. water, at temperatures e.g. between 20 and 80° C., e.g. at about 60° C. The result is a compound of the formula XVIIB,

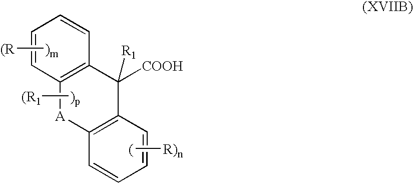

which falls under compound XVII and is thus a starting material for a corresponding compound of the formula III.

If for the reaction described above between a compound of the formula XVIII and a compound of the formula XX instead of a compound of the formula XX a compound of the formula XXI,

R1ₐ-Hal (XXI)

is used wherein Hal is halogen, especially bromo or iodo, and R1ₐ is alkenyl, preferably C₃-C₇-alkenyl, with a double bond preferably not at the carbon at which Hal is bound, under conditions as with a compound of the formula XX, it is possible to obtain a compound of the formula XXII,

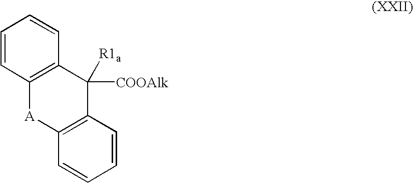

wherein R1ₐ is as just described and Alk is defined as for formula XX, the moiety R1ₐ can then be converted into the corresponding hydroxyalkyl moiety by reaction e.g. with 9-borabicyclo[3.3.1]nonane, preferably under an inert gas, e.g. Argon, in an appropriate solvent, e.g. tetrahydrofurane, at temperatures from −10 to 60° C., followed by addition of H₂O₂ and an alkali metal hydroxide, e.g. sodium hydroxide; the hydroxy group can then, if desired, be converted into a C₁-C₇-alkyloxy group by reaction with a corresponding C₁-C₇-alkylhalogenide, e.g. iodide, in the presence of a strong base, such as sodium hydride, in an appropriate solvent, e.g. N,N-di-(methyl)-formamide, preferably at temperatures from −20 to 30° C., e.g. at about 0° C. This and subsequent hydrolysis of the —COOAlk as described for the manufacture of a compound of the formula XIXB above results in a corresponding compound of the formula XVIIB wherein R₁ is C₁-C₇-alkoxy-alkyl.

Starting materials of the formula VIII can, for example, be obtained by converting (either in situ or in an independent reaction) the carboxyl function in a compound of the formula XVII, e.g. with a corresponding acid anhydride, into the compound of the formula VIII wherein E is carbonyl or by reduction, e.g. by reducing it to a hydroxymethyl group with an appropriate complex hydride, such as borane dimethylsulfide complex, in an appropriate solvent, e.g. THF, at lower temperatures, e.g. from −50 to 10° C., into the corresponding hydroxymethyl compound wherein the hydroxy moiety can then be replaced with LG according to well-known procedures to give a corresponding compound of the formula VIII wherein E is methylene.

Starting materials of the formula IX wherein T is carbonyl (C(═O)) can, for example, be obtained as shown below under “Examples” in Scheme VI under reaction conditions analogous to those given in the respective Examples. The acid anhydride starting materials of the formula XXIII can, for example, be obtained as shown in Scheme 7 in the Examples and under the reaction conditions for the synthesis of the compound of the formula XXIII.

Other starting materials, e.g. of the formula VI, IX, X or XI, their synthesis or analogous methods for their synthesis are known in the art, they are commercially available, and/or they can be found in or derived from the Examples.

### General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their introduction and removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Novel starting materials, especially those that lead to preferred compounds of the formula I, also form a preferred embodiment according to the invention. Special preference is given to reaction conditions that are identical or analogous to those mentioned in the Examples.

### Pharmaceutical Use, Pharmaceutical Preparations and Methods

As described above, the compounds of the formula I (also occasionally called “compounds of the invention” hereinafter) are inhibitors of renin activity and, thus, may be employed for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like.

The present invention further provides pharmaceutical compositions comprising a therapeutically effective amount of a pharmacologically active compound of the formula I (or a pharmaceutically acceptable salt thereof), alone or in combination with one or more pharmaceutically acceptable carriers.

The pharmaceutical compositions according to the present invention are those suitable for enteral, such as oral or rectal, transdermal and parenteral administration to mammals, including man, to inhibit renin activity, and for the treatment of conditions associated with (especially inappropriate) renin activity. Such conditions include hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders and the like.

Thus, the pharmacologically active compounds of the invention may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbants, colorants, flavors and sweeteners.

Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and pre-determined rate over a prolonged period of time, and means to secure the device to the skin.

Accordingly, the present invention provides pharmaceutical compositions as described above for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, as well as methods of their use.

The pharmaceutical compositions may contain a therapeutically effective amount of a compound of the formula I as defined herein, either alone or in a combination with another therapeutic agent, e.g., each at an effective therapeutic dose as reported in the art. Such therapeutic agents include:

a) antidiabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) anti-obesity agents such as orlistat; and
d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibittors; ACE/NEP inhibitors such as omapatrilat, sampatrilat and fasidotril; angiotensin II antagonists such as candesartan, eprosartan, irbesartan, losartan, telmisartan and valsartan, in particular valsartan; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors.

Other specific anti-diabetic compounds are described by Patel Mona in Expert Opin Investig Drugs, 2003, 12(4), 623-633, in the FIGS. 1 to 7, which are herein incorporated by reference. A compound of the present invention may be administered either simultaneously, before or after the other active ingredient, either separately by the same or different route of administration or together in the same pharmaceutical formulation.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention alone or in combination with a therapeutically effective amount of another therapeutic agent, preferably selected from antidiabetics, hypolipidemic agents, anti-obesity agents or anti-hypertensive agents, most preferably from antidiabetics, anti-hypertensive agents or hypolipidemic agents as described above.

The present invention further relates to pharmaceutical compositions as described above for use as a medicament.

The present invention further relates to use of pharmaceutical compositions or combinations as described above for the preparation of a medicament for the treatment of conditions mediated by (especially inappropriate) renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders, and the like.

Thus, the present invention also relates to a compound of formula I for use as a medicament, to the use of a compound of formula I for the preparation of a pharmaceutical composition for the prevention and/or treatment of conditions mediated by (especially inappropriate) renin activity, and to a pharmaceutical composition for use in conditions mediated by (especially inappropriate) renin activity comprising a compound of formula I, or a pharmaceutically acceptable salt thereof, in association with a pharmaceutically acceptable diluent or carrier material therefore.

The present invention further provides a method for the prevention and/or treatment of conditions mediated by (especially inappropriate) renin activity, which comprises administering a therapeutically effective amount of a compound of the present invention to a warm-blooded animal, especially a human, in need of such treatment.

A unit dosage for a mammal of about 50-70 kg may contain between about 1 mg and 1000 mg, advantageously between about 5-600 mg of the active ingredient. The therapeutically effective dosage of active compound is dependent on the species of warm-blooded animal (especially mammal, more especially human), the body weight, age and individual condition, on the form of administration, and on the compound involved.

In accordance with the foregoing the present invention also provides a therapeutic combination, e.g., a kit, kit of parts, e.g., for use in any method as defined herein, comprising a compound of formula I, or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition comprising a compound of the formula I according to the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula I, or a pharmaceutically acceptable salt thereof, and at least a second drug substance, said second drug substance preferably being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to a modulation of (especially inappropriate) renin activity.

Preferably, the condition associated with (especially inappropriate) renin activity is selected from hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth and/or hyperaldosteronism, and/or further cognitive impairment, alzheimers, dementia, anxiety states and cognitive disorders.

Finally, the present invention provides a method or use which comprises administering a compound of formula I in combination with a therapeutically effective amount of an anti-diabetic agent, a hypolipidemic agent, an anti-obesity agent or an anti-hypertensive agent.

Ultimately, the present invention provides a method or use which comprises administering a compound of formula I in the form of a pharmaceutical composition as described herein.

The above-cited properties are demonstrable in vitro and in vivo tests using advantageously mammals, e.g., mice, rats, rabbits, dogs, monkeys or isolated organs, tissues and preparations thereof. Said compounds can be applied in vitro in the form of solutions, e.g., preferably aqueous solutions, and in vivo either enterally, parenterally, advantageously intravenously, e.g., as a suspension or in aqueous solution. The concentration level in vitro may range between about 10⁻³ molar and 10⁻¹⁰ molar concentrations. A therapeutically effective amount in vivo may range depending on the route of administration, between about 0.001 and 500 mg/kg, preferably between about 0.1 and 100 mg/kg.

As described above, the compounds of the present invention have enzyme-inhibiting properties. In particular, they inhibit the action of the natural enzyme renin. Renin passes from the kidneys into the blood where it effects the cleavage of angiotensinogen, releasing the decapeptide angiotensin I which is then cleaved in the lungs, the kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume which increase can be attributed to the action of angiotensin II. Inhibitors of the enzymatic activity of renin lead to a reduction in the formation of angiotensin I, and consequently a smaller amount of angiotensin II is produced. The reduced concentration of that active peptide hormone is a direct cause of the hypotensive effect of renin inhibitors.

The action of renin inhibitors may be demonstrated inter alia experimentally by means of in vitro tests, the reduction in the formation of angiotensin I being measured in various systems (human plasma, purified human renin together with synthetic or natural renin substrate).

Inter alia the following in vitro tests may be used:

Recombinant human renin (expressed in Chinese Hamster Ovary cells and purified using standard methods) at 7.5 nM concentration is incubated with test compound at various concentrations for 1 h at RT in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 2 μM and increase in fluorescence is recorded at an excitation wave-length of 350 nm and at an emission wave-length of 500 nm in a microplate spectro-fluorimeter. IC₅₀ values are calculated from percentage of inhibition of renin activity as a function of test compound concentration (Fluorescence Resonance Energy Transfer, FRET, assay). Compounds of the formula I, in this assay, preferably can show IC₅₀ values in the range from 1 nM to 15 μM.

Alternatively, recombinant human renin (expressed in Chinese Hamster Ovary cells and purified using standard methods) at 0.5 nM concentration is incubated with test compound at various concentrations for 2 h at 37° C. in 0.1 M Tris-HCl buffer, pH 7.4, containing 0.05 M NaCl, 0.5 mM EDTA and 0.05% CHAPS. Synthetic peptide substrate Arg-Glu(EDANS)-Ile-His-Pro-Phe-His-Leu-Val-Ile_His_Thr-Lys(DABCYL)-Arg9 is added to a final concentration of 4 μM and increase in fluorescence is recorded at an excitation wave-length of 340 nm and at an emission wave-length of 485 nm in a microplate spectro-fluorimeter. IC50 values are calculated from percentage of inhibition of renin activity as a function of test compound concentration (Fluorescence Resonance Energy Transfer, FRET, assay). Compounds of the formula I, in this assay, preferably can show IC₅₀ values in the range from 1 nM to 15 μM.

In another assay, human plasma spiked with recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC50 values are calculated from percentage of inhibition of renin activity as a function of test compound concentration. Compounds of the formula I, in this assay, preferably can show IC₅₀ values in the range from 1 nM to 15 μM.

In another assay, recombinant human renin (expressed in Chinese Hamster Ovary cells and purified using standard methods) at 0.8 nM concentration is incubated with test compound at various concentrations for 2 h at 37° C. in 0.1 M Tris/HCl pH 7.4 containing 0.05 M NaCl, 0.5 mM EDTA and 0.025% (w/v) CHAPS. Synthetic peptide substrate Ac-Ile-His-Pro-Phe-His-Leu-Val-Ile-His-Asn-Lys-[DY-505-X5] is added to a final concentration of 2.5 μM. The enzyme reaction is stopped by adding an excess of a blocking inhibitor. The product of the reaction is separated by capillary electrophoresis and quantified by spectrophotometric measurement at 505 nM wave-length. IC₅₀ values are calculated from percentage of inhibition of renin activity as a function of test compound concentration. Compounds of the formula I, in this assay, preferably show IC₅₀ values in the range from 1 nM to 15 μM.

In animals deficient in salt, renin inhibitors bring about a reduction in blood pressure. Human renin may differ from the renin of other species. In order to test inhibitors of human renin, primates, e.g., marmosets (Callithrix jacchus) may be used, because human renin and primate renin are substantially homologous in the enzymatically active region. Inter alia the following in vivo tests may be used:

Compounds can be tested in vivo in primates as described in the literature (see for example by Schnell C R et al. Measurement of blood pressure and heart rate by telemetry in conscious, unrestrained marmosets. Am. J. Physiol. 264 (Heart Circ. Physiol. 33). 1993: 1509-1516; or Schnell C R et al. Measurement of blood pressure, heart rate, body temperature, ECG and activity by telemetry in conscious, unrestrained marmosets. Proceedings of the fifth FELASA symposium: Welfare and Science. Eds. BRIGHTON. 1993.

The following Examples serve to illustrate the invention without limiting the scope thereof:

### ABBREVIATIONS

abs. absolute
Ac acetyl
aq. aqueous
Bn benzyl
Boc tert-butoxycarbonyl
Bop-Cl bis(2-oxo-3-oxazolidinyl)phosphinic chloride
Brine sodium chloride solution saturated at RT
NBu n-butyl
Celite=Celite® (The Celite Corporation)=filtering aid based on diatomaceous earth
c-hexane cyclohexane
DIPEA N,N-diisopropyl-N-ethylamine
dist. distilled
DMP Dess-Martin periodinane
eq. equivalent
Et ethyl
Fmoc 9H-fluoren-9-ylmethoxycarbonyl
h hour(s)
HCTU O-(1H-6-chlorobenzotriazole-1-yl)-1,1,3,3-tetramethyluronium hexafluorophosphate
HMPA hexamethylphosphoroamide
HPLC High Performance Liquid Chromatography
LC-MS Liquid Chromatography-Mass Spectroscopy
Me methyl
min minute(s)
MS Mass Spectroscopy
NMP 1-methyl-2-pyrrolidinone
Nucleodur=Nucleodur®, (Macherey & Nagel, Düren, FRG; HPLC column material based on highly pressure and pH resistant silica)
Nucleosil Nucleosil® (Macherey & Nagel, Düren, FRG; HPLC-column material based on silica gel)
PyBOP (benzotriazol-1-yloxy)tripyrrolidinophosphonium-hexafluorophosphate
Rf Ratio of fronts in TLC
Rp reversed phase
RT room temperature
sat. saturated
TFA trifluoroacetic acid
THF tetrahydrofuran
TLC thin layer chromatography
t_{R} retention time
   TLC conditions: R_{f} values for TLC are measured on 5×10 cm TLC plates, silica gel F₂₅₄, Merck, Darmstadt, Germany.
   HPLC conditions:

### Condition-A

### Column: Nucleosil 100-3 C18 HD, 125×4.0 mm.

Flow rate: 1.0 ml/min
Mobile phase: A) TFA/water (0.1/100, v/v), B) TFA/acetonitrile (0.1/100, v/v)
Gradient: linear gradient from 20% B to 100% B in 7 min

### Detection: UV at 254 nm

### Condition-B

### Column: ACQUITY UPLC™ BEH C₁₈ 1.7 μm, 50×2.1 mm.

Flow rate: 0.5 ml/min
Mobile phase: A) TFA/water (0.1/100, v/v), B) TFA/acetonitrile (0.1/100, v/v)
Gradient: linear gradient from 5% B to 100% B in 2 min then 100% B in 1 min

### Detection: UV at 254 nm

Temperatures are measured in degrees Celsius. Unless otherwise indicated, the reactions take place at about RT.

Note that (if the names of the compounds do not indicate the contrary or indicated otherwise) formulae of intermediates and compounds of the formula I represent only one enantiomer or racemic mixtures.

### Starting Materials

### Intermediate 1

### 5-tert-Butoxycarbonylamino-1-piperidine-3-carboxylate, ammonium salt

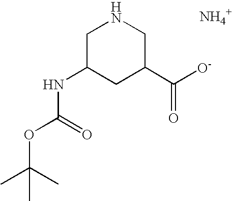

A mixture of 5-tert-butoxycarbonylamino-nicotinic acid (31.8 g, 0.133 mol), Nishimura's catalyst [Rh(III)oxide/Pt(IV) oxide hydrate] (6.37 g) in dist. H₂O (445 mL) and 25% NH₄OH solution (125 mL) is shaken at RT under H₂ for 65 h. After addition of a second portion of catalyst (6.37 g) shaking is continued for 25 h. The reaction mixture is filtered through Celite and evaporated in vacuo to yield the title compound as a white powder.

MS: 245.1 [M+H]⁺

### Intermediate 2

### (3S*,5R*)-5-tert-Butoxycarbonylamino-piperidine-1,3-dicarboxylic acid 1-(9H-fluoren-9-ylmethyl) ester and (3R*,5R*)-5-tert-Butoxycarbonylamino-piperidine-1,3-dicarboxylic acid 1-(9H-fluoren-9-ylmethyl) ester

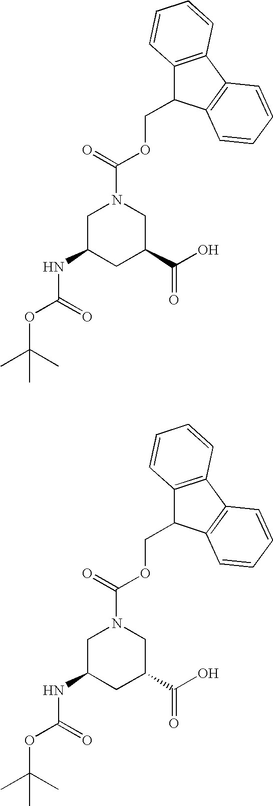

To a stirred mixture of 5-tert-butoxycarbonylamino-1-piperidine-3-carboxylate, ammonium salt (31.76 g, 0.122 mol), NaHCO₃ (10.22 g, 0.122 mol), dist. H₂O (145 mL) and THF (290 mL), N-(9-fluorenylmethoxycarbonyloxy)-succinimide (49.25 g, 0.146 mol) is added in several portions. The reaction mixture is stirred for 22 h at RT and the pH value then adjusted to 6 by the addition of 1M aqueous HCl. The mixture is diluted with H₂O and extracted with ethyl acetate. The organic phase is washed twice with brine, dried (Na₂SO₄) and evaporated. Crystallisation of the residue from ethyl acetate/hexane yields (3S*,5R*)-5-tert-butoxycarbonylamino-piperidine-1,3-dicarboxylic acid 1-(9H-fluoren-9-ylmethyl) ester as a white powder. t_{R} (HPLC, Nucleosil C18, 5-100% CH₃CN+0.1% TFA/H₂O+0.1% TFA for 8 min, 100% CH₃CN+0.1% TFA for 2 min, flow 1.5 ml/min): 6.64 min. For MS, a sample is treated with TFA/CH₂Cl₂ for 10 min. MS: 367.0 [M+H—C₅H₈O₂]⁺

The filtrate comprises an about 1:1 mixture of cis and trans isomers. Separation of the isomers by preparative HPLC (Nucleodur C18, 40-100% CH₃CN+0.1% TFA/H₂O+0.1% TFA for 36 min) affords, besides the above described (3S*,5R*)-cis-isomer, the trans isomer (3R*,5R*)-5-tert-butoxycarbonylamino-piperidine-1,3-dicarboxylic acid 1-(9H-fluoren-9-ylmethyl) ester as a white powder. t_{R} (HPLC, Nucleosil C18, 5-100% CH₃CN+0.1% TFA/H₂O+0.1% TFA for 8 min, 100% CH₃CN+0.1% TFA for 2 min, flow 1.5 ml/min): 6.58 min

### Preparation of Amines:

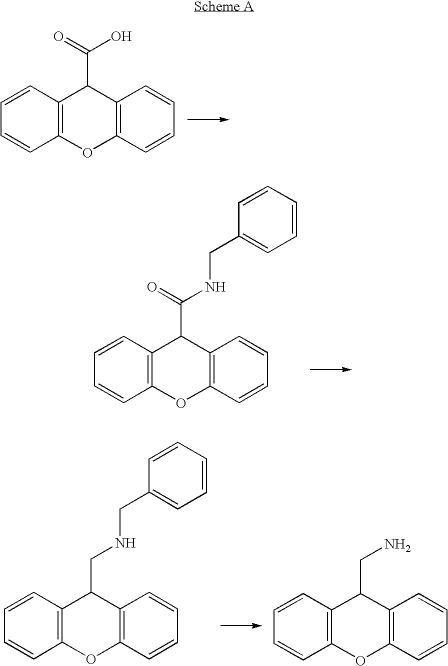

### A1) 9H-Xanthene-9-carboxylic acid benzylamide

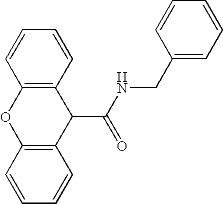

To a solution of 9H-Xanthene-9-carboxylic acid (10 g, 44.2 mmol) in CH₂Cl₂ (100 mL), 2-ethoxy-1-ethoxycarbonyl-1,2-dihydrochinolin (16.4 g, 66 mmol) and after 30 min benzylamine (19.3 mL, 177 mmol) are added. The mixture is stirred at room temperature overnight. The mixture is washed with aqueous 1 N HCl, saturated aqueous NaHCO₃ and brine. The organic layer is dried over Na₂SO₄, filtered and concentrated under reduced pressure to give a solid. Ethyl acetate is added to the solid, and the suspension is stirred for 30 min, filtered, washed with ethyl acetate and dried under vacuum overnight to give the title compound as a white solid. MS (LC-MS): 316 [M+H]⁺; TLC, Rf (hexane/AcOEt 1/1)=0.75.

### A2) Benzyl-(9H-xanthen-9-ylmethyl)-amine

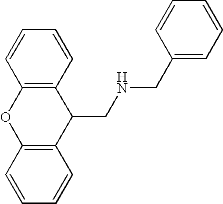

Aluminium trichloride (7.1 g, 53 mmol) is added in small portions to a suspension of LiAlH₄ (6.7 g, 178 mmol) in THF (30 mL) at 0° C. The mixture is stirred for 10 min at 0° C., before a solution of 9H-xanthene-9-carboxylic acid benzylamide (5.6 g, 17.8 mmol) in THF (26 mL) is added dropwise at 0° C. The mixture is heated to 50° C. for 5 h before it is cooled to RT and treated with aqueous NaOH (15%) and filtered. The filter cake is washed with ethyl acetate, and the solution is washed with saturated NaHCO₃. The aqueous phase is extracted three times with ethyl acetate and the combined organic phases are dried over Na₂SO₄, filtered and evaporated. The resulting residue is purified by flash chromatography on silica gel (eluent: CH₂Cl₂ to CH₂Cl₂/MeOH 98:2) to give the title compound as a yellow oil. MS (LC-MS): 302 [M+H]⁺; TLC, Rf (dichloromethane)=0.39.

### A3) Amine A: C-(9H-Xanthen-9-yl)-methylamine

The title compound is prepared according to Scheme A

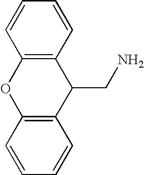

A mixture of benzyl-(9H-xanthen-9-ylmethyl)-amine (3.8 g, 12.6 mmol), palladium on charcoal (1 g, 10%) and ethanol (40 ml) is stirred at atmospheric pressure under hydrogen overnight. The mixture is filtered and the solvent evaporated to yield the title compound as a yellow oil. MS (LC-MS): 212 [M+H]⁺; TLC, Rf (dichloromethane/MeOH 95/5)=0.23.

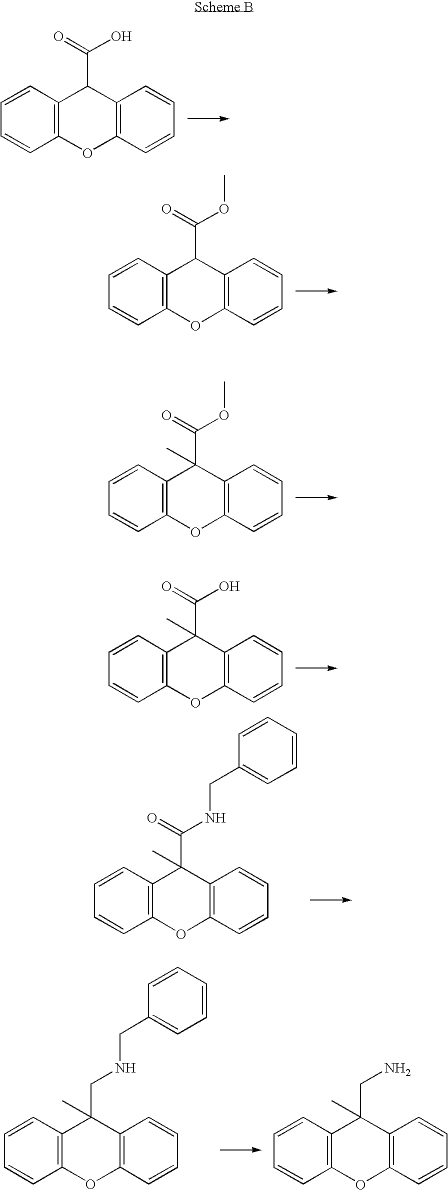

### B1) 9H-Xanthene-9-carboxylic acid methyl ester

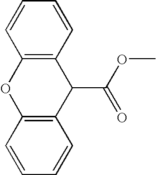

To a suspension of 9H-xanthene-9-carboxylic acid (20 g, 88.4 mmol) and Cs₂CO₃ (34.5 g, 106 mmol) in dimethyl formamide (300 mL), methyl iodide (8.3 mL, 133 mmol) is added dropwise at RT. The reaction is stirred for 1 h before it is quenched with water and extracted with diethyl ether. The organic phase is washed twice with water and with brine, dried over Na₂SO₄, filtered and evaporated to yield the title compound as a yellow solid. TLC, Rf (hexane/ethyl acetate 2/1)=0.72.

### B2) 9-Methyl-9H-xanthene-9-carboxylic acid methyl ester

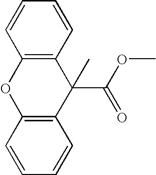

A solution of 9H-xanthene-9-carboxylic acid methyl ester (5 g, 20.8 mmol) in THF (15 mL) is added dropwise at −78° C. to a freshly prepared solution of lithium diisopropylamine (22 mmol) in THF (103 mL), and the mixture is stirred for 30 min at −78° C. Hexamethyl phosphotriamide (7.3 mL, 41.6 mmol) is added, and the reaction is stirred at −78° C. for 30 min. Methyl iodide is added at −78° C. and the reaction is stirred at −78° C. for 1 h. The reaction mixture is quenched with saturated aqueous NH₄Cl solution and extracted with diethyl ether. The organic phase is washed with water, dried over Na₂SO₄ filtered and evaporated. The resulting residue is purified by flash chromatography on silica gel (eluent: hexane/ethyl acetate 4:2) to give the title compound as white crystals. TLC, Rf (hexane/ethyl acetate 4/1)=0.62.

### B3) 9-Methyl-9H-xanthene-9-carboxylic acid

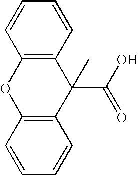

A mixture of 9-methyl-9H-xanthene-9-carboxylic acid methyl ester (500 mg, 1.97 mmol) in dioxane (2 mL) and aqueous sodium hydroxide (2 M, 2 mL) is stirred at 60° C. overnight. The solvent is evaporated, then dichloromethane and aqueous HCl (1 M) are added. The organic layer is washed with brine, dried over Na₂SO₄, filtered and evaporated to give the title compound as a white solid which is used directly and without characterization in the next step.

### B4) 9-Methyl-9H-xanthene-9-carboxylic acid benzylamide

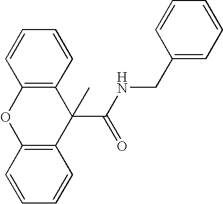

To a solution of 9-methyl-9H-xanthene-9-carboxylic acid (300 mg, 1.25 mmol) in dimethylformamide (4 mL) at 0° C., PyBOP (975 mg, 1.87 mmol) and after 5 min a solution of benzyl amine (0.27 ml, 2.5 mmol) in dimethylformamide (1 mL) are added. The reaction mixture is stirred at room temperature overnight, treated with saturated aqueous NaHCO₃ solution and extracted with dichloromethane. The organic layer is dried over Na₂SO₄, filtered and concentrated under reduced pressure to give a solid. The resulting residue is purified by flash chromatography on silica gel (eluent: hexane/ethyl acetate 9/1 to 4/1) to give the title compound as a white solid. MS (LC-MS): 330 [M+H]⁺; TLC, Rf (hexane/ethyl acetate 9/1)=0.16.

### B5) Benzyl-(9-methyl-9H-xanthen-9-ylmethyl)-amine

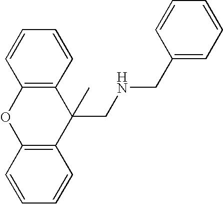

A solution of 9-methyl-9H-xanthene-9-carboxylic acid benzylamide (1.6 g, 4.8 mmol) and borane dimethylsulfide complex (2 M, 6.1 mL) in THF (20 mL) is heated in a microwave oven to 150° C. for 15 min before it is quenched with water at room temperature. Aqueous HCl (1 M) is added and the mixture is stirred overnight before it is neutralized with saturated NaHCO₃ solution and extracted with ethyl acetate. The organic layer is dried over Na₂SO₄, filtered and concentrated under reduced pressure to give a solid, which is purified by flash chromatography on silica gel (eluent: dichloromethane to dichloromethane/methanol 98/2) to give the title compound as a light yellow oil. MS (LC-MS): 316 [M+H]⁺; TLC, Rf (dichloromethane/methanol 98/2)=0.28.

### B6) C-(9-Methyl-9H-xanthen-9-yl)-methylamine

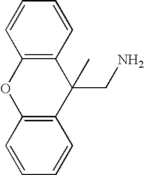

A mixture of benzyl-(9-methyl-9H-xanthen-9-ylmethyl)-amine (200 mg, 0.63 mmol), palladium on charcoal (10%, 68 mg) and ammonium formate in methanol (2.3 mL) is stirred at 60° C. for 1 h before it is filtered and evaporated to give the title compound as a solid. MS (LC-MS): 226 [M+H]⁺.

### C) Amine C: C-[9-(3-Methoxy-propyl)-9H-xanthen-9-yl]-methylamine

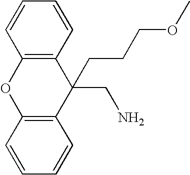

The title compound is prepared analogously as described for C-(9-methyl-9H-xanthen-9-yl)-methylamine using 3-methoxy-1-bromo-propane instead of methyl iodide in step B2. MS: 284 [M+H]⁺.

### D) Amine D: C-[9-(4-Methoxy-butyl)-9H-xanthen-9-yl]-methylamine

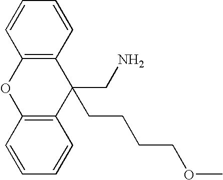

### D1. 9-But-3-enyl-9H-xanthene-9-carboxylic acid methyl ester

9-But-3-enyl-9H-xanthene-9-carboxylic acid methyl ester is prepared analogously as described for C-(9-methyl-9H-xanthen-9-yl)-methylamine under B2 from 9H-xanthene-9-carboxylic acid methyl ester and 4-bromo-1-butene.

### D2. 9-(4-Hydroxy-butyl)-9H-xanthene-9-carboxylic acid methyl ester

A solution of 9-but-3-enyl-9H-xanthene-9-carboxylic acid methyl ester (9.96 g, 33.8 mmol) in THF (85 mL) is added to 9-borabicyclo[3.3.1]nonane under argon and stirred at 40° C. for 5 min until a clear solution is obtained. The mixture is stirred at room temperature for 1 h before it is cooled to 0° C. and water (5 mL) is added dropwise. The reaction is kept at 0° C. while aqueous H₂O₂ (30%, 21.6 mL, 210 mmol) and subsequently aqueous NaOH (2 M, 68 mL) are added dropwise. The resulting mixture is stirred at room temperature for 30 min, before it is diluted with ethyl acetate and washed with aqueous NaHSO₄, NaHSO₃, NaHCO₃ and NaCl. The organic phase is dried over Na₂SO₄, filtered and evaporated. The resulting solid is purified by flash chromatography on silica gel (eluent: hexane/ethyl acetate 4/1 to hexane/ethyl acetate 1/1) to give the title compound as a white solid. TLC, Rf (hexane/ethyl acetate 1/1)=0.51. MS (LC-MS): 313 [M+H]⁺.

### D3. 9-(4-Methoxy-butyl)-9H-xanthene-9-carboxylic acid methyl ester

NaH (55%, 1.13 g, 25.9 mmol) is added to a solution of 9-(4-hydroxy-butyl)-9H-xanthene-9-carboxylic acid methyl ester (5.4 g, 17.3 mmol) and methyl iodide (3.3 mL, 52 mmol) in DMF (50 mL) at 0° C. The mixture is stirred at room temperature overnight before it is quenched with water and extracted with ethyl acetate. The organic phase is dried over Na₂SO₄, filtered and evaporated to give the title compound as an oil. TLC, Rf (hexane/ethyl acetate 4/1)=0.5. MS (LC-MS): 327 [M+H]⁺

### D4. C-[9-(4-Methoxy-butyl)-9H-xanthen-9-yl]-methylamine

C-[9-(4-Methoxy-butyl)-9H-xanthen-9-yl]-methylamine is prepared analogously as described for C-(9-Methyl-9H-xanthen-9-yl)-methylamine (Amine B) under B3 to B6 6 from 9-(4-methoxy-butyl)-9H-xanthene-9-carboxylic acid methyl ester. TLC, Rf (dichloromethane/methanol 95:5)=0.24. MS (LC-MS): 298 [M+H]⁺

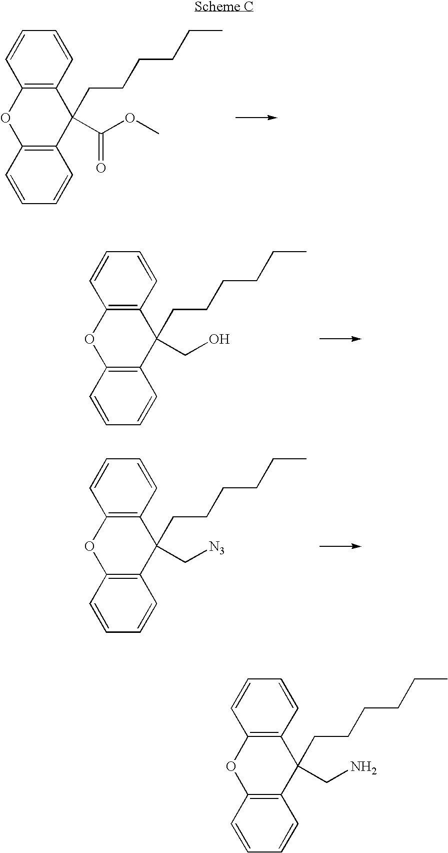

### E1. (9-Hexyl-9H-xanthen-9-yl)-methanol

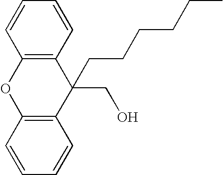

To a suspension of lithium aluminum hydride (222 mg, 5.86 mmol) in dry THF was added a solution of 9-hexyl-9H-xanthene-9-carboxylic acid methyl ester (1.90 g, 5.86 mmol) in tetrahydrofuran (10 mL) at room temperature. After stirred for 1.5 hours, the reaction mixture was quenched with addition of Na₂SO₄ 10H₂O (2 g) at 0° C. The mixture was filtered and washed with ethylacetate. The filtrate was concentrated under reduced pressure to afford the titled compound as a colorless oil which was used directly and without characterization in the next step.

### E2. [9-(3-Ethoxy-propyl)-9H-xanthen-9-yl]-methanol

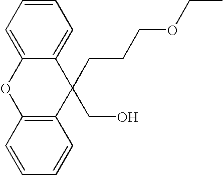

The title compound was prepared analogously as described for (9-hexyl-9H-xanthen-9-yl)methanol using 9-(3-ethoxy-propyl)-9H-xanthene-9-carboxylic acid methyl ester instead of 9-hexyl-9H-xanthene-9-carboxylic acid methyl ester. HPLC (Condition-B) t_{R}=1.96 min; MS: 281 [M+H]⁺.

### E3. 9-Azidomethyl-9-hexyl-9H-xanthene

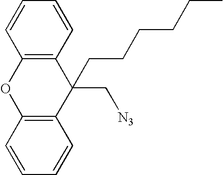

To a mixture of (9-hexyl-9H-xanthen-9-yl)-methanol (1.60 g, 5.40 mmol) and triphenylphosphine (1.70 g, 6.48 mmol) in THF (20 mL) was added azodicarboxylic acid diethyl ester (40% in toluene, 2.94 mL, 6.48 mmol) at room temperature. After the mixture was stirred for 10 minutes, diphenylphosphoryl azide (1.78 g, 6.48 mmol) was added at room temperature. The mixture was stirred for 24 hours, and then water was added. The product was extracted with ethylacetate and the organic layer was washed with brine. After dried over Na₂SO₄ and filtered, the solvent was removed under reduced pressure. The resulted residue was purified by silicagel column chromatography (ethylacetate/hexane=1/1) to isolate the titled compound which was used directly and without characterization in the next step.

### E4. C-(9-Hexyl-9H-xanthen-9-yl)-methylamine

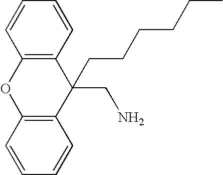

To a solution of 9-azidomethyl-9-hexyl-9H-xanthene (1.10 g, 3.42 mmol) dissolved in THF (20 mL) was added triphenylphosphine (1.80 g, 6.85 mmol) followed by water (0.62 mL). The mixture was stirred at room temperature for 16 hours. After adding water, the mixture was extracted with ethylacetate. The organic layer was washed with water, dried over Na₂SO₄, filtered, and concentrated under reduced pressure. The obtained residue was purified by silica gel column chromatography to afford the titled compound. HPLC (condition-B) t_{R}=1.80 min; MS; 296 [M+H]⁺.

### E5. C-[9-(3-Ethoxy-propyl)-9H-xanthen-9-yl]-methylamine

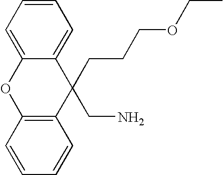

The title compound was prepared analogously as described for C-(9-hexyl-9H-xanthen-9-yl)methylamine using [9-(3-ethoxy-propyl)-9H-xanthen-9-yl]-methanol instead of C-(9-hexyl-9H-xanthen-9-yl)-methylamine in step E3 and 9-azidomethyl-9-(3-ethoxy-propyl)-9H-xanthene instead of 9-Azidomethyl-9-hexyl-9H-xanthene in step E4. HPLC (Condition-B) t_{R}=1.56 min, MS; 298 [M+H]⁺.

### E6. C-[9-(2-Methoxy-ethoxymethyl)-9H-xanthen-9-yl]-methylamine

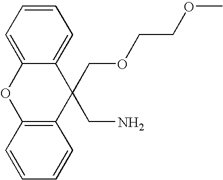

The title compound was prepared analogously as described for C-(9-hexyl-9H-xanthen-9-yl)methylamine using [9-(3-methoxy-ethyoxymethyl)-9H-xanthen-9-yl]-methanol instead of C-(9-hexyl-9H-xanthen-9-yl)-methylamine in step E3 and 9-azidomethyl-9-(2-methoxyethoxymethyl)-9H-xanthene instead of 9-azidomethyl-9-hexyl-9H-xanthene in step E4. HPLC (Condition-B) t_{R}=1.65 min, MS: 300 [M+H]⁺.

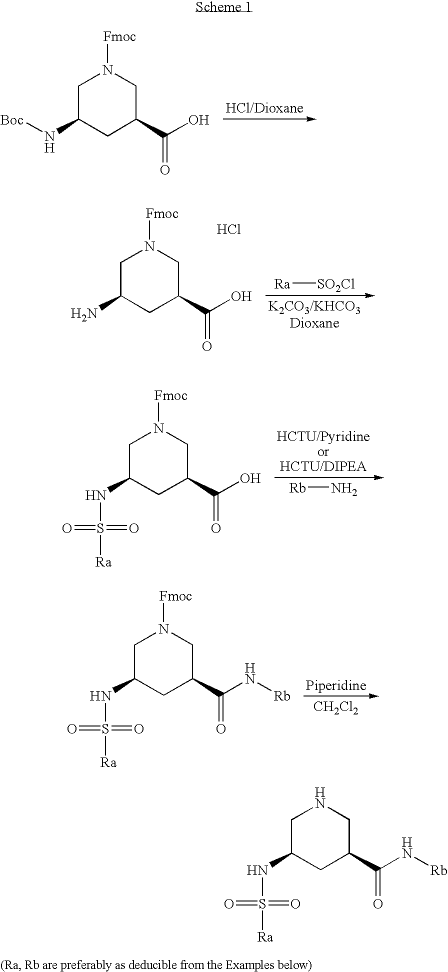

### Example 1

### (3S*,5R*)-5-(Toluene-4-sulfonylamino)-piperidine-3-carboxylic acid (9H-xanthen-9-ylmethyl)-amide

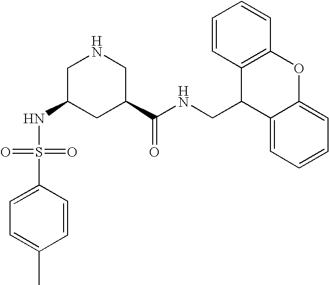

A solution of (3R*,5S*)-(toluene-4-sulfonylamino)-5-[(9H-xanthen-9-ylmethyl)-carbamoyl]-piperidine-1-carboxylic acid 9H-fluoren-9-ylmethyl ester in CH₂Cl₂/piperidine (14 mL) is stirred for 1 h at RT. After evaporation in vacuo, the residue is purified by flash chromatography (CH₂Cl₂/MeOH/NH₃ 50/6/1) to afford the title compound as a white solid. MS: 492 [M+H]⁺, TLC Rf (CH₂Cl₂/MeOH/NH3 50/6/1)=0.33.

The starting material is prepared as follows:

### 1.A. (3S*,5R*)-5-Amino-piperidine-1,3-dicarboxylic acid 1-(9H-fluoren-9-ylmethyl) ester, hydrochloride

To a mixture of (3S*,5R*)-5-tert-butoxycarbonylamino-piperidine-1,3-dicarboxylic acid 1-(9H-fluoren-9-ylmethyl) ester (Intermediate 2) (4.7 g, 10.1 mmol) in dioxane (25 mL), HCl (4M in dioxane, 25 mL, 100 mmol) is added and the reaction mixture is stirred for 16 h RT. Hexane (50 mL) is added and the crystals are filtered off, washed with hexane and dried in vacuo to afford the title compound as a white solid. MS: 367.4 [M+H]⁺; t_{R} (HPLC, Nucleosil C18; 5-100% CH₃CN+0.1% TFA/H₂O+0.1% TFA for 8 min, flow 1.5 ml/min): 4.48 min.

### 1.B. (3S*,5R*)-5-(Toluene-4-sulfonylamino)-piperidine-1,3-dicarboxylic acid 1-(9H-fluoren-9-ylmethyl) ester

To a stirred, ice-cooled mixture of (3S*,5R*)-5-amino-piperidine-1,3-dicarboxylic acid 1-(9H-fluoren-9-ylmethyl) ester hydrochloride (9.67 g, 24 mmol), K₂CO₃ (4.98 g, 36 mmol in 50 mL of H₂O), KHCO₃ (3.6 g, 36 mmol in 36 mL of H₂O and dioxane (85 mL), 4-toluenesulfonyl chloride (5.03 g, 26.4 mmol) is added in several portions at a temperature of 0-5° C. Stirring is continued at this temperature for 1 h. The reaction mixture is diluted with H₂O and acidified with HCl to pH 2. The aqueous phase is extracted three times with ethyl acetate. After washing with brine, the combined organic extracts are dried (Na₂SO₄) and the solvent is evaporated in vacuo to afford the title compound as a white solid. MS: 521.1 [M−H]⁻; t_{R} (HPLC, Waters Symmetry C18; 5-100% CH₃CN+0.05% TFA/H₂O+0.05% TFA for 6 min, flow 1.5 ml/min): 4.66 min.

### 1.C. (3R*,5S*)-(toluene-4-sulfonylamino)-5-[(9H-xanthen-9-ylmethyl)-carbamoyl]-piperidine-1-carboxylic acid 9H-fluoren-9-ylmethyl ester

To a stirred, ice-cooled solution of (3S*,5R*)-5-(toluene-4-sulfonylamino)-piperidine-1,3-dicarboxylic acid 1-(9H-fluoren-9-ylmethyl) ester (300 mg, 0.576 mmol) in CH₂Cl₂ (6 mL), DIPEA (49.3 mL, 0.288 mmol) is added, followed by HCTU (260 mg, 0.628 mmol) in CH₃CN (6 mL). The mixture is stirred for 15 min at 0° C. After the addition of C-(9H-xanthen-9-yl)methylamine (121.7 mg, 0.576 mmol) stirring is continued for 1 h at 0° C. and then for 14 h at RT. The suspension is filtered and the filtrate evaporated. The residue thus obtained is distributed between saturated NaHCO₃ solution and ethyl acetate. The aqueous layer is extracted with ethyl acetate. The combined organic layers are washed with 2N HCl and brine, dried over Na₂SO₄, filtered and evaporated. Flash chromatography (CH₂Cl₂/MeOH) affords the title compound as a beige solid. TLC, Rf (CH₂Cl₂/MeOH 9/1)=0.35.

### Example 2

### (3S*,5R*)-5-(Toluene-4-sulfonylamino)-piperidine-3-carboxylic acid (9-methyl-9H-xanthen-9-ylmethyl)-amide

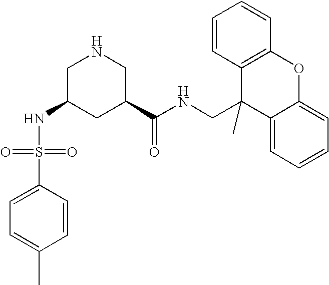

The title compound is prepared analogously as described in Example 1 using C-(9-methyl-9H-xanthen-9-yl)-methylamine instead of C-(9H-xanthen-9-yl)-methylamine. MS: 506 [M+H]⁺ TLC, Rf (CH₂Cl₂/MeOH/NH3 50/6/1)=0.53.

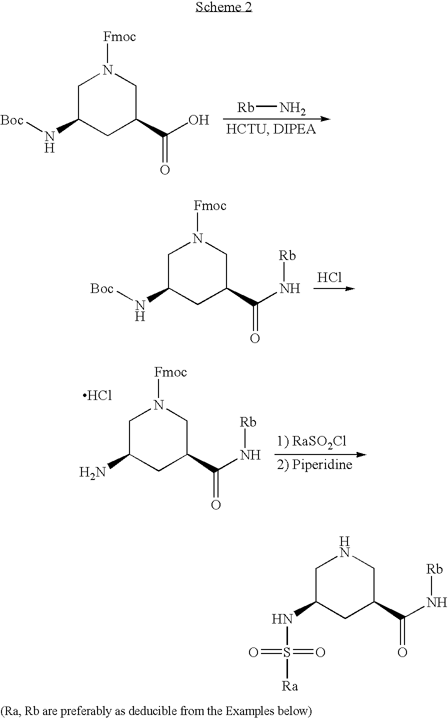

### General Procedure, Scheme 2

To a stirred, ice-cooled mixture of (3S*,5R*)-5-tert-butoxycarbonylamino-piperidine-1,3-dicarboxylic acid 1-(9H-fluoren-9-ylmethyl) ester (1 eq.) in CH₂Cl₂, N-ethyldiisopropylamine (0.5 eq.) is added, followed by O-(1H-6-chlorobenzotriazole-1-yl)-1,1,3,3-tetramethyluronium hexafluorophosphate (1.09 eq.) in CH₃CN. The mixture is stirred for 15 min at 0° C. After the addition of the corresponding amine (1 eq.), stirring is continued for 1 h at 0° C. and then for 14 h at RT. The reaction mixture is distributed between saturated NaHCO₃ solution and ethyl acetate. The aqueous layer is extracted with ethyl acetate. The combined organic layers are washed with 2N HCl and brine, dried over Na₂SO₄, filtered and evaporated. A mixture of the product thus obtained and HCl (4M in dioxane, ˜30 eq.) is stirred for 1 h at RT. The mixture is evaporated to dryness. The remaining product is dissolved in pyridine and cooled in an ice bath. After addition of 4-dimethylaminopyridine (0.3 eq.) and the corresponding sulfonyl chloride (4 eq.), the ice bath is removed, and the mixture is stirred at RT for 14 h. The reaction mixture is diluted with H₂O and acidified with 1N HCl to pH 2, and the aqueous layer is extracted three times with ethyl acetate. The combined organic layers are washed with brine, dried (Na₂SO₄) and evaporated to afford the title compound as a white amorphous solid. The crude compound is stirred during 1 h at RT with a freshly prepared solution of CH₂Cl₂/piperidine 4:1. The reaction mixture is evaporated in vacuo and the residue purified by preparative HPLC chromatography (YMC-Pack Pro C18 column, 150 mm×30 mm, 5 μM; 10-100% CH₃CN+0.1% TFA/H₂O+0.1% TFA, 20 min, flow 20 ml/min).

### Example 3

### (3S*,5R*)-5-(3-Chloro-benzenesulfonylamino)-piperidine-3-carboxylic acid [9-(3-methoxy-propyl)-9H-xanthen-9-ylmethyl]-amide

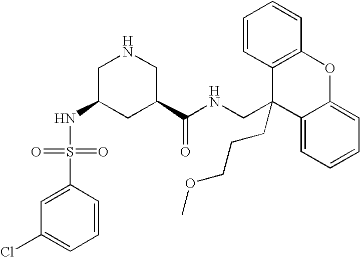

The title compound is prepared as described under “General Procedure, Scheme 2” using C-[9-(3-methoxy-propyl)-9H-xanthen-9-yl]-methylamine and 3-chlorobenzenesulfonyl chloride. MS: 585 [M+H]⁺; TLC, Rf CH₂Cl₂/MeOH/NH₃ 50/6/1)=0.5.

### Example 4

### (3S*,5R*)-5-(Toluene-4-sulfonylamino)-piperidine-3-carboxylic acid [9-(4-methoxy-butyl)-9H-xanthen-9-ylmethyl]-amide

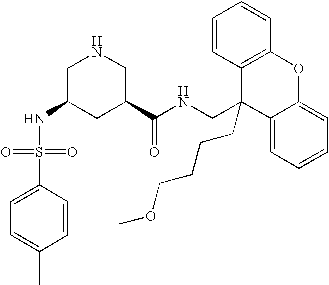

The title compound is prepared as described under “General Procedure, Scheme 2” using C-[9-(4-methoxy-butyl)-9H-xanthen-9-yl]-methylamine and 4-toluenesulfonyl chloride. MS: [M+H]⁺ 587; TLC, Rf CH₂Cl₂/MeOH/NH3 50/6/1)=0.57.

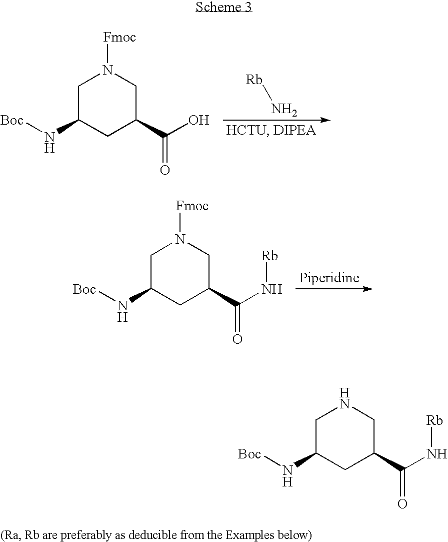

### General Procedure, Scheme 3

To a stirred, ice-cooled mixture of (3S*,5R*)-5-tert-butoxycarbonylamino-piperidine-1,3-dicarboxylic acid 1-(9H-fluoren-9-ylmethyl) ester (1 eq.) in CH₂Cl₂, DIPEA (0.5 eq.) is added, followed by HCTU (1.09 eq.) in CH₃CN. The mixture is stirred for 15 min at 0° C. After the addition of the corresponding amine (1 eq.), stirring is continued for 1 h at 0° C. and then for 14 h at RT. The reaction mixture is distributed between saturated NaHCO₃ solution and ethyl acetate. The aqueous layer is extracted with ethyl acetate. The combined organic layers are washed with 2N HCl and brine, dried over Na₂SO₄, filtered and evaporated. The product thus obtained is stirred in a mixture of CH₂Cl₂/piperidine 4:1 for 1.5 h at RT. The reaction mixture is evaporated and the residue purified by preparative HPLC chromatography (YMC-Pack Pro C18 column, 150 mm×30 mm, 5 μM; 10-100% CH₃CN+0.1% TFA/H₂O+0.1% TFA, 20 min, flow 20 ml/min).

### Example 5

### ((3R*,5S*)-5-[(9H-Xanthen-9-ylmethyl)-carbamoyl]-piperidin-3-yl}-carbamic acid tert-butyl ester

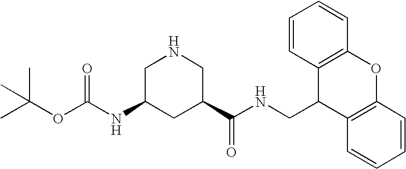

The title compound is prepared as described under “General Procedure, Scheme 3” using C-(9H-xanthen-9-yl)-methylamine. MS: 438 [M+H]⁺; TLC, Rf (CH₂Cl₂/MeOH/NH3 50/6/1)=0.38.

### Example 6

### {(3R*,5S*)-5-[(9-Phenethyl-9H-xanthen-9-ylmethyl)-carbamoyl]-piperidin-3-yl}-carbamic acid tert-butyl ester, formate

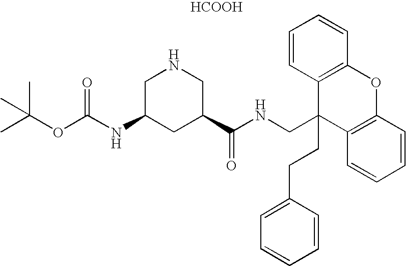

The title compound is prepared as described under “General Procedure, Scheme 3” using C-(9-phenethyl-9H-xanthen-9-yl)-methylamine. MS: [M+H]⁺ 543; t_{R} (HPLC, Nucleosil C18; 5-100% CH₃CN+0.1% TFA/H₂O+0.1% TFA for 8 min, flow 1.5 ml/min): 5.3 min.

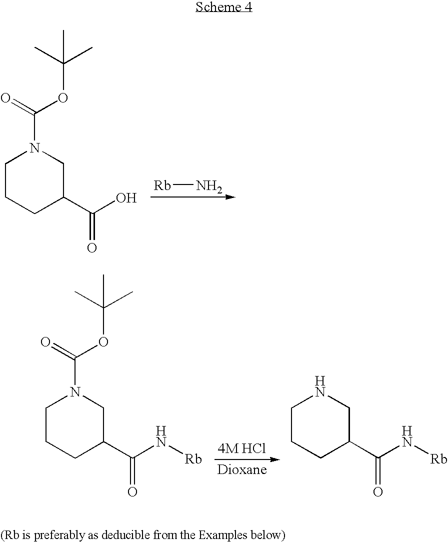

### Example 7

### Piperidine-3-carboxylic acid (9-phenethyl-9H-xanthen-9-ylmethyl)-amide

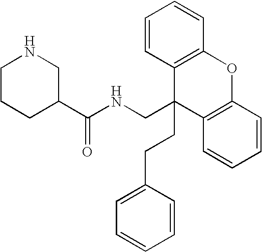

The title compound is prepared analogously as described in Example 8 using C-(9-phenethyl-9H-xanthen-9-yl)-methylamine. MS: [M+H]⁺ 409; t_{R} (HPLC, Nucleosil C18; 5-100% CH₃CN+0.1% TFA/H₂O+0.1% TFA for 8 min, flow 1.5 ml/min): 5.04 min.

### Example 8

### Piperidine-3-carboxylic acid [9-(4-methoxy-butyl)-9H-xanthen-9-ylmethyl]-amide

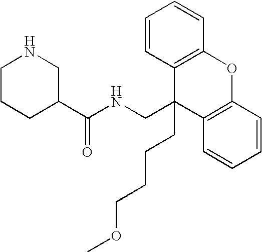

To a mixture of 3-{[9-(4-methoxy-butyl)-9H-xanthen-9-ylmethyl]-carbamoyl}-piperidine-1-carboxylic acid tert-butyl ester (260 mg, 0.5 mmol) in dioxane (3 mL), HCl (4M in dioxane, 1 mL) is added and the reaction mixture is stirred for 3 h at room temperature before it is treated with sat. NaHCO₃ and extracted with dichloromethane. The organic layer is dried over Na₂SO₄, filtered and concentrated under reduced pressure to give a yellow solid. MS (LC-MS): 409 [M+H]⁺; t_{R} (HPLC, Nucleosil C18; 5-100% CH₃CN+0.1% TFA/H₂O+0.1% TFA for 8 min, flow 1.5 ml/min): 3.2 min.

The starting material is prepared as follows:

### 3-{[9-(4-Methoxy-butyl)-9H-xanthen-9-ylmethyl]-carbamoyl}-piperidine-1-carboxylic acid tert-butyl ester

To a stirred, ice-cooled mixture of piperidine-1,3-dicarboxylic acid 1-tert-butyl ester (Aldrich, Buchs, Switzerland) (231 mg, 1 mmol) in CH₂Cl₂ (1.5 mL), O-(1H-6-chlorobenzotriazole-1-yl)-1,1,3,3-tetramethyluronium hexafluorophosphate (342 mg, 0.8 mmol) and after 5 min C-[9-(4-methoxy-butyl)-9H-xanthen-9-yl]-methylamine (200 mg, 0.67 mmol) and triethylamine (0.9 mL) in CH₃CN (1.5 mL) are added. The reaction mixture is stirred at room temperature overnight, before sat. NaHCO₃ is added. The aqueous layer is extracted with dichloromethane. The organic layer is dried over Na₂SO₄, filtered and concentrated under reduced pressure to give a residue, which is purified by flash chromatography on silica gel (eluent: hexane/ethyl acetate 1/1 to 0/1) to give the title compound as a colorless solid. MS (LC-MS): 453 [M+H]⁺; TLC, Rf (hexane/ethyl acetate 1/1)=0.21.

### Example 9

### (9-Phenethyl-9H-xanthen-9-ylmethyl)piperidin-3-ylmethyl-amine

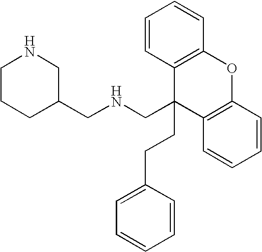

To a mixture of 3-{[9-phenethyl-9H-xanthen-9-ylmethyl]-carbamoyl}-piperidine-1-carboxylic acid tert-butyl ester (280 mg, 0.5 mmol) in dioxane (3 mL) is added HCl (4M in dioxane, 2 mL) and the reaction mixture is stirred for 2 h at 60° C. before it is treated with sat. NaHCO₃ and extracted with dichloromethane. The organic layer is dried over Na₂SO₄, filtered and concentrated under reduced pressure to give a colorless oil. MS (LC-MS): 413 [M+H]⁺; t_{R} (HPLC, Nucleosil C18; 5-100% CH₃CN+0.1% TFA/H₂O+0.1% TFA for 8 min, flow 1.5 ml/min): 4.4 min.

The starting materials are prepared as follows:

### A) 3-{[9-phenethyl-9H-xanthen-9-ylmethyl]-carbamoyl}-piperidine-1-carboxylic acid tert-butyl ester

A mixture of 3-formyl-piperidine-1-carboxylic acid tert-butyl ester (130 mg, 0.6 mmol) (Arch Corporation, New Brunswick, USA), C-[9-phenethyl-9H-xanthen-9-yl]-methylamine (283 mg, 0.9 mmol) and sodium triacetoxyborohydride (330 mg, 1.5 mmol) in 1,2-dichloroethane (2 mL) is stirred at room temperature overnight. The crude mixture is purified by flash chromatography on silica gel (eluent: cyclohexane/ethyl acetate 9/1 to 1/1) to give the title compound as a colorless oil. MS (LC-MS): 514 [M+H]⁺; TLC, Rf (hexane/ethyl acetate 1/1)=0.5.

### B) C-(9-Phenethyl-9H-xanthen-9-yl)-methylamine

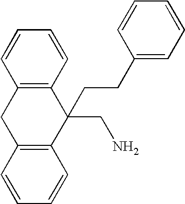

The title compound is prepared analogously as described for C-(9-methyl-9H-xanthen-9-yl)methylamine using phenylethyl bromide instead of methyl iodide in step B2. MS: 316 [M+H]⁺.

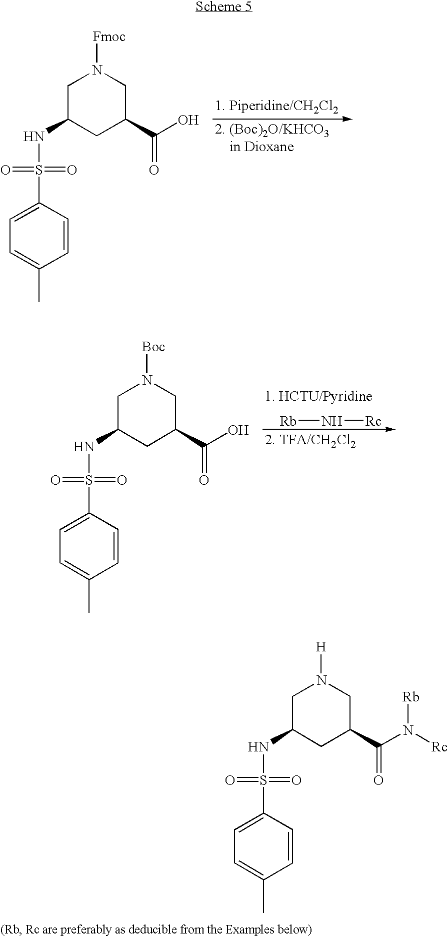

### Example 10

### (3S*,5R*)-5-(Toluene-4-sulfonylamino)-piperidine-3-carboxylic acid (10-methyl-5H-dibenzo[a,d]cyclohepten-5-ylmethyl)-amide, trifluoroacetate

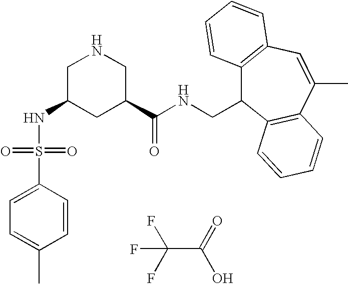

A small part of (3S*,5R*)-5-(toluene-4-sulfonylamino)-piperidine-1,3-dicarboxylic acid 1-tert-butyl ester obtained as described below (40 mg, 0.1 mmol) is dissolved in pyridine (0.5 mL). The solution is cooled to 2° C., treated with O-(1H-6-chlorobenzotriazole-1-yl)-1,1,3,3-tetramethyluronium hexafluorophosphate and stirred at 2° C. for 1 h. The resulting mixture is added to a pre-cooled solution of C-(10-methyl-5-H-dibenzo[a,d]cyclohepten-5-yl)-methylamine (see e.g. CH 478754) (23.5 mg, 0.1 mmol) in pyridine (0.4 mL). The reaction mixture is stirred at 4° C. for 14 h, evaporated in an air stream, and the residue is then evaporated twice after addition of CH₂Cl₂. The crude product is dissolved in CH₂Cl₂ (2 mL) and put on a 3 mL Isolute® HM-N cartridge (Argonaut Technologies, Inc., Mid Glamorgan, U.K.) pretreated with an aqueous 10% K₂CO₃ solution (2 mL). The compound is eluted with CH₂Cl₂ (2×6 mL). The organic layer is evaporated and dried at RT. A solution of CH₂Cl₂/trifluoroacetic acid (1:1) is added to the residue, the mixture is shaken for 1 h at RT and evaporated. The residue is purified by preparative HPLC (YMC-Pack Pro C18 column, 150 mm×30 mm, 5 μM; 10-100% CH₃CN+0.1% TFA/H₂O+0.1% TFA, 20 min, flow 20 ml/min) to afford the title compound. MS (LC-MS): 516.5 [M+H]⁺. t_{R} (HPLC, Nucleosil C18; 5-100% CH₃CN+0.1% TFA/H₂O+0.1% TFA for 8 min, flow 1.5 ml/min): 5.51 min.

The starting material is prepared as follows:

### A) (3S*,5R*)-5-(Toluene-4-sulfonylamino)-piperidine-1,3-dicarboxylic acid 1-tert-butyl ester

A mixture of (3S*,5R*)-5-(toluene-4-sulfonylamino)-piperidine-1,3-dicarboxylic acid 1-(9H-fluoren-9-ylmethyl) ester (11 g, 21.1 mmol), piperidine (62.6 mL, 633 mmol) and CH₂Cl₂ (170 mL) is stirred for 1 h at RT. The solution is evaporated and the residue is distributed between CH₂Cl₂ and aqueous 10% KHCO₃ solution. After separation, the aqueous layer is washed a second time with CH₂Cl₂. The combined organic layers are extracted with 10% KHCO₃ solution. Total amount of KHCO₃ solution: 170 ml (10% solution). The combined aqueous solutions are treated with dioxane (170 mL) and di-tert-butyl dicarbonate (27.3 mL, 120 mmol), and the resulting mixture is stirred for 16 h at RT. After addition of K₂CO₃ solution (10%, 50 mL), the mixture is washed twice with tert-butyl methyl ether. The aqueous layer is slowly acidified to pH 2 with a 10% NaHSO₄ solution. The aqueous layer is extracted three times with CH₂Cl₂. The combined organic layers are washed with H₂O, dried (Na₂SO₄) and evaporated to yield the title compound as an amorphous solid. MS (LC-MS): 299 [M+H—C₅H₈O₂], 343 [M+H—C₄H₈]; t_{R} (HPLC, Symmetry C18 (3×150 mm); 5-100% CH₃CN+0.05% TFA/H₂O+0.05% TFA for 6 min, flow 1.5 ml/min): 3.99 min.

### Example 11

### (3S*,5R*)-5-(Toluene-4-sulfonylamino)-piperidine-3-carboxylic acid [2-(3-chloro-10,11-dihydro-dibenzo[b,f]azepin-5-yl)-ethyl]-amide, trifluoroacetate

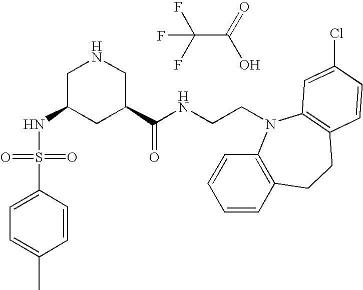

The title compound is prepared analogously as described in Example 10 using 2-(3-chloro-10,11-dihydro-dibenzo[b,f]azepin-5-yl)-ethylamine (see e.g. Bickel, M. H., Brodie B. B., Intern. J. Neuropharmacol. (1964), 3, 611-21) instead of 10-aminomethyl-9,10-dihydro-9,10-ethanoanthracen-11-one. MS (LC-MS): 553.5/555.2 [M+H]⁺; t_{R} (HPLC, Symmetry C18; 5-100% CH₃CN+0.05% TFA/H₂O+0.05% TFA for 6 min, flow 1.5 ml/min): 3.79 min.

### Example 12

### (3S*,5R*)-5-(Toluene-4-sulfonylamino)-piperidine-3-carboxylic acid (10,11-dihydro-5H-dibenzo[a,d]cyclohepten-5-ylmethyl)amide, trifluoroacetate

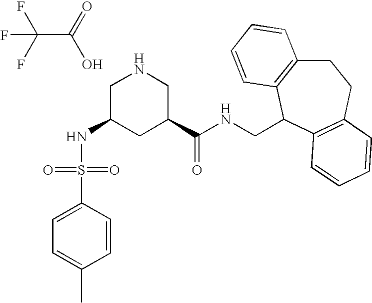

The title compound is prepared analogously as described in Example 10 using C-(10,11-dihydro-5H-dibenzo[a,d]cyclohepten-5-yl)-methylamine (see e.g. Humber, L. G., Davis, M. A.; Fr. (1967), FR 1491687) instead of 10-aminomethyl-9,10-dihydro-9,10-ethano-anthracen-11-one. MS (LC-MS): 504.6 [M+H]⁺; t_{R} (HPLC, Nucleosil C18; 5-100% CH₃CN+0.1% TFA/H₂O+0.1% TFA for 8 min, flow 1.5 ml/min): 5.25 min.

### Example 13

### (3S*,5R*)-5-(Toluene-4-sulfonylamino)-piperidine-3-carboxylic acid [3-(10,11-dihydro-dibenzo[b,f]azepin-5-yl)-2-hydroxy-propyl]-methyl-amide, trifluoroacetate

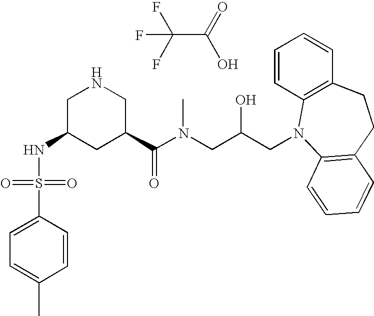

The title compound is prepared analogously as described in Example 10 using 1-(10,11-dihydro-dibenzo[b,f]azepin-5-yl)-3-methylamino-propan-2-ol (see e.g. EP 0 107 134) instead of 10-aminomethyl-9,10-dihydro-9,10-ethano-anthracen-11-one. MS (LC-MS): 563.6 [M+H]⁺; t_{R} (HPLC, Nucleosil C18; 5-100% CH₃CN+0.1% TFA/H₂O+0.1% TFA for 8 min, flow 1.5 ml/min): 5.48 and 5.53 min (diastereomers).

### Example 14

### (3S*,5R*)-5-(Toluene-4-sulfonylamino)-piperidine-3-carboxylic acid (6,11-dihydro-5H-dibenzo[b,e]azepin-6-ylmethyl)-methyl-amide, trifluoroacetate

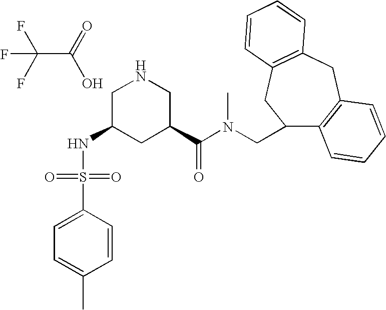

The title compound is prepared analogously as described in Example 10 using (6,11-dihydro-5H-dibenzo[b,e]azepin-6-ylmethyl)-methyl-amine (see e.g. Van der Burg, W. J., Bonta, I. L., Delobelle, J., Ramon, C., Vargaftig, B.; J. Med. Chem. (1970), 13, 35-9) instead of 10-aminomethyl-9,10-dihydro-9,10-ethano-anthracen-11-one. MS (LC-MS): 519.1 [M+H]⁺; t_{R} (HPLC, Nucleosil C18; 5-100% CH₃CN+0.1% TFA/H₂O+0.1% TFA for 8 min, flow 1.5 ml/min): 5.24 and 5.34 min (diastereomers).

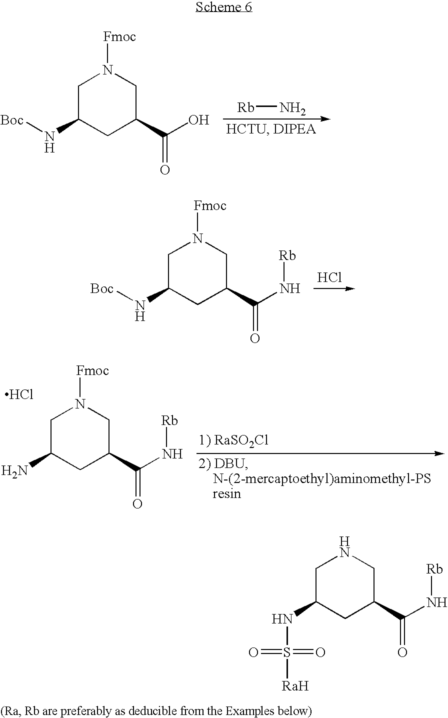

### General Procedure, Scheme 6

To a stirred, ice-cooled mixture of (3S,5R)-5-tert-butoxycarbonylamino-piperidine-1,3-dicarboxylic acid 1-(9H-fluoren-9-ylmethyl) ester (1 eq.) in CH₂Cl₂, N-ethyldiisopropylamine (0.5 eq.) is added, followed by O-(1H-6-chlorobenzotriazole-1-yl)-1,1,3,3-tetramethyluronium hexafluorophosphate (1.09 eq.) in CH₃CN. The mixture is stirred for 15 min at 0° C. After the addition of the corresponding amine (1 eq.), stirring is continued for 1 h at 0° C. and then for 14 h at RT. The reaction mixture is distributed between saturated NaHCO₃ solution and ethyl acetate. The aqueous layer is extracted with ethyl acetate. The combined organic layers are washed with 2N HCl and brine, dried over Na₂SO₄, filtered and evaporated. A mixture of the product thus obtained and HCl (4M in dioxane, ˜30 eq.) is stirred for 1 h at RT. The mixture is evaporated to dryness. The remaining product is dissolved in pyridine and cooled in an ice bath. After addition of 4-dimethylaminopyridine (0.3 eq.) and the corresponding sulfonyl chloride (4 eq.), the ice bath is removed, and the mixture is stirred at RT for 14 h. The reaction mixture is diluted with H₂O and acidified with 1N HCl to pH 2, and the aqueous layer is extracted three times with ethyl acetate. The combined organic layers are washed with brine, dried (Na₂SO₄) and evaporated to afford the title compound as a white amorphous solid. To a solution of the crude compound in THF, N-(2-mercaptoethyl)aminomethyl-PS resin (6.3 eq.) and DBU (0.5 eq.) are added. After stirring at RT for 2 h, the resin is removed by filtration. The filtrate is evaporated in vacuo and the residue is purified by preparative HPLC chromatography (XTerra Prep MS C18 column, 100 mm×30 mm, 5 μM; 5-100% CH₃CN+0.1% TFA/H₂O+0.1% TFA, 32 min, flow rate 30 ml/min).

### Example 15

### (3S,5R)-5-(Toluene-3-sulfonylamino)-piperidine-3-carboxylic acid [9-(3-methoxy-propyl)-9H-xanthen-9-ylmethyl]-amide

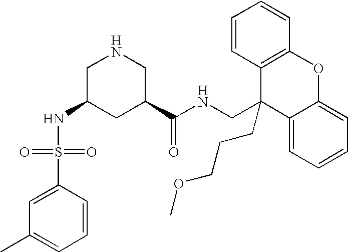

The title compound is prepared as described under “General Procedure, Scheme 6” using C-[9-(3-methoxy-propyl)-9H-xanthen-9-yl]-methylamine and 3-methyl-benzenesulfonyl chloride. MS: 578 [M+H]⁺; HPLC (Condition-B) t_{R}=2.35 min

### (3S,5R)-3-{[9-(4-Methoxy-butyl)-9H-xanthen-9-ylmethyl]-carbamoyl}-5-(toluene-2-sulfonylamino)-piperidine-1-carboxylic acid 9H-fluoren-9-ylmethyl ester

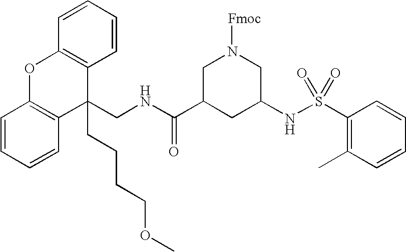

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and α-toluenesulfonyl chloride (26 μL, 0.18 mmol) analogously to the preparation of “general procedure, scheme Z”. White amorphous material; ES-MS: M+H=800; HPLC (Condition-A): t_{R}=4.84 min.

### (3S,5R)-3-{[9-(4-Methoxy-butyl)-9H-xanthen-9-ylmethyl]-carbamoyl}-5-phenylmethanesulfonylamino-piperidine-1-carboxylic acid 9H-fluoren-9-ylmethyl ester

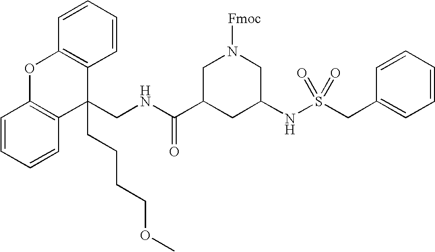

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and α-toluenesulfonyl chloride (34 mg, 0.18 mmol) analogously to the preparation of “general procedure, scheme Z”. White amorphous material; ES-MS: M+H=800; HPLC (Condition-A): t_{R}=4.72 min.

### (3S,5R)-3-{[9-(4-Methoxy-butyl)-9H-xanthen-9-ylmethyl]-carbamoyl}-5-(2-trifluoromethoxy-benzenesulfonylamino)-piperidine-1-carboxylic acid 9H-fluoren-9-ylmethyl ester

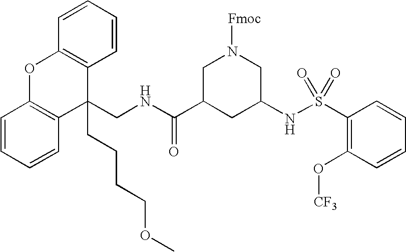

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and 2-(trifluoromethyl)benzenesulfonyl chloride (47 mg, 0.18 mmol) analogously to the preparation of “general procedure, scheme Z”. White amorphous material; ES-MS: M+H=870; HPLC (Condition-A): t_{R}=4.92 min.

### (3R,5S)-3-Methanesulfonylamino-5-{[9-(4-methoxy-butyl)-9H-xanthen-9-ylmethyl]-carbamoyl}-piperidine-1-carboxylic acid 9H-fluoren-9-ylmethyl ester

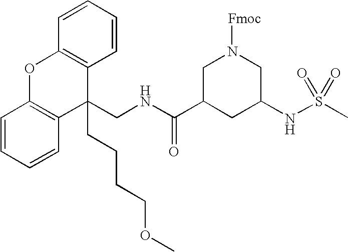

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and methanesulfonyl chloride (14 μL, 0.18 mmol) analogously to the preparation of “general procedure, scheme Z”. White amorphous material; ES-MS: M+H=724; HPLC (Condition-A): t_{R}=4.32 min.

### (3R,5S)-3-Cyclopropanesulfonylamino-5-{[9-(4-methoxy-butyl)-9H-xanthen-9-ylmethyl]-carbamoyl}-piperidine-1-carboxylic acid 9H-fluoren-9-ylmethyl ester

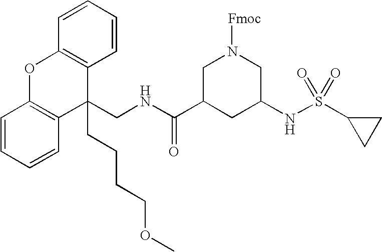

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and cyclopropanesulfonyl chloride (18 μL, 0.18 mmol) analogously to the preparation of “general procedure, scheme Z”. White amorphous material; ES-MS: M+H=750; HPLC (Condition-A): t_{R}=4.45 min.

### (3R,5S)-3-(3,4-Dimethoxy-benzenesulfonylamino)-5-{[9-(4-methoxy-butyl)-9H-xanthen-9-ylmethyl]-carbamoyl}-piperidine-1-carboxylic acid 9H-fluoren-9-ylmethyl ester

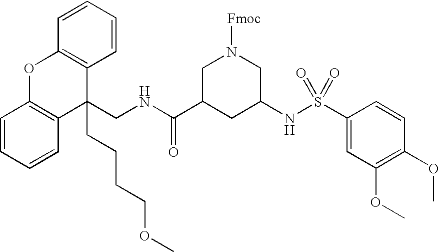

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and 3,4-dimethoxybenzenesulfonyl chloride (43 mg, 0.18 mmol) analogously to the preparation of “general procedure, scheme Z”. White amorphous material; ES-MS: M+H=846; HPLC (Condition-A): t_{R}=4.55 min.

### (3S,5R)-3-{[9-(4-Methoxy-butyl)-9H-xanthen-9-ylmethyl]-carbamoyl}-5-(2,2,2-trifluoroethanesulfonylamino)-piperidine-1-carboxylic acid 9H-fluoren-9-ylmethyl ester

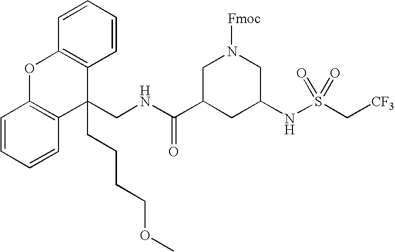

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and 2,2,2-trifluoroethanesulfonyl chloride (20 μL, 0.18 mmol) analogously to the preparation of “general procedure, scheme Z”. White amorphous material; ES-MS: M+H=792; HPLC (Condition-A): t_{R}=4.62 min.

### (3R,5S)-3-(4-Cyano-benzenesulfonylamino)-5-{[9-(4-methoxy-butyl)-9H-xanthen-9-ylmethyl]-carbamoyl}piperidine-1-carboxylic acid 9H-fluoren-9-ylmethyl ester

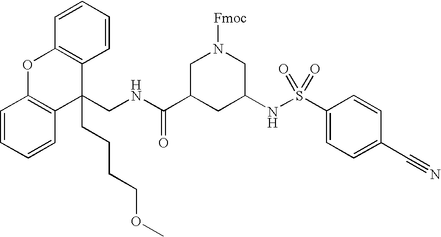

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and 4-cyanobenzenesulfonyl chloride (36 mg, 0.18 mmol) analogously to the preparation of “general procedure, scheme Z”. White amorphous material; ES-MS: M+H=811; HPLC (Condition-A): t_{R}=4.60 min.

### (3S,5R)-3-{[9-(4-Methoxy-butyl)-9H-xanthen-9-ylmethyl]-carbamoyl}-5-(2,4,6-trimethyl-benzenesulfonylamino-piperidine-1-carboxylic acid 9H-fluoren-9-ylmethyl ester

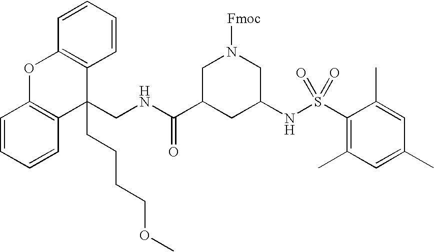

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and 2-mesitylenesulfonyl chloride (39 mg, 0.18 mmol) analogously to the preparation of “general procedure, scheme Z” White amorphous material; ES-MS: M+H=828; HPLC (Condition-B): t_{R}=2.46 min.

### (3R,5S)-3-(4-Fluoro-benzenesulfonylamino)-5-{[9-(4-methoxy-butyl)-9H-xanthen-9-ylmethyl]-carbamoyl}-piperidine-1-carboxylic acid 9H-fluoren-9-ylmethyl ester

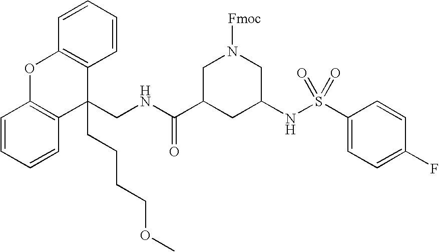

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and 4-fluorobenzenesulfonyl chloride (35 mg, 0.18 mmol) analogously to the preparation of “general procedure, scheme Z”. White amorphous material; ES-MS: M+H=804; HPLC (Condition-B): t_{R}=2.40 min.

### (3R,5S)-3-(2,5-Dimethyl-thiophene-3-sulfonylamino)-5-{[9-(4-methoxy-butyl)-9H-xanthen-9-ylmethyl]-carbamoyl}-piperidine-1-carboxylic acid 9H-fluoren-9-ylmethyl ester

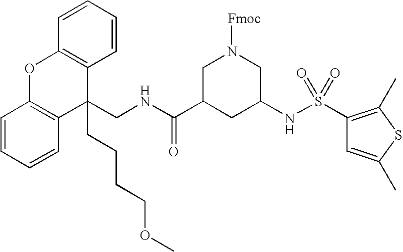

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and 2,5-dimethyl-3-thiophenesulfonyl chloride (38 mg, 0.18 mmol) analogously to the preparation of “general procedure, scheme Z”. White amorphous material; ES-MS: M+H=820; HPLC (Condition-B): t_{R}=2.47 min.

### (3S,5R)-3-{[9-(4-Methoxy-butyl)-9H-xanthen-9-ylmethyl]-carbamoyl}-5-(pyridine-2-sulfonylamino-piperidine-1-carboxylic acid 9H-fluoren-9-ylmethyl ester

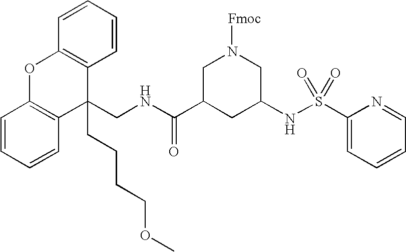

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (102 mg, 0.15 mmol) and 2-pyridinesulfonyl chloride (39 mg, 0.18 mmol) analogously to the preparation of “general procedure, scheme Z”. White amorphous material; ES-MS: M+H=787; HPLC (Condition-B): t_{R}=2.18 min.

### (3R,5S)-3-(4-Hydroxy-3-methoxy-benzenesulfonylamino)-5-{[9-(4-methoxy-butyl)-9H-xanthen-9-ylmethyl]-carbamoyl}-piperidine-1-carboxylic acid 9H-fluoren-9-ylmethyl ester

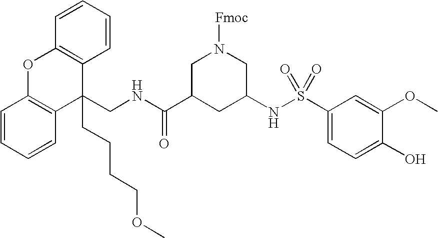

To a stirred solution of 4-hydroxy-3-methoxybenzenesulfonyl chloride (134 mg, 0.6 mmol) in CH₂Cl₂, N,O-bis(trimethylsilyl)acetamide (161 μL, 0.66 mmol) is added at room temperature and stirred for 0.5 hours under N₂. After the mixture is cooled down to 0° C., (3R,5S)-3-amino-5-{[9-(4-methoxy-butyl)-9H-xanthen-9-ylmethyl]-carbamoyl}-piperidine-1-carboxylic acid 9H-fluoren-9-ylmethyl ester (341 mg, 0.5 mmol) and Et₃N (139 μL, 1.0 mmol) are added, and the reaction is stirred for 12 hours. The mixture is diluted with saturated NaHCO₃ solution, and the aqueous layer is extracted with EtOAc. The combined organic layers are washed with brine, dried (Na₂SO₄), filtration through silica gel, and evaporated to afford the title compound as a white powder; ES-MS: M+H=832; HPLC (Condition-A): t_{R}=4.35 min.

### (3R,5S)-3-[4-(2-Dimethylamino-ethoxy)-3-methoxy-benzenesulfonylamino]-5-{[9-(4-methoxybutyl)-9H-xanthen-9-ylmethyl]-carbamoyl}-piperidine-1-carboxylic acid 9H fluoren-9-ylmethyl ester

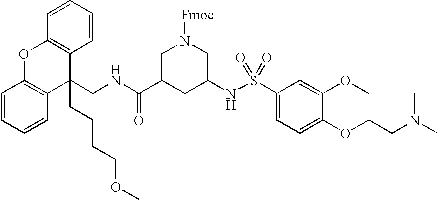

To a solution of (3R,5S)-3-(4-Hydroxy-3-methoxy-benzenesulfonylamino)-5-{[9-(4-methoxybutyl)-9H-xanthen-9-ylmethyl]-carbamoyl}-piperidine-1-carboxylic acid 9H-fluoren-9-ylmethyl ester (92 mg, 0.11 mmol), 2-(dimethylamino) ethanol (14 μL, 0.13 mmol) and PPh₃ (58 mg, 0.22 mmol) are added. After cooling down to 0° C., DEAD (26 μL, 0.17 mmol) is added. The reaction mixture is allowed to warm to room temperature and stirred for 2.5 hours. The mixture is diluted with H₂O, and the aqueous layer is extracted with EtOAc. The combined organic layers are washed with brine, dried over Na₂SO₄, filtered through silica gel, and evaporated to give the title compound; ES-MS: M+H=903; HPLC (Condition-A): t_{R}=3.63 min.

### (3R,5S)-3-[4-(3-Hydroxy-propoxy)-3-methoxy-benzenesulfonylamino]-5-{[9-(4-methoxy-butyl)-9H-xanthen-9-ylmethyl]-carbamoyl}-piperidine-1-carboxylic acid 9H-fluoren-9-ylmethyl ester

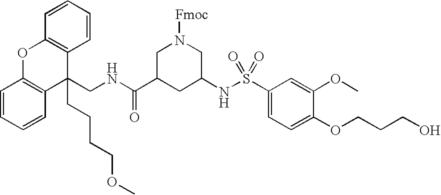

To a solution of (3R,5S)-3-(4-Hydroxy-3-methoxy-benzenesulfonylamino)-5-{[9-(4-methoxy-butyl)-9H-xanthen-9-ylmethyl]-carbamoyl}-piperidine-1-carboxylic acid 9H-fluoren-9-ylmethyl ester (92 mg, 0.11 mmol) in DMF (2 mL), 3-bromopropanol (13 μL, 0.144 mmol) and K₂CO₃ (50 mg, 0.36 mmol) are added at rt. After stirring for 3.5 hours, the reaction mixture is diluted with H₂O and extracted with EtOAc. The combined organic phases are washed with H₂O and dried over Na₂SO₄. Concentration under reduced pressure and filtration through silica gel the title compound; ES-MS: M+H=768; HPLC (Condition-A): t_{R}=3.72 min.

### (3R,5S)-3-(4-Methanesulfonyloxymethyl-benzenesulfonylamino)-5-{[9-(4-methoxy-butyl)-9H-xanthen-9-ylmethyl]-carbamoyl}-piperidine-1-carboxylic acid tert-butyl ester

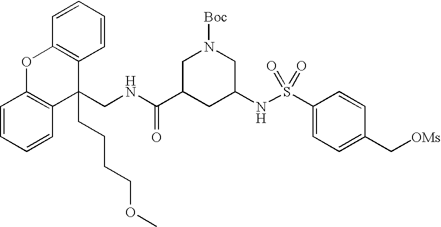

To a stirred, ice-cooled solution of (3R,5S)-3-(4-hydroxymethyl-benzenesulfonylamino)-5-{[9-(4-methoxy-butyl)-9H-xanthen-9-ylmethyl]-carbamoyl}-piperidine-1-carboxylic acid tert-butyl ester (230 mg, 0.33 mmol) in CH₂Cl₂ (5 mL), Et₃N (69 μL, 0.4 mmol) is added, followed by methanesulfonyl chloride (31 μL, 0.4 mmol). After stirring at the temperature for 4 hours under N₂, the reaction mixture is diluted with saturated NaHCO₃ solution, and the aqueous layer is extracted with EtOAc. The combined organic layers are washed with brine, dried (Na₂SO₄), filtration, and evaporated to afford the title compound as a white amorphous solid. The crude product is used without purification; ES-MS: M+H=772; HPLC (Condition-A): t_{R}=4.00 min.

### (3R,5S)-3-(4-Hydroxymethyl-benzenesulfonylamino)-5-{[9-(4-methoxy-butyl)-9H-xanthen-9-ylmethyl]-carbamoyl}-piperidine-1-carboxylic acid tert-butyl ester

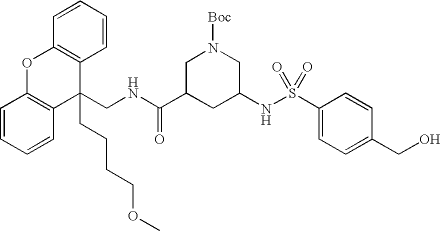

To a stirred, ice-cooled solution of crude material, (3R,5S)-3-(4-carboxy-benzenesulfonylamino)-5-([9-(4-methoxy-butyl)-9H-xanthen-9-ylmethyl]-carbamoyl)-piperidine-1-carboxylic acid 9H-fluoren-9-ylmethyl ester in MeOH (5 mL), NaBH₄ (19 mg, 0.5 mmol) is added. After stirring for 1 hour, the reaction mixture is diluted with H₂O, and the aqueous layer is extracted with EtOAc. The combined organic layers are washed with brine, dried (Na₂SO₄), filtration, and evaporated to afford the title compound as a white powder. The crude product is used without purification; ES-MS: M+H=694; HPLC (Condition-A): t_{R}=3.72 min.

### (3R,5S)-3-(4-Formyl-benzenesulfonylamino)-5-{[9-(4-methoxy-butyl)-9H-xanthen-9-ylmethyl]-carbamoyl}-piperidine-1-carboxylic acid tert-butyl ester

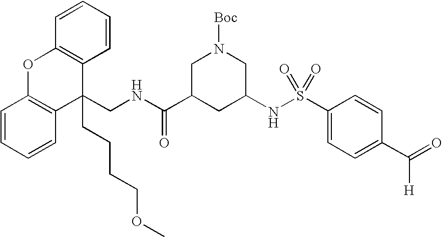

To a solution of (3S,5R)-5-(4-Formyl-benzenesulfonylamino)-piperidine-3-carboxylic acid [9-(4-methoxy-butyl)-9H-xanthen-9-ylmethyl]-amide in THF (5 mL), Boc₂O (19 mg, 0.5 mmol) and Et₃N (70 μL, 0.5 mmol) are added, and the resulting reaction mixture is stirred under N₂ at RT for 2 h. After adding H₂O, the reaction mixture is extracted with EtOAc. The combined organic layers are washed with brine, dried (Na₂SO₄). Concentration under reduced pressure and silica gel flash chromatography give the title compound as colorless amorphous; ES-MS: M+H=692; HPLC (Condition-A): t_{R}=4.05 min.

### (3S,5R)-5-(4-Formyl-benzenesulfonylamino)-piperidine-3-carboxylic acid [9-(4-methoxybutyl)-9H-xanthen-9-ylmethyl]-amide

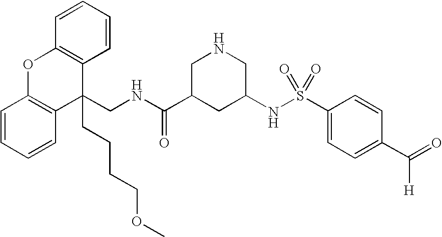

The title compound is synthesized by of removal of Fmoc group of (3R,5S)-3-(4-Formyl-benzenesulfonylamino)-5-{[9-(4-methoxy-butyl)-9H-xanthen-9-ylmethyl]-carbamoyl}piperidine-1-carboxylic acid 9H-fluoren-9-ylmethyl ester analogously to the preparation of “general procedure scheme Z”; ES-MS: M+H=592; HPLC (Condition-A): t_{R}=2.85 min.

### (3R,5S)-3-(4-Formyl-benzenesulfonylamino)-5-{[9-(4-methoxy-butyl)-9H-xanthen-9-ylmethyl]-carbamoyl}-piperidine-1-carboxylic acid 9H-fluoren-9-ylmethyl ester

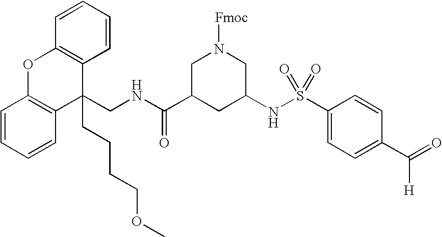

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (341 mg, 0.5 mmol) with 4-formylbenzenesulfonyl chloride (123 mg, 0.6 mmol) analogously to the preparation of the general procedure Z. White amorphous material; ES-MS: M+H=814; HPLC (Condition-A): t_{R}=4.57 min.

### (3R,5S)-3-[4-(3-Dimethylamino-propyl)-benzenesulfonylamino]-5-{[9-(4-methoxy-butyl)-9H-xanthen-9-ylmethyl]-carbamoyl}-piperidine-1-carboxylic acid tert-butyl ester

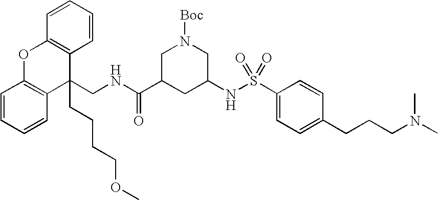

To a solution of (3R,5S)-3-[4-(3-methanesulfonyloxy-propyl)-benzenesulfonylamino]-5-{[9-(4-methoxy-butyl)-9H-xanthen-9-ylmethyl]-carbamoyl}-piperidine-1-carboxylic acid tert-butyl ester (110 mg, 0.14 mmol), 2 M solution of dimethylamine in THF (5 mL) is added at rt. After stirring over night, the reaction mixture is diluted with H₂O and extracted with EtOAc. The combined organic phases are washed with H₂O and dried over Na₂SO₄, and concentration under reduced pressure. The combined organic residue is purified by column chromatography to give the title compound; ES-MS: M+H=749; HPLC (Condition-A): t_{R}=3.30 min.

### (3R,5S)-3-[4-(3-Methanesulfonyloxy-propyl)-benzenesulfonylamino]-5-{[9-(4-methoxy-butyl)-9H-xanthen-9-ylmethyl]-carbamoyl}-piperidine-1-carboxylic acid tert-butyl ester

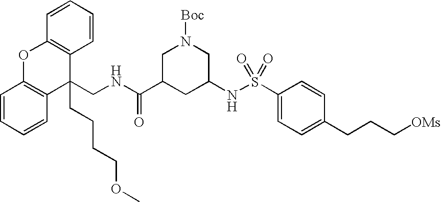

The title compound is synthesized by methanesulfonylation of (3R,5S)-3-[4-(3-Hydroxypropyl)-benzenesulfonylamino]-5-{[9-(4-methoxy-butyl)-9H-xanthen-9-ylmethyl]-carbamoyl}-piperidine-1-carboxylic acid tert-butyl ester (328 mg, 0.45 mmol) analogously to the preparation of (3R,5S)-3-(4-Methanesulfonyloxymethyl-benzenesulfonylamino)-5-{[9-(4-methoxy-butyl)-9H-xanthen-9-ylmethyl]-carbamoyl}-piperidine-1-carboxylic acid tert-butyl ester. White powder; ES-MS: M+H=800; HPLC (Condition-A): t_{R}=4.09 min.

### (3R,5S)-3-[4-(3-Hydroxy-propyl)-benzenesulfonylamino]-5-{[9-(4-methoxy-butyl)-9H-xanthen-9-ylmethyl]-carbamoyl}-piperidine-1-carboxylic acid tert-butyl ester

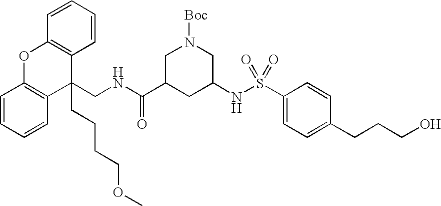

To a solution of (3R,5S)-3-[4-(2-Carboxy-ethyl)-benzenesulfonylamino]-5-{[9-(4-methoxybutyl)-9H-xanthen-9-ylmethyl]-carbamoyl}-piperidine-1-carboxylic acid tert-butyl ester (100 mg, 0.14 mmol) in THF (3.0 mL), Et₃N (0.023 mL, 0.16 mmol) and isobutyl chloroformate (0.02 mL, 0.15 mmol) are added at 0° C. After stirring for 0.5 h at the same temperature, the resulting precipitate is filtered off and the filtrate is concentrated. The residue is dissolved in THF (3 mL), LiBH₄ (3 mg, 0.14 mmol) is added at room temperature. After stirring for 1.5 h, the reaction is quenched with H₂O. The resulting mixture is extracted with EtOAc, washed with brine, dried (Na₂SO₄), and concentrated. Purification by silica gel column chromatography gives the title compound; M+H=716; HPLC (Condition-A): t_{R}=4.99 min.

### (3R,5S)-3-[4-(2-Carboxy-ethyl)-benzenesulfonylamino]-5-{[9-(4-methoxy-butyl)-9H-xanthen-9-ylmethyl]-carbamoyl}-piperidine-1-carboxylic acid tert-butyl ester

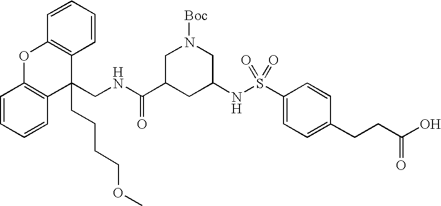

To a solution of crude product of (3S,5R)-3-{[9-(4-Methoxy-butyl)-9H-xanthen-9-ylmethyl]-carbamoyl}-5-[4-(2-methoxycarbonyl-ethyl)-benzenesulfonylamino]-piperidine-1-carboxylic acid tert-butyl ester in MeOH/H₂O/1,4-dioxane (4.0 mL/4.0 mL/2.0 mL), LiOH (0.072 mg, 3.0 mmol) is added at rt. After stirring for 4.0 hours, the reaction mixture is quenched with 1N HCl aq. and extracted with EtOAc. The combined organic phases are washed with H₂O and dried over Na₂SO₄, and concentration under reduced pressure. The combined organic residue is purified by column chromatography to give the title compound; ES-MS: M+H=736; HPLC (Condition-A): t_{R}=3.77 min.

### (3S,5R)-3-{[9-(4-Methoxy-butyl)-9H-xanthen-9-ylmethyl]-carbamoyl}-5-[4-(2-methoxycarbonyl-ethyl)-benzenesulfonylamino]-piperidine-1-carboxylic acid tert-butyl ester

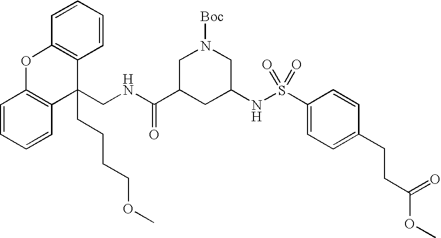

The title compound is synthesized by Boc protection of 3-[4-((3R,5S)-5-{[9-(4-Methoxybutyl)-9H-xanthen-9-ylmethyl]-carbamoyl}-piperidin-3-ylsulfamoyl)-phenyl]-propionic acid methyl ester using Boc₂O (1.2 eq.) and Et₃N (2.4 eq.). White amorphous material; ES-MS: M+H=750; HPLC (Condition-A): t_{R}=4.17 min.

### 3-[4-((3R,5S)-5-{[9-(4-Methoxy-butyl)-9H-xanthen-9-ylmethyl]-carbamoyl}-piperidin-3-ylsulfamoyl)-phenyl]-propionic acid methyl ester

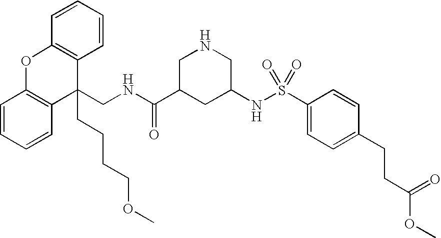

The title compound is synthesized by of removal of Fmoc group of (3S,5R)-3-{[9-(4-methoxybutyl)-9H-xanthen-9-ylmethyl]-carbamoyl}-5-[4-(2-methoxycarbonyl-ethyl)-benzenesulfonylamino]-piperidine-1-carboxylic acid 9H-fluoren-9-ylmethyl ester analogously to the preparation of “general procedure, scheme Z”. The material was used for next step without further purification.

### (3S,5R)-3-{[9-(4-Methoxy-butyl)-9H-xanthen-9-ylmethyl]-carbamoyl}-5-[4-(2-methoxycarbonyl-ethyl)-benzenesulfonylamino]-piperidine-1-carboxylic acid 9H-fluoren-9-ylmethyl ester

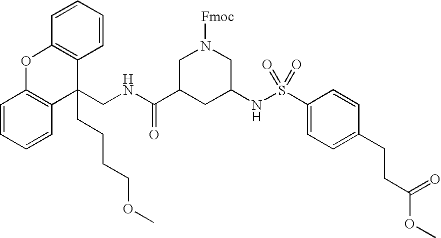

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (410 mg, 0.6 mmol) and methyl 3-(4-chlorosulfonyl)phenyl propionate (174 mg, 0.66 mmol) analogously to the preparation of “general procedure, scheme Z”. White amorphous material; ES-MS: M+H=872; HPLC (Condition-A): t_{R}=4.65 min.

### (3R,5S)-3-(4-Isopropyl-benzenesulfonylamino)-5-{[9-(4-methoxy-butyl)-9H-xanthen-9-ylmethyl]-carbamoyl}-piperidine-1-carboxylic acid 9H-fluoren-9-ylmethyl ester

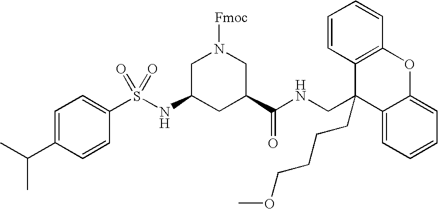

The title compound is synthesized by sulfonylation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (100 mg, 0.15 mmol) and 4-isopropyl benzenesulfonyl chloride (31.5 μl, 0.18 mmol) analogously to the preparation of “general procedure, scheme Z”. White amorphous material ES-MS: M+H=828; HPLC (Condition-A): t_{R}=5.14 min.

### (3R,5S)-3-(4-Isopropoxy-benzenesulfonylamino)-5-{[9-(4-methoxy-butyl)-9H-xanthen-9-ylmethyl]-carbamoyl}-piperidine-1-carboxylic acid 9H-fluoren-9-ylmethyl ester

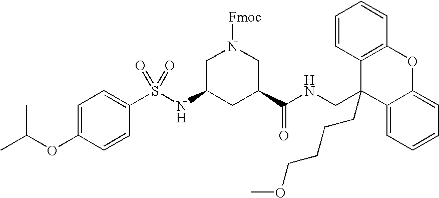

The title compound is synthesized by sulfonylation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (107.6 mg, 0.16 mmol) and 4-isopropoxy benzenesulfonyl chloride (37.5 mg, 0.16 mmol) analogously to the preparation of “general procedure, scheme Z”. White amorphous material White amorphous material; ES-MS: M+H=844; HPLC (Condition-B): t_{R}=2.32 min.

### (3R,5S)-3-(4-Methoxy-benzenesulfonylamino)-5-{[9-(4-methoxy-butyl)-9H-xanthen-9-ylmethyl]-carbamoyl}-piperidine-1-carboxylic acid 9H-fluoren-9-ylmethyl ester

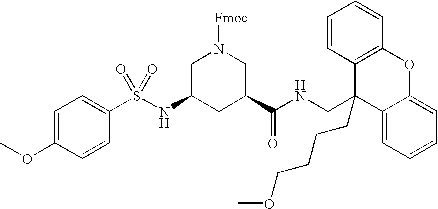

The title compound is synthesized by sulfonylation of (3R,5S)-3-amino-5-{[9-(4-methoxybutyl)-9H-xanthen-9-ylmethyl]-carbamoyl}piperidine-1-carboxylic acid 9H-fluoren-9-ylmethyl ester (103.5 mg, 0.15 mmol) and 4-methoxy benzenesulfonyl chloride (36.3 mg, 0.18 mmol) analogously to the preparation of “general procedure, scheme Z”. White amorphous material ES-MS: M+H=816; HPLC (Condition-A): t_{R}=4.67 min.

### Example 16

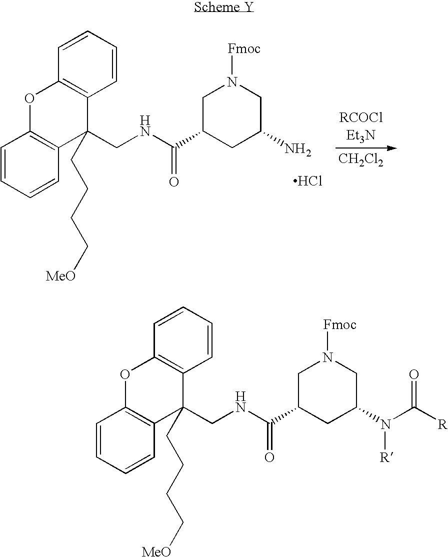

### General Procedure, Scheme Y

To a stirred, ice-cooled solution of (3R,5S)-3-amino-5-{[9-(4-methoxy-butyl)-9H-xanthen-9-ylmethyl]-carbamoyl}-piperidine-1-carboxylic acid 9H-fluoren-9-ylmethyl ester in CH₂Cl₂, Et₃N (2.5 eq) is added, followed by the corresponding acid chloride (1.1 eq). After stirring at the temperature under N₂, the reaction mixture is diluted with saturated NaHCO₃ solution, and the aqueous layer is extracted with EtOAc. The combined organic layers are washed with brine, dried (Na₂SO₄), filtration, and evaporated to afford the title compound as a white amorphous solid. The crude product is used without purification.

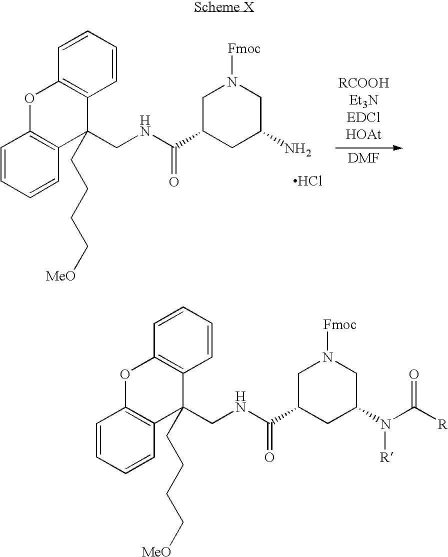

### General Procedure, Scheme X

To a stirred, ice-cooled solution of (3R,5S)-3-amino-5-{[9-(4-methoxy-butyl)-9H-xanthen-9-ylmethyl]-carbamoyl}-piperidine-1-carboxylic acid 9H-fluoren-9-ylmethyl ester in DMF, Et₃N (1.1 eq) and the corresponding carboxylic acid (1.1 eq) are added, followed by EDCl (1.5 eq) and HOAt (1.5 eq). After stirring at 0° C. to rt under N₂ over night, the reaction mixture is diluted with saturated NaHCO₃ solution, and the aqueous layer is extracted with EtOAc. The combined organic layers are washed with brine, dried (Na₂SO₄), filtration, and evaporated to afford the title compound as a white amorphous.

### (3S,5R)-3-{[9-(4-Methoxy-butyl)-9H-xanthen-9-ylmethyl]-carbamoyl}-5-[2-(4-methoxyphenyl)-acetylamino]-piperidine-1-carboxylic acid 9H-fluoren-9-ylmethyl ester

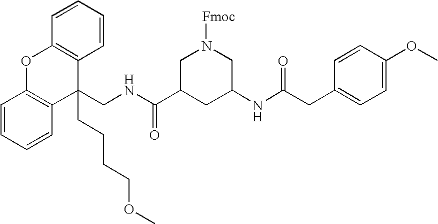

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (96 mg, 0.14 mmol) and 4-methoxyphenylacetyl chloride (0.024 mL, 0.15 mmol) analogously to the preparation of “general procedure, scheme Y”. White powder; ES-MS: M+H=794; HPLC (Condition-A): t_{R}=4.57 min.

### (3S,5R)-3-{[9-(4-Methoxy-butyl)-9H-xanthen-9-ylmethyl]-carbamoyl}-5-(3-methylbutylamino)-piperidine-1-carboxylic acid 9H-fluoren-9-ylmethyl ester

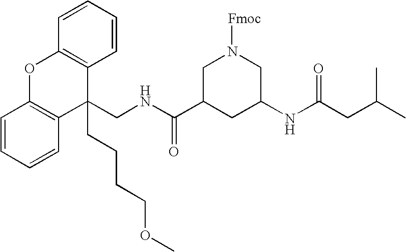

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (96 mg, 0.14 mmol) and isovaleryl chloride (0.019 mL, 0.15 mmol) analogously to the preparation of “general procedure, scheme Y”. White powder; ES-MS: M+H=730; HPLC (Condition-A): t_{R}=4.59 min.

### (3R,5S)-3-(Ethyl-phenylacetyl-amino)-5-{[9-(4-methoxy-butyl)-9H-xanthen-9-ylmethyl]-carbamoyl}-piperidine-1-carboxylic acid 9H-fluoren-9-ylmethyl ester

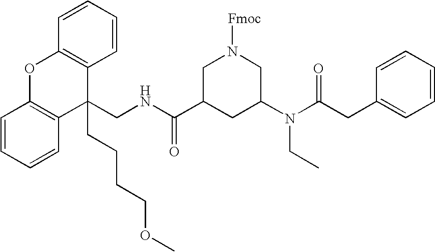

The title compound is synthesized by condensation of (3R,5S)-3-Ethylamino-5-{[9-(4-methoxy-butyl)-9H-xanthen-9-ylmethyl]-carbamoyl}-piperidine-1-carboxylic acid 9H-fluoren-9-ylmethyl ester (100 mg, 0.15 mmol) and phenylacetyl chloride (0.024 mL, 0.18 mmol) analogously to the preparation of “general procedure, scheme Y”. White powder; ES-MS: M+H=792; HPLC (Condition-A): t_{R}=4.90 min.

### (3R,5S)-3-Ethylamino-5-{[9-(4-methoxy-butyl)-9H-xanthen-9-ylmethyl]-carbamoyl}-piperidine-1-carboxylic acid 9H-fluoren-9-ylmethyl ester

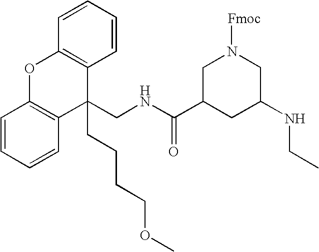

To a mixture of (3R,5S)-3-amino-5-{[9-(4-methoxy-butyl)-9H-xanthen-9-ylmethyl]-carbamoyl}-piperidine-1-carboxylic acid 9H-fluoren-9-ylmethyl ester (205 mg, 0.3 mmol) and sodiumcyano borohydride (20 mg, 0.3 mmol) in THF (3.0 mL), acetaldehyde (120 mL, 2.0 mmol) is slowly added dropwise at rt. After stirring for 3 hours, the reaction solution is diluted with H₂O, extracted with EtOAc, washed with brine, dried (Na₂SO₄), and concentrated. Purification by silica gel column chromatography gives the title compound; M+H=674; HPLC (Condition-A): t_{R}=3.70 min.

### (3R,5S)-3-Diethylamino-5-{[9-(4-methoxy-butyl)-9H-xanthen-9-ylmethyl]-carbamoyl}-piperidine-1-carboxylic acid tert-butyl ester

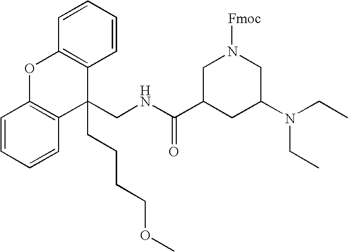

The titled compound is synthesized by reductive amination of (3R,5S)-3-Ethylamino-5-{[9-(4-methoxy-butyl)-9H-xanthen-9-ylmethyl]-carbamoyl}-piperidine-1-carboxylic acid tert-butyl ester and acetaldehyde using sodiumcyano borohydride analogously to the preparation of above procedure. White powder; ES-MS: M+H=674; HPLC (Condition): to =3.79 min

### (3S,5R)-3-{[9-(4-Methoxy-butyl)-9H-xanthen-9-ylmethyl]-carbamoyl}-5-(2-p-tolyl-acetylamino)-piperidine-1-carboxylic acid 9H-fluoren-9-ylmethyl ester

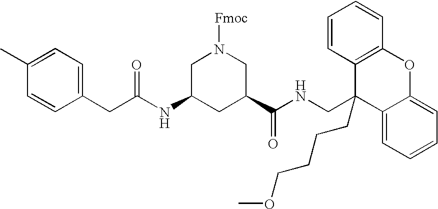

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (100 mg, 0.15 mmol) and p-tolyl acetic acid (26.4 mg, 0.18 mmol) analogously to the preparation of “general procedure, scheme X”. White amorphous material ES-MS: M+H=778; HPLC (Condition-A): t_{R}=4.78 min.

### (3S,5R)-3-{[9-(4-Methoxy-butyl)-9H-xanthen-9-ylmethyl]-carbamoyl}-5-phenylacetylamino-piperidine-1-carboxylic acid 9H-fluoren-9-ylmethyl ester

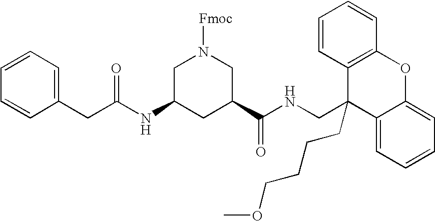

The title compound is synthesized by condensation of (3R,5S)-3-amino-5-{[9-(4-methoxybutyl)-9H-xanthen-9-ylmethyl]-carbamoyl}-piperidine-1-carboxylic acid 9H-fluoren-9-ylmethyl ester (100 mg, 0.15 mmol) and phenyl acetyl chloride (21.5 μL, 0.16 mmol) analogously to the preparation of “general procedure, scheme Y”. White amorphous material ES-MS: M+H=764; HPLC (Condition-B): t_{R}=2.34 min.

### (3R,5S)-3-(Ethyl-methanesulfonyl-amino)-5-{[9-(4-methoxy-butyl)-9H-xanthen-9-ylmethyl]-carbamoyl}piperidine-1-carboxylic acid tert-butyl ester

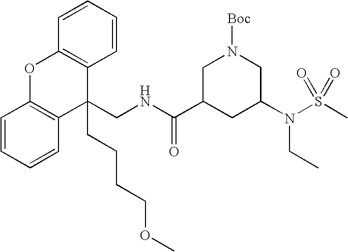

To a solution of (3R,5S)-3-Methanesulfonylamino-5-{[9-(4-methoxy-butyl)-99H-xanthen-9-ylmethyl]-carbamoyl}-piperidine-1-carboxylic acid tert-butyl ester (100 mg, 0.17 mmol) in DMF (1.0 mL), EtI (0.08 mL, 0.97 mmol) and K₂CO₃ (134 mg, 0.97 mmol) are added at rt. After stirring for 3 hours at 40° C., the reaction solution is purified silica gel column chromatography to give the titled compound; M+H=630; HPLC (condition-A): t_{R}=4.05 min.

### (3R,5S)-3-(Methyl-methanesulfonyl-amino)-5-{[9-(4-methoxy-butyl)-99H-xanthen-9-ylmethyl]-carbamoyl}piperidine-1-carboxylic acid tert-butyl ester

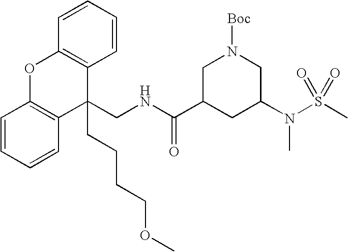

The titled compound is synthesized by methylation of (3R,5S)-3-Methanesulfonylamino-5-{[9-(4-methoxy-butyl)-99H-xanthen-9-ylmethyl]-carbamoyl}-piperidine-1-carboxylic acid tert-butyl ester (100 mg, 0.17 mmol) by MeI (0.05 mL, 0.83 mmol) analogously to the preparation of ‘above procedure’; M+H=616; HPLC (Condition-A): t_{R}=3.92 min.

### (3R,5S)-3-(Acetyl-ethyl-amino)-5-{[9-(4-methoxy-butyl)-9H-xanthen-9-ylmethyl]-carbamoyl}-piperidine-1-carboxylic acid tert-butyl ester

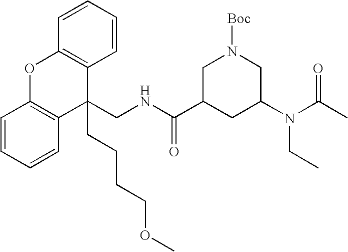

The titled compound is synthesized by condensation of (3R,5S)-3-ethylamino-5-{[9-(4-methoxy-butyl)-9H-xanthen-9-ylmethyl]-carbamoyl}-piperidine-1-carboxylic acid tert-butyl ester (55 mg, 0.1 mmol) and acetyl chloride (0.008 mL, 0.11 mmol) analogously to the preparation of ‘the general procedure-Y’. White amorphous material; ES-MS: M+H=594; HPLC (Condition-A): t_{R}=3.79 min.

### (3R,5S)-3-[Ethyl-(2-pyridin-4-yl-acetyl)-amino]-5-{[9-(4-methoxy-butyl)-9H-xanthen-9-ylmethyl]-carbamoyl}-piperidine-1-carboxylic acid tert-butyl ester

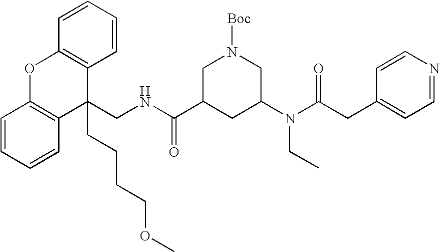

The titled compound is synthesized by condensation of (3R,5S)-3-ethylamino-5-{[9-(4-methoxy-butyl)-9H-xanthen-9-ylmethyl]-carbamoyl}-piperidine-1-carboxylic acid tert-butyl ester (55 mg, 0.1 mmol) and 4-pyridylacetic acid hydrochloride (21 mg, 0.12 mmol) using Et₃N (0.017 mL, 0.12 mmol) analogously to the preparation of the general procedure-Y. White amorphous material; ES-MS: M+H=671; HPLC (Condition-A): t_{R}=3.20 min.

### (3R,5S)-3-[Ethyl-(tetrahydro-pyran-4-yloxycarbonyl)-amino]-5-{[9-(4-methoxy-butyl)-9H-xanthen-9-ylmethyl]-carbamoyl}-piperidine-1-carboxylic acid tert-butyl ester

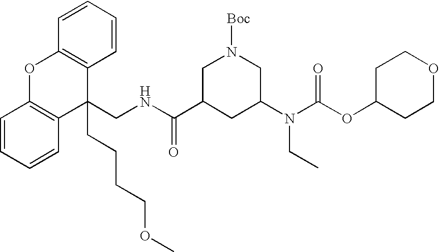

The titled compound is synthesized by condensation of (3R,5S)-3-ethylamino-5-{[9-(4-methoxy-butyl)-9H-xanthen-9-ylmethyl]-carbamoyl}-piperidine-1-carboxylic acid tert-butyl ester (90 mg, 0.17 mmol) and chloroformic acid tetrahydropyran-4-yl ester (41 mg, 0.25 mmol) analogously to the preparation of the general procedure-Y. White amorphous material; ES-MS: M+H=680; HPLC (condition-A): t_{R}=4.24 min.

### Mixture of equal parts of (3R,5S)-3-{Ethyl-[(R)-1-(tetrahydro-furan-2-yl)methoxy-carbonyl]-amino}-5-{[9-(4-methoxy-butyl)-9H-xanthen-9-yl-methyl]-carbamoyl}-piperidine-1-carboxylic acid tert-butyl ester and (3R,5S)-3-{Ethyl-[(S)-1-(tetrahydro-furan-2-yl)methoxycarbonyl]-amino}-5-{[9-(4-methoxy-butyl)-9H-xanthen-9-ylmethyl]-carbamoyl}-piperidine-1-carboxylic acid tert-butyl ester

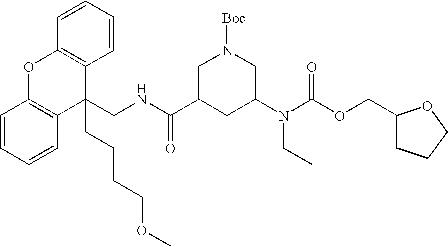

The titled compound is synthesized by condensation of (3R,5S)-3-ethylamino-5-{[9-(4-methoxy-butyl)-9H-xanthen-9-ylmethyl]-carbamoyl}-piperidine-1-carboxylic acid tert-butyl ester (90 mg, 0.17 mmol) and tetrahydrofurfuryl chloroformate (41 mg, 0.25 mmol) analogously to the preparation of the general procedure-Y. White amorphous material; ES-MS: M+H=680; HPLC (Condition-A): t_{R}=4.30 min.

### (3S,5R)-3-{[9-(4-Methoxy-butyl)-9H-xanthen-9-ylmethyl]-carbamoyl}-5-[propyl-(2-pyridin-4-yl-acetyl)-amino]-piperidine-1-carboxylic acid tert-butyl ester

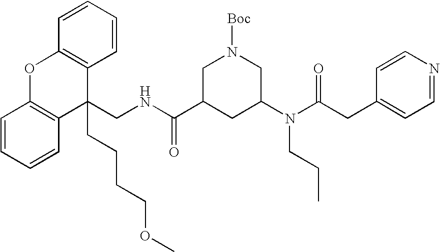

The titled compound is synthesized by condensation of (3S,5R)-3-{[9-(4-Methoxy-butyl)-9H-xanthen-9-ylmethyl]-carbamoyl}-5-propylamino-piperidine-1-carboxylic acid tert-butyl ester (80 mg, 0.14 mmol) and 4-pyridylacetic acid hydrochloride (30 mg, 0.17 mmol) analogously to the preparation of the general procedure-Y. The crude product is used without purification.

### (3S,5R)-3-{[9-(4-Methoxy-butyl)-9H-xanthen-9-ylmethyl]-carbamoyl}-5-propylamino-piperidine-1-carboxylic acid tert-butyl ester

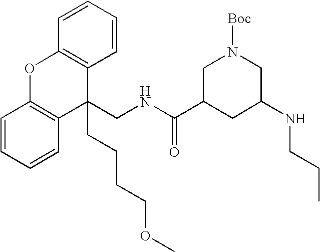

To a solution of (3S,5R)-3-{[9-(4-Methoxy-butyl)-9H-xanthen-9-ylmethyl]-carbamoyl}-5-(2-nitro-benzenesulfonylamino)-piperidine-1-carboxylic acid tert-butyl ester (100 mg, 0.14 mmol) in DMF (2 mL), 1-bromopropane (13 μL, 0.21 mmol) and K₂CO₃ (58 mg, 0.42 mmol) are added at rt. After stirring at 50° C. over night, the reaction mixture is diluted with H₂O and extracted with EtOAc. The combined organic phases are washed with H₂O and dried over Na₂SO₄. Concentration under reduced pressure and filtration through silica gel give crude product. To a solution of the crude product in DMF (2 mL), thioglycolic acid (20 μL, 0.28 mmol) and LiOH (14 mg, 0.56 mmol) are added at rt. After stirring for 4 hours, the reaction mixture is diluted with H₂O and extracted with EtOAc. The combined organic phases are washed with H₂O, dried over Na₂SO₄, and Concentrated under reduced pressure and purified with silica gel to give the titled compound; ES-MS: M+H=566; HPLC (Condition-A): t_{R}=3.38 min

### (3S,5R)-3-{[9-(4-Methoxy-butyl)-9H-xanthen-9-ylmethyl]-carbamoyl}-5-(2-nitro-benzenesulfonylamino)-piperidine-1-carboxylic acid tert-butyl ester

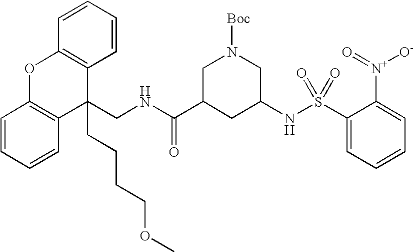

To a solution of (3S,5R)-3-{[9-(4-Methoxy-butyl)-9H-xanthen-9-ylmethyl]-carbamoyl}-5-(2-nitro-benzenesulfonylamino)-piperidine-1-carboxylic acid 9H-fluoren-9-ylmethyl ester (1.2 g, 1.46 mmol) in DMF (10 mL), KF (420 mg, 7.3 mmol), Et₃N (0.41 mL, 2.9 mmol), and Boc₂O (410 mg, 1.9 mmol) are added at rt. After stirring for 2.5 hours at rt, the reaction mixture is diluted with H₂O and extracted with EtOAc. The combined organic phases are washed with H₂O and dried over Na₂SO₄. Purification by column chromatography gives the titled compound; ES-MS: M+H=709; HPLC (Condition-A): t_{R}=4.18 min

### (3S,5R)-3-{[9-(4-Methoxy-butyl)-9H-xanthen-9-ylmethyl]-carbamoyl}-5-(2-nitro-benzenesulfonylamino)-piperidine-1-carboxylic acid 9H-fluoren-9-ylmethyl ester

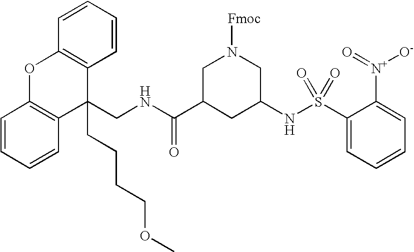

To a solution of (3R,5S)-3-Amino-5-{[9-(4-methoxy-butyl)-9H-xanthen-9-ylmethyl]-carbamoyl}-piperidine-1-carboxylic acid 9H-fluoren-9-ylmethyl ester (1.0 g, 1.5 mmol) in CH₂Cl₂/H₂O (5 mL/5 mL), 2-nitrobenzenesulfonyl chloride (400 mg, 1.8 mmol), sodium bicarbonate (380 mg, 3.6 mmol) are added at rt. After stirring for 3 hours at the temperature, the reaction mixture is diluted with H₂O and extracted with EtOAc. The combined organic phases are washed with H₂O and dried over Na₂SO₄. Purification by column chromatography gives the titled compound; ES-MS: M+H=831; HPLC (Condition-A): t_{R}=4.72 min

### (3R,5S)-3-[Cyclopropylmethyl-(2-pyridin-4-yl-acetyl)-amino]-5-{[9-(4-methoxy-butyl)-9H-xanthen-9-ylmethyl]-carbamoyl}-piperidine-1-carboxylic acid tert-butyl ester

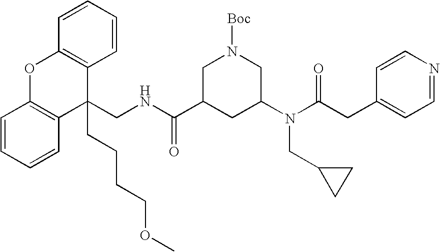

The titled compound is synthesized by condensation of (3R,5S)-3-(Cyclopropylmethylamino)-5-([9-(4-methoxy-butyl)-9H-xanthen-9-ylmethyl]-carbamoyl)-piperidine-1-carboxylic acid tert-butyl ester (80 mg, 0.14 mmol) and 4-pyridylacetic acid hydrochloride (30 mg, 0.17 mmol) analogously to the preparation of the general procedure-Y. White amorphous material; ES-MS: M+H=697; HPLC (Condition-A): t_{R}=3.35, 3.42 min. (Two rotamers were observed.)

### (3R,5S)-3-(Cyclopropylmethyl-amino)-5-[9-(4-methoxy-butyl)-9H-xanthen-9-ylmethyl]-carbamoyl)-piperidine-1-carboxylic acid tert-butyl ester

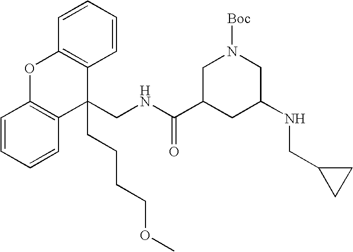

The titled compound is synthesized by alkylation of (3S,5R)-3-{[9-(4-Methoxy-butyl)-9H-xanthen-9-ylmethyl]-carbamoyl}-5-(2-nitro-benzenesulfonylamino)-piperidine-1-carboxylic acid tert-butyl ester (100 mg, 0.14 mmol), followed by de-nosylation using bromocyclopropane (0.02 mL, 0.21 mmol) analogously to the preparation of (3S,5R)-3-{[9-(4-Methoxybutyl)-9H-xanthen-9-ylmethyl]-carbamoyl}-5-propylamino-piperidine-1-carboxylic acid tert-butyl ester. White amorphous material; ES-MS: M+H=578; HPLC (Condition-A): t_{R}=3.43 min.

### (3R,5S)-3-[Ethyl-(3-methyl-butyryl)-amino]-5-{[9-(4-methoxy-butyl)-9H-xanthen-g-ylmethyl]-carbamoyl}-piperidine-1-carboxylic acid tert-butyl ester

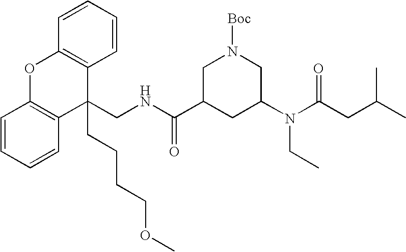

The titled compound is synthesized by condensation of (3R,5S)-3-Ethylamino-5-{[9-(4-methoxy-butyl)-9H-xanthen-9-ylmethyl]-carbamoyl}-piperidine-1-carboxylic acid tert-butyl ester (92 mg, 0.16 mmol) and 4-pyridylacetic acid hydrochloride (0.02 mL, 0.18 mmol) analogously to the preparation of the general procedure-Y. White amorphous material; ES-MS: M+H=636; HPLC (Condition-A): t_{R}=5.39 min.

### Examples 17 to 79

The following Examples enlisted in Table 1 are synthesized by deprotection of Boc or Fmoc group, analogously to the Example 15 as hereinafter or hereinbefore described. As far as not being commercially available by synthesis analogous to methods or as described hereinbefore. The Asterisk (*) indicates the end of the bond at which the respective moiety is bound to the rest of the molecule falling under the following formula:

TABLE 1

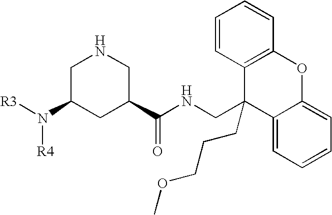

| | | | t_{R} | MS |
|---|---|---|---|---|
| No. | R4 | R3 | (Condition) | (M + H) |
| 17 | —H | 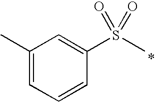 | 2.35 (B) | 578 |
| 18 | —H | 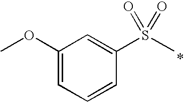 | 2.38 (B) | 594 |
| 19 | —H | 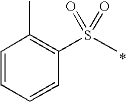 | 2.98 (A) | 578 |
| 20 | —H | 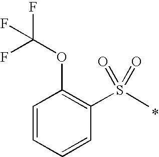 | 3.12 (A) | 648 |
| 21 | —H | 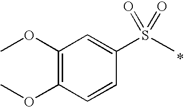 | 2.84 (A) | 624 |
| 22 | —H | 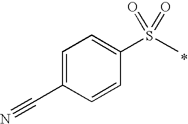 | 2.88 (A) | 589 |
| 23 | —H | 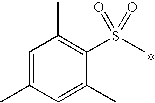 | 3.22 (A) | 606 |
| 24 | —H | 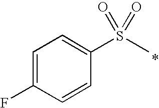 | 2.97 (A) | 582 |
| 25 | —H | 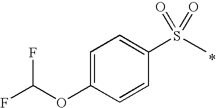 | 1.76 (B) | 630 |
| 26 | —H | 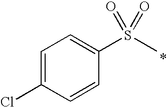 | 1.90 (B) | 598 |
| 27 | —H | 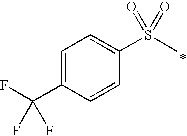 | 1.88 (B) | 632 |
| 28 | —H | 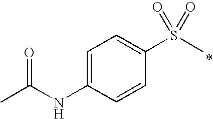 | 1.59 (B) | 621 |
| 29 | —H | 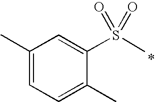 | 1.79 (B) | 592 |
| 30 | —H | 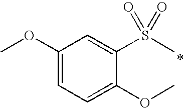 | 1.81 (B) | 624 |
| 31 | —H | 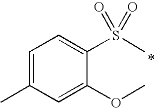 | 1.85 (B) | 608 |
| 32 | —H | 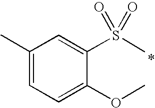 | 2.06 (B) | 608 |
| 33 | —H | 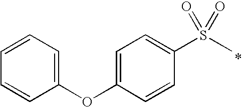 | 1.84 (B) | 656 |
| 34 | —H | 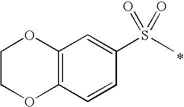 | 1.67 (B) | 621 |
| 35 | —H | 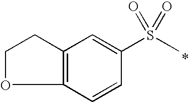 | 2.85 (A) | 606 |
| 36 | —H | 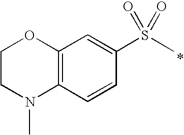 | 2.90 (A) | 635 |
| 37 | —H | 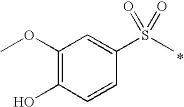 | 2.72 (A) | 610 |
| 38 | —H | 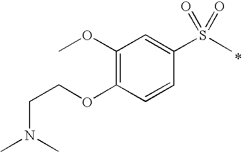 | 2.40 (A) | 681 |
| 39 | —H | 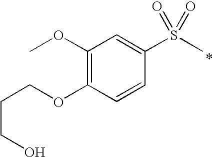 | 2.67 (A) | 668 |
| 40 | —H | 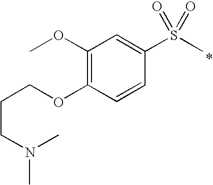 | 2.40 (A) | 695 |
| 41 | —H | 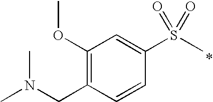 | 2.40 (A) | 621 |
| 42 | —H | 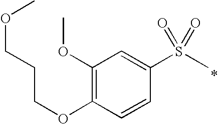 | 2.97 (A) | 682 |
| 43 | —H | 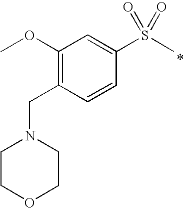 | 2.43 (A) | 663 |
| 44 | —H | 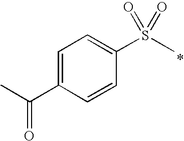 | 1.66 (B) | 606 |
| 45 | —H | 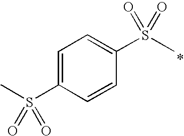 | 1.63 (B) | 642 |
| 46 | —H | 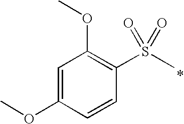 | 1.69 (B) | 624 |
| 47 | —H | 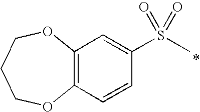 | 1.71 (B) | 636 |
| 48 | —H | 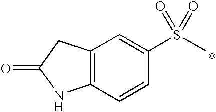 | 1.55 (B) | 619 |
| 49 | —H | 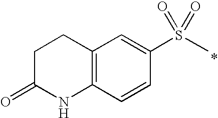 | 1.57 (B) | 633 |
| 50 | —H | 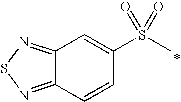 | 1.71 (B) | 622 |
| 51 | —H | 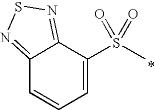 | 1.68 (B) | 622 |
| 52 | —H | 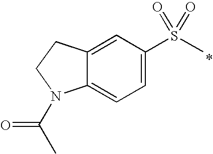 | 1.62 (B) | 647 |
| 53 | —H | 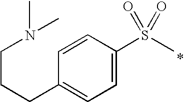 | 2.43 (A) | 649 |
| 54 | —H | 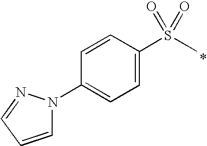 | 2.95 (A) | 630 |
| 55 | —H | 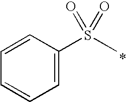 | 1.69 (B) | 564 |
| 56 | —H | 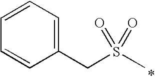 | 2.95 (A) | 578 |
| 57 | —H | 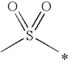 | 2.55 (A) | 502 |
| 58 | —H | 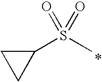 | 2.68 (A) | 528 |
| 59 | —H | 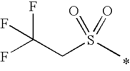 | 2.88 (A) | 570 |
| 60 | —H | 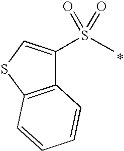 | 3.09 (A) | 620 |
| 61 | —H | 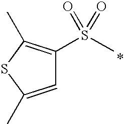 | 3.09 (A) | 598 |
| 62 | —H | 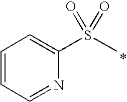 | 1.61 (B) | 565 |
| 63 | —H | 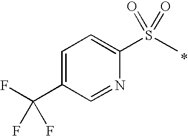 | 3.02 (A) | 633 |
| 64 | —H | 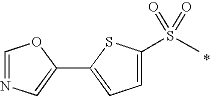 | 2.82 (A) | 637 |
| 65 | —H | 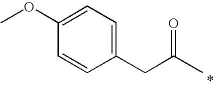 | 2.92 (A) | 572 |
| 66 | —H | 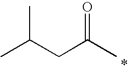 | 2.84 (A) | 508 |
| 67 | -Et | 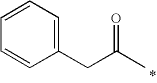 | 3.10 (A) | 570 |
| 68 | -Me | 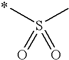 | 2.70 (A) | 516 |
| 69 | -Et | 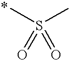 | 2.79 (A) | 530 |
| 70 | -Et | -Et | 2.35 (A) | 480 |
| 71 | -Et | 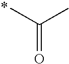 | 2.63 (A) | 494 |
| 72 | -Et | 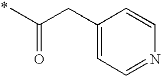 | 2.34 (A) | 571 |
| 73 | -Et | 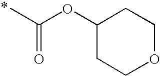 | 2.88 (A) | 580 |
| 74 | -Et | 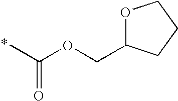 | 2.93 (A) | 580 |
| 75 | -nPr | 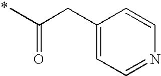 | 2.44, 2.50 (A)two rotamers | 585 |
| 76 | 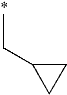 | 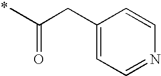 | 1.51 (B) | 597 |
| 77 | -Et | 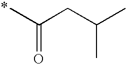 | 2.95 (A) | 536 |
| 78 | -Me | 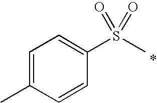 | 1.81 (B) | 592 |
| 79 | -Me | 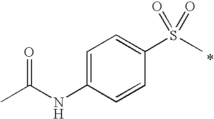 | 1.60 (B) | 635 |

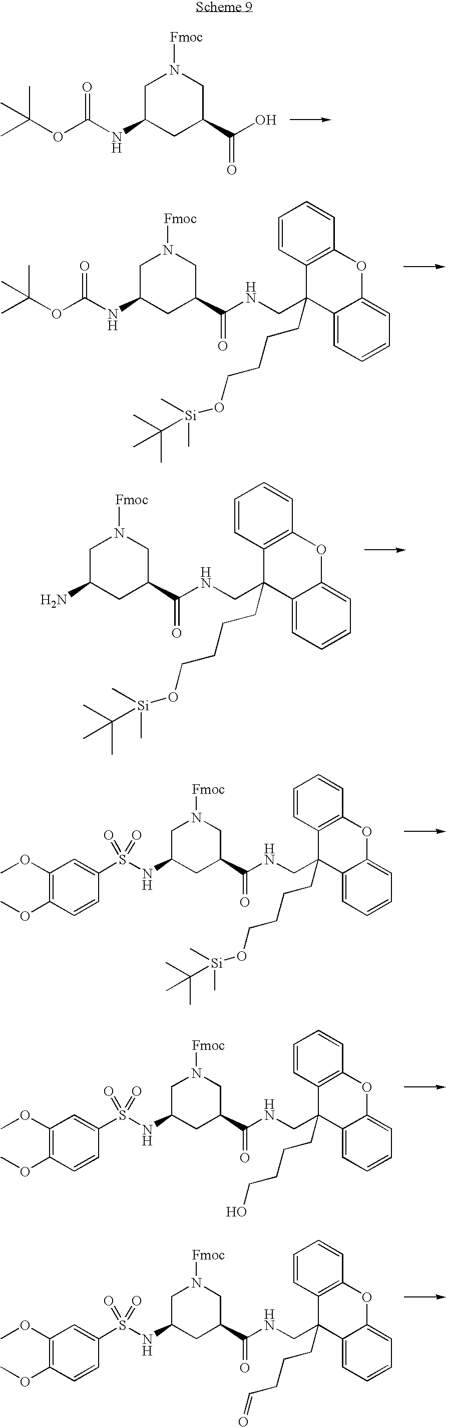

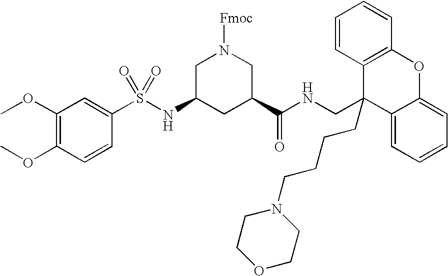

### F1. (3R,5S)-3-tert-Butoxycarbonylamino-5-({9-[4-(tert-butyl-dimethyl-silanyloxy)-butyl]-9H-xanthen-9-ylmethyl}-carbamoyl)-piperidine-1-carboxylic acid 9H-fluoren-9-ylmethyl ester

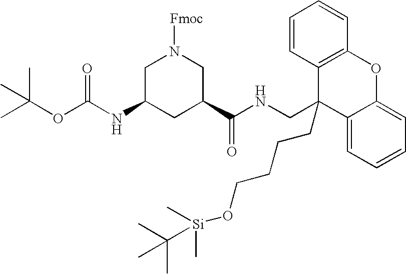

To a solution of (3S,5R)-5-tert-butoxycarbonylamino-piperidine-1,3-dicarboxylic acid 1-(9H-fluoren-9-ylmethyl) ester (1 g, 2 mmol) in DMF (5 mL) under N₂ was added, at room temperature, EDCl.HCl (456 mg, 2 mmol) and HOAT (272 mg, 2 mmol). Reaction mixture is stirred at room temperature for few minutes. Then, C-{9-[4-(tert-butyl-dimethyl-silanyloxy)butyl]-9H-xanthen-9-yl}-methylamine (795 mg, 2 mmol) was added. After stirring at room temperature for 2 hours, H₂O was added. Reaction mixture was extracted with ethylacetate, dried over Na₂SO₄, concentrated under reduced pressure and subjected to silica gel chromatography to give the titled compound as white amorphous material. MS (M+H)=846; HPLC (Condition-A): t_{R}=6.17 min.

### F2. (3R,5S)-3-Amino-5-({9-[4-(tert-butyl-dimethyl-silanyloxy)-butyl]-9H-xanthen-9-ylmethyl}-carbamoyl)-piperidine-1-carboxylic acid 9H-fluoren-9-ylmethyl ester

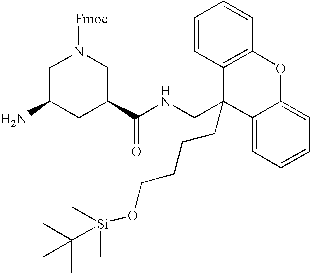

To the solution of (3R,5S)-3-tert-butoxycarbonylamino-5-({9-[4-(tert-butyl-dimethylsilanyloxy)-butyl]-9H-xanthen-9-ylmethyl}-carbamoyl)-piperidine-1-carboxylic acid 9H-fluoren-9-ylmethyl ester (1.14 g, 1.3 mmol) in CH₂Cl₂ (5 mL) under N₂ was added 2,6-lutidine (0.98 mL, 8.4 mmol) and TMSOTf (0.78 mL, 4.2 mmol) at 0° C. The resulting solution is warmed to room temperature and stirred for 4 hours. Then, sat. NaHCO₃ aq. and MeOH were added, concentrated under reduced pressure to give the titled compound. MS (M+H)=746; HPLC (Condition-A): t_{R}=4.77 min.

### F3. (3S,5R)-3-({9-[4-(tert-Butyl-dimethyl-silanyloxy)-butyl]-9H-xanthen-9-ylmethyl}-carbamoyl)-5-(3,4-dimethoxy-benzenesulfonylamino)-piperidine-1-carboxylic acid 9H-fluoren-9-ylmethyl ester

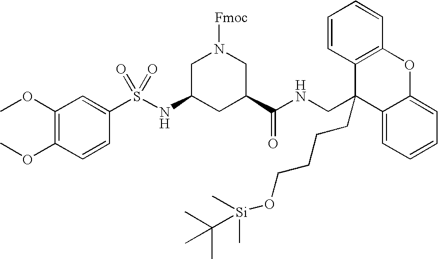

To a solution of (3R,5S)-3-amino-5-({9-[4-(tert-butyl-dimethyl-silanyloxy)-butyl]-9H-xanthen-9-ylmethyl}-carbamoyl)-piperidine-1-carboxylic acid 9H-fluoren-9-ylmethyl ester in CH₂Cl₂ (5 mL) under N₂ was added 3,4-dimethoxy benzenesulfonyl chloride (555 mg, 2.3 mmol) and cat. DMAP at room temperature. The resulting solution was stirred at room temperature for 1 hour, and H₂O was added. Reaction mixture was extracted with CH₂Cl₂, dried over Na₂SO₄, concentrated under reduced pressure and subjected to reverse-phase chromatography to give the titled compound (1.05 g, 1.1 mmol) as white amorphous material. MS (M+H)=946; HPLC (Condition-A): t_{R}=5.75 min.

### F4. (3R,5S)-3-(3,4-Dimethoxy-benzenesulfonylamino)-5-{[9-(4-hydroxy-butyl)-9H-xanthen-9-ylmethyl]-carbamoyl}-piperidine-1-carboxylic acid 9H-fluoren-9-ylmethyl ester

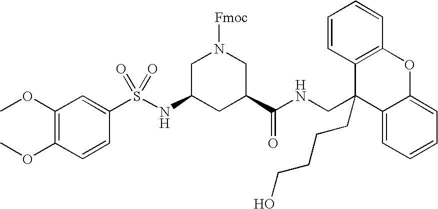

To a solution of (3S,5R)-3-({9-[4-(tert-butyl-dimethyl-silanyloxy)-butyl]-9H-xanthen-9-ylmethyl}-carbamoyl)-5-(3,4-dimethoxy-benzenesulfonylamino)-piperidine-1-carboxylic acid 9H-fluoren-9-ylmethyl ester (1.05 g, 1.1 mmol) in dioxane (5 mL) was added 1N HCl aq. (4 mL) at room temperature. After stirring at room temperature for 3 hours, 5 N HCl aq. (1 mL) was added. The resulting mixture was stirred at room temperature for few minutes. NaHCO₃ aq. was added, the reaction mixture was extracted with CH₂Cl₂, dry over Na₂SO₄, concentrated under reduced pressure and subjected to silica chromatography to give the titled compound in quantitative yield as white amorphous material. MS (M+H)=832; HPLC (Condition-A): t_{R}=3.98 min.

### F5. (3R,5S)-3-(3,4-Dimethoxy-benzenesulfonylamino)-5-{[9-(4-oxo-butyl)-9H-xanthen-9-ylmethyl]-carbamoyl}-piperidine-1-carboxylic acid 9H-fluoren-9-ylmethyl ester

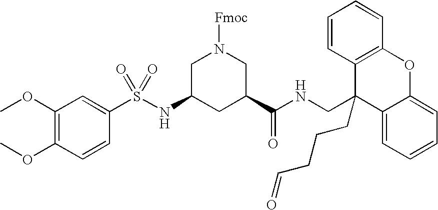

To a solution of (3R,5S)-3-(3,4-dimethoxy-benzenesulfonylamino)-5-{[9-(4-hydroxy-butyl)-9H-xanthen-9-ylmethyl]-carbamoyl}-piperidine-1-carboxylic acid 9H-fluoren-9-ylmethyl ester (132.1 mg, 0.16 mmol) in CH₂Cl₂ (5 mL) was added under N₂, Dess-Martin periodinane (94.6 mg, 0.22 mmol) at room temperature. The reaction mixture was stirred at room temperature for 30 min. and filtered by using silica gel to gave the titled compound (151.7 mg, 0.155 mmol). MS (M+H)=830; HPLC (Condition-A): t_{R}=4.24 min.

### F6. (3R,5S)-3-(3,4-Dimethoxy)-benzenesulfonylamino)-5-{[9-(4-morpholin-4-yl-butyl)-9H-xanthen-9-ylmethyl]-carbamoyl}-piperidine-1-carboxylic acid 9H-fluoren-9-ylmethyl ester

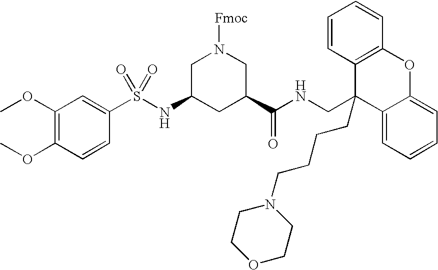

To a solution of (3R,5S)-3-(3,4-dimethoxy-benzenesulfonylamino)-5-{[9-(4-oxo-butyl)-9H-xanthen-9-ylmethyl]-carbamoyl}piperidine-1-carboxylic acid 9H-fluoren-9-ylmethyl ester (151.7 mg, 0.155 mmol) in CH₂Cl₂ (3 mL) was added under N₂, acetic acid (20 μL, 0.35 mmol) and morpholine (19.9 μL, 0.23 mmol) at 0° C. After stirring at room temperature for few minutes, sodium triacetoxyborohydride (62.5 mg, 0.29 mmol) was added to the reaction mixture. The resulting solution was warmed to room temperature and stirred for 50 min. After NaHCO₃ aq. was added, the reaction mixture was extracted with CH₂Cl₂, dried over Na₂SO₄, and concentrated under reduced pressure to give the titled compound. ES-MS: (M+H)=901; HPLC (Condition-A): t_{R}=3.37 min.

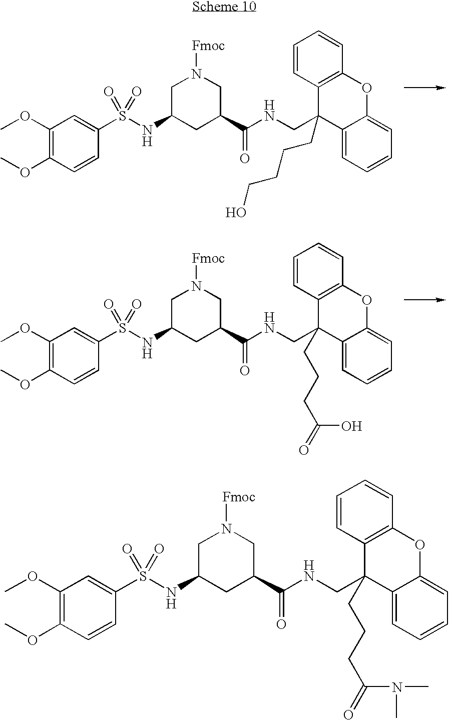

### G1. (3S,5R)-3-{[9-(3-Carboxy-propyl)-9H-xanthen-9-ylmethyl]-carbamoyl}-5-(3,4-dimethoxy-benzenesulfonylamino)-piperidine-1-carboxylic acid 9H-fluoren-9-ylmethyl ester

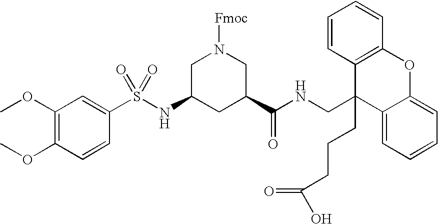

To a solution of (3R,5S)-3-(3,4-dimethoxy-benzenesulfonylamino)-5-{[9-(4-hydroxy-butyl)-9H-xanthen-9-ylmethyl]-carbamoyl}-piperidine-1-carboxylic acid 9H-fluoren-9-ylmethyl ester (699.2 mg, 0.84 mmol) in CH₂Cl₂ (5 mL) was added under N₂, Dess-Martin periodinane (534.6 mg, 1.26 mmol) at room temperature. The reaction mixture was stirred at room temperature for 1.5 hours and filtered through a pad of silica gel, and the solvent was evaporated under reduced pressure. Next, to the obtained residue (346.2 mg), 2-methyl-2-butene (0.44 ml, 4.1 mmol) and NaH₂PO₄ (250 mg, 2.1 mmol) in a mixture of tert-BuOH and H₂O (5/1) was added NaClO₂ (235 mg, 2.1 mmol, 80% purity) at room temperature. After stirred at room temperature for 1.5 hours, brine was added. The reaction mixture was extracted with CH₂Cl₂, dried over Na₂SO₄, concentrated under reduced pressure to afford the titled compound (172.8 mg) as white amorphous material. MS (M+H)=846; HPLC (Condition-A): t_{R}=3.95 min.

### G2. (3R,5S)-3-(3,4-Dimethoxy-benzenesulfonylamino)-5-{[9-(3-dimethylcarbamoyl-propyl)-9H-xanthen-9-ylmethyl]-carbamoyl}-piperidine-1-carboxylic acid 9H-fluoren-9-ylmethyl ester

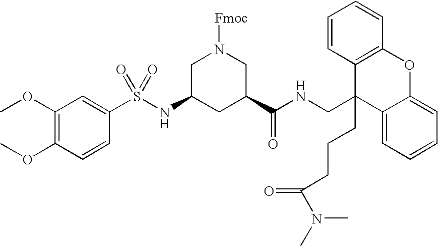

To a solution of (3S,5R)-3-{[9-(3-carboxy-propyl)-9H-xanthen-9-ylmethyl]-carbamoyl}-5-(3,4-dimethoxy-benzenesulfonylamino)-piperidine-1-carboxylic acid 9H-fluoren-9-ylmethyl ester in CH₂Cl₂ (5 mL) was added under N₂, EDCl.HCl (54.7 mg, 0.24 mmol), HOAT (32.6 mg, 0.24 mmol) and triethyl amine (160 μL, 1.16 mmol) at room temperature. Reaction mixture was is stirred at room temperature for few minutes. Then, dimethyl amine HCl salt (160 mg, 1.96 mmol) was added. After stirred at room temperature overnight, H₂O was added. The reaction mixture was extracted with CH₂Cl₂, dried over Na₂SO₄, and concentrated under reduced pressure to give the titled compound as white amorphous material. MS M+H=873; HPLC (Condition-A): t_{R}=4.05 min.

### G3. (3R,5S)-3-tert-Butoxycarbonylamino-5-{[9-(2-methoxy-ethoxymethyl)-9H-xanthen-9-ylmethyl]-carbamoyl}-Piperidine-1-carboxylic acid 9H-fluoren-9-ylmethyl ester

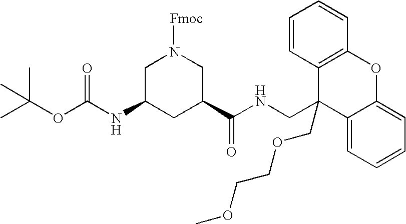

The title compound is synthesized by condensation of (3S,5R)-5-tert-butoxycarbonyl-aminopiperidine-1,3-dicarboxylic acid 1-(9H-fluoren-9-ylmethyl) ester (202.9 mg, 0.43 mmol) and C-[9-(2-methoxy-ethoxymethyl)-9H-xanthen-9-yl]-methylamine (133.2 mg, 0.44 mmol) analogously to the preparation of (3R,5S)-3-tert-butoxycarbonylamino-5-({9-[4-(tert-butyldimethyl-silanyloxy)-butyl]-9H-xanthen-9-ylmethyl}-carbamoyl)-piperidine-1-carboxylic acid 9H-fluoren-9-ylmethyl ester in F1 as white amorphous material. ES-MS: M+H=748; HPLC (Condition-A): t_{R}=4.68 min.

### G4. (3R,5S)-3-Amino-5-{[9-(2-methoxy-ethoxymethyl)-9H-xanthen-9-ylmethyl]-carbamoyl}-piperidine-1-carboxylic acid 9H-fluoren-9-ylmethyl ester

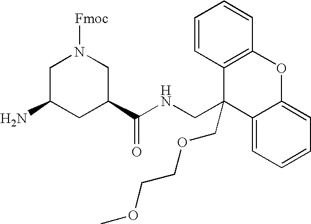

The title compound is synthesized by deprotection of (3R,5S)-3-tert-butoxycarbonylamino-5-{[9-(2-methoxy-ethoxymethyl)-9H-xanthen-9-ylmethyl]-carbamoyl}-piperidine-1-carboxylic acid 9H-fluoren-9-ylmethyl ester analogues “General procedure, Scheme 6”. white amorphous material. ES-MS: M+H=648; HPLC (Condition-A): t_{R}=3.35 min.

### (3S,5R)-3-{[9-(2-Methoxy-ethoxymethyl)-9H-xanthen-9-ylmethyl]-carbamoyl}-5-(toluene-4-sulfonylamino)piperidine-1-carboxylic acid 9H-fluoren-9-ylmethyl ester

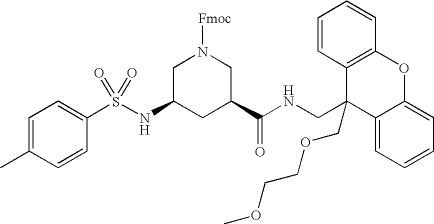

The title compound is synthesized by sulfonylation of (3R,5S)-3-amino-5-{[9-(2-methoxyethoxymethyl)-9H-xanthen-9-ylmethyl]-carbamoyl}-piperidine-1-carboxylic acid 9H-fluoren-9-ylmethyl ester (336.8 mg, 0.52 mmol) and p-toluene sulfonyl chloride (118 mg, 0.62 mmol) analogously to the preparation of “General procedure, Scheme 6”. White amorphous material ES-MS: M+H=802 (Condition-A); HPLC: t_{R}=4.70 min.

### (3R,5S)-3-(4-Fluoro-benzenesulfonylamino)-5-{[9-(2-methoxy-ethoxymethyl)-9H-xanthen-9-ylmethyl]-carbamoyl}-piperidine-1-carboxylic acid 9H-fluoren-9-ylmethyl ester

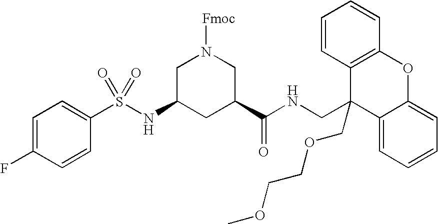

The title compound is synthesized by sulfonylation (3R,5S)-3-amino-5-{[9-(2-methoxyethoxymethyl)-9H-xanthen-9-ylmethyl]-carbamoyl}-piperidine-1-carboxylic acid 9H-fluoren-9-ylmethyl ester (100 mg, 0.15 mmol) and 4-fluoro benzenesulfonyl chloride (40 mg, 0.21 mmol) analogously to the preparation of “General procedure, Scheme 6”. White amorphous material ES-MS: M+H=806; HPLC (Condition-A): t_{R}=4.47 min.

### (3S,5R)-3-{[9-(2-Methoxy-ethoxymethyl)-9H-xanthen-9-ylmethyl]-carbamoyl}-5-(4-trifluoromethyl-benzenesulfonylamino)-piperidine-1-carboxylic acid 9H-fluoren-9-ylmethyl ester

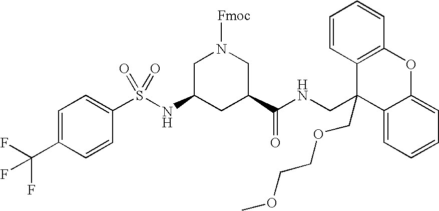

The title compound is synthesized by sulfonylation of (3R,5S)-3-amino-5-{[9-(2-methoxyethoxymethyl)-9H-xanthen-9-ylmethyl]-carbamoyl}-piperidine-1-carboxylic acid 9H-fluoren-9-ylmethyl ester (104.7 mg, 0.15 mmol) and 4-trifluoromethyl benzenesulfonyl chloride (69.5 mg, 0.28 mmol) analogously to the preparation of “General procedure, Scheme 6”. White amorphous material ES-MS: M+H=856; HPLC (Condition-A): t_{R}=4.74 min.

### (3S,5R)-3-tert-Butoxycarbonylamino-5-{[9-(2-ethoxy-Propyl)-9H-xanthen-9-ylmethyl]-carbamoyl}-piperidine-1-carboxylic acid 9H-fluoren-9-ylmethyl ester

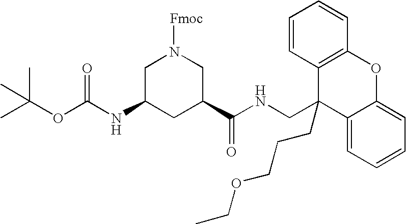

The title compound is synthesized by condensation of (3S,5R)-5-tert-butoxycarbonylamino-piperidine-1,3-carboxylic acid 1-(9H-fluoren-9-ylmethyl) ester (376.8 mg, 0.807 mmol) and C-[9-(3-Ethoxy-propyl)-9H-xanthen-9-yl]-methylamine (240.2 mg, 0.807 mmol) analogously to the preparation of “General procedure, Scheme 6”. white amorphous material. ES-MS: M+H=746; HPLC (Condition-B): t_{R}=2.36 min.

### (3S,5R)-3-Amino-5-{[9-(3-ethoxy-propyl)-9H-xanthen-9-ylmethyl]-carbamoyl}-piperidine-1-carboxylic acid 9H-fluoren-9-ylmethyl ester

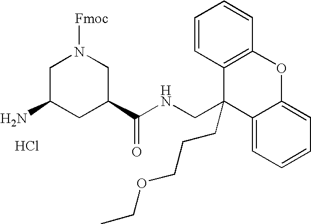

The title compound is synthesized by deprotection of (3S,5R)-3-tert-butoxycarbonylamino-5-{[9-(2-ethoxy-propyl)-9H-xanthen-9-ylmethyl]-carbamoyl}-piperidine-1-carboxylic acid 9H-fluoren-9-ylmethyl ester (448.7 mg, 0.60 mmol) analogously “General procedure, Scheme 6”. white amorphous material. ES-MS: M+H=646; HPLC (Condition-B): t_{R}=1.96 min.

### (3R,5S)-3-(3,4-Dimethoxy-benzenesulfonylamino)-5-{[9-(3-ethoxy-propyl)-9H-xanthen-9-ylmethyl]-carbamoyl}-piperidine-1-carboxylic acid 9H-fluoren-9-ylmethyl ester

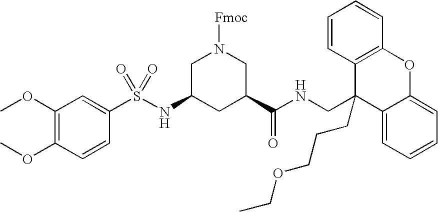

The title compound is synthesized by sulfonylation of (3S,5R)-3-amino-5-{[9-(3-ethoxypropyl)-9H-xanthen-9-ylmethyl]-carbamoyl}-piperidine-1-carboxylic acid 9H-fluoren-9-ylmethyl ester (190.3 mg, 0.28 mmol) and 3,4-dimethoxy sulfonylchloride (72.6 mg, 0.31 mmol) analogously to the preparation of “General procedure, Scheme 6”. White amorphous material; a white solid; ES-MS: M+H=846; HPLC (Condition-B): t_{R}=2.24 min.

### (3S,5R)-3-{[9-(3-Ethoxy-propyl)-9H-xanthen-9-ylmethyl]-carbamoyl}-5-(4-fluoro-benzenesulfonylamino)-piperidine-1-carboxylic acid 9H-fluoren-9-ylmethyl ester

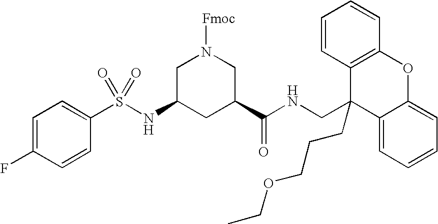

Intermediate TAI701.1 is synthesized by sulfonylation of intermediate TAI699.2 (262.1 mg, 0.38 mmol) and 4-fluoro sulfonylchloride (74.8 mg, 0.38 mmol) analogously to the preparation of “General procedure, Scheme 6”. White amorphous material; a white solid; ES-MS: M+H=804; HPLC (Condition-B): t_{R}=2.28 min.

### Examples 80 to 92

The following Example enlisted in Table 2 are synthesized by deprotection of Fmoc group, analogously to the Example 15, as hereinafter or hereinbefore described. As far as not being commercially available by synthesis analogous to methods or as described hereinbefore. The Asterisk (*) indicates the end of the bond at which the respective moiety is bound to the rest of the molecule falling under the following formula:

TABLE 2

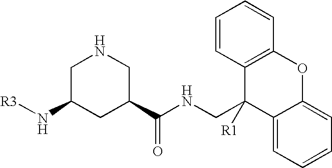

| | | | t_{R} min. | MS |
|---|---|---|---|---|
| No. | R1 | R3 | (HPLC condition) | [M + H]⁺ |
| 80 | 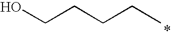 | 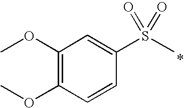 | 2.40 (A) | 610 |
| 81 | 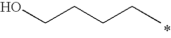 | 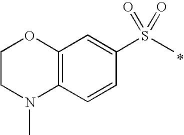 | 2.54 (A) | 621 |
| 82 | 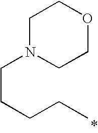 | 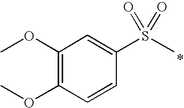 | 2.09 (B) | 679 |
| 83 | 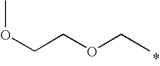 | 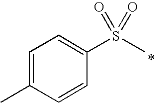 | 2.87 (B) | 580 |
| 84 | 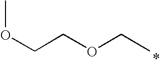 | 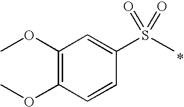 | 1.53 (B) | 626 |
| 85 | 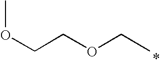 | 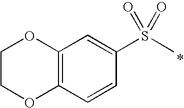 | 1.60 (B) | 624 |
| 86 | 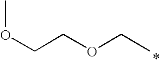 | 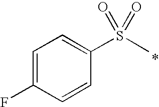 | 2.73 (B) | 584 |
| 87 | 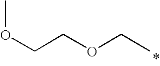 | 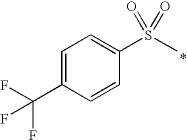 | 3.02 (B) | 634 |
| 88 | 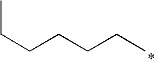 | 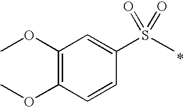 | 1.89 (B) | 622 |
| 89 | 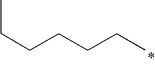 | 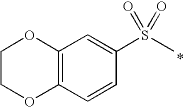 | 1.94 (B) | 620 |
| 90 | 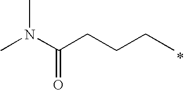 | 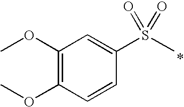 | 2.50 (B) | 651 |
| 91 | 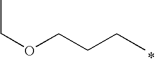 | 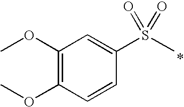 | 1.67 (B) | 624 |
| 92 | 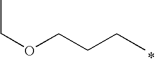 | 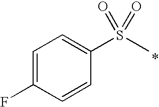 | 1.74 (B) | 582 |

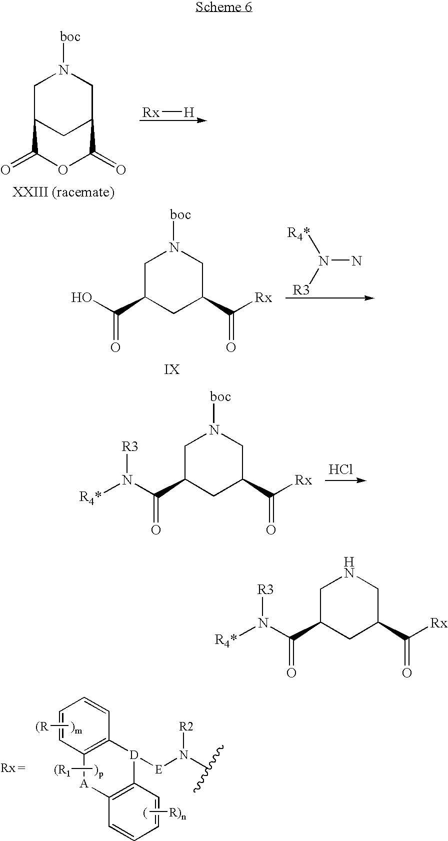

R3 and R₄* are as defined in the starting materials, preferably they are deducible from the following examples, as is Rx:

### Example 93

### (General procedure, Scheme 6): (3S*,5R*)-Piperidine-3,5-dicarboxylic acid 3-methylamide 5-[(9H-xanthen-9-ylmethyl)-amide]

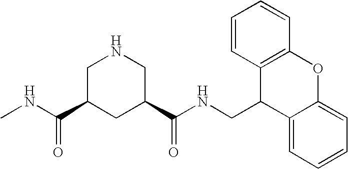

HCl in dioxane (4 M, 0.1 mL is added at rt to a solution of (3R*,5S*)-3-methylcarbamoyl-5-[(9H-xanthen-9-ylmethyl)-carbamoyl]-piperidine-1-carboxylic acid tert-butyl ester in dioxane and the resulting solution is stirred at rt for 3 h. At this time it is frozen and lyophilised to give a white powder. MS: [M+H]⁺ 380. t_{R} (HPLC, Nucleosil C18; 5-100% CH₃CN+0.1% TFA/H₂O+0.1% TFA for 8 min, flow 1.5 ml/min): 3.0 min.

The starting material is prepared as follows:

### A) (3R*,5S*)-5-[(9H-Xanthen-9-ylmethyl)-carbamoyl]-piperidine-1,3-dicarboxylic acid 1-tert-butyl ester

A solution of 2,4-dioxo-3-oxa-7-aza-bicyclo[3.3.1]nonane-7-carboxylic acid tert-butyl ester (1.45 g, 5.7 mmol) (see below in and under Scheme 7), dimethyl-4-aminopyridine (70 mg) and triethylamine (1.6 mL, 11 mmol) in dichloromethane (10 mL) is added dropwise a solution of C-(9H-xanthen-9-yl)-methylamine in dichloromethane (5 mL) at 0° C. The reaction mixture is warmed to rt and stirred at rt for 1 h before it is diluted with dichloromethane and extracted with 1M aq. HCl. The organic phase is washed with brine, dried over Na₂SO₄, filtered and evaporated to leave a residue, which is purified by flash chromatography on silica gel (eluent: CH₂Cl₂/MeOH 95:5 to 9:1) to give the title compound as a yellow solid.

MS (LC-MS): 410 [M+H—C(CH₃)₃]⁺

TLC, Rf (CH₂Cl₂/MeOH 9:1)=0.48.

### B) (3R*,5S*)-3-Methylcarbamoyl-5-[(9H-xanthen-9-ylmethyl)-carbamoyl]-piperidine-1-carboxylic acid tert-butyl ester

A solution of (3R*,5S*)-5-[(9H-Xanthen-9-ylmethyl)-carbamoyl]-piperidine-1,3-dicarboxylic acid 1-tert-butyl ester (380 mg, 0.8 mmol), O-(1H-6-chlorobenzotriazole-1-yl)-1,1,3,3-tetramethyluronium hexafluorophosphate (425 mg, 1 mmol) and N-ethyldiisopropylamine (0.28 mL, 1.6 mmol) in CH₂Cl₂ and acetonitrile is stirred for 10 min at rt. Then methyl amine hydrochloride salt (55 mg, 0.8 mmol) is added and the reaction is stirred overnight at rt. The suspension is filtered and the solvents are evaporated to yield a residue, which is partitioned between ethyl acetate and sat. aq. NaHCO₃. The aqueous phase is extracted again with ethyl acetate before the combined organic phases are washed with 2N aq. HCl and brine, dried over Na₂SO₄, filtered and evaporated to yield a residue, which is purified by flash chromatography on silica gel (eluent: CH₂Cl₂/MeOH 95:5) to give the title compound as a yellow solid. MS (LC-MS): 480 [M+H]⁺ TLC, Rf (CH₂Cl₂/MeOH 95:5)=0.25.

### Example 94

### (3S*,5R*)-Piperidine-3,5-dicarboxylic acid 3-cyclohexylmethyl-amide 5-[(9H-xanthen-9-ylmethyl)-amide]

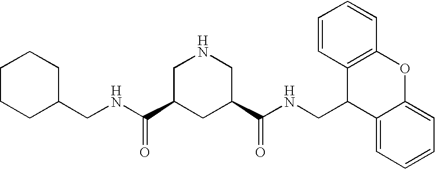

The title compound is prepared analogously as described in Example 15 using cyclohexyl-methylamine. MS: [M+H]⁺ 462. t_{R} (HPLC, Nucleosil C18; 5-100% CH₃CN+0.1% TFA/H₂O+0.1% TFA for 8 min, flow 1.5 ml/min): 3.5 min.

### Example 95

### (3S*,5R*)-Piperidine-3,5-dicarboxylic acid 3-{[9-(4-methoxy-butyl)-9H-xanthen-9-ylmethyl]-amide}5-methylamide

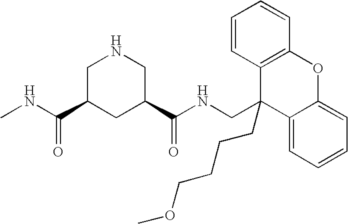

The title compound is prepared analogously as described in Example X using C-[9-(4-Methoxy-butyl)-9H-xanthen-9-yl]-methylamine. MS: [M+H]⁺ 466. t_{R} (HPLC, Nucleosil C18; 5-100% CH₃CN+0.1% TFA/H₂O+0.1% TFA for 8 min, flow 1.5 ml/min): 4.5 min.

### Example 96

### (3S*,5R*)-Piperidine-3,5-dicarboxylic acid 3-cyclohexylmethyl-amide 5-{[9-(4-methoxy-butyl)-9H-xanthen-9-ylmethyl]-amide}

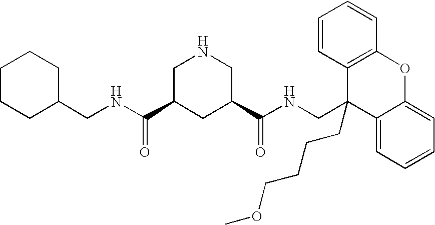

The title compound is prepared analogously as described in Example 15 using C-[9-(4-Methoxy-butyl)-9H-xanthen-9-yl]-methylamine and cyclohexyl-methylamine. MS: [M+H]⁺ 548. t_{R} (HPLC, Nucleosil C18; 5-100% CH₃CN+0.1% TFA/H₂O+0.1% TFA for 8 min, flow 1.5 ml/min): 5.2 min.

Preparation of cyclic anhydride XXIII shown as intermediate in Scheme 6:

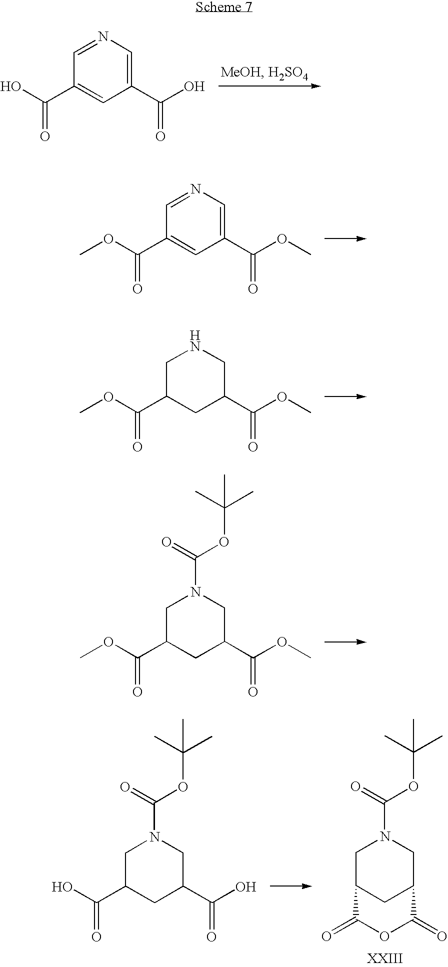

### A: Pyridine-3,5-dicarboxylic acid dimethyl ester

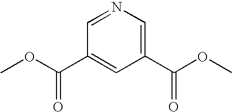

3,5-Pyridinedicarboxylic acid (1.5 g, 63 mmol) and conc. H₂SO₄ (0.9 mL) in MeOH (15 mL) are heated in a microwave oven at 120° C. for 2 h. The solvent is evaporated to give a residue with is partitioned between ethyl acetate and sat. aq. NaHCO₃ The organic phase is washed with brine, dried over Na₂SO₄, filtered and evaporated to give a light yellow solid.

MS (LC-MS): 196 [M+H]⁺ TLC, Rf (ethyl acetate/hexane 1:1)=0.56.

### B: Piperidine-3,5-dicarboxylic acid dimethyl ester

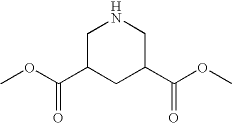

Pyridine-3,5-dicarboxylic acid dimethyl ester (5.3 g, 27 mmol) and Rh/PtO₂ (0.5 g) in MeOH (200 mL) are stirred under hydrogen overnight. The resulting mixture is filtered and the solvents are evaporated to leave a brown oil. MS (LC-MS): 202 [M+H]⁺

### C: Piperidine-1,3,5-tricarboxylic acid 1-tert-butyl ester 3,5-dimethyl ester

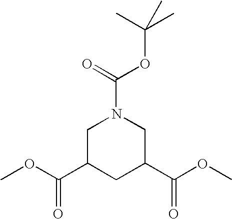

A solution of piperidine-3,5-dicarboxylic acid dimethyl ester (5.4 g, 26.8 mmol) in CH₂Cl₂ (55 mL) is treated with Boc₂O (6.4 g, 29.5 mmol) and the reaction stirred at rt overnight. The reaction is quenched with 0.1N aq. HCl and the organic phase washed with 0.1N aq HCl. The combined aqueous phases are extracted 2 times with CH₂Cl₂/MeOH (9/1) before the combined organic phases are dried over Na₂SO₄, filtered and evaporated. The resulting residue is purified by flash chromatography on silica gel (eluent: CH₂Cl₂/MeOH 95:5) to give the title compound as a yellow solid. MS (LC-MS): 302 [M+H]⁺ TLC, Rf (CH₂Cl₂/MeOH 95:5)=0.5.

### D: Piperidine-1,3,5-tricarboxylic acid 1-tert-butyl ester

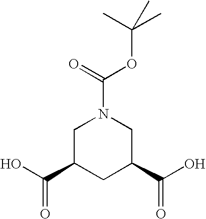

To a solution of piperidine-1,3,5-tricarboxylic acid 1-tert-butyl ester 3,5-dimethyl ester (6.8 g, 22.5 mmol) in MeOH/water (4:1, 120 mL), K₂CO₃ (9.4 g, 68 mmol) is added. The reaction is stirred at reflux overnight. The MeOH is evaporated and the residue extracted with dichloromethane and 1N aq. HCl. The organic phase is dried over Na₂SO₄, filtered and evaporated to give a light yellow solid. MS (LC-MS): 274 [M+H]⁺.

### E: 2,4-Dioxo-3-oxa-7-aza-bicyclo[3.3.1]nonane-7-carboxylic acid tert-butyl ester

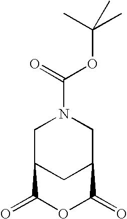

A suspension of piperidine-1,3,5-tricarboxylic acid 1-tert-butyl ester (1 g, 3.6 mmol) in acetic anhydride (20 mL) is heated at reflux for 2 h. The reaction mixture is evaporated 3× times with toluene before it is dried under high vacuum at rt overnight to give a yellow solid. MS (LC-MS): 278 [M+Na]⁺.

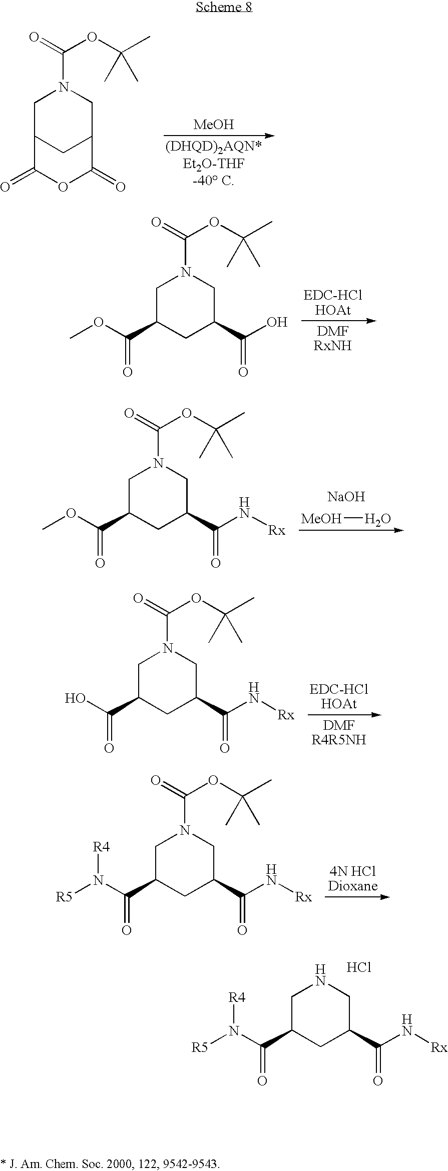

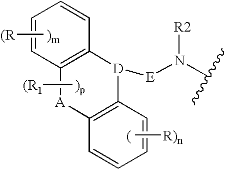

R₅ and R₆ are as defined in the starting materials, preferably they are deducible from the following examples, as is Rx:

### Asymmetric Desymmetrization

### i) 3S,5R)-Piperidine-1,3,5-tricarboxylic acid 1-tert-butyl ester 3-methyl ester

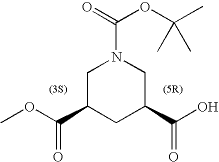

To the solution of 2,4-Dioxo-3-oxa-7-aza-bicyclo[3.3.1]nonane-7-carboxylic acid tert-butyl ester (401.5 mg, 1.57 m mol) and commercially available (DHQD)₂AQN (423.6 mg, 0.47 mmol, 95% purity)^{a} dissolved in Et₂O (60 mL) and THF (20 mL) under N₂, MeOH (0.64 mL, 15.67 mmol) is added at −40° C. After stirring at that temperature for 24 hr and sat. citric acid aq. is added. The reaction mixture is extracted with EtOAc. Organic phase is washed with brine, dried over Na₂SO₄ and subjected to silica chromatography to give the titled compound (404.3 mg) in 89% yield as 98% ee. White amorphous material ES-MS: M+H-tBu=232; HPLC: t_{R}=2.73 min. chiral HPLC (column: CHIRALPAH AD-H (0.46 cm×25 cm), eluent: hexane/i-PrOH=95/5, flow rate: 0.5 mL/min, detection: UV 210 nm, temperature: RT) t_{R}=33.25 min for (3R,5S)-Piperidine-1,3,5-tricarboxylic acid 1-tert-butyl ester 3-methyl ester, 35.56 min for (3S,5R)-Piperidine-1,3,5-tricarboxylic acid 1-tert-butyl ester 3-methyl ester, a Chen, Y.; Tian, S-K.; Deng, Li. J. Am. Chem. Soc. 2000, 122, 9542-9543.

### ii) (3R,5S)-5-{[9-(4-Methoxy-butyl)-9H-xanthen-9-ylmethyl]-carbamoyl}-piperidin dicarboxylic acid 1-tert-butyl ester 3-methyl ester

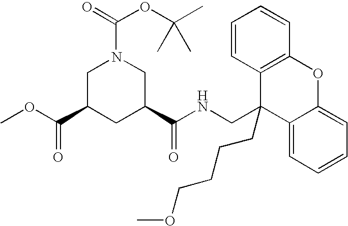

The titled compound is synthesized by condensation of (3S,5R)-piperidine-1,3,5-tricarboxylic acid 1-tert-butyl ester 3-methyl ester (2.13 g, 7.4 mmol) and C-[9-(4-methoxybutyl)-9H-xanthen-9-yl]-methylamine (2.20 g, 7.4 mmol) analogously to the preparation of “General procedure, Scheme X without triethylamine”. white amorphous material. ES-MS: M+H=567; HPLC (Condition-B): t_{R}=2.07 min.

### iii) (3R,5S)-5-{[9-(4-Methoxy-butyl)-9H-xanthen-9-ylmethyl]-carbamoyl}-piperidine-1,3-dicarboxylic acid 1-tert-butyl ester

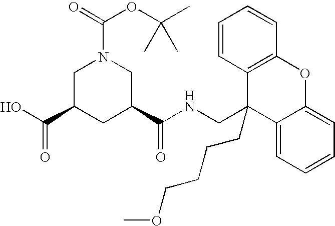

To a solution of (3R,5S)-5-{[9-(4-Methoxy-butyl)-9H-xanthen-9-ylmethyl]-carbamoyl}-piperidine-1,3-dicarboxylic acid 1-tert-butyl ester 3-methyl ester (4.00 g, 7.10 mmol) in dioxane (30 mL) under N₂, 1N NaOH aq. (10 mL) is added at 0° C. After stirring at that temperature for 3 hr, 1N HCl aq. (10 mL) and sat. KHSO₄ aq. are added to the solution. The reaction mixture is extracted with CH₂Cl₂, dried over Na₂SO₄, concentrated under reduced pressure to give the titled compound (3.90 g, 7.1 mmol). White amorphous material. ES-MS: M+H=553; HPLC (Condition-B): t_{R}=1.94 min.

### iv) (3R,5S)-3-Isobutylcarbamoyl-5-{[9-(4-methoxy-butyl)-9H-xanthen-9-ylmethyl]-carbamoyl}-piperidine-1-carboxylic acid tert-butyl ester

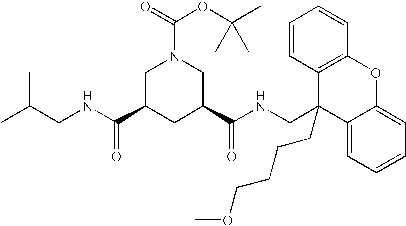

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and isobutylamine (17.9 μL, 0.18 mmol) analogously to the preparation of “general procedure, scheme 8-ii)”. White amorphous material. ES-MS: M+H=608; HPLC (Condition-B): t_{R}=3.97 min.

### (3S,5R)-3-{[9-(4-Methoxy-butyl)-9H-xanthen-9-ylmethyl]-carbamoyl}-5-(3-methyl-butylcarbamoyl)-piperidine-1-carboxylic acid tert-butyl ester

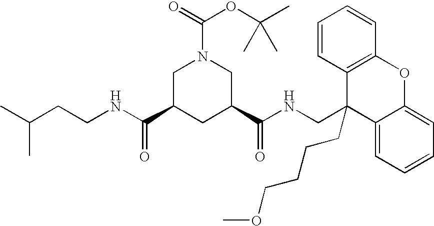

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and 3-Methyl-butylamine (25 μL, 0.21 mmol) analogously to the preparation of “general procedure, scheme 8-ii)”. white amorphous material. ES-MS: M+H=622; HPLC (Condition-B): t_{R}=20.10 min.

### (3S,5R)-3-{[9-(4-Methoxy-butyl)-9H-xanthen-9-ylmethyl]-carbamoyl}-5-phenethylcarbamoyl-piperidine-1-carboxylic acid tert-butyl ester

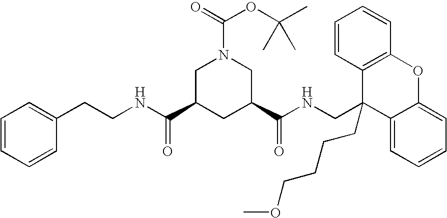

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and Phenethylamine (26.3 mL, 0.21 mmol) analogously to the preparation of “general procedure, scheme 8-ii)”. white amorphous material. ES-MS: M+H=656; HPLC (Condition-B): t_{R}=2.08 min.

### (3R,5S)-3-(3-Hydroxy-propylcarbamoyl)-{[9-(4-methoxy-butyl)-9H-xanthen-9-ylmethyl]carbamoyl}-piperidine-1-carboxylic acid tert-butyl ester

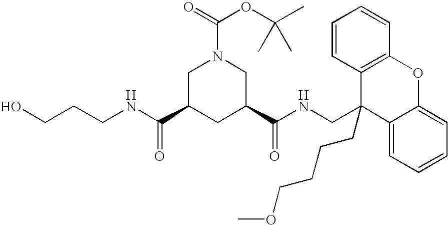

The titled compound is synthesized by condensation of (3R,5S)-5-{[9-(4-Methoxy-butyl)-9H-xanthen-9-ylmethyl]-carbamoyl-}-piperidine-1,3-dicarboxylic acid 1-tert-butyl ester (100 mg, 0.18 mmol) and 3-Amino-propan-1-ol (16.1 μL, 0.21 mmol) analogously to the preparation of “general procedure, scheme 8-ii)”. white amorphous material. ES-MS: M+H=610; HPLC (Condition-B): t_{R}=1.83 min.

### Mixture of equal parts of (3R,5S)-3-((S)-2-Hydroxy-1-methyl-ethylcarbamoyl)-5-{[9-(4-methoxy-butyl)-9H-xanthen-9-ylmethyl]-carbamoyl}-piperidine-1-carboxylic acid tert-butyl ester and (3R,5S)-3-((R)-2-Hydroxy-1-methyl-ethylcarbamoyl)-5-{[9-(4-methoxy-butyl)-9H-xanthen-9-ylmethyl]-carbamoyl}-piperidine-1-carboxylic acid tert-butyl ester

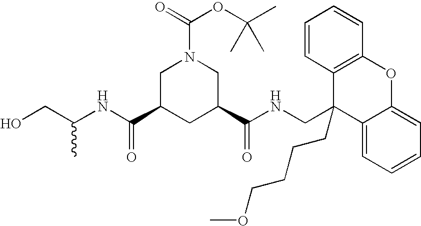

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and DL-2-Amino-propan-1-ol (16.3 μL, 0.21 mmol) analogously to the preparation of “general procedure, scheme 8-ii)”. white amorphous material. ES-MS: M+H=610; HPLC (Condition-B): t_{R}=1.85 min.

### (3R,5S)-3-(3,3-Dimethyl-butylcarbamoyl)-5-{[9-(4-methoxy-butyl)-9H-xanthen-9-ylmethyl]-carbamoyl}-piperidine-1-carboxylic acid tert-butyl ester

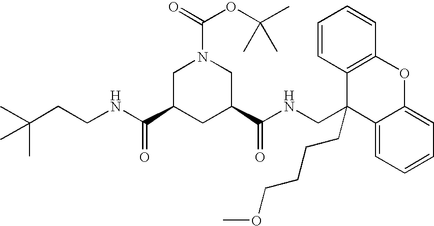

The titled compound is synthesized by condensation of (3R,5S)-5-{[9-(4-Methoxy-butyl)-9H-xanthen-9-ylmethyl]-carbamoyl}piperidine-1,3-dicarboxylic acid 1-tert-butyl ester (100 mg, 0.18 mmol) and 3,3-Dimethyl-butylamine (28.2 μL, 0.21 mmol) analogously to the preparation of “general procedure, scheme 8-ii)”. white amorphous material. ES-MS: M+H=636; HPLC (Condition-B): t_{R}=2.14 min.

### Mixture of equal parts of (3R,5S)-3-((R)-3-Hydroxy-butylcarbamoyl)-5-{[9-(4-methoxy-butyl)-9H-xanthen-9-ylmethyl]-carbamoyl}-piperidine-1-carboxylic acid tert-butyl ester and (3R,5S)-3-((S)-3-Hydroxy-butylcarbamoyl)-5-{[9-(4-methoxy-butyl)-9H-xanthen-9-ylmethyl]-carbamoyl}-piperidine-1-carboxylic acid tert-butyl ester

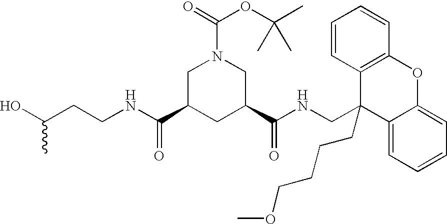

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and DL-1-Amino-propan-2-ol (16.2 μL, 0.21 mmol) analogously to the preparation of “general procedure, scheme 8-ii)”. white amorphous material. ES-MS: M+H=610; HPLC (Condition-B): t_{R}=1.84 min.

### (3R,5S)-3-((S)-1-Hydroxymethyl-3-methyl-butylcarbamoyl)-5-{[9-(4-methoxy-butyl)-9H-xanthen-9-ylmethyl]-carbamoyl}-piperidine-1-carboxylic acid tert-butyl ester

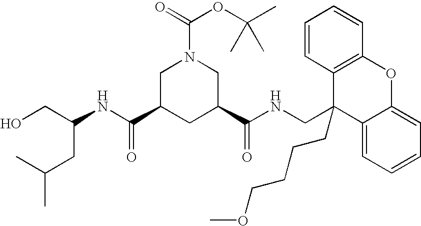

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and (S)-Leucinol (26.8 μL, 0.21 mmol) analogously to the preparation of “general procedure, scheme 8-ii)”. white amorphous material. ES-MS: M+H=652; HPLC (Condition-B): t_{R}=1.99 min.

### (3R,5S)-3-((R)-1-Hydroxymethyl-3-methyl-butylcarbamoyl)-5-{[9-(4-methoxy-butyl)-9H-xanthen-9-ylmethyl]-carbamoyl}-piperidine-1-carboxylic acid tert-butyl ester

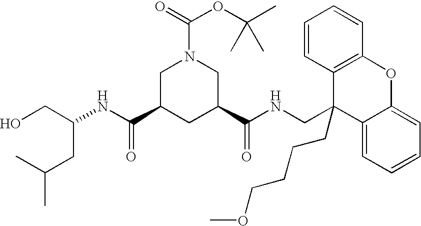

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and (R)-Leucinol (26.8 μL, 0.21 mmol) analogously to the preparation of “general procedure, scheme 8-ii)”. white amorphous material. ES-MS: M+H=652; HPLC (Condition-B): t_{R}=1.96 min.

### (3S,5R)-3-{[9-(4-Methoxy-butyl)-9H-xanthen-9-ylmethyl]-carbamoyl}-5-[methyl-(2-pyridin-4-ylethyl)-carbamoyl]-piperidine-1-carboxylic acid tert-butyl ester

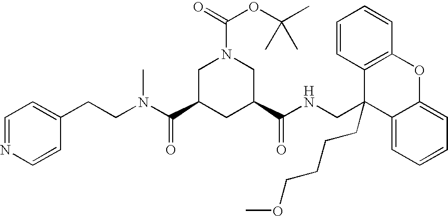

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and Methyl-(2-pyridin-4-yl-ethyl)-amine 2HCl salt (45 mg, 0.22 mmol) analogously to the preparation of “general procedure, scheme 8-ii)”. yellow amorphous material. ES-MS: M+H=671; HPLC (Condition-A): t_{R}=3.09 min.

### (3R,5S)-3-[Ethyl-(2-pyridin-4-yl-ethyl)-carbamoyl]-5-{[9-(4-methoxy-butyl)-9H-xanthen-9-ylmethyl]-carbamoyl}-piperidine-1-carboxylic acid tert-butyl ester

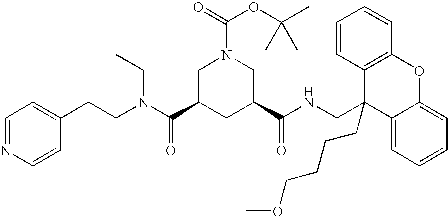

The titled compound is synthesized by condensation of (3R,5S)₅-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and Ethyl-(2-pyridin-4-yl-ethyl)-amine 2HCl salt (91.3 mg, 0.41 mmol) analogously to the preparation of “general procedure, scheme 8-ii)”. yellow amorphous material. ES-MS: M+H=685; HPLC (Condition-A): t_{R}=3.17 min.

### (3R,5S)-3-Benzylcarbamoyl-5-{[9-(4-methoxy-butyl)-9H-xanthen-9-ylmethyl}-carbamoyl]-piperidine-1-carboxylic acid tert-butyl ester

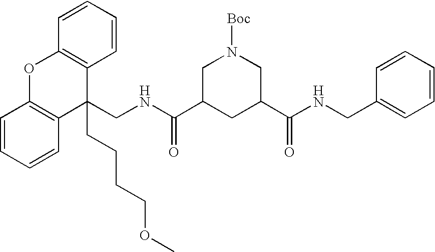

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (83 mg, 0.15 mmol) and benzyl amine (20 μL, 0.18 mmol) analogously to the preparation of the “general procedure, scheme X without triethylamine”. Colorless amorphous material; ES-MS: M+H=642; HPLC (Condition-A): t_{R}=4.03 min.

### (3S*,5R*)-3-{[9-(4-Methoxy-butyl)-9H-xanthen-9-ylmethyl]-carbamoyl}-5-phenethylcarbamoyl-piperidine-1-carboxylic acid tert-butyl ester

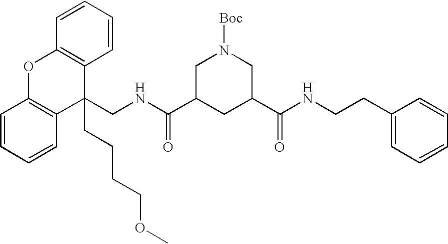

The titled compound is synthesized by condensation of (3R*,5S*)-5-{[9-(4-Methoxy-butyl)-9H-xanthen-9-ylmethyl]-carbamoyl}-piperidine-1,3-dicarboxylic acid 1-tert-butyl ester (83 mg, 0.15 mmol) and phenethyl amine (23 μL, 0.18 mmol) analogously to the preparation of the “general procedure, scheme X without triethylamine”. Colorless amorphous material; ES-MS: M+H=656; HPLC (Condition-A): t_{R}=4.15 min.

### (3S*,5R*)-3-{[9-(4-Methoxy-butyl)-9H-xanthen-9-ylmethyl]-carbamoyl}-5-(methyl-phenethylcarbamoyl)-piperidine-1-carboxylic acid tert-butyl ester

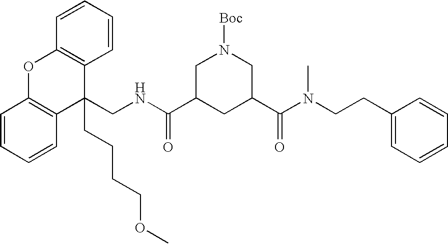

The titled compound is synthesized by condensation of (3R*,5S*)-5-{[9-(4-Methoxy-butyl)-9H-xanthen-9-ylmethyl]-carbamoyl}-piperidine-1,3-dicarboxylic acid 1-tert-butyl ester (83 mg, 0.15 mmol) and N-methylphenethyl amine (26 μL, 0.18 mmol) analogously to the preparation of the “general procedure, scheme X without triethylamine”. Colorless amorphous material; ES-MS: M+H=670; HPLC (Condition-A): t_{R}=4.32 min.

### (3R,5S)-3-(Benzyl-methyl-carbamoyl)-5-{[9-(4-methoxy-butyl)-9H-xanthen-9-ylmethyl]-carbamoyl}-piperidine-1-carboxylic acid tert-butyl ester

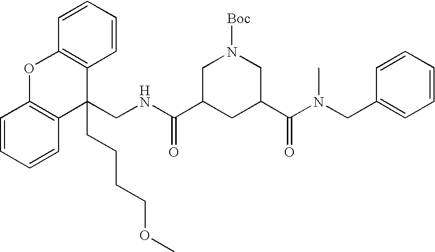

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and N-methylbenzyl amine (0.026 mL, 0.2 mmol) analogously to the preparation of “general procedure, scheme X without triethylamine”. Colorless amorphous material; ES-MS: M+H=656; HPLC (Condition-A): t_{R}=4.24 min.

### (3R,5S)-3-(Benzyl-ethyl-carbamoyl)-5-{[9-(4-methoxy-butyl)-9H-xanthen-9-ylmethyl]-carbamoyl}-piperidine-1-carboxylic acid tert-butyl ester

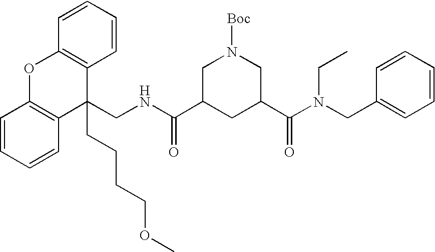

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and commercially available N-ethylbenzyl amine (0.03 mL, 0.2 mmol) analogously to the preparation of the “general procedure scheme X without triethylamine”. Colorless amorphous material; ES-MS: M+H=670; HPLC (Condition-A): t_{R}=4.39 min.

### (3R,5S)-3-(Isobutyl-methyl-carbamoyl)-5-{[9-(4-methoxy-butyl)-9H-xanthen-9-Ylmethyl]-carbamoyl}-piperidine-1-carboxylic acid tert-butyl ester

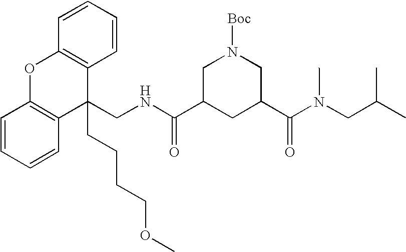

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and known material, N-isobutyl methyl amine hydrochloride (25 mg, 0.2 mmol) analogously to the preparation of the “general procedure scheme X without triethylamine”. Colorless amorphous material; ES-MS: M+H=622; HPLC (Condition-A): t_{R}=4.17 min.

### (3R,5S)-3-(Ethyl-isobutyl-carbamoyl)-5-{[9-(4-methoxy-butyl)-9H-xanthen-9-ylmethyl]-carbamoyl}-piperidine-1-carboxylic acid tert-butyl ester

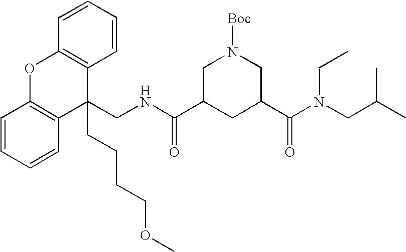

The titled compound is synthesized by condensation of (3R,5S)₅-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and known material, N-isobutyl ethyl amine (27 mg, 0.2 mmol) analogously to the preparation of the “general procedure scheme X without triethylamine”. Colorless amorphous material; ES-MS: M+H=636; HPLC (Condition-A): t_{R}=4.34 min.

### (3S,5R)-3-{[9-(4-Methoxy-butyl)-9H-xanthen-9-ylmethyl]-carbamoyl}-5-(methyl-pyridin-2-ylmethyl-carbamoyl)-piperidine-1-carboxylic acid tert-butyl ester

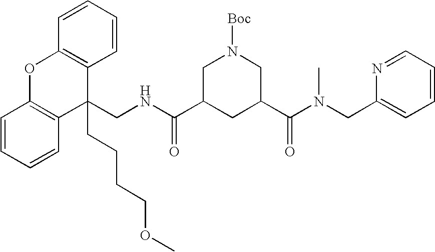

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and commercially available N-methyl-N-(2-pyridylmethyl) amine (32 mg, 0.20 mmol) analogously to the preparation of the “general procedure scheme X without triethylamine”. White powder; ES-MS: M+H=657; HPLC (Condition-A): t_{R}=3.15 min.

### (3R,5S)-3-(Ethyl-pyridin-2-ylmethyl-carbamoyl)-5-{[9-(4-methoxy-butyl)-9H-xanthen-9-ylmethyl]-carbamoyl}-piperidine-1-carboxylic acid tert-butyl ester

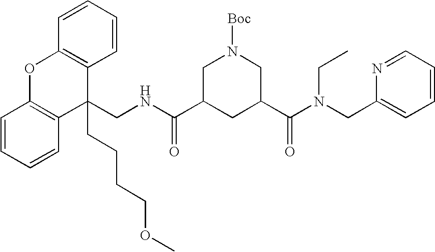

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and commercially available N-ethyl-N-(2-pyridylmethyl) amine (40 mg, 0.20 mmol) analogously to the preparation of the “general procedure scheme X without triethylamine”. White powder; ES-MS: M+H=671; HPLC (Compound-A): t_{R}=3.22 min.

### (3R,5S)-3-[Ethyl-(6-methoxy-pyridin-2-ylmethyl)-carbamoyl]-5-{[9-(4-methoxy-butyl)-9H-xanthen-9-ylmethyl]-carbamoyl}-piperidine-1-carboxylic acid tert-butyl ester

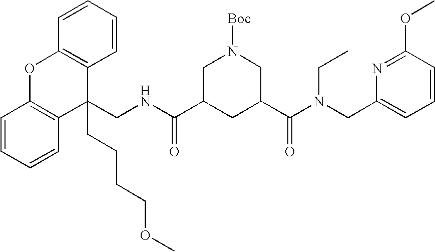

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and Ethyl-(6-methoxy-pyridin-2-ylmethyl)-amine (61 mg, 0.37 mmol) analogously to the preparation of the “general procedure scheme X without triethylamine”. White amorphous; ES-MS: M=701; HPLC (Condition-B): t_{R}=2.11 min.

### Ethyl-(6-methoxy-pyridin-2-ylmethyl)-amine

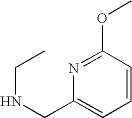

The titled compound is synthesized by amination of 2-chloromethyl-6-methoxy-pyridine (126 mg, 0.80 mmol) and 2M Ethylamine in THF (8 mL, 16.0 mmol) analogously to the preparation of the Example A. yellow oil; ES-MS: M+H=167; HPLC (Condition-B): t_{R}=1.16 min.

### 2-Chloromethyl-6-methoxy-pyridine

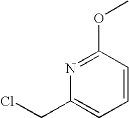

A solution of (6-methoxy-pyridin-2-yl)-methanol (334 mg, 2.40 mmol) and Et₃N (0.50 ml, 3.60 mmol) in CH₂Cl₂ (10 mL) is added dropwise MsCl (0.22 mL, 2.88 mmol) at 0° C. The reaction mixture is warmed to rt and stirred for 18 h before it is diluted with CH₂Cl₂ and extracted with H₂O. The organic phase is dried over MgSO₄, filtered and evaporated to leave a residue, which is purified by flash chromatography on silica gel (eluting: EtOAc:Hexane 3:7) to give the titled compound as a yellow oil; ES-MS: M+H=167; HPLC (Condition-B): t_{R}=1.16 min.

### (3R,5S)-3-[Ethyl-(5-methoxy-pyridin-2-ylmethyl)-carbamoyl]-5-{[9-(4-methoxy-butyl)-9H-xanthen-9-ylmethyl]-carbamoyl}-piperidine-1-carboxylic acid tert-butyl ester

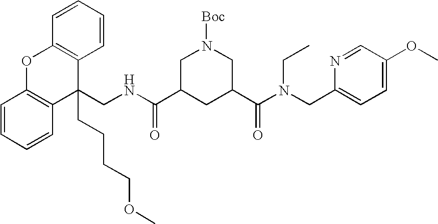

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and ethyl-(5-methoxy-pyridin-2-ylmethyl)-amine (33 mg, 0.20 mmol) analogously to the preparation of the “general procedure scheme X without triethylamine”. White amorphous; ES-MS: M=701; HPLC (Condition-B): t_{R}=1.83 min.

### Ethyl-(5-methoxy-pyridin-2-ylmethyl)-amine

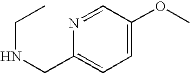

Ethyl-(5-methoxy-pyridin-2-ylmethyl)-amine is synthesized by amination of 2-chloromethyl-5-methoxy-pyridine (Inorganic Chemistry, 42(8), 2639-2653; 2003) (215 mg, 1.31 mmol) and 2M Ethylamine in THF (3.4 mL, 6.65 mmol) analogously to the preparation of the Example A. brown oil; ES-MS: M+H=167; HPLC (Condition-B): t_{R}=1.19 min.

### (3S,5R)-3-{[9-(4-Methoxy-butyl)-9H-xanthen-9-ylmethyl]-carbamoyl}-5-[methyl-(3-methylbutyl)-carbamoyl]-piperidine-1-carboxylic acid tert-butyl ester

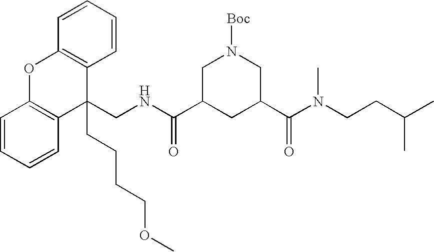

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and N-methyl(3-methylbutyl) amine hydrochloride (27 mg, 0.2 mmol) analogously to the preparation of the “general procedure scheme X without triethylamine”. Colorless amorphous material; ES-MS: M+H=636; HPLC (Condition-A): t_{R}=4.39 min.

### (3R,5S)-3-[Ethyl-(3-methyl-butyl)-carbamoyl]-5-{[9-(4-methoxy-butyl)-9H-xanthen-9-ylmethyl]-carbamoyl}-piperidine-1-carboxylic acid tert-butyl ester

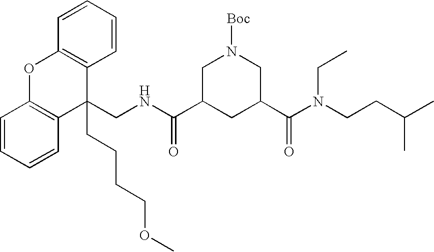

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and N-ethyl(3-methylbutyl) amine hydrochloride (34 mg, 0.22 mmol) analogously to the preparation of the “general procedure scheme X without triethylamine”. Colorless amorphous material; ES-MS: M+H=650; HPLC (Condition-A): t_{R}=4.55 min.

### (3R,5S)-3-[Isopropyl-(3-methyl-butyl)-carbamoyl]-5-{[9-(4-methoxy-butyl)-9H-xanthen-9-ylmethyl]-carbamoyl}-piperidine-1-carboxylic acid tert-butyl ester

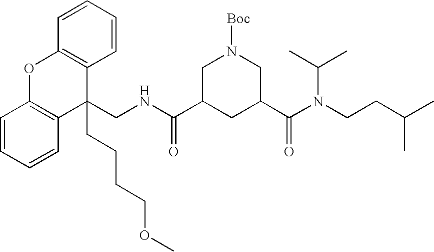

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (90 mg, 0.16 mmol) and known material, N-isopropyl(3-methylbutyl) amine hydrochloride (27 mg, 0.16 mmol) analogously to the preparation of the “general procedure scheme X without triethylamine”. Colorless amorphous material; ES-MS: M+H=664; HPLC (Condition-A): t_{R}=4.75 min.

### (3R,5S)-3-[Cyclopropyl-(3-methyl-butyl)-carbamoyl]-5-{[9-(4-methoxy-butyl)-9H-xanthen-9-ylmethyl]-carbamoyl}piperidine-1-carboxylic acid tert-butyl ester

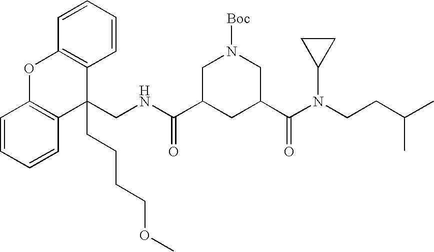

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and Cyclopropyl-(3-methyl-butyl)-amine (65 mg, 0.5 mmol) analogously to the preparation of the “general procedure scheme X without triethylamine”. Colorless amorphous material; ES-MS: M+H=662; HPLC (Condition-A): t_{R}=4.67 min.

### Example A

### Cyclopropyl-(3-methyl-butyl)-amine

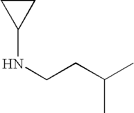

To a solution of cyclopropylamine (0.7 mL, 10 mmol) in DMF (40 mL), 1-bromo-3-methylbutane (0.4 mL, 3.3 mmol) is added at 0° C. After stirring for 7 hours, the reaction mixture is diluted with H₂O and extracted with n-Hexane. The combined organic phases are washed with H₂O and dried over Na₂SO₄. Concentration under reduced pressure and filtration give the titled compound; ES-MS: M+H=127; Rf=0.18 (MeOH: CH₂Cl₂=1:4).

### (3S,5R)-3-{[9-(4-Methoxy-butyl)-9H-xanthen-9-ylmethyl]-carbamoyl}-5-(methyl-pyridin-3-ylmethyl-carbamoyl)-piperidine-1-carboxylic acid tert-butyl ester

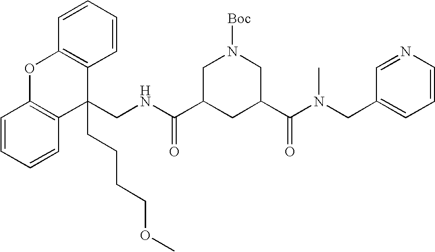

The title compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (118 mg, 0.21 mmol) and commercially available N-methyl-N-(3-pyridylmethyl) amine (40 mg, 0.32 mmol) analogously to the preparation of the “general procedure scheme X without triethylamine”. White powder; ES-MS: M+H=657; HPLC (Condition-A): t_{R}=3.09 min.

### (3R,5S)-3-(Cyclopropyl-pyridin-2-ylmethyl-carbamoyl)-5-{[9-(4-methoxy-butyl)-9H-xanthen-9-ylmethyl]-carbamoyl}-piperidine-1-carboxylic acid tert-butyl ester

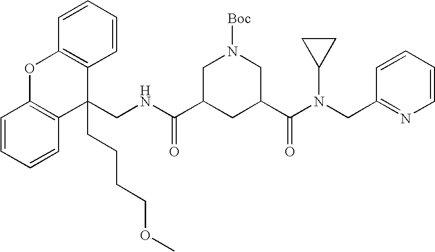

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (138 mg, 0.25 mmol) and commercially available N-(pyridin-2-ylmethyl)cyclopropanamine (41 mg, 0.33 mmol) analogously to the preparation of “general procedure scheme X without triethylamine”. White powder; ES-MS: M+H=683; HPLC (Condition-A): t_{R}=3.27 min.

### (3R,5S)-3-[Ethyl-(2-hydroxy-2-methyl-propyl)-carbamoyl]-5-{[9-(4-methoxy-butyl)-9H-xanthen-9-ylmethyl]-carbamoyl}-piperidine-1-carboxylic acid tert-butyl ester

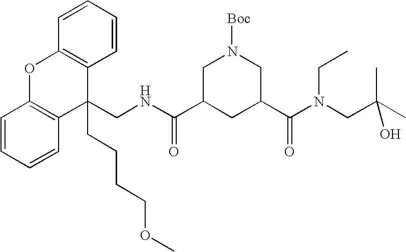

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (118 mg, 0.21 mmol) and known material 1-ethylamino-2-methyl-propan-2-ol (50 mg, 0.33 mmol) analogously to the preparation of “general procedure scheme X without triethylamine”. White powder; ES-MS: M+H=652; HPLC (Condition-A): t_{R}=3.72 min.

### (3R,5S)-3-[Ethyl-(3-hydroxy-3-methyl-butyl)-carbamoyl]-5-{[9-(4-methoxy-butyl)-9H-xanthen-9-ylmethyl]-carbamoyl}-piperidine-1-carboxylic acid tert-butyl ester

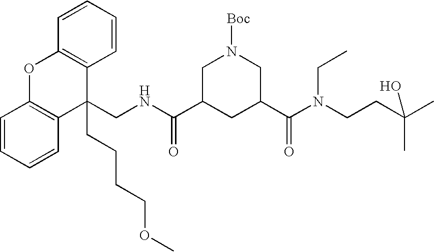

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (129 mg, 0.23 mmol) and known material 4-Ethylamino-2-methyl-butan-2-ol (50 mg, 0.28 mmol) analogously to the preparation of the “general procedure scheme X without triethylamine”. White powder; ES-MS: M+H=666; HPLC (Condition-A): t_{R}=3.72 min.

### 4-Ethylamino-2-methyl-butan-2-ol

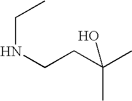

Ethyl-(3-hydroxy-3-methyl-butyl)-carbamic acid tert-butyl ester (65 mg, 0.28 mmol) is treated with 4N HCl solution in 1,4-dioxane (3 mL) at RT for 1 h. the reaction mixture are concentrated under reduced pressure to give the titled compound. The material is used in next step without further purification.

### Ethyl-(3-hydroxy-3-methyl-butyl)-carbamic acid tert-butyl ester

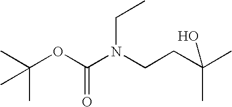

2-Ethyl-pentanedioic acid 1-tert-butyl ester 5-ethyl ester (200 mg, 0.82 mmol) in Et₂O (1 mL) is treated with MeMgBr (2.7 mL, 2.5 mmol) for 1 h at rt. The reaction mixture is poured into aq. HCl with ice, and the aqueous layer is extracted with EtOAc. The combined organic layers are washed with brine and dried (Na₂SO₄). The combined organic residue is purified by column chromatography to afford the titled compound as a colorless oil; ES-MS: M+H=232; HPLC (Condition-A): t_{R}=2.95 min.

### (3R,5S)-3-(Cyclopropyl-pyridin-4-ylmethyl-carbamoyl)-5-{[9-(4-methoxy-butyl)-9H-xanthen-9-ylmethyl]-carbamoyl}-piperidine-1-carboxylic acid tert-butyl ester

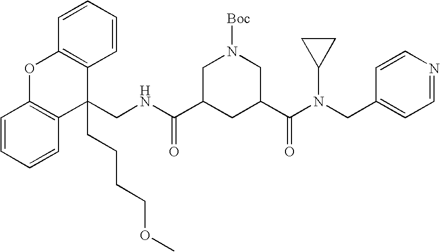

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (110 mg, 0.20 mmol) and commercially available N-(pyridin-4-ylmethyl)cyclopropanamine (50 mg, 0.33 mmol) analogously to the preparation of “general procedure scheme X without triethylamine”. White powder; ES-MS: M+H=683; HPLC (Condition-A): t_{R}=3.27 min.

### (3R,5S)-3-[Cyclopropyl-(2-methoxy-pyridin-4-ylmethyl)-carbamoyl]-5-{[9-(4-methoxy-butyl)-9H-xanthen-9-ylmethyl]-carbamoyl}-piperidine-1-carboxylic acid tert-butyl ester

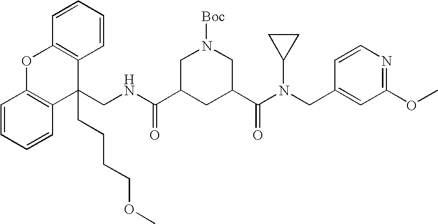

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (110 mg, 0.20 mmol) and Cyclopropyl-(2-methoxy-pyridin-4-ylmethyl)-amine (50 mg, 0.3 mmol) analogously to the preparation of “general procedure scheme X without triethylamine”. White powder; ES-MS: M+H=713; HPLC (Condition-A): t_{R}=3.70 min.

### Cyclopropyl-(2-methoxy-pyridin-4-ylmethyl)-amine

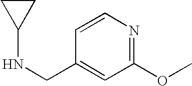

The titled compound is synthesized by alkylation of cyclopropylamine (0.54 mL, 6.9 mmol) by known material 4-chloromethyl-2-methoxy-pyridine (265 mg, 1.4 mmol) analogously to the preparation of Example A. Colorless oil. The crude product is used without purification.

### (3R,5S)-3-(Cyclohexylmethyl-ethyl-carbamoyl)-5-{[9-(4-methoxy-butyl)-9H-xanthen-9-ylmethyl]-carbamoyl}-piperidine-1-carboxylic acid tert-butyl ester

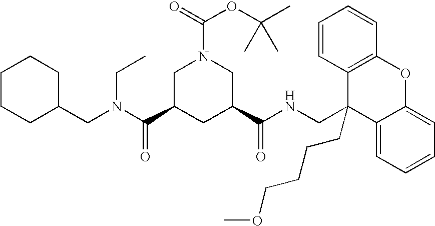

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and Cyclohexanemethylamine (51 mg, 0.36 mmol) (see e.g. J. Am. Chem. Soc. 1939, 61, 91.) analogously to the preparation of “general procedure scheme X without triethylamine”. White amorphous material; ES-MS: M+H=676; HPLC (Condition-B): t_{R}=2.29 min.

### (3R,5S)-3-[Ethyl-(tetrahydro-pyran-4-ylmethyl)-carbamoyl]-5-{[9-(4-methoxy-butyl)-9H-xanthen-9-ylmethyl]-carbamoyl}-piperidine-1-carboxylic acid tert-butyl ester

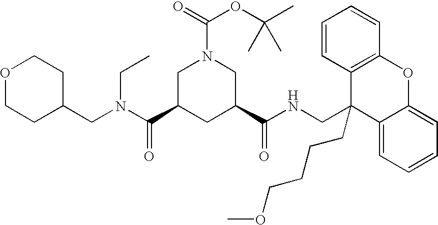

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and Ethyl-(tetrahydro-pyran-4-ylmethyl) amine hydrochloride (52 mg, 0.36 mmol) analogously to the preparation of “general procedure scheme X without triethylamine”. white amorphous material. ES-MS: M+H=678; HPLC (Condition-B): t_{R}=2.08 min.

### Ethyl-(tetrahydro-pyran-4-ylmethyl) amine hydrochloride

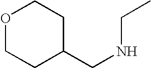

A solution of N-ethyl-N-(tetrahydro-pyran-4-ylmethyl)-carbamic acid tert-butyl ester in 4N hydrochloric acid in 1,4-dioxane (10 mL) is stirred at RT for 1 h. The dioxane is removed under reduced pressure to give title compound. White amorphous material; ES-MS: M+H=144; HPLC (Condition-B): t_{R}=1.41 min.

### N-ethyl-N-(tetrahydro-pyran-4-ylmethyl)-carbamic acid tert-butyl ester

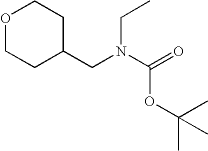

To a solution of N-(tetrahydro-pyran-4-ylmethyl)-carbamic acid tert-butyl ester (175 mg, 0.80 mmol) (see e.g. WO2004018433) in THF is added 1.0 M THF solution of NHMDS (1.62 ml, 1.62 mmol) at RT under nitrogen. The reaction is stirred for 0.5 h at RT then Ethyl iodide (623 mg, 4.0 mmol) is added. After stirring RT for over night, the reaction is quenched with H₂O. The resulting mixture is extracted with AcOEt, washed with brine, dried (MgSO₄), and concentrated to afford N-ethyl-N-(tetrahydro-pyran-4-ylmethyl)-carbamic acid tert-butyl ester. White amorphous material; ES-MS: M+H=244; HPLC (Condition-B): t_{R}=2.08 min.

### (3R,5S)-3-{Ethyl-[(R)-1-(tetrahydro-furan-2-yl)methyl]-carbamoyl}-5-{[9-(4-methoxy-butyl)-9H-xanthen-9-ylmethyl]-carbamoyl}-piperidine-1-carboxylic acid tert-butyl ester

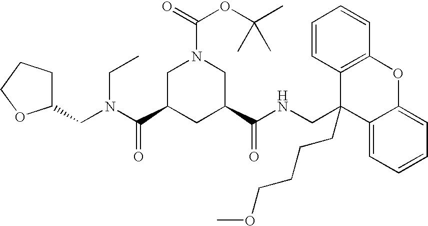

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (100 mg, 0.18 mmol) and Ethyl-[(R)-1-(tetrahydro-furan-2-yl)methyl]-amine hydrochloride (60 mg, 0.27 mmol) analogously to the preparation of “general procedure scheme X without triethylamine”. white amorphous material. ES-MS: M+H=664; HPLC (Condition-B): t_{Rt}=2.13 min.

### Ethyl-[(R)-1-(tetrahydro-furan-2-yl)methyl]-amine hydrochloride

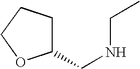

The title compound is synthesized by the deprotection of [(R)-1-(Tetrahydro-furan-2-yl)methyl]-carbamic acid tert-butyl ester (455 mg, 0.79 mmol) analogously to the preparation of Ethyl-(tetrahydro-pyran-4-ylmethyl) amine hydrochloride. White amorphous material; ES-MS: M+H=130; HPLC (Condition-A): t_{R}=1.41 min.

### Ethyl-[(R)-1-(tetrahydro-furan-2-yl)methyl]-carbamic acid tert-butyl ester

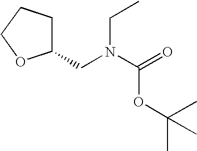

The title compound is synthesized by alkylation of [(R)-1-(Tetrahydro-furan-2-yl)methyl]-carbamic acid tert-butyl ester (455 mg, 0.79 mmol) analogously to the preparation of N-ethyl-N-(tetrahydro-pyran-4-ylmethyl)-carbamic acid tert-butyl ester. White amorphous material; ES-MS: M+H=230; HPLC (Condition-B): t_{R}=2.10 min.

### [(R)-1-(Tetrahydro-furan-2-yl)methyl]-carbamic acid tert-butyl ester

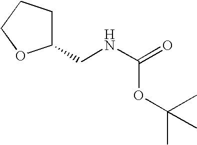

To a solution of C—[(R)-1-(Tetrahydro-furan-2-yl)]-methylamine (1.0 g, 9.9 mmol) in dichloromethane (20 mL) at 0° C. is added triethyl amine (1.58 mL, 11.9 mmol) followed by a solution of di-tert-butyl dicarbonate (2.16 g, 9.9 mmol) in dichloromethane (5 mL). After stirring RT for 1 h, the reaction is quenched with H₂O. The resulting mixture is washed with sat. KHSO₄ (aq.) (20 mL), brine, dried (MgSO₄), and concentrated to give [(R)-1-(Tetrahydrofuran-2-yl)methyl]-carbamic acid tert-butyl ester. Colorless oil; ES-MS: M+H=202; HPLC (Condition-B): t_{R}=1.90 min.

### (3S,5R)-3-{[9-(4-Methoxy-butyl)-9H-xanthen-9-ylmethyl]-carbamoyl}-5-[(3-methyl-butyl)propyl-carbamoyl]-piperidine-1-carboxylic acid tert-butyl ester

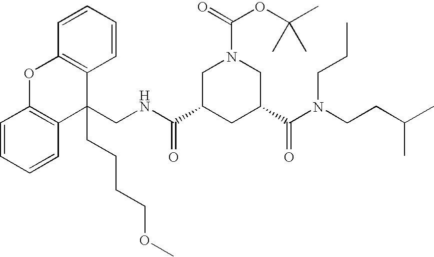

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (121.6 mg, 0.22 mmol) and N-propylisoamylamine hydrochloride (0.22 mmol) (J. Am. Chem. Soc. 1944, 66, 82) analogously to the preparation of the “general procedure scheme X without triethylamine”. White amorphous material; ES-MS: M=663; HPLC (Condition-B): t_{R}=2.28 min.

### (3R,5S)-3-[(3,4-Dimethoxy-benzyl)-ethyl-carbamoyl]-5-{[9-(4-methoxy-butyl)-9H-xanthen-9-ylmethyl]-carbamoyl}-piperidine-1-carboxylic acid tert-butyl ester

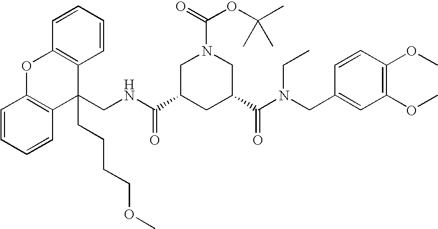

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (176.9 mg, 0.32 mmol) and N-ethyl-3,4-dimethoxybenzenemethaneamine (0.32 mmol) analogously to the preparation of the “general procedure scheme X without triethylamine”. White amorphous material; ES-MS: M=729; HPLC (Condition-B): t_{R}=2.07 min.

### (3R,5S)-3-[(2,3-Dihydro-benzo[1,4]dioxin-6-ylmethyl)-ethyl-carbamoyl]-5-{[9-(4-methoxybutyl)-9H-xanthen-9-ylmethyl]-carbamoyl}-piperidine-1-carboxylic acid tert-butyl ester

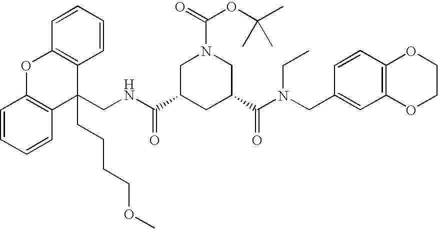

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (117.8 mg, 0.21 mmol) and (2,3-Dihydro-benzo[1,4]dioxin-6-ylmethyl)-ethyl-amine (0.43 mmol) analogously to the preparation of the general “general procedure scheme X without triethylamine”. White amorphous material; ES-MS: M=727; HPLC (Condition-B): t_{R}=2.11 min.

### Example B

### (2,3-Dihydro-benzo[1,4]dioxin-6-ylmethyl)-ethyl-amine

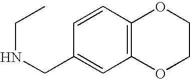

A mixture of 3,4-ethylenedioxybenzaldehyde (514.7 mg, 3.14 mmol), ethylamine 2M in THF (1.56 mL, 3.14 mmol) in THF (16 mL) is stirred under N2 at RT for 5 h, then NaBH₄ (731 mg, 3.45 mmol) is added at 0° C. After stirring at RT for 18 h, the reaction mixture is in vacuo, added 1N HCl at 0° C. and extracted with Et₂O. The combined inorganic phases are added 1N NaOH at 0° C., extracted with Et₂O and dried (Na₂SO₄). Concentration under reduced pressure, added 4N HCl-dioxane, and in vacuo to give the titled compound as white amorphous material; ES-MS: M=193; HPLC (Condition-B): t_{R}=1.20 min.

### (3R,5S)-3-[(2-[1,3]Dioxolan-2-yl-ethyl)-ethyl-carbamoyl]-5-{[9-(4-methoxy-butyl)-9H-xanthen-9-ylmethyl]-carbamoyl}-piperidine-1-carboxylic acid tert-butyl ester

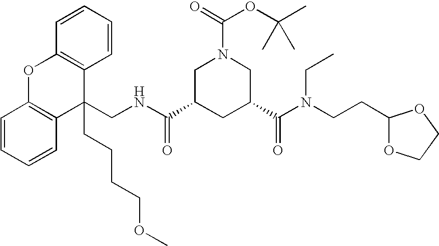

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (175.6 mg, 0.32 mmol) and N-ethyl-1,3-dioxolane-2-ethanamine (0.32 mmol) (see e.g. US1989/0905) analogously to the preparation of “general procedure scheme X without triethylamine”. White amorphous material; ES-MS: M=679; HPLC (Condition-B): t_{R}=2.00 min.

### (3R,5S)-3-[Cyclopropylmethyl-(3-methyl-butyl)-carbamoyl]-5-{[9-(4-methoxy-butyl)-9H-xanthen-9-ylmethyl]-carbamoyl}-piperidine-1-carboxylic acid tert-butyl ester

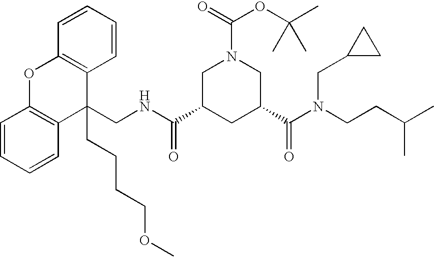

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (143.7 mg, 0.26 mmol) and Cyclopropylmethyl-(3-methyl-butyl)-amine (46.2 mg, 0.26 mmol) analogously to the preparation of the “general procedure scheme X without triethylamine”. White amorphous material; ES-MS: M=675; HPLC (Condition-B): t_{R}=2.29 min.

### Cyclopropylmethyl-(3-methyl-butyl)-amine

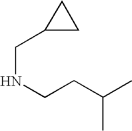

The titled compound is synthesized by condensation of isoamylamine (500 mg, 5.74 mmol), cyclopropanecarboxaldehyde (428 μL, 5.74 mmol) analogously to the preparation of the Example B. White amorphous material; ES-MS: M=679; HPLC (Condition-B): t_{R}=2.00 min.

### (3R,5S)-3-[(3-Dimethylamino-2,2-dimethyl-propyl)-ethyl-carbamoyl]-5-{[9-(4-methoxy-butyl)-9H-xanthen-9-ylmethyl]-carbamoyl}-piperidine-1-carboxylic acid tert-butyl ester

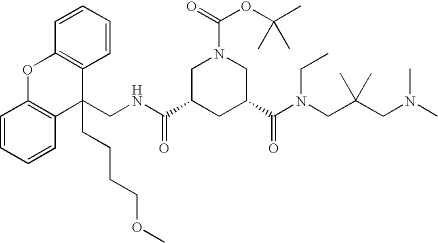

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (126.3 mg, 0.23 mmol) and N′-Ethyl-2,2,N,N-tetramethyl-propane-1,3-diamine (44.5 mg, 0.23 mmol) analogously to the preparation of “general procedure scheme X without triethylamine”. White amorphous material; ES-MS: M=692; HPLC (Condition-B): t_{R}=1.83 min.

### N′-Ethyl-2,2,N,N-tetramethyl-propane-1,3-diamine

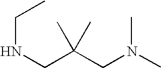

The titled compound is synthesized by condensation of N,N,2,2-tetramethyl-1,3-propanediamine (500 mg, 3.84 mmol), acetaldehyde (215 μL, 3.84 mmol) analogously to the preparation of the Example B. White amorphous material; ES-MS: M=679; HPLC (Condition-B): t_{R}=2.00 min

### Examples 97 to 127

The following Example enlisted in Table 3 are synthesized by deprotection of Boc group analogously to the Example 8, as hereinafter or hereinbefore described. As far as not being commercially available by synthesis analogous to methods or as described hereinbefore. The Asterisk (*) indicates the end of the bond at which the respective moiety is bound to the rest of the molecule falling under the following formula:

TABLE 3

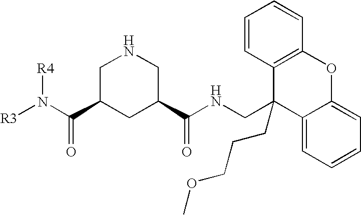

| | | | tᵣₑₜ | MS |
|---|---|---|---|---|
| No. | R4 | R3 | (Condition) | (M + H) |
| 97 | —H | 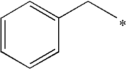 | 2.97 (A) | 542 |
| 98 | —H | 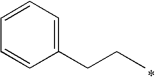 | 3.02 (A) | 556 |
| 99 | -Me | 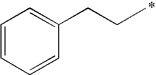 | 3.07 (A) | 570 |
| 100 | -Me | 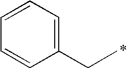 | 3.02 (A) | 556 |
| 101 | -Et | 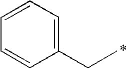 | 3.13 (A) | 570 |
| 102 | -Me | 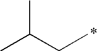 | 2.92 (A) | 522 |
| 103 | -Et | 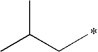 | 3.02 (A) | 536 |
| 104 | -Me | 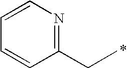 | 2.30 (A) | 557 |
| 105 | -Et | 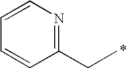 | 2.37 (A) | 571 |
| 106 | -Me | 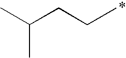 | 3.07 (A) | 536 |
| 107 | -Et | 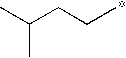 | 3.20 (A) | 550 |
| 108 | -iPr | 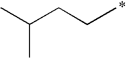 | 3.37 (A) | 564 |
| 109 | -cPr | 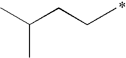 | 3.30 (A) | 562 |
| 110 | -Me | 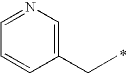 | 2.27 (A) | 557 |
| 111 | -Et | 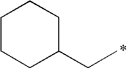 | 1.84 (B) | 576 |
| 112 | -Et | 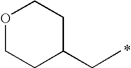 | 1.77 (B) | 578 |
| 113 | -Et | 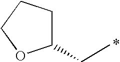 | 1.79 (B) | 564 |
| 114 | -Et | 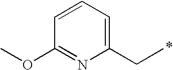 | 3.00 (A) | 601 |
| 115 | -Et | 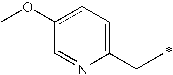 | 2.52 (A) | 601 |
| 116 | -nPr | 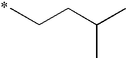 | 1.84 (B) | 563(M+) |
| 117 | -Et | 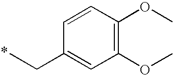 | 1.68 (B) | 629(M+) |
| 118 | -Et | 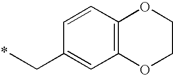 | 1.73 (B) | 627(M+) |
| 119 | -Et | 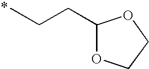 | 1.61 (B) | 579(M+) |
| 120 | 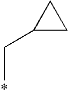 | 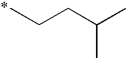 | 1.86 (B) | 575(M+) |
| 121 | -Et | 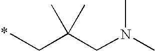 | 1.48 (B) | 592(M+) |
| 122 | -Me | 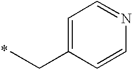 | 2.27 (A) | 557 |
| 123 | -cPr | 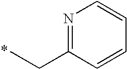 | 2.34 (A) | 583 |
| 124 | -Et | 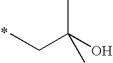 | 2.68 (A) | 552 |
| 125 | -Et | 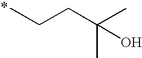 | 2.72 (A) | 566 |
| 126 | -cPr | 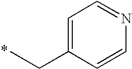 | 2.34 (A) | 583 |
| 127 | -cPr | 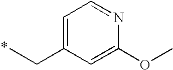 | 2.75 (A) | 613 |

### (3R,5S)-3-((R)-2-Hydroxymethyl-pyrrolidine-1-carbonyl)-5-{[9-(4-methoxy-butyl)-9H-xanthen-9-ylmethyl]-carbamoyl}-piperidine-1-carboxylic acid tert-butyl ester

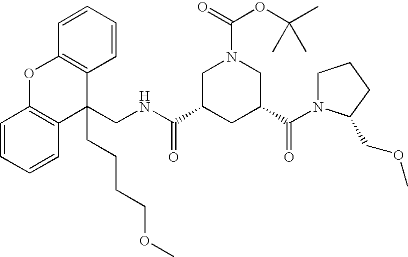

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (102.3 mg, 0.19 mmol) and (R)-2-(methoxymethyl)pyrrolidine (22.9 μL, 0.19 mmol) analogously to the preparation of “general procedure scheme X without triethylamine”. White amorphous material; ES-MS: M=649; HPLC (Condition-B): t_{R}=2.03 min.

### (3S,5R)-3-{[9-(4-Methoxy-butyl)-9H-xanthen-9-ylmethyl]-carbamoyl}-5-((R)-2-methoxycarbonyl-pyrrolidine-1-carbonyl)-piperidine-1-carboxylic acid tert-butyl ester

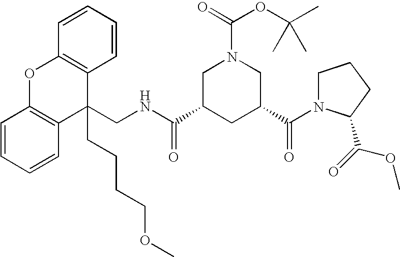

The titled compound is synthesized by condensation of (3R,5S)-5-{[9-(4-Methoxy-butyl)-9H-xanthen-9-ylmethyl]-carbamoyl}-piperidine-1,3-dicarboxylic acid 1-tert-butyl ester (102.3 mg, 0.19 mmol) and H-D-PRO-OME HCL (30.7 mg, 0.19 mmol) analogously to the preparation of the “general procedure scheme X without triethylamine”. White amorphous material; ES-MS: M=663; HPLC (Condition-B): t_{R}=2.00 min.

### Examples 128 to 131

The following Example enlisted in Table 4 are synthesized by deprotection of Boc group, analogously to the Example 8, as hereinafter or hereinbefore described. As far as not being commercially available by synthesis analogous to methods or as described hereinbefore. The Asterisk (*) indicates the end of the bond at which the respective moiety is bound to the rest of the molecule falling under the following formula:

TABLE 4

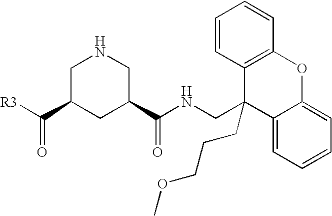

| | | tᵣₑₜ | MS |
|---|---|---|---|
| No. | R3 | (Condition) | (M+) |
| 128 | 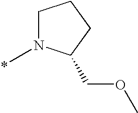 | 1.75 (B) | 549 |
| 129 | 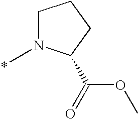 | 1.76 (B) | 563 |
| 130 | 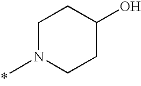 | 2.38 (A) | 536(M + H) |
| 131 | 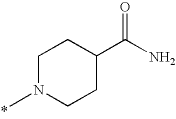 | 2.32 (A) | 563(M + H) |

Preparation of trans-amide

### (3R*,5R*)-Piperidine-1,3,5-tricarboxylic acid 1-tert-butyl ester 3,5-dimethyl ester

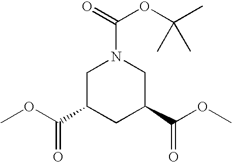

To a solution of the mixture of (3R*,5R*)-Piperidine-3,5-dicarboxylic acid dimethyl ester and (3R*,5R*)-Piperidine-3,5-dicarboxylic acid dimethyl ester (4.97 g, 25 mmol; from Scheme 7-c) in CH₂Cl₂ (70 mL) under N₂, triethyl amine (5.2 mL, 37.5 mmol), Boc₂O (5.7 g, 26.1 mmol) and DMAP (116.6 mg, 0.95 mmol) are added at 0° C. The resulting solution is stirred at RT overnight. And then, sat. NH₄Cl aq. is added. The reaction mixture is extracted with CH₂Cl₂, dried over Na₂SO₄, concentrated under reduced pressure and subjected to silica chromatography to give the titled compound (2.16 g, 7.1 mmol) as white syrup material. ES-MS: M+H-tBu=246; HPLC (Condition-A): t_{R}=3.17 min for trans isomer, 3.30 min for cis isomer. (Ref. Tetrahedron: Asymmetry 2003, 14, 1541-1545.)

### (3S*,5S*)-Piperidine-1,3,5-tricarboxylic acid 1-tert-butyl ester 3-methyl ester

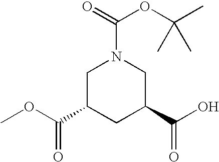

To a solution of (3R*,5R*)-Piperidine-1,3,5-tricarboxylic acid 1-tert-butyl ester 3,5-dimethyl ester (702.3 mg, 2.33 mmol) in MeOH (4.8 mL)-H₂O (1.2 mL) under N₂, Ba(OH)₂.8H₂O (367 mg, 1.16 mmol) is added at RT. After stirring at that temperature for 50 min, H₂O and sat. KHSO₄ aq. is added. The reaction mixture is extracted with CH₂Cl₂, dried over Na₂SO₄, concentrated under reduced pressure and subjected to silica chromatography to give the titled compound (411.5 mg, 1.43 mmol) in 61% as white syrup material. ES-MS: M+H-tBu=232; HPLC (Condition-A): t_{R}=2.50 min for trans isomer. (Ref. Aust., J. Chem. 1986, 39, 2061.)

### (3S*,5S*)-5-{[9-(4-Methoxy-butyl)-9H-xanthen-9-ylmethyl]-carbamoyl}-piperidine-1,3-dicarboxylic acid 1-tert-butyl ester 3-methyl ester

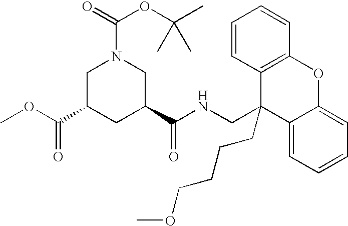

The titled compound is synthesized by condensation of (3S*,5S*)-Piperidine-1,3,5-tricarboxylic acid 1-tert-butyl ester 3-methyl ester (411.5 mg, 1.43 mmol) and C-[9-(4-Methoxy-butyl)-9H-xanthen-9-yl]-methylamine (425 mg, 1.43 mmol) analogously to the preparation of “general procedure, scheme 6”. white amorphous material. ES-MS: M+H=567; HPLC (Condition-A): t_{R}=4.05 min.

### (3S*,5S*)-5-{[9-(4-Methoxy-butyl)-9H-xanthen-9-ylmethyl]-carbamoyl}-piperidine-1,3-dicarboxylic acid 1-tert-butyl ester

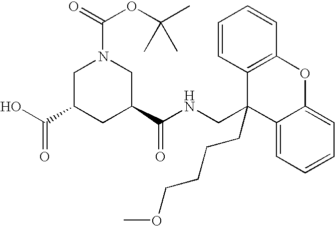

The titled compound is synthesized by hydrolysis of (3S*,5S*)-5-{[9-(4-Methoxy-butyl)-9H-xanthen-9-ylmethyl]-carbamoyl}-piperidine-1,3-dicarboxylic acid 1-tert-butyl ester 3-methyl ester (589.7 mg, 1.04 mmol) analogously to the preparation of “General procedure, scheme 8-iii)”. white amorphous material. ES-MS: M+H=553; HPLC (Condition-A): t_{R}=3.60 min.

### (3S*,5S*)-3-{[9-(4-Methoxy-butyl)-9H-xanthen-9-ylmethyl]-carbamoyl}-5-(3-methyl-butylcarbamoyl)-piperidine-1-carboxylic acid tert-butyl ester

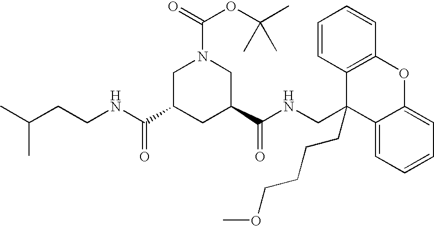

The titled compound is synthesized by condensation of (3S*,5S*)-5-{[9-(4-Methoxy-butyl)-9H-xanthen-9-ylmethyl]-carbamoyl}-piperidine-1,3-dicarboxylic acid 1-tert-butyl ester (152.8 mg, 0.28 mmol) and 3-Methyl-butylamine (32 μL, 0.28 mmol) analogously to the preparation of “General procedure, scheme 8-iii)”. white amorphous material. ES-MS: M+H=622; HPLC (condition-A): t_{R}=4.39 min.

### (3S*,5S*)-3-isobutylcarbamoyl-5-{[9-(4-methoxy-butyl)-9H-xanthen-9-ylmethyl]-carbamoyl}-piperidine-1-carboxylic acid tert-butyl ester

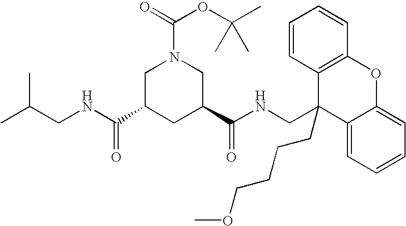

The titled compound is synthesized by condensation of (3S*,5S*)-5-{[9-(4-Methoxy-butyl)-9H-xanthen-9-ylmethyl]-carbamoyl}piperidine-1,3-dicarboxylic acid 1-tert-butyl ester (107.1 mg, 0.19 mmol) and isobutylamine (24.7 μL, 0.25 mmol) analogously to the preparation of “General procedure, scheme 8-iii)”. white amorphous material. ES-MS: M+H=608; HPLC (Condition-A): t_{R}=4.17 min.

### (3S*,5S*)-3-{[9-(4-Methoxy-butyl)-9H-xanthen-9-ylmethyl]-carbamoyl}-5-phenethylcarbamoyl-piperidine-1-carboxylic acid tert-butyl ester

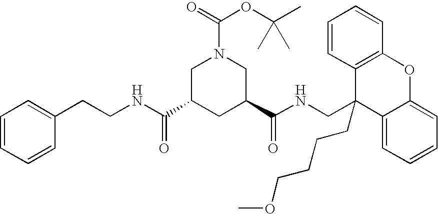

The titled compound is synthesized by condensation of (3S*,5S*)-5-{[9-(4-Methoxy-butyl)-9H-xanthen-9-ylmethyl]-carbamoyl}-piperidine-1,3-dicarboxylic acid 1-tert-butyl ester (166.9 mg, 0.30 mmol) and Phenethylamine (44.6 μL, 0.36 mmol) analogously to the preparation of “General procedure, scheme 8-iii)” white amorphous material. ES-MS: M+H=656; HPLC (Condition-A): t_{R}=4.32 min.

### (3S*,5S*)-3-(3-Hydroxy-propylcarbamoyl)-5-{[9-(4-methoxy-butyl)-9H-xanthen-9-ylmethyl]-carbamoyl}-piperidine-1-carboxylic acid tert-butyl ester

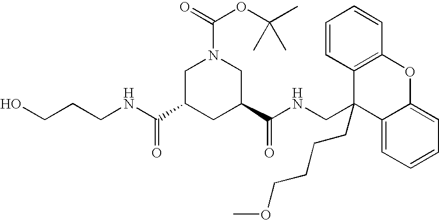

The titled compound is synthesized by condensation of (3S*,5S*)-5-{[9-(4-Methoxy-butyl)-9H-xanthen-9-ylmethyl]-carbamoyl}-piperidine-1,3-dicarboxylic acid 1-tert-butyl ester (109-3 mg, 0.20 mmol) and 3-Amino-propan-1-ol (15.1 μL, 0.20 mmol) analogously to the preparation of “General procedure, scheme 8-iii)”. white amorphous material. ES-MS: M+H=610; HPLC (Condition-A): t_{R}=3.37 min.

### Examples 132 to 135

The following Example enlisted in Table 5 are synthesized by deprotection of Boc group analogously to the Example 8, as hereinafter or hereinbefore described. As far as not being commercially available by synthesis analogous to methods or as described hereinbefore. The Asterisk (*) indicates the end of the bond at which the respective moiety is bound to the rest of the molecule falling under the following formula:

TABLE 5

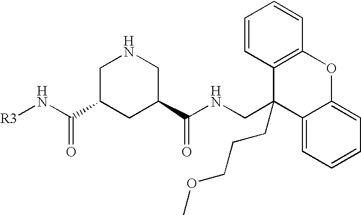

| | | tᵣₑₜ | MS |
|---|---|---|---|
| No. | R3 | (Condition) | (M + H) |
| 132 | 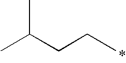 | 30.9 (A) | 522 |
| 133 | 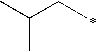 | 2.92 (B) | 508 |
| 134 | 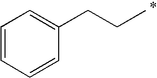 | 3.07 (A) | 556 |
| 135 | 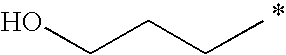 | 2.43 (A) | 510 |

### Example 136

### (3S,5R)-5-(Toluene-4-sulfonylamino)-piperidine-3-carboxylic acid [9-(4-methoxy-butyl)-9H-xanthen-9-ylmethyl]-amide

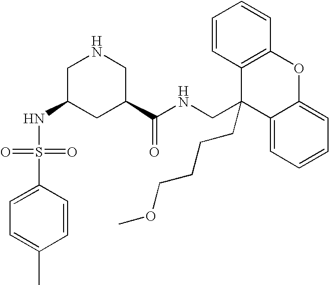

(Note that here the formula immediately above represents the pure enantiomer, not only one of the two possible enantiomers as in the other examples above if not mentioned otherwise.)

The enantiomerically pure title compound is prepared as described under “General Procedure, Scheme 2” using enantiopure (3S,5R)-5-tert-butoxycarbonylamino-piperidine-1,3-dicarboxylic acid 1-(9H-fluoren-9-ylmethyl) ester, C-[9-(4-methoxy-butyl)-9H-xanthen-9-yl]-methylamine and 4-toluenesulfonyl chloride. MS: [M+H]⁺ 587; TLC, Rf CH₂Cl₂/MeOH/NH3 50/6/1)=0.57.

The starting material is prepared as follows:

### A) (3S,5R)-5-tert-Butoxycarbonylamino-piperidine-1,3-dicarboxylic acid 1-(9H-fluoren-9-ylmethyl) ester

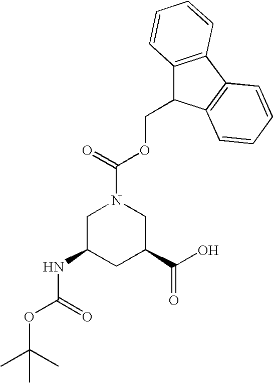

The two enantiomers of racemic (3S*,5R*)-5-tert-butoxycarbonylamino-piperidine-1,3-dicarboxylic acid 1-(9H-fluoren-9-ylmethyl) ester are separated by chiral, preparative HPLC (HPLC, Chiralcel OJ, 10 um, n-hexane/ethanol 4:1+0.1% TFA) and afford the title compound ((3S,5R)-enantiomer) as a white powder: t_{R} (HPLC, Chiralcel OJ, 10 um, 250-4.6 mm, (Nr 1064), n-hexane/ethanol 4:1+0.1% TFA, flow 1 ml/min) 8.67 min (peak 1); MS: [M+H]⁺ 465.3. The other enantiomer (3R,5S)-5-tert-butoxycarbonylamino-piperidine-1,3-dicarboxylic acid 1-(9H-fluoren-9-ylmethyl) ester is also isolated as a white powder t_{R} (HPLC, Chiralcel OJ, 10 um, 250-4.6 mm, (Nr 1064), n-hexane/ethanol 4:1+0.1% TFA, flow 1 ml/min) 19.8 min (peak 2); MS: [M+H]⁺ 465.3.

### Example 137

### Soft Capsules

5000 soft gelatin capsules, each comprising as active ingredient 0.05 g of any one of the compounds of formula I mentioned in any one of the preceding Examples, are prepared as follows:

1. Composition

| | | |
|---|---|---|
| Active ingredient | 250 | g |
| Lauroglycol | 2 | liters |

Preparation process: The pulverized active ingredient is suspended in Lauroglykol® (propylene glycol laurate, Gattefossé S.A., Saint Priest, France) and ground in a wet pulverizer to produce a particle size of about 1 to 3 μm. 0.419 g portions of the mixture are then introduced into soft gelatin capsules using a capsule-filling machine.

### Example 138

### Tablets Comprising Compounds of the Formula I

Tablets, comprising, as active ingredient, 100 mg of any one of the compounds of formula I in any one of the preceding Examples are prepared with the following composition, following standard procedures:

Composition

| | |
|---|---|
| Active Ingredient | 100 mg |
| crystalline lactose | 240 mg |
| Avicel | 80 mg |
| PVPPXL | 20 mg |
| Aerosil | 2 mg |
| magnesium stearate | 5 mg |
| | 447 mg |

Manufacture: The active ingredient is mixed with the carrier materials and compressed by means of a tabletting machine (Korsch EKO, stamp diameter 10 mm).

Avicel® is microcrystalline cellulose (FMC, Philadelphia, USA). PVPPXL is polyvinylpolypyrrolidone, cross-linked (BASF, Germany). Aerosil® is silicon dioxide (Degussa, Germany).

What is claimed is:

1. A compound of the formula I, 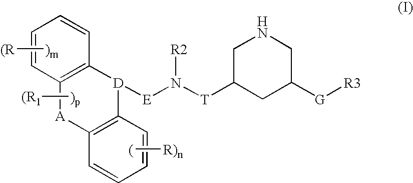
wherein
each R₁, independently of the others, (present if p>0) is a substituent selected from the group consisting of
a substituent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below;
C₂-C₇-alkenyl, C₂-C₇-alkynyl, phenyl, naphthyl, heterocyclyl, phenyl- or naphthyl- or heterocyclyl-C₁-C₇-alkyl or —C₁-C₇-alkyloxy, di-(naphthyl- or phenyl)-amino-C₁-C₇-alkyl, di(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties; phenyl- or naphthyl-C₁-C₇-alkylsulfonyl amino-C₁-C₇-alkyl, carboxy-C₁-C₇-alkyl, halo, hydroxy, phenyl-C₁-C₇-alkoxy wherein phenyl is unsubstituted or substituted by C₁-C₇-alkoxy and/or halo, halo-C₁-C₇-alkoxy, phenyl- or naphthyloxy, phenyl- or naphthyl-C₁-C₇-alkyloxy, phenyl- or naphthyloxy-C₁-C₇-alkyloxy, benzoyl- or naphthoyloxy, halo-C₁-C₇-alkylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfonylamino, carboxyl, (N,N-) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N-) di-(C₁-C₇-alkyl)amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇-alkyl)-aminocarbonyl, cyano, sulfenyl, sulfinyl, C₁-C₇-alkylsulfinyl, phenyl- or naphthylsulfinyl wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)aminosulfonyl;
R2 is hydrogen, unsubstituted or substituted alkyl, unsubstituted or substituted alkenyl, unsubstituted or substituted alkynyl, unsubstituted or substituted aryl, unsubstituted or substituted heterocyclyl, unsubstituted or substituted cycloalkyl, or acyl;
R3 is hydrogen, unsubstituted or substituted alkyl, substituted or unsubstituted aryl, unsubstituted or substituted heterocyclyl, unsubstituted or substituted cycloalkyl, unsubstituted or substituted aryl-alkyl, unsubstituted or substituted heterocyclo-alkyl, unsubstituted or substituted cycloalkyl-alkyl, or, if G is oxy, thio or unsubstituted or substituted imino, has one of the meanings just mentioned or is acyl;
R is (if more than one R is present, independently of each other) selected from C₁-C₇-alkyl, halo-C₁-C₇-alkyl, halo, hydroxy, C₁-C₇-alkoxy, phenoxy, phenyl-C₁-C₇-alkyloxy, C₁-C₇-alkanoyloxy, amino, N-mono- or N,N-di-(C₁-C₇-alkyl, alkanoyl, benzoyl, phenyl and/or phenyl-C₁-C₇-alkyl)-amino, carboxy, C₁-C₇-alkyloxycarbonyl, phenoxycarbonyl, phenyl-C₁-C₇-alkyloxycarbonyl, carbamoyl, N-mono- or N,N-di-(C₁-C₇-alkyl, phenyl and/or phenyl-C₁-C₇-alkyl)carbamoyl, sulfamoyl, N-mono- or N,N-di-(C₁-C₇-alkyl, phenyl and/or phenyl-C₁-C₇-alkyl)sulfamoyl, nitro and cyano; where, if p is zero, at least one R can instead alternatively be a moiety selected from those mentioned for R₁ as defined above;
A is NH, CH₂, S(O)₀₋₂, O, CH═CH, CH₂CH₂, CH₂O, CH₂S(O)₀₋₂, CH₂NH, C(═O)NH or SO₂NH, where in each case H is unreplaced or one or two can be replaced by a moiety R₁ as defined above if p is 1 or 2;
D is N, CH, CH═C, CH₂CH, CHO, CHS(O)₀₋₂, CH₂N, NHCH, C(═O)N or SO₂N, where in each case a H if present is unreplaced or one can be replaced by a moiety R₁ as defined above if p is 1;
E is carbonyl or unsubstituted or (halo, hydroxy, C₁-C₇-alkyloxy, phenoxy, phenyl-C₁-C₇-alkyloxy, C₁-C₇-alkanoyloxy or benzoyloxy)-substituted C₁-C₇-alkylene;
T is carbonyl or methylene;
G is a oxy, thio or unsubstituted or substituted (NR4) imino, C(═O)NH or C(═O)NR4, wherein R4 is an imino substituent;
or G-R3 together is hydrogen;
m is 0 (zero) to 4; n is 0 (zero) to 4; and p is 0 (zero) or 1;
or a salt thereof.

2. A compound of the formula I according to claim 1, where the general expressions given in claim 1 have the following meanings:
halo or halogen is fluoro, chloro, bromo or iodo, most preferably fluoro, chloro or bromo;
a substituent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O—, —NV—CO—; —CO—NV—; —NV—SO₂—, —SO₂—NV; —NV—CO—NV—, —NV—CO—O—, —O—CO—NV—, —NV—SO₂—NV— wherein V is hydrogen or unsubstituted or substituted alkyl as defined below, especially selected from C₁-C₇-alkyl, phenyl, naphthyl, phenyl- or naphthyl-C₁-C₇-alkyl and halo-C₁-C₇-alkyl; is preferably C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, C₁-C₇-alkyloxycarbonyl-C₁-C₇-alkyl, amino-C₁-C₇-alkyl, such as aminomethyl, (N-) mono- or (N,N-) di-(C₁-C₇-alkyl)-amino-C₁-C₇-alkyl, C₁-C₇-alkoxy-C₁-C₇-alkylamino-C₁-C₇-alkyl, mono-(naphthyl- or phenyl)-amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoylamino-C₁-C₇-alkyloxy, carboxy-C₁-C₇-alkyloxy, C₁-C₇-alkyloxycarbonyl-C₁-C₇-alkoxy, mono- or di-(C₁-C₇-alkyl)-aminocarbonyl-C₁-C₇-alkyloxy, C₁-C₇-alkanoyloxy, mono- or di-(C₁-C₇-alkyl)-amino, mono-di(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N-) mono- or (N,N-) di-(C₁-C₇-alkyl)-amino-C₁-C₇-alkylcarbonyl, C₁-C₇-alkanoylamino-C₁-C₇-alkylcarbonyl, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N-) mono-(C₁-C₇-alkyl)-amino-C₁-C₇-alkoxycarbonyl, C₁-C₇-alkanoylamino-C₁-C₇-alkoxycarbonyl, N-mono- or N,N-di-(C₁-C₇-alkyl)-aminocarbonyl, N—C₁-C₇-alkoxy-C₁-C₇-alkylcarbamoyl or N-mono- or N,N-di-(C₁-C₇-alkyl)-aminosulfonyl;
unsubstituted or substituted alkyl is C₁-C₂₀-alkyl, more preferably C₁-C₇-alkyl, that is straight-chained or branched one or, if desired and possible, more times, and is unsubstituted or substituted by one or more, e.g. up to three moieties independently selected from unsubstituted or substituted aryl as described below, especially phenyl or naphthyl each of which is unsubstituted or substituted as described below for unsubstituted or substituted aryl, unsubstituted or substituted heterocyclyl as described below, especially pyrrolyl, furanyl, thienyl, pyrazolyl, triazolyl, tetrazolyl, oxetidinyl, 3-(C₁-C₇-alkyl)-oxetidinyl, pyridyl, pyrimidinyl, morpholino, thiomorpholino, piperidinyl, piperazinyl, pyrrolidinyl, tetrahydrofuran-onyl, tetrahydropyranyl, indolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl and benzo[1,2,5]oxadiazolyl each of which is unsubstituted or substituted as described below for unsubstituted or substituted heterocyclyl, unsubstituted or substituted cycloalkyl as described below, especially cyclopropyl, cyclobutyl, cyclopentyl or cyclohexyl, each of which is unsubstituted or substituted as described below for unsubstituted or substituted cycloalkyl, halo, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkylthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di(C₁-C₇-alkyl and/or C₁-C₇-alkoxy-C₁-C₇alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl-, naphthyl- and/or phenyl-C₁-C₇-alkyl)-aminocarbonyl, cyano, C₁-C₇-alkenylene or alkynylene, C₁-C₇-alkylenedioxy, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfonyl, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl, naphthyl, phenyl-C₁-C₇-alkyl or naphthyl-C₁-C₇-alkyl)-aminosulfonyl;
unsubstituted or substituted alkenyl preferably has 2 to 20 carbon atoms and includes one or more double bonds, and is more preferably C₂-C₇-alkenyl that is unsubstituted or substituted as described above for unsubstituted or substituted alkyl, where preferred are vinyl or allyl;
unsubstituted or substituted alkynyl preferably has 2 to 20 carbon atoms and includes one or more triple bonds, and is more preferably C₂-C₇-alkynyl that is unsubstituted or substituted as described above for unsubstituted or substituted alkyl, where prop-2-ynyl is preferred;
unsubstituted or substituted aryl is a mono- or polycyclic, especially monocyclic, bicyclic or tricyclic aryl moiety with 6 to 22 carbon atoms, especially phenyl, naphthyl, indenyl, fluorenyl, acenapthylenyl, phenylenyl or phenanthryl, and is unsubstituted or substituted by one or more, especially one to three, moieties, preferably independently selected from the group consisting of
a substituent of the formula —(C₀-C₇-alkylene)-(K)ₚ—(C₁-C₇-alkylene)-(L)_{q}-(C₀-C₇-alkylene)-H where C₀-alkylene means that a bond is present instead of bound alkylene, p and q, each independently of the other, are 0 or 1 and each of K and L, if present and independently of the others, is —O—, —NM-, —S—, —C(═O)—, —C(S), —O—CO—, —CO—O—, —NM-CO—; —CO—NM-; —NM-SO₂—, —SO₂—NM; —NM-CO—NM-, —NM-CO—O—, —O—CO—NM-, —NM-SO₂—NM- wherein M is hydrogen or unsubstituted or substituted alkyl as defined below; especially selected from C₁-C₇-alkyl, phenyl, naphthyl, phenyl- or naphthyl-C₁-C₇-alkyl and halo-C₁-C₇-alkyl; e.g. C₁-C₇-alkyl, such as methyl, ethyl, n-propyl, isopropyl, n-butyl, isobutyl, sec-butyl or tert-butyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, C₁-C₇-alkanoyloxy-C₁-C₇-alkyl, C₁-C₇-alkyloxycarbonyl-C₁-C₇-alkyl, amino-C₁-C₇-alkyl, such as aminomethyl, (N-) mono- or (N,N-) di-(C₁-C₇-alkyl)amino-C₁-C₇-alkyl, C₁-C₇-alkoxy-C₁-C₇-alkylamino-C₁-C₇-alkyl, mono-(naphthyl- or phenyl)amino-C₁-C₇-alkyl, mono-(naphthyl- or phenyl-C₁-C₇-alkyl)-amino-C₁-C₇-alkyl, C₁-C₇-alkanoylamino-C₁-C₇-alkyl, C₁-C₇-alkyl-O—CO—NH—C₁-C₇-alkyl, C₁-C₇-alkylsulfonylamino-C₁-C₇-alkyl, C₁-C₇-alkyl-NH—CO—NH—C₁-C₇-alkyl, C₁-C₇-alkyl-NH—SO₂—NH—C₁-C₇-alkyl, C₁-C₇-alkoxy, hydroxy-C₁-C₇-alkoxy, C₁-C₇-alkoxy-C₁-C₇-alkoxy, C₁-C₇-alkanoylamino-C₁-C₇-alkyloxy, carboxy-C₁-C₇-alkyloxy, C₁-C₇-alkyloxycarbonyl-C₁-C₇-alkoxy, mono- or di-(C₁-C₇-alkyl)-aminocarbonyl-C₁-C₇-alkyloxy, C₁-C₇-alkanoyloxy, mono- or di-(C₁-C₇-alkyl)-amino, mono-di-(naphthyl- or phenyl-C₁-C₇-alkyl)-amino, N-mono-C₁-C₇-alkoxy-C₁-C₇-alkylamino, C₁-C₇-alkanoylamino, C₁-C₇-alkylsulfonylamino, C₁-C₇-alkyl-carbonyl, halo-C₁-C₇-alkylcarbonyl, hydroxy-C₁-C₇-alkylcarbonyl, C₁-C₇-alkoxy-C₁-C₇-alkylcarbonyl, amino-C₁-C₇-alkylcarbonyl, (N-) mono- or (N,N-) di-(C₁-C₇-alkyl)-amino-C₁-C₇-alkylcarbonyl, C₁-C₇-alkanoylamino-C₁-C₇-alkylcarbonyl, C₁-C₇-alkoxy-carbonyl, hydroxy-C₁-C₇-alkoxycarbonyl, C₁-C₇-alkoxy-C₁-C₇-alkoxycarbonyl, amino-C₁-C₇-alkoxycarbonyl, (N-) mono-(C₁-C₇-alkyl)amino-C₁-C₇-alkoxycarbonyl, C₁-C₇-alkanoylamino-C₁-C₇-alkoxycarbonyl, N-mono- or N,N-di(C₁-C₇-alkyl)-aminocarbonyl, N—C₁-C₇-alkoxy-C₁₋₇-alkylcarbamoyl or N-mono- or N,N-di-(C₁-C₇-alkyl)-aminosulfonyl;
from C₂-C₇-alkenyl, C₂-C₇-alkynyl, phenyl, naphthyl, heterocyclyl, especially as defined below for heterocyclyl, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, 3-C₁-C₇-alkyl-oxetidinyl, pyridyl, pyrimidinyl, morpholino, piperidinyl, piperazinyl, pyrrolidinyl, tetrahydrofuran-onyl, tetrahydropyranyl, indolyl, indazolyl, 1H-indazolyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, benzo[1,2,5]oxadiazolyl or 2H,3H-1,4-benzodioxinyl, phenyl- or naphthyl- or heterocyclyl-C₁-C₇-alkyl or —C₁-C₇-alkyloxy wherein heterocyclyl is as defined below, preferably selected from pyrrolyl, furanyl, thienyl, pyrimidinyl, pyrazolyl, pyrazolidinonyl, N—(C₁-C₇-alkyl, phenyl, naphthyl, phenyl-C₁-C₇-alkyl or naphthyl-C₁-C₇-alkyl)-pyrazolidinonyl, triazolyl, tetrazolyl, oxetidinyl, pyridyl, pyrimidinyl, morpholino, piperidinyl, piperazinyl, tetrahydrofuran-onyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl- or benzo[1,2,5]oxadiazolyl; such as benzyl or naphthylmethyl, halo-C₁-C₇-alkyl, such as trifluoromethyl, phenyloxy- or naphthyloxy-C₁-C₇-alkyl, phenyl-C₁-C₇-alkoxy- or naphthyl-C₁-C₇-alkoxy-C₁-C₇-alkyl, di-(naphthyl- or phenyl)-amino-C₁-C₇-alkyl, di-(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, carboxy-C₁-C₇-alkyl, halo, especially fluoro or chloro, hydroxy, phenyl-C₁-C₇-alkoxy wherein phenyl is unsubstituted or substituted by C₁-C₇-alkoxy and/or halo, halo-C₁-C₇-alkoxy, such as trifluoromethoxy, phenyl- or naphthyloxy, phenyl- or naphthyl-C₁-C₇-alkyloxy, phenyl- or naphthyl-oxy-C₁-C₇-alkyloxy, benzoyl- or naphthoyloxy, halo-C₁-C₇-alkylthio, such as trifluoromethylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)amino, benzoyl- or naphthoylamino, phenyl- or naphthylsulfonylamino wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonylamino, carboxyl, (N,N-) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N-) di-(C₁-C₇-alkyl)-amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇-alkyl)-aminocarbonyl, cyano, C₁-C₇-alkylene which is unsubstituted or substituted by up to four C₁-C₇-alkyl substituents and bound to two adjacent ring atoms of the aryl moiety, C₂-C₇-alkenylene or -alkynylene which are bound to two adjacent ring atoms of the aryl moiety, sulfenyl, sulfinyl, C₁-C₇-alkylsulfinyl, phenyl- or naphthylsulfinyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more, especially one to three, C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)aminosulfonyl; where aryl is especially phenyl or naphthyl, each of which is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxycarbonyl-C₁-C₇-alkyl, halo, especially fluoro, chloro or bromo, hydroxy, C₁-C₇-alkoxy, hydroxy-C₁-C₇-alkoxy C₁-C₇-alkoxy-C₁-C₇-alkoxy, amino-C₁-C₇-alkoxy, N—C₁-C₇-alkanoylamino-C₁-C₇-alkoxy, carboxyl-C₁-C₇-alkyloxy, C₁-C₇-alkoxycarbonyl-C₁-C₇-alkyloxy, carbamoyl-C₁-C₇-alkoxy, N-mono- or N,N-di-(C₁-C₇-alkyl)-carbamoyl-C₁-C₇-alkoxy, morpholino-C₁-C₇-alkoxy, pyridyl-C₁-C₇-alkoxy, amino, C₁-C₇-alkanoylamino, C₁-C₇-alkanoyl, C₁-C₇-alkoxy-C₁-C₇-alkanoyl, carboxy, carbamoyl, N—(C₁-C₇-alkoxy-C₁-C₇-alkyl)-carbamoyl, pyrazolyl, pyrazolyl-C₁-C₇-alkoxy, 4-C₁-C₇-alkylpiperidin-1-yl, nitro and cyano;
unsubstituted or substituted heterocyclyl is preferably a mono- or polycyclic, preferably a mono- or bi- or tricyclic-, unsaturated, partially saturated or saturated ring system with preferably 3 to 22 (more preferably 3 to 14) ring atoms and with one or more, preferably one to four, heteroatoms independently selected from nitrogen, oxygen and sulfur, and is unsubstituted or substituted by one or more, e.g. up to three, substitutents preferably independently selected from the substitutents mentioned above for aryl and from oxo; where preferably, heterocyclyl which is unsubstituted or substituted as just mentioned is selected from the following moieties wherein the asterisk marks the end of the bond binding to the rest of the molecule of formula I: 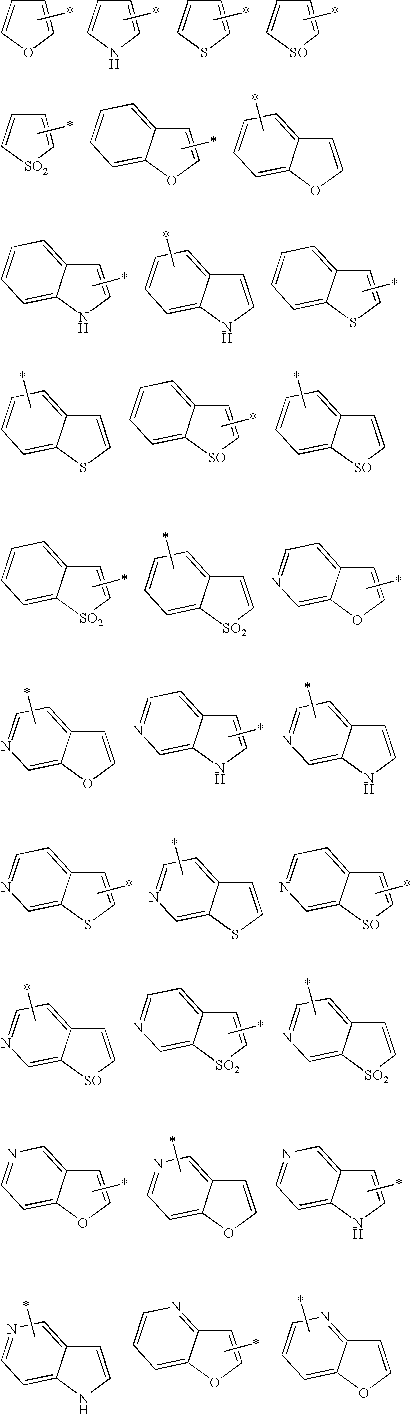 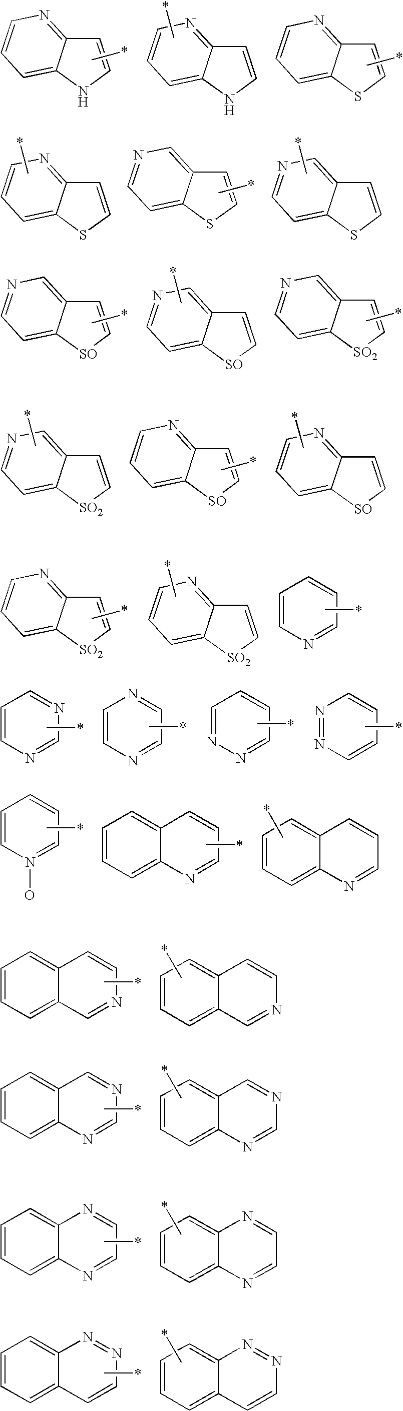 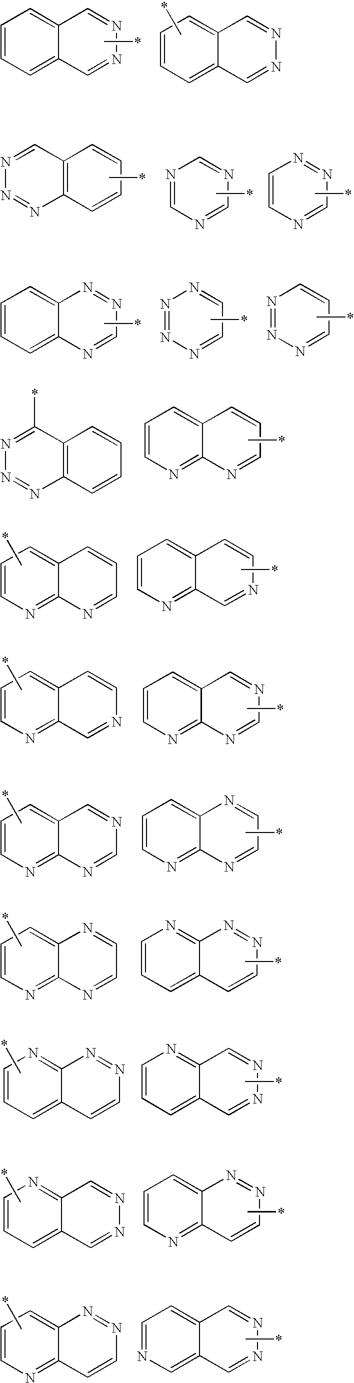 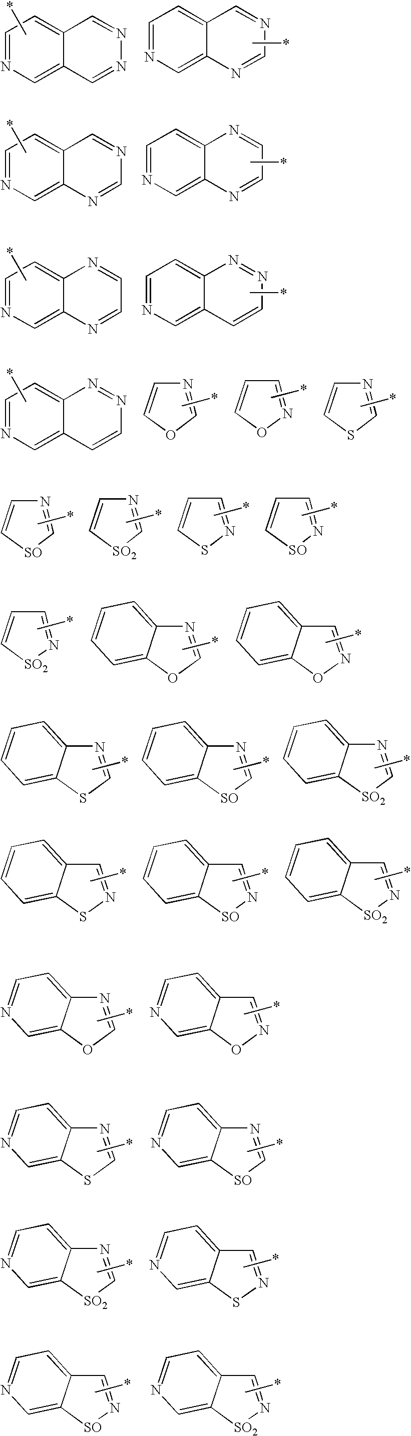 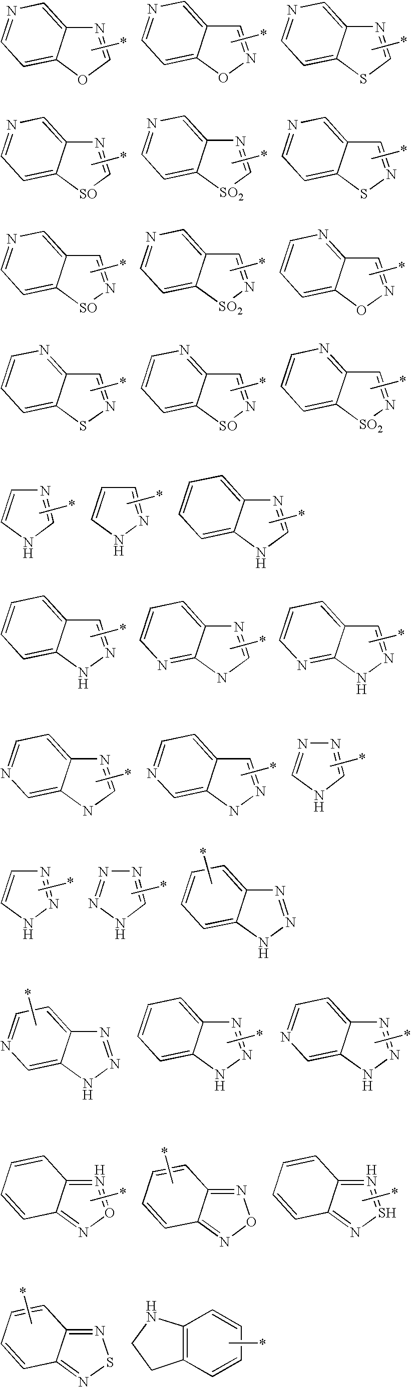 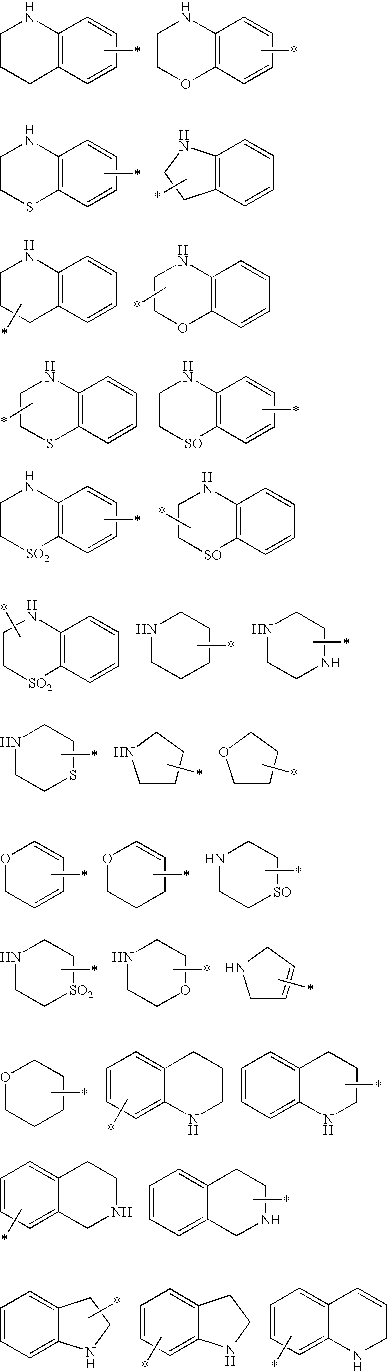 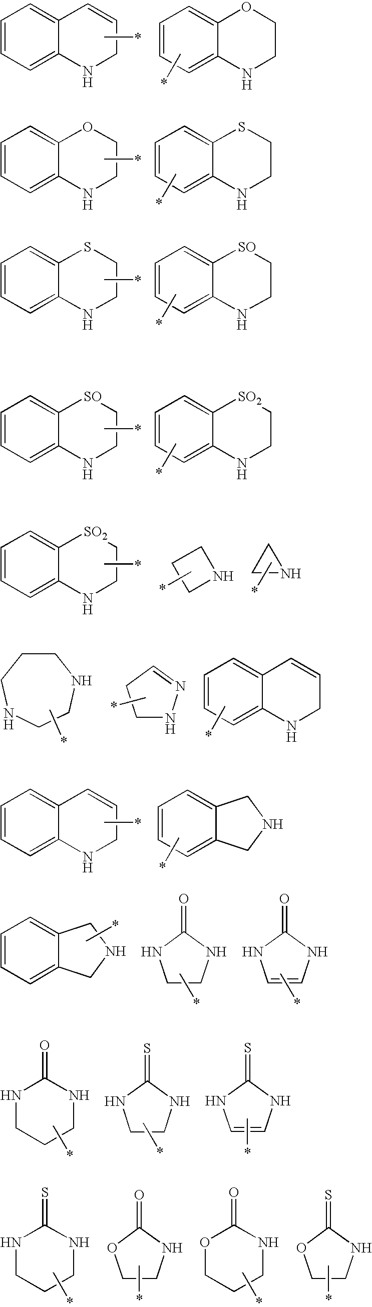 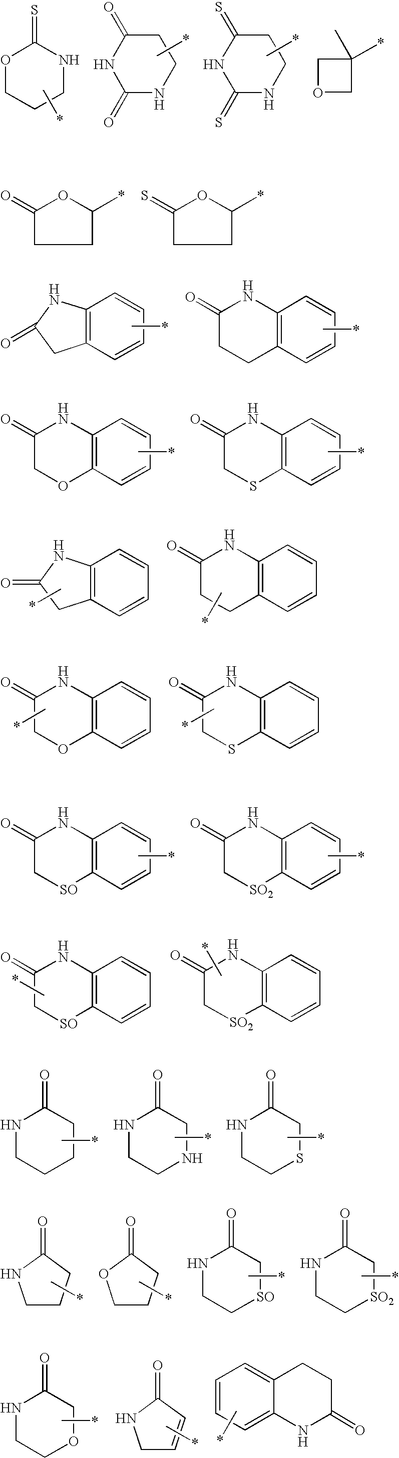 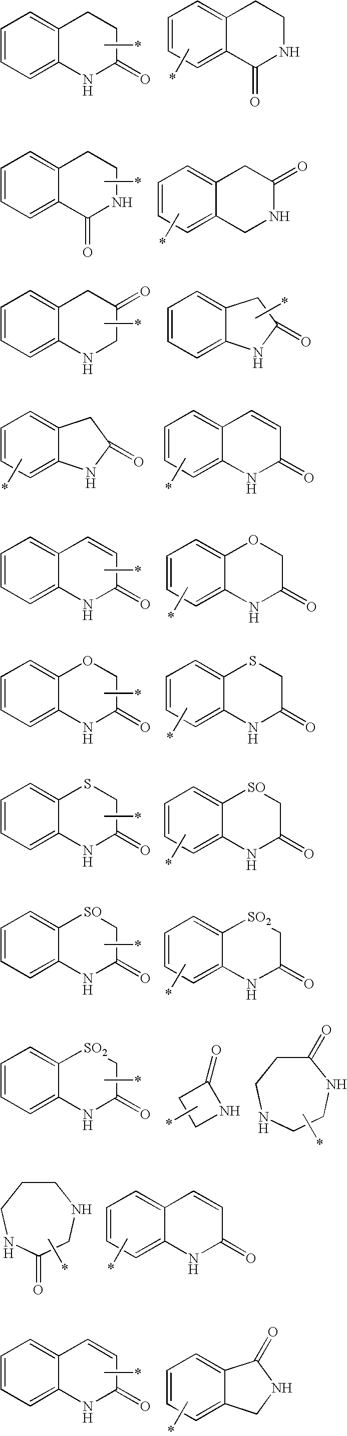 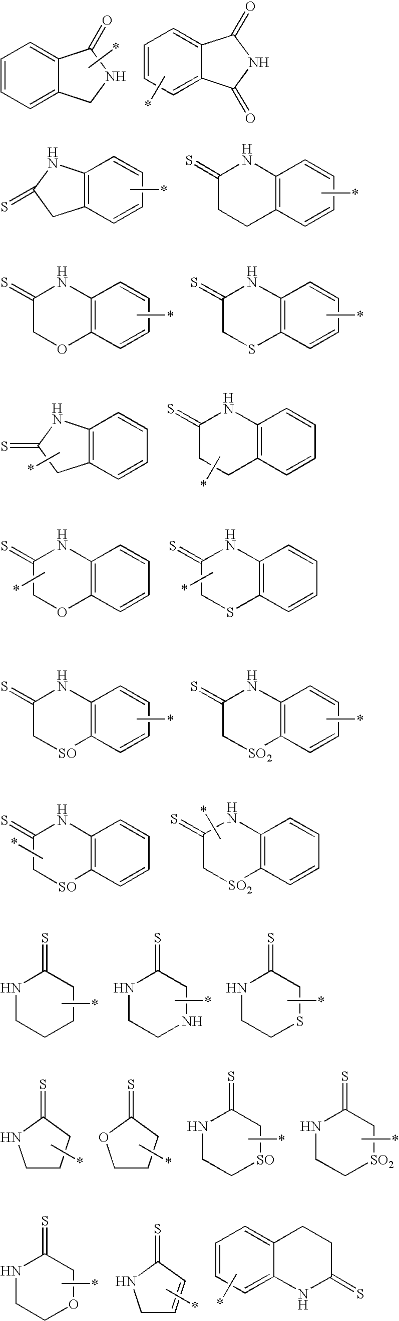 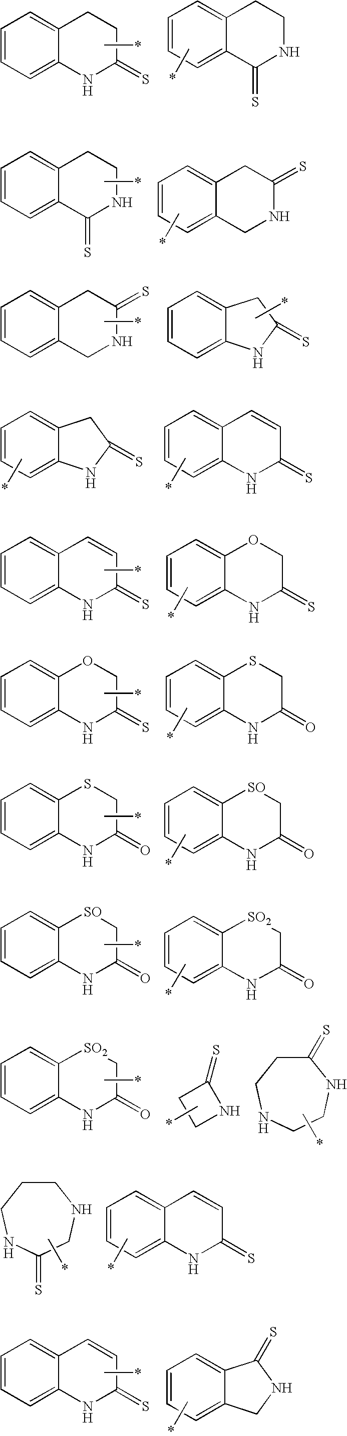 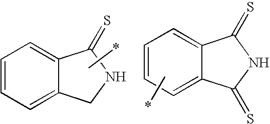
where the binding may be via a carbon or in each case where an NH is present the bond with the asterisk connecting the respective heterocyclyl moiety to the rest of the molecule the H may be replaced with said bond and/or the H may be replaced by a substituent, preferably as defined above: especially preferred as heterocyclyl is pyrrolyl, furanyl, thienyl, pyrimidinyl, pyrazolyl, pyrazolidinonyl, triazolyl, tetrazolyl, 1,3-oxazolyl, oxetidinyl, pyridyl, pyrimidinyl, morpholino, piperidinyl, piperazinyl, pyrrolidinyl, tetrahydrofuran-onyl (=oxo-tetrahydrofuranyl), tetrahydro-pyranyl, indolyl, indazolyl, 1H-indazanyl, benzofuranyl, benzothiophenyl, quinolinyl, isoquinolinyl, 1,2,3,4-tetrahydro-1,4-benzoxazinyl, 2H-1,4-benzoxazin-3(4H)-onyl, 2H,3H-1,4-benzodioxinyl, benzo[1,2,5]oxadiazolyl, thiophenyl, pyridyl, indolyl, 1H-indazolyl, quinolyl, isoquinolyl or 1-benzothiophenyl; each of which is unsubstituted or substituted by one or more, e.g. up to three, substituents as mentioned above for substituted aryl, preferably independently selected from the group consisting of C₁-C₇-alkyl, hydroxy-C₁-C₇-alkyl, C₁-C₇-alkoxy-C₁-C₇-alkyl, C₁-C₇-alkoxy-C₁-C₇-alkoxy-C₁-C₇-alkyl, amino-C₁-C₇-alkyl, C₁-C₇-alkoxy-C₁-C₇-alkylamino-C₁-C₇-alkyl, carboxy-C₁-C₇-alkyl, C₁-C₇-alkoxy-C₁-C₇-alkyl, halo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N—C₁-C₇-alkylcarbamoyl-C₁-C₇-alkoxy, C₁-C₇-alkanoyl, C₁-C₇-alkoxy-C₁-C₇-alkanoyl, carboxy, carbamoyl and N—C₁-C₇-alkoxy-C₁-C₇-alkylcarbamoyl. In the case of heterocycles including an NH ring member, the substitutents, as far as bound via a carbon or oxygen atom, can preferably be bound at the nitrogen instead of the H;
unsubstituted or substituted cycloalkyl is mono- or polycyclic, more preferably monocyclic, C₃-C₁₀-cycloalkyl which may include one or more double and/or triple bonds, and is unsubstituted or substituted by one or more, e.g. one to three substitutents preferably independently selected from those mentioned above as substituents for aryl; where cyclopropyl, cyclobutyl, cyclopentyl, cyclohexyl or cycloheptyl is especially preferred;
acyl is unsubstituted or substituted aryl-carbonyl or -sulfonyl, unsubstituted or substituted heterocyclylcarbonyl or -sulfonyl, unsubstituted or substituted cycloalkylcarbonyl or -sulfonyl, formyl or unsubstituted or substituted alkylcarbonyl or -sulfonyl, or (especially if S is oxy or preferably if it is NR4, especially imino (NH)) in the case of acyl R3 unsubstituted or substituted alkyloxycarbonyl or -oxysulfonyl, unsubstituted substituted aryl-oxycarbonyl or -oxysulfonyl, unsubstituted or substituted heterocyclyloxycarbonyl or -oxysulfonyl, unsubstituted or substituted cycloalkyloxycarbonyl or -oxysulfonyl, carbamoyl (less preferred), N-mono- or N,N-di-(unsubstituted or substituted aryl, unsubstituted or substituted heterocyclyl, unsubstituted or substituted cycloalkyl or unsubstituted or substituted alkyl)-aminocarbonyl, sulfamoyl or N-mono- or N,N-di-(unsubstituted or substituted aryl, unsubstituted or substituted heterocyclyl, unsubstituted or substituted cycloalkyl or unsubstituted or substituted alkyl)-aminosulfonyl; with the proviso that in cases of -oxycarbonyl bound moieties G is NR4, preferably NH; wherein unsubstituted or substituted aryl, unsubstituted or substituted heterocyclyl, unsubstituted or substituted cycloalkyl and unsubstituted or substituted alkyl are as described above; preferred acyl is C₁-C₇-alkanoyl, unsubstituted or mono-, di- or tri-(halo)-substituted benzoyl or naphthoyl, unsubstituted or phenyl-substituted pyrrolidinylcarbonyl, especially phenyl-pyrrolidinocarbonyl, C₁-C₇-alkylsulfonyl or (unsubstituted, halo- or C₁-C₇-alkyl-substituted) phenylsulfonyl, C₁-C₇-alkoxycarbonyl or phenyl-C₁-C₇-alkyloxycarbonyl;
alkylene is especially C₁-C₇-alkylene and can be branched or linear; preferred is methylene (CH₂), ethylene (CH₂CH₂), trimethylene (CH₂CH₂CH₂) or propylene (CH₃—CHCH₂).
in unsubstituted or substituted aryl-alkyl, unsubstituted or substituted heterocyclyl-alkyl or unsubstituted or substituted cycloalkyl-alkyl, the alkyl part is preferably C₁-C₇-alkyl, e.g. in aryl-C₁-C₇-alkyl, heterocyclyl-C₁-C₇-alkyl or cycloalkyl-C₁-C₇-alkyl; and
in substituted imino NR4, an imino substituent R4 is preferably selected from acyl, especially C₁-C₇-alkanoyl, phenylcarbonyl, C₁-C₇-alkylsulfonyl or phenylsulfonyl wherein phenyl is unsubstituted or substituted by one to 3 C₁-C₇-alkyl groups, and from one or two moieties selected from alkyl, alkenyl, alkynyl, aryl, heterocyclyl and cycloalkyl each of which is unsubstituted or substituted and is preferably as described above for the corresponding unsubstituted or substituted moieties; where C₁-C₇-alkanoylimino, mono- or di-(phenyl, naphthyl, C₁-C₇-alkoxy-phenyl, C₁-C₇-alkoxynaphthyl, naphthyl-C₁-C₇-alkyl or phenyl-C₁-C₇-alkyl)-carbonylimino, or especially mono- or di-(C₁-C₇-alkyl and/or C₁-C₇-alkoxy-C₁-C₇-alkyl)imino or mono- or di-(phenyl, naphthyl, C₁-C₇-alkoxy-phenyl, C₁-C₇-alkoxynaphthyl, phenyl-C₁-C₇-alkyl, naphthyl-C₁-C₇-alkyl, C₁-C₇-alkoxy-naphthyl-C₁-C₇-alkyl or C₁-C₇-alkoxy-phenyl-C₁-C₇-alkyl)-imino are preferred.

3. A compound of the formula I according to claim 1 wherein
R₁ if present is preferably a substituent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, —NV—, —S—, —C(═O)—, —C(═S), —O—CO—, —CO—O— —NV—CO—; —CO—NV—; —NV—SO₂—, —SO₂—NV; —NV—CO—NV—, —NV—CO—O—, —O—CO—NV—, —NV—SO₂—NV— wherein V is hydrogen, C₁-C₇-alkyl or phenyl- or naphthyl-C₁-C₇-alkyl;
or is C₂-C₇-alkenyl, C₂-C₇-alkynyl, phenyl, naphthyl, heterocyclyl, phenyl- or naphthyl- or heterocyclyl-C₁-C₇-alkyl or —C₁-C₇-alkyloxy, di-(naphthyl- or phenyl)-amino-C₁-C₇-alkyl, di(naphthyl- or phenyl-C₁-C₇-alkyl)-amino-C₁-C₇-alkyl, benzoyl- or naphthoylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties; phenyl- or naphthyl-C₁-C₇-alkylsulfonylamino-C₁-C₇-alkyl, carboxy-C₁-C₇-alkyl, halo, hydroxy, phenyl-C₁-C₇-alkoxy wherein phenyl is unsubstituted or substituted by C₁-C₇-alkoxy and/or halo, halo-C₁-C₇-alkoxy, phenyl- or naphthyloxy, phenyl- or naphthyl-C₁-C₇-alkyloxy, phenyl- or naphthyloxy-C₁-C₇-alkyloxy, benzoyl- or naphthoyloxy, halo-C₁-C₇-alkylthio, phenyl- or naphthylthio, phenyl- or naphthyl-C₁-C₇-alkylthio, benzoyl- or naphthoylthio, nitro, amino, di-(naphthyl- or phenyl-C₁-C₇-alkyl)-amino, benzoyl- or naphthoylamino, phenyl- or naphthylsulfonylamino wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfonylamino, carboxyl, (N,N) di-(C₁-C₇-alkyl)-amino-C₁-C₇-alkoxycarbonyl, halo-C₁-C₇-alkoxycarbonyl, phenyl- or naphthyloxycarbonyl, phenyl- or naphthyl-C₁-C₇-alkoxycarbonyl, (N,N-) di-(C₁-C₇-alkyl)amino-C₁-C₇-alkoxycarbonyl, carbamoyl, N-mono or N,N-di-(naphthyl-, phenyl-, C₁-C₇-alkyloxyphenyl and/or C₁-C₇-alkyloxynapthtyl-)aminocarbonyl, N-mono- or N,N-di-(naphthyl- or phenyl-C₁-C₇-alkyl)-aminocarbonyl, cyano, sulfenyl, sulfinyl, C₁-C₇-alkylsulfinyl, phenyl- or naphthylsulfinyl wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfinyl, sulfonyl, C₁-C₇-alkylsulfonyl, halo-C₁-C₇-alkylsulfonyl, hydroxy-C₁-C₇-alkylsulfonyl, C₁-C₇-alkoxy-C₁-C₇-alkylsulfonyl, amino-C₁-C₇-alkylsulfonyl, (N,N-) di-(C₁-C₇-alkyl)-amino-C₁-C₇-alkylsulfonyl, C₁-C₇-alkanoylamino-C₁-C₇-alkylsulfonyl, phenyl- or naphthylsulfonyl wherein phenyl or naphthyl is unsubstituted or substituted by one or more C₁-C₇-alkoxy-C₁-C₇-alkyl or C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl, phenyl-C₁-C₇-alkyl and/or naphthyl-C₁-C₇-alkyl)aminosulfonyl;
R2 is hydrogen, C₁-C₇-alkyl or phenyl-C₁-C₇-alkyl wherein phenyl is unsubstituted or substituted by halo;
R3 is unsubstituted or substituted aryl, especially phenyl, unsubstituted or substituted C₃-C₈-cycloalkyl-C₁-C₇-alkyl, alkyl, especially C₁-C₇-alkyl, or, if G is NH, is unsubstituted or substituted arylsulfonyl, especially (C₁-C₇-alkyl)-, halo- or (halo-C₁-C₇-alkyl)-phenylsulfonyl, or alkoxycarbonyl, especially C₁-C₇-alkyloxycarbonyl;
R is C₁-C₄-alkyl, halo-C₁-C₄-alkyl, hydroxy, C₁-C₄-alkoxy, amino, N-mono- or N,N-di-(C₁-C₄-alkyl and/or alkanoyl)-amino, carbamoyl, sulfamoyl, cyano or especially halo; or, if p is zero, one R if present can instead be R₁ as defined above;
R is or most preferably halo; or if p is zero, at least one R, preferably not more than one R, can be R₁ as defined above;
A is O, CH₂ or CH₂CH₂, where in each case an H is unreplaced or one H can be replaced by a moiety Rx selected from C₁-C₄-alkyl, hydroxy-C₁-C₄-alkyl, C₁-C₄-alkoxy-C₁-C₄-alkyl, hydroxy, halo, C₁-C₄-alkoxy, halo-C₁-C₄-alkyl, amino, N-mono- or N,N-di-(C₁-C₄-alkyl)-amino, C₁-C₄-alkoxycarbonyl, C₃-C₇-cycloalkyl or C₃-C₇-cycloalkyl-C₁-C₄-alkyl;
D is N, CH, CH═C or NHCH, where in each case an H is unreplaced or can be replaced by a moiety X, as defined above if p is 1;
E is carbonyl or unsubstituted or (hydroxy or C₁-C₇-alkoxy)-substituted C₁-C₇-alkylene;
T is carbonyl or methylene;
G is imino (NH) or C(═O)NH or C(═O)NR4 wherein R4 is C₁-C₇-alkyl or phenyl-C₁-C₇-alkyl;
or G-R3 together is hydrogen;
m is 0 or 1;
n is 0 or 1;
and p is 0 or 1;
or a pharmaceutically acceptable salt thereof.

4. A compound of the formula I according to claim 1, wherein
R₁ if present (present if p 1) is C₁-C₇-alkyl, C₁-C₇-alkoxy-C₁-C₇-alkyl or phenyl-C₁-C₇-alkyl; where if present R₁ is preferably bound as shown in formula I* above;
R2 is hydrogen or C₁-C₇-alkyl;
R3 is C₃-C₈-cycloalkyl-C₁-C₇-alkyl, especially cyclohexylmethyl, C₁-C₇-alkyl, especially methyl, or, if G is NH, is (C₁-C₇-alkyl)-, halo- or (halo-C₁-C₇-alkyl)-phenylsulfonyl or C₁-C₇-alkoxycarbonyl;
R is halo, especially chloro;
A is O, CH₂ or CH₂CH₂;
D is N, CH, CH═C or NHCH, where in each case an H is unreplaced or can be replaced by a moiety R₁ as defined above if p is 1;
E is carbonyl or unsubstituted or (hydroxy or C₁-C₇-alkoxy)-substituted C₁-C₇-alkylene;
T is carbonyl or methylene;
G is imino (NH) or C(═O)NH;
m is 0 or 1;
n is 0 or 1;
and p is 0 or 1;
or a pharmaceutically acceptable salt thereof.

5. A compound of the formula I according to claim 1 wherein
each R₁, independently of the others, (present if p>0) is a substituent selected from the group consisting of
a substituent of the formula —(C₀-C₇-alkylene)-(X)ᵣ—(C₁-C₇-alkylene)-(Y)ₛ—(C₀-C₇-alkylene)-H where C₀-alkylene means that a bond is present instead of bound alkylene, r and s, each independently of the other, are 0 or 1 and each of X and Y, if present and independently of the others, is —O—, NV—, —CO—NV— wherein V is hydrogen or unsubstituted or substituted alkyl as defined below; or
phenyl- or naphthyl- or heterocyclyl-C₁-C₇-alkyl;
R2 is hydrogen or unsubstituted or substituted alkyl;
R3 is unsubstituted or substituted alkyl, unsubstituted or substituted heterocyclyl, unsubstituted or substituted aryl-alkyl, unsubstituted or substituted heterocyclyl-alkyl, unsubstituted or substituted cycloalkyl alkyl or, if G is unsubstituted or substituted imino, has one of the meanings just mentioned or is acyl;
A is CH₂, O, CH═CH, or CH₂CH₂, where in each case H is unreplaced or one or two can be replaced by a moiety R₁ as defined above if p is 1;
D is N, CH, or NHCH, where in each case a H if present is unreplaced or one can be replaced by a moiety R₁ as defined above if p is 1;
E is unsubstituted or (halo, hydroxy, C₁-C₇-alkyloxy, phenoxy, phenyl-C₁-C₇-alkyloxy, C₁-C₇-alkanoyloxy or benzoyloxy)-substituted C₁-C₇-alkylene;
T is carbonyl or methylene;
G is oxy, unsubstituted or substituted (NR4) imino, C(═O)NH or C(═O)NR4, wherein R4 is an imino substituent;
or G-R3 together is hydrogen;
m is 0; n is 0; and p is 0 (zero) or 1;
or a salt thereof.

6. A compound of the formula I according to claim 1 wherein R3 is one of the following:
an acyl group as set forth below in embodiments (a) to (g):
(a) unsubstituted or substituted aryl sulfonyl;
(b) unsubstituted or substituted heterocyclyl sulfonyl;
(c) unsubstituted or substituted alkyl sulfonyl,
(d) unsubstituted or substituted cycloalkyl sulfonyl;
(e) unsubstituted or substituted alkyl carbonyl;
(f) unsubstituted or substituted alkyloxycarbonyl;
(g) unsubstituted or substituted heterocyclyloxycarbonyl;
unsubstituted or substituted alkyl, unsubstituted or substituted cycloalkyl alkyl, unsubstituted or substituted aryl alkyl, unsubstituted or substituted heterocyclyl alkyl, or unsubstituted or substituted heterocyclyl.

7. A compound of the formula I according to claim 1, selected from the group of compounds with the following names:
(3S*,5R*)-5-(toluene-4-sulfonylamino)-piperidine-3-carboxylic acid (9H-xanthen-9-ylmethyl)amide;
(3S*,5R*)-5-(toluene-4-sulfonylamino)-piperidine-3-carboxylic acid (9-methyl-9H-xanthen-9-ylmethyl)-amide;
(3S*,5R*)-5-(3-chloro-benzenesulfonylamino)-piperidine-3-carboxylic acid [9-(3-methoxy-propyl)-9H-xanthen-9-ylmethyl]-amide;
(3S*,5R*)-5-(toluene-4-sulfonylamino)-piperidine-3-carboxylic acid [9-(4-methoxy-butyl)-9H-xanthen-9-ylmethyl]-amide;
{(3R*,5S*)-5-[(9H-xanthen-9-ylmethyl)-carbamoyl]-piperidin-3-yl}-carbamic acid tert-butyl ester;
{(3R*,5S*)-5-[(9-phenethyl-9H-xanthen-9-ylmethyl)-carbamoyl]-piperidin-3-yl}-carbamic acid tert-butyl ester;
piperidine-3-carboxylic acid (9-phenethyl-9H-xanthen-9-ylmethyl)-amide;
piperidine-3-carboxylic acid [9-(4-methoxy-butyl)-9H-xanthen-9-ylmethyl]-amide;
(9-phenethyl-9H-xanthen-9-ylmethyl)-piperidin-3-ylmethyl-amine;
(3S*,5R*)-5-(toluene-4-sulfonylamino)-piperidine-3-carboxylic acid (10-methyl-5H-dibenzo[a,d]cyclohepten-5-ylmethyl)-amide;
(3S*,5R*)-5-(toluene-4-sulfonylamino)-piperidine-3-carboxylic acid [2-(3-chloro-10,11-dihydro-dibenzo[b,f]azepin-5-yl)-ethyl]-amide;
(3S*,5R*)-5-(toluene-4-sulfonylamino)-piperidine-3-carboxylic acid (10,11-dihydro-5H-dibenzo[a,d]cyclohepten-5-ylmethyl)-amide;
(3S*,5R*)-5-(toluene-4-sulfonylamino)-piperidine-3-carboxylic acid [3-(10,11-dihydro-dibenzo[b,f]azepin-5-yl)-2-hydroxy-propyl]-methyl-amide;
(3S*,5R*)-5-(toluene-4-sulfonylamino)-piperidine-3-carboxylic acid (6,11-dihydro-5H-dibenzo[b,e]azepin-6-ylmethyl)-methyl-amide;
(3S*,5R*)-piperidine-3,5-dicarboxylic acid 3-methylamide 5-[(9H-xanthen-9-ylmethyl)-amide];
(3S*,5R*)-piperidine-3,5-dicarboxylic acid 3-cyclohexylmethyl-amide 5-[(9H-xanthen-9-ylmethyl)-amide];
(3S*,5R*)-piperidine-3,5-dicarboxylic acid 3-{[9-(4-methoxy-butyl)-9H-xanthen-9-ylmethyl]-amide}5-methylamide;
(3S*,5R*)-piperidine-3,5-dicarboxylic acid 3-cyclohexylmethyl-amide 5-{[9-(4-methoxy-butyl)-9H-xanthen-9-ylmethyl]-amide}; and
(3S,5R)-5-(toluene-4-sulfonylamino)-piperidine-3-carboxylic acid [9-(4-methoxy-butyl)-9H-xanthen-9-ylmethyl]-amide
or a pharmaceutically acceptable salt thereof.

8. A compound of the formula I according to claim 1 with the following configuration 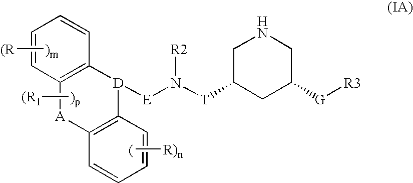
wherein R₁, R2, R3, R, A, D, E, T, G, m, n and p are as defined for a compound of the formula I in any one of claims 1 to 4 or as deducible from the names of the compounds of the formula I named in claim 7, or a pharmaceutically acceptable salt thereof.

9.-12. (canceled)

13. A pharmaceutical formulation, comprising a compound of the formula I, or a pharmaceutically acceptable salt thereof, according to claim 1 and at least one pharmaceutically acceptable carrier material.

14. A method of treatment a disease that depends on activity of renin, comprising administering to a warm-blooded animal, especially a human, in need of such treatment a pharmaceutically effective amount of a compound of the formula I, or a pharmaceutically acceptable salt thereof, according to claim 1.

15. A process for the manufacture of a compound of the formula I, or a pharmaceutically acceptable salt thereof, according to claim 1, comprising
(A) reacting a carbonic acid of the formula II, 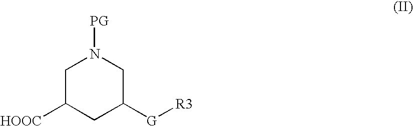
or a reactive derivative thereof, wherein R3 and G are as defined for a compound of the formula I and PG is a protecting group with an amine of the formula III, 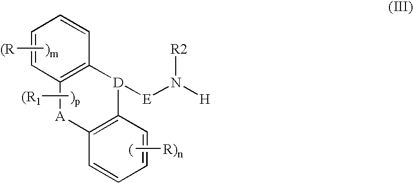
wherein R₁, R2, R, A, D, E, n, m and p are as defined for a compound of the formula I; or
(B) for the synthesis of a compound of the formula I wherein T is methylene and R₁, R2, R3, R, A, D, A, G, m, n and p have the meanings given above or below for a compound of the formula I, reacting an aldehyde of the formula IV, 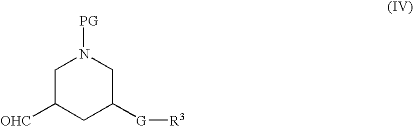
wherein R3 and G are as defined for a compound of the formula I and PG is a protecting group, especially tert-butoxycarbonyl or 9H-fluoren-9-ylmethoxycarbonyl, with an amino compound of the formula III as defined above under conditions for reductive amination; or
(C) for the synthesis of a compound of the formula I wherein G is imino, oxo or thio, reacting a compound of the formula V, 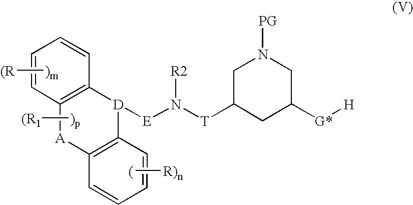
wherein R₁, R2, R, A, DA F, T, n, m and p are as defined for a compound of the formula I, G* is imino, oxy or thio and PG is a protecting group with a compound of the formula VI,
R3-LG (VI)
wherein R3 is as defined for a compound of the formula I and LG is a leaving group, or
(D) reacting a compound of the formula VII, 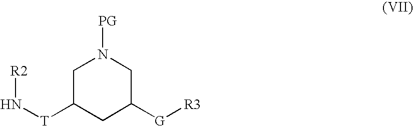
wherein R2, R3, G and T are as defined for a compound of the formula I and PG is a protecting group, with a compound of the formula VIII, 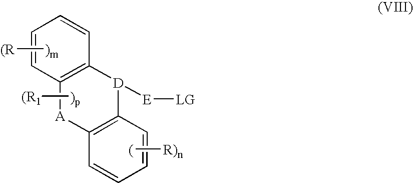
wherein R₁, R, A, D, E, m, n and p are as defined for a compound of the formula I and LG is a leaving group;
or
(E) for the synthesis of a compound of the formula I wherein G is C(═O)NR4 or C(═O)NH and T is carboxy, reacting a compound of the formula IX, 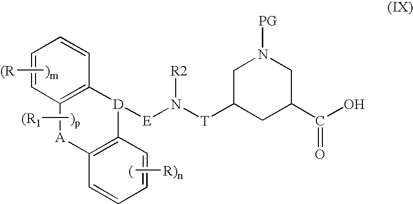
wherein R₁, R2, R, A, D, E, T, m, n and p are as defined for a compound of the formula I, with an amine of the formula X, 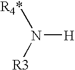
wherein R₄* is hydrogen or R4 as defined for a compound of the formula I and R3 is as defined for a compound of the formula I;
and, if desired, subsequent to any one or more of the process variants mentioned above converting an obtainable compound of the formula I or a protected form thereof into a different compound of the formula I, converting a salt of an obtainable compound of formula I into the free compound or a different salt, converting an obtainable free compound of formula I into a salt thereof, and/or separating an obtainable mixture of isomers of a compound of formula I into individual isomers;
where in any of the starting materials (especially of the formulae II to IV), in addition to specific protecting groups mentioned, further protecting groups may be present, and any protecting groups are removed at an appropriate stage in order to obtain a corresponding compound of the formula I, or a salt thereof.
